(19) United States
(12) Patent Application Publication
(10) Pub. No.: US 2022/0127612 A1
(43) Pub. Date: Apr. 28, 2022

(54) METHODS AND COMPOSITIONS FOR MODULATING SPLICING AND TRANSLATION

(71) Applicant: Stoke Therapeutics, Inc., Bedford, MA (US)

(72) Inventors: Isabel Aznarez, Boston, MA (US); Juergen Scharner, Arlington, MA (US)

(21) Appl. No.: 17/518,209

(22) Filed: Nov. 3, 2021

Related U.S. Application Data
(63) Continuation of application No. PCT/US2020/029897, filed on Apr. 24, 2020.
(60) Provisional application No. 62/838,010, filed on Apr. 24, 2019.

(51) Int. Cl. C12N 15/113; A61P 25/00

(52) U.S. Cl. CPC C12N 15/113; C12N 2310/11; C12N 2320/33; A61P 25/00

(57) ABSTRACT

Alternative splicing events in genes can lead to non-productive or less productive mRNA transcripts, and therapeutic agents which can target the alternative splicing events in genes can modulate the expression level of functional proteins in patients and/or inhibit aberrant protein expression. Provided herein are compositions and methods for modulating expression level of a target peptide sequence by modulating splicing of a pre-mRNA. Also provided herein are compositions and methods for treating a disease or condition caused by a deficient amount or activity of a functional target protein by modulating splicing of a pre-mRNA.

### CROSS-REFERENCE

This application is a continuation of International Application No. PCT/US2020/029897, filed Apr. 24, 2020, which claims the benefit of U.S. Provisional Application No. 62/838,010, filed Apr. 24, 2019, each of which is incorporated herein by reference in its entirety.

### REFERENCE TO SEQUENCE LISTING

The instant application contains a Sequence Listing which has been submitted electronically in ASCII format and is hereby incorporated by reference in its entirety. Said ASCII copy, created on Nov. 3, 2021, is named 47991_725_301_SL.txt and is 4,882,078 bytes in size.

### BACKGROUND

Alternative splicing events in genes can lead to non-productive or less productive mRNA transcripts. Therapeutic agents that can target the alternative splicing events in genes can modulate the expression level of functional proteins in patients and/or inhibit aberrant protein expression. Such therapeutic agents can be used to treat a condition or disease caused by protein deficiency.

### SUMMARY

Disclosed herein, in some aspects, is a method of modulating expression of a target peptide sequence by a cell having a pre-mRNA that encodes the target peptide sequence and comprises an inefficient translation region, the method comprising contacting the cell with a therapeutic agent that binds to a targeted region of the pre-mRNA encoding the target peptide sequence, whereby splicing of the inefficient translation region from the pre-mRNA encoding the target peptide sequence is modulated, thereby modulating a level of a first processed mRNA that is devoid of the inefficient translation region and encodes a first protein comprising the target peptide sequence, and thereby modulating the expression of the target peptide sequence in the cell, wherein the first processed mRNA has a higher translation efficiency for producing the first protein comprising the target peptide sequence in the cell as compared to translation efficiency of a second processed mRNA for producing a second protein comprising the target peptide sequence, and wherein the second processed mRNA comprises the inefficient translation region.

Disclosed herein, in some aspects, is a method of treating a disease or a condition in a subject in need thereof by modulating expression of a target peptide sequence in a cell of the subject, the cell having a pre-mRNA that encodes the target peptide sequence and comprises an inefficient translation region, the method comprising: contacting the cell of the subject with a therapeutic agent that modulates splicing of the inefficient translation region from the pre-mRNA encoding the target peptide sequence, wherein the therapeutic agent binds to a targeted region of the pre-mRNA, whereby splicing of the inefficient translation region from the pre-mRNA is modulated, thereby modulating a level of a first processed mRNA that is devoid of the inefficient translation region and encodes a first protein comprising the target peptide sequence, and thereby modulating expression of the target peptide sequence in the cell of the subject, wherein the first processed mRNA has a higher translation efficiency for producing the first protein comprising the target peptide sequence in the cell as compared to translation efficiency of a second processed mRNA for producing a second protein comprising the target peptide sequence, and wherein the second processed mRNA comprises the inefficient translation region.

In some cases, the second processed mRNA is otherwise identical to the first processed mRNA but comprises the inefficient translation region. In some cases, the pre-mRNA encoding the target peptide sequence comprises a first start codon, a second start codon, and a premature termination codon (PTC) located downstream of the first start codon and upstream of the second start codon, and wherein the inefficient translation region comprises the premature termination codon (PTC) and the second start codon. In some cases, the inefficient translation region comprises a region that encodes a proline-rich peptide sequence.

Disclosed herein, in some aspects, is a method of modulating expression of a target peptide sequence by a cell having a pre-mRNA that encodes the target peptide sequence and comprises a first start codon, a second start codon, and a premature termination codon (PTC) located downstream of the first start codon and upstream of the second start codon, the method comprising contacting the cell with a therapeutic agent that binds to a targeted portion of the pre-mRNA encoding the target peptide sequence, whereby splicing of the PTC and the second start codon from the pre-mRNA is modulated, thereby modulating a level of a first processed mRNA that is devoid of the PTC and the second start codon and encodes a first protein comprising the target peptide sequence, and thereby modulating the expression of the target peptide sequence in the cell.

Disclosed herein, in some aspects, is a method of treating a disease or a condition in a subject in need thereof by modulating expression of a target peptide sequence in a cell of the subject, the cell having a pre-mRNA that encodes the target peptide sequence and comprises a first start codon, a second start codon, and a premature termination codon (PTC) located downstream of the first start codon and upstream of the second start codon, the method comprising: contacting the cell of the subject with a therapeutic agent that modulates splicing of the PTC and the second start codon from the pre-mRNA encoding the target peptide sequence, wherein the therapeutic agent binds to a targeted region of the pre-mRNA, whereby splicing of the PTC and the second start codon from the pre-mRNA is modulated, thereby modulating a level of first processed mRNA that is devoid of the PTC and the second start codon and encodes a first protein comprising the target peptide sequence, and thereby modulating the expression of the target peptide sequence in the cell of the subject.

In some cases, the target peptide sequence has at least 10, 20, 30, 40, 50, 60, 70, 80, 90, 100, 200, 300, 400, 500, 750, or 1000 amino acids. In some cases, the therapeutic agent is a small molecule. In some cases, the therapeutic agent is an antisense oligomer (ASO) complementary to the targeted region of the pre-mRNA. In some cases, the therapeutic agent is an ASO that is at least 75%, at least 80%, at least 85%, at least 90%, at least 95%, at least 98%, at least 99%, or 100%, complementary to the targeted region of the pre-mRNA encoding the target peptide sequence. In some cases, at least a portion of the targeted region of the pre-mRNA is upstream of the inefficient translation region or an exon comprising the PTC and the second start codon. In some cases, at least a portion of the targeted region of the pre-mRNA is downstream of the inefficient translation region or an exon comprising the PTC and the second start codon. In some cases, at least a portion of the targeted region of the pre-mRNA is within the inefficient translation region or an exon comprising the PTC and the second start codon. In some cases, the cell has a second processed mRNA that comprises sequence of the first processed mRNA, the PTC, and the second start codon and encodes a second protein comprising the target peptide sequence. In some cases, the first processed mRNA and the second processed mRNA are both splicing products of the pre-mRNA in the cell. In some cases, the second processed mRNA comprises sequence of the first processed mRNA, the PTC, and the second start codon. In some cases, the PTC and the second start codon have a distance of at least 2 nt on the pre-mRNA. In some cases, the PTC and the second start codon have a distance of at most 75 nt on the pre-mRNA. In some cases, the PTC and the second start codon have a distance of 2 nt to 75 nt, 5 nt to 70 nt, 10 nt to 65 nt, 15 nt to 60 nt, 20 nt to 55 nt, 25 nt to 50 nt, 30 nt to 45 nt, 40 nt to 50 nt, 45 nt to 55 nt, 50 nt to 60 nt, 55 nt to 70 nt, or 60 nt to 75 nt to on the pre-mRNA. In some cases, the PTC and the second start codon have a distance of 2 nt to 75 nt on the pre-mRNA.

In some cases, the first protein comprising the target peptide sequence is translated starting from the first start codon on the first processed mRNA, and the second protein is translated starting from the second start codon on the second processed mRNA. In some cases, the first processed mRNA has a higher translation efficiency for producing the first protein as compared to translation efficiency of the second processed mRNA for producing the second protein.

In some cases, exclusion of the inefficient translation region or exclusion of the PTC and the second start codon from the pre-mRNA encoding the target peptide sequence is increased. In some cases, the therapeutic agent increases the level of the first processed mRNA encoding the target peptide sequence in the cell. In some cases, the therapeutic agent increases the level of the target peptide sequence in the cell.

In some cases, the target peptide sequence is a portion of a full length protein. In some cases, the target peptide sequence produced is a fully functional protein. In some cases, the target peptide sequence is an N-terminal portion of a full length protein. In some cases, the target peptide sequence is coded by a portion of the pre-mRNA upstream of the inefficient translation region. In some cases, the inefficient translation region is in a 3′ untranslated region (3′ UTR) of the second processed mRNA.

In some cases, the target peptide sequence is a C-terminal portion of a full length protein. In some cases, the target peptide sequence is coded by a portion of the pre-mRNA downstream of the inefficient translation region or the PTC.

In some cases, the target peptide sequence is a portion of an MeCP2 protein isoform. In some cases, the target peptide sequence is translated from a sequence selected from the group consisting of SEQ ID NOs: 32-34. In some cases, the therapeutic agent decreases level of e2 isoform of MeCP2 mRNA and increases level of e1 isoform of MeCP2 mRNA in the cell. In some cases, the therapeutic agent decreases level of e2 isoform of MeCP2 protein and increases level of e1 isoform of MeCP2 protein in the cell. In some cases, the pre-mRNA comprises a sequence with at least about 80%, 85%, 90%, 95%, 96%, 97%, 98%, 99% or 100% sequence identity to any one selected from the group consisting of SEQ ID NOs: 2-27. In some cases, the therapeutic agent binds to a targeted region of a MeCP2 pre-mRNA, wherein the targeted region is within a sequence selected from the group consisting of SEQ ID NOs: 28-31. In some cases, the therapeutic agent binds to a targeted region of a MeCP2 pre-mRNA, wherein the targeted region is within a sequence SEQ ID NO: 28. In some cases, the therapeutic agent is an antisense oligomer complementary to a sequence with at least about 80%, 85%, 90%, 95%, 96%, 97%, 98%, 99% or 100% sequence identity to a sequence selected from the group consisting of SEQ ID NOs: 28-31. In some cases, the therapeutic agent is an antisense oligomer complementary to a sequence with 100% sequence identity to a sequence selected from the group consisting of SEQ ID NOs: 28-31. In some cases, the therapeutic agent is an antisense oligomer complementary to a sequence with 100% sequence identity to SEQ ID NO: 28. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 35-129. In some cases, the therapeutic agent is an antisense oligomer that has a sequence with 100% sequence identity to a sequence selected from the group consisting of SEQ ID NOs: 35-129. In some cases, the disease or the condition is Rett syndrome.

In some cases, the target peptide sequence is a portion of a protein selected from the group consisting of: ACIN1, ACTN1, ACTN2, ADH4, AKR1C3, ALDH1A2, ALG8, AMPD2, AMT, ANKRD11, ANKS3, APOL4, APPL1, ARCN1, ARMC8, ARNTL, BOC, BRCA1, C8B, CALM1, CALM3, CASP5, CAST, CEP19, CEP57, CHEK1, CIB2, COX4I1, DCLRE1C, DECR1, DHFR, DKK2, DLG2, DOK2, DPF3, DTNA, DYRK1A, FOLH1, FUZ, GANAB, GEMIN4, GGA3, GOSR2, GPM6A, GRB10, GSN, HDAC8, HIVEP1, HK1, IFNGR1, IKBKB, ISCU, KARS, KIAA0319, KIAA0586, KIZ, KLK6, LMNA, LMNTD1, LRRC7, LRTOMT, LZTFL1, MAGI2, MECP2, MERTK, MFSD2A, MLF1, MOB1B, MSRB3, NR1I3, NT5C3A, NUTM1, OTOF, PDCD4, PDPK1, PHKB, PIGA, PLOD2, POLR2F, PPP6C, PRKN, PRUNE1, PSMA6, PSMC3IP, PTPN1, RAB11B, RBPJ, RNPS1, RPL5, RPS20, SECISBP2, SELENBP1, SEPT11, SIRT2, SLC25A26, SMARCE1, SMC1A, SMG1, SPACA7, SPRED1, SRP54, SRPK2, STK36, STRADA, SUMO1, TBL1XR1, TLR8, TXNRD2, UBE3A, UFM1, WAC, WDR81, YME1L1, and ZMYND10. In some cases, the therapeutic agent increases level of the first processed mRNA encoding the first protein having a sequence selected from the group consisting of: SEQ ID NOs: 8096-8297, and decreases level of the second processed mRNA encoding the second protein having a sequence selected from the group consisting of: SEQ ID NOs: 332-533.

In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of a sequence selected from the group consisting of SEQ ID NOs: 130-331. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of a sequence selected from the group consisting of SEQ ID NOs: 130-331.

In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of a genomic site selected from the group consisting of: GRCh38/hg38: chr14 23071186, GRCh38/hg38: chr14 68925672, GRCh38/hg38: chr1 236735635, GRCh38/hg38: chr4 99143305, GRCh38/hg38: chr4 99143305, GRCh38/hg38: chr6 32182698, GRCh38/hg38: chr10 5077900, GRCh38/hg38: chr15 58058108, GRCh38/hg38: chr11 78138767, GRCh38/hg38: chr1 109620914, GRCh38/hg38: chr3 49422257, GRCh38/hg38: chr3 49422257, GRCh38/hg38: chr3 49422257, GRCh38/hg38: chr16 89317078, GRCh38/hg38: chr16 89317078, GRCh38/hg38: chr16 89317078, GRCh38/hg38: chr16 89317075, GRCh38/hg38: chr16 89317078, GRCh38/hg38: chr16 89317078, GRCh38/hg38: chr16 89317075, GRCh38/hg38: chr16 89317078, GRCh38/hg38: chr16 89317078, GRCh38/hg38: chr16 89317075, GRCh38/hg38: chr16 89317078, GRCh38/hg38: chr16 89317075, GRCh38/hg38: chr16 89317078, GRCh38/hg38: chr16 89317078, GRCh38/hg38: chr16 4726780, GRCh38/hg38: chr22 36201796, GRCh38/hg38: chr3 57230714, GRCh38/hg38: chr11 118573620, GRCh38/hg38: chr3 138188455, GRCh38/hg38: chr3 138188455, GRCh38/hg38: chr11 13355226, GRCh38/hg38: chr11 13355226, GRCh38/hg38: chr3 113249722, GRCh38/hg38: chr3 113249722, GRCh38/hg38: chr3 113249722, GRCh38/hg38: chr3 113249722, GRCh38/hg38: chr17 43106533, GRCh38/hg38: chr17 43106533, GRCh38/hg38: chr1 56959657, GRCh38/hg38: chr1 56959657, GRCh38/hg38: chr14 90398991, GRCh38/hg38: chr19 46608205, GRCh38/hg38: chr11 105009053, GRCh38/hg38: chr5 96726754, GRCh38/hg38: chr3 196708727, GRCh38/hg38: chr11 95795516, GRCh38/hg38: chr11 125627607, GRCh38/hg38: chr15 78111276, GRCh38/hg38: chr15 78111276, GRCh38/hg38: chr16 85804941, GRCh38/hg38: chr10 14939869, GRCh38/hg38: chr8 90005292, GRCh38/hg38: chr8 90015541, GRCh38/hg38: chr5 80649494, GRCh38/hg38: chr4 106925949, GRCh38/hg38: chr11 83651930, GRCh38/hg38: chr8 21910857, GRCh38/hg38: chr14 72879947, GRCh38/hg38: chr14 72879947, GRCh38/hg38: chr18 34757158, GRCh38/hg38: chr18 34757158, GRCh38/hg38: chr21 37456130, GRCh38/hg38: chr11 49206874, GRCh38/hg38: chr19 49812335, GRCh38/hg38: chr11 62639110, GRCh38/hg38: chr17 749909, GRCh38/hg38: chr17 75243570, GRCh38/hg38: chr17 46924261, GRCh38/hg38: chr4 175812249, GRCh38/hg38: chr7 50710990, GRCh38/hg38: chr9 121300056, GRCh38/hg38: chrX 72572109, GRCh38/hg38: chr6 12020248, GRCh38/hg38: chr10 69300769, GRCh38/hg38: chr10 69300769, GRCh38/hg38: chr6 137215377, GRCh38/hg38: chr8 42272083, GRCh38/hg38: chr8 42272083, GRCh38/hg38: chr8 42272083, GRCh38/hg38: chr8 42272083, GRCh38/hg38: chr12 108564060, GRCh38/hg38: chr16 75644462, GRCh38/hg38: chr16 75644462, GRCh38/hg38: chr6 24600709, GRCh38/hg38: chr14 58429363, GRCh38/hg38: chr14 58429363, GRCh38/hg38: chr20 21132097, GRCh38/hg38: chr19 50963549, GRCh38/hg38: chr1 156126736, GRCh38/hg38: chr12 25552989, GRCh38/hg38: chr12 25552989, GRCh38/hg38: chr1 69716142, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr3 45838051, GRCh38/hg38: chr7 78554671, GRCh38/hg38: chrX 154092307, GRCh38/hg38: chrX 154092307, GRCh38/hg38: chr2 111944960, GRCh38/hg38: chr1 39965211, GRCh38/hg38: chr3 158592434, GRCh38/hg38: chr3 158592434, GRCh38/hg38: chr3 158592434, GRCh38/hg38: chr3 158592434, GRCh38/hg38: chr3 158592434, GRCh38/hg38: chr4 70950700, GRCh38/hg38: chr12 65308529, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr7 33035988, GRCh38/hg38: chr7 33035988, GRCh38/hg38: chr15 34345857, GRCh38/hg38: chr2 26477749, GRCh38/hg38: chr2 26477749, GRCh38/hg38: chr10 110876670, GRCh38/hg38: chr16 2566356, GRCh38/hg38: chr16 47463899, GRCh38/hg38: chr16 47463882, GRCh38/hg38: chrX 15331992, GRCh38/hg38: chrX 15331992, GRCh38/hg38: chr3 146124229, GRCh38/hg38: chr22 37958374, GRCh38/hg38: chr9 125172001, GRCh38/hg38: chr6 162262765, GRCh38/hg38: chr1 151027233, GRCh38/hg38: chr14 35308914, GRCh38/hg38: chr17 42573623, GRCh38/hg38: chr20 50564969, GRCh38/hg38: chr19 8402086, GRCh38/hg38: chr4 26320700, GRCh38/hg38: chr4 26320700, GRCh38/hg38: chr16 2264760, GRCh38/hg38: chr16 2264760, GRCh38/hg38: chr16 2264760, GRCh38/hg38: chr16 2264760, GRCh38/hg38: chr1 92833545, GRCh38/hg38: chr8 56073768, GRCh38/hg38: chr9 89325427, GRCh38/hg38: chr1 151369978, GRCh38/hg38: chr1 151369978, GRCh38/hg38: chr4 76995781, GRCh38/hg38: chr19 38893867, GRCh38/hg38: chr19 38893867, GRCh38/hg38: chr19 38893867, GRCh38/hg38: chr19 38893867, GRCh38/hg38: chr19 38893867, GRCh38/hg38: chr3 66243203, GRCh38/hg38: chr3 66243203, GRCh38/hg38: chr17 40644459, GRCh38/hg38: chrX 53422040, GRCh38/hg38: chr16 18900044, GRCh38/hg38: chr13 112382278, GRCh38/hg38: chr15 38299373, GRCh38/hg38: chr14 35000936, GRCh38/hg38: chr7 105268869, GRCh38/hg38: chr2 218673665, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr2 202214429, GRCh38/hg38: chr3 177065022, GRCh38/hg38: chr3 177065022, GRCh38/hg38: chr3 177065022, GRCh38/hg38: chr3 177065022, GRCh38/hg38: chrX 12910306, GRCh38/hg38: chr22 19880945, GRCh38/hg38: chr15 25408684, GRCh38/hg38: chr15 25408684, GRCh38/hg38: chr13 38349999, GRCh38/hg38: chr10 28535562, GRCh38/hg38: chr17 1727990, GRCh38/hg38: chr10 27153281, and GRCh38/hg38: chr3 50345232.

In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of a genomic site selected from the group consisting of: GRCh38/hg38: chr14 23071186, GRCh38/hg38: chr14 68925672, GRCh38/hg38: chr1 236735635, GRCh38/hg38: chr4 99143305, GRCh38/hg38: chr4 99143305, GRCh38/hg38: chr6 32182698, GRCh38/hg38: chr10 5077900, GRCh38/hg38: chr15 58058108, GRCh38/hg38: chr11 78138767, GRCh38/hg38: chr1 109620914, GRCh38/hg38: chr3 49422257, GRCh38/hg38: chr3 49422257, GRCh38/hg38: chr3 49422257, GRCh38/hg38: chr16 89317078, GRCh38/hg38: chr16 89317078, GRCh38/hg38: chr16 89317078, GRCh38/hg38: chr16 89317075, GRCh38/hg38: chr16 89317078, GRCh38/hg38: chr16 89317078, GRCh38/hg38: chr16 89317075, GRCh38/hg38: chr16 89317078, GRCh38/hg38: chr16 89317078, GRCh38/hg38: chr16 89317075, GRCh38/hg38: chr16 89317078, GRCh38/hg38: chr16 89317075, GRCh38/hg38: chr16 89317078, GRCh38/hg38: chr16 89317078, GRCh38/hg38: chr16 4726780, GRCh38/hg38: chr22 36201796, GRCh38/hg38: chr3 57230714, GRCh38/hg38: chr11 118573620, GRCh38/hg38: chr3 138188455, GRCh38/hg38: chr3 138188455, GRCh38/hg38: chr11 13355226, GRCh38/hg38: chr11 13355226, GRCh38/hg38: chr3 113249722, GRCh38/hg38: chr3 113249722, GRCh38/hg38: chr3 113249722, GRCh38/hg38: chr3 113249722, GRCh38/hg38: chr17 43106533, GRCh38/hg38: chr17 43106533, GRCh38/hg38: chr1 56959657, GRCh38/hg38: chr1 56959657, GRCh38/hg38: chr14 90398991, GRCh38/hg38: chr19 46608205, GRCh38/hg38: chr11 105009053, GRCh38/hg38: chr5 96726754, GRCh38/hg38: chr3 196708727, GRCh38/hg38: chr11 95795516, GRCh38/hg38: chr11 125627607, GRCh38/hg38: chr15 78111276, GRCh38/hg38: chr15 78111276, GRCh38/hg38: chr16 85804941, GRCh38/hg38: chr10 14939869, GRCh38/hg38: chr8 90005292, GRCh38/hg38: chr8 90015541, GRCh38/hg38: chr5 80649494, GRCh38/hg38: chr4 106925949, GRCh38/hg38: chr11 83651930, GRCh38/hg38: chr8 21910857, GRCh38/hg38: chr14 72879947, GRCh38/hg38: chr14 72879947, GRCh38/hg38: chr18 34757158, GRCh38/hg38: chr18 34757158, GRCh38/hg38: chr21 37456130, GRCh38/hg38: chr11 49206874, GRCh38/hg38: chr19 49812335, GRCh38/hg38: chr11 62639110, GRCh38/hg38: chr17 749909, GRCh38/hg38: chr17 75243570, GRCh38/hg38: chr17 46924261, GRCh38/hg38: chr4 175812249, GRCh38/hg38: chr7 50710990, GRCh38/hg38: chr9 121300056, GRCh38/hg38: chrX 72572109, GRCh38/hg38: chr6 12020248, GRCh38/hg38: chr10 69300769, GRCh38/hg38: chr10 69300769, GRCh38/hg38: chr6 137215377, GRCh38/hg38: chr8 42272083, GRCh38/hg38: chr8 42272083, GRCh38/hg38: chr8 42272083, GRCh38/hg38: chr8 42272083, GRCh38/hg38: chr12 108564060, GRCh38/hg38: chr16 75644462, GRCh38/hg38: chr16 75644462, GRCh38/hg38: chr6 24600709, GRCh38/hg38: chr14 58429363, GRCh38/hg38: chr14 58429363, GRCh38/hg38: chr20 21132097, GRCh38/hg38: chr19 50963549, GRCh38/hg38: chr1 156126736, GRCh38/hg38: chr12 25552989, GRCh38/hg38: chr12 25552989, GRCh38/hg38: chr1 69716142, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr11 72089029, GRCh38/hg38: chr3 45838051, GRCh38/hg38: chr7 78554671, GRCh38/hg38: chrX 154092307, GRCh38/hg38: chrX 154092307, GRCh38/hg38: chr2 111944960, GRCh38/hg38: chr1 39965211, GRCh38/hg38: chr3 158592434, GRCh38/hg38: chr3 158592434, GRCh38/hg38: chr3 158592434, GRCh38/hg38: chr3 158592434, GRCh38/hg38: chr3 158592434, GRCh38/hg38: chr4 70950700, GRCh38/hg38: chr12 65308529, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr1 161236598, GRCh38/hg38: chr7 33035988, GRCh38/hg38: chr7 33035988, GRCh38/hg38: chr15 34345857, GRCh38/hg38: chr2 26477749, GRCh38/hg38: chr2 26477749, GRCh38/hg38: chr10 110876670, GRCh38/hg38: chr16 2566356, GRCh38/hg38: chr16 47463899, GRCh38/hg38: chr16 47463882, GRCh38/hg38: chrX 15331992, GRCh38/hg38: chrX 15331992, GRCh38/hg38: chr3 146124229, GRCh38/hg38: chr22 37958374, GRCh38/hg38: chr9 125172001, GRCh38/hg38: chr6 162262765, GRCh38/hg38: chr1 151027233, GRCh38/hg38: chr14 35308914, GRCh38/hg38: chr17 42573623, GRCh38/hg38: chr20 50564969, GRCh38/hg38: chr19 8402086, GRCh38/hg38: chr4 26320700, GRCh38/hg38: chr4 26320700, GRCh38/hg38: chr16 2264760, GRCh38/hg38: chr16 2264760, GRCh38/hg38: chr16 2264760, GRCh38/hg38: chr16 2264760, GRCh38/hg38: chr1 92833545, GRCh38/hg38: chr8 56073768, GRCh38/hg38: chr9 89325427, GRCh38/hg38: chr1 151369978, GRCh38/hg38: chr1 151369978, GRCh38/hg38: chr4 76995781, GRCh38/hg38: chr19 38893867, GRCh38/hg38: chr19 38893867, GRCh38/hg38: chr19 38893867, GRCh38/hg38: chr19 38893867, GRCh38/hg38: chr19 38893867, GRCh38/hg38: chr3 66243203, GRCh38/hg38: chr3 66243203, GRCh38/hg38: chr17 40644459, GRCh38/hg38: chrX 53422040, GRCh38/hg38: chr16 18900044, GRCh38/hg38: chr13 112382278, GRCh38/hg38: chr15 38299373, GRCh38/hg38: chr14 35000936, GRCh38/hg38: chr7 105268869, GRCh38/hg38: chr2 218673665, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr17 63714108, GRCh38/hg38: chr2 202214429, GRCh38/hg38: chr3 177065022, GRCh38/hg38: chr3 177065022, GRCh38/hg38: chr3 177065022, GRCh38/hg38: chr3 177065022, GRCh38/hg38: chrX 12910306, GRCh38/hg38: chr22 19880945, GRCh38/hg38: chr15 25408684, GRCh38/hg38: chr15 25408684, GRCh38/hg38: chr13 38349999, GRCh38/hg38: chr10 28535562, GRCh38/hg38: chr17 1727990, GRCh38/hg38: chr10 27153281, and GRCh38/hg38: chr3 50345232.

In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of a sequence selected from the group consisting of: SEQ ID NOs: 130-331. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of a sequence selected from the group consisting of: SEQ ID NOs: 130-331.

In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of a genomic site selected from the group consisting of: GRCh38/hg38: chr14 23071125, GRCh38/hg38: chr14 68925558, GRCh38/hg38: chr1 236735720, GRCh38/hg38: chr4 99143166, GRCh38/hg38: chr4 99143166, GRCh38/hg38: chr6 32182568, GRCh38/hg38: chr10 5077977, GRCh38/hg38: chr15 58058012, GRCh38/hg38: chr11 78138710, GRCh38/hg38: chr1 109621266, GRCh38/hg38: chr3 49422104, GRCh38/hg38: chr3 49422104, GRCh38/hg38: chr3 49422104, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 4726659, GRCh38/hg38: chr22 36201700, GRCh38/hg38: chr3 57230767, GRCh38/hg38: chr11 118573783, GRCh38/hg38: chr3 138188530, GRCh38/hg38: chr3 138188530, GRCh38/hg38: chr11 13355296, GRCh38/hg38: chr11 13355296, GRCh38/hg38: chr3 113249899, GRCh38/hg38: chr3 113249899, GRCh38/hg38: chr3 113249899, GRCh38/hg38: chr3 113249899, GRCh38/hg38: chr17 43106456, GRCh38/hg38: chr17 43106456, GRCh38/hg38: chr1 56959529, GRCh38/hg38: chr1 56959529, GRCh38/hg38: chr14 90399087, GRCh38/hg38: chr19 46608340, GRCh38/hg38: chr11 105008807, GRCh38/hg38: chr5 96726859, GRCh38/hg38: chr3 196708528, GRCh38/hg38: chr11 95795559, GRCh38/hg38: chr11 125627830, GRCh38/hg38: chr15 78111165, GRCh38/hg38: chr15 78111165, GRCh38/hg38: chr16 85805104, GRCh38/hg38: chr10 14939810, GRCh38/hg38: chr8 90005369, GRCh38/hg38: chr8 90015666, GRCh38/hg38: chr5 80649389, GRCh38/hg38: chr4 106925799, GRCh38/hg38: chr11 83651839, GRCh38/hg38: chr8 21910673, GRCh38/hg38: chr14 72879804, GRCh38/hg38: chr14 72879804, GRCh38/hg38: chr18 34757288, GRCh38/hg38: chr18 34757288, GRCh38/hg38: chr21 37456308, GRCh38/hg38: chr11 49206778, GRCh38/hg38: chr19 49812251, GRCh38/hg38: chr11 62638983, GRCh38/hg38: chr17 749814, GRCh38/hg38: chr17 75243447, GRCh38/hg38: chr17 46924468, GRCh38/hg38: chr4 175812191, GRCh38/hg38: chr7 50710875, GRCh38/hg38: chr9 121300126, GRCh38/hg38: chrX 72572057, GRCh38/hg38: chr6 12020418, GRCh38/hg38: chr10 69300861, GRCh38/hg38: chr10 69300861, GRCh38/hg38: chr6 137215267, GRCh38/hg38: chr8 42272205, GRCh38/hg38: chr8 42272205, GRCh38/hg38: chr8 42272205, GRCh38/hg38: chr8 42272205, GRCh38/hg38: chr12 108564155, GRCh38/hg38: chr16 75644283, GRCh38/hg38: chr16 75644283, GRCh38/hg38: chr6 24600619, GRCh38/hg38: chr14 58429433, GRCh38/hg38: chr14 58429433, GRCh38/hg38: chr20 21132159, GRCh38/hg38: chr19 50963302, GRCh38/hg38: chr1 156126915, GRCh38/hg38: chr12 25552871, GRCh38/hg38: chr12 25552871, GRCh38/hg38: chr1 69716218, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr3 45837927, GRCh38/hg38: chr7 78554567, GRCh38/hg38: chrX 154092184, GRCh38/hg38: chrX 154092184, GRCh38/hg38: chr2 111945060, GRCh38/hg38: chr1 39965334, GRCh38/hg38: chr3 158592581, GRCh38/hg38: chr3 158592581, GRCh38/hg38: chr3 158592581, GRCh38/hg38: chr3 158592581, GRCh38/hg38: chr3 158592581, GRCh38/hg38: chr4 70950811, GRCh38/hg38: chr12 65308655, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr7 33035934, GRCh38/hg38: chr7 33035934, GRCh38/hg38: chr15 34346035, GRCh38/hg38: chr2 26477649, GRCh38/hg38: chr2 26477649, GRCh38/hg38: chr10 110876753, GRCh38/hg38: chr16 2566453, GRCh38/hg38: chr16 47463993, GRCh38/hg38: chr16 47463993, GRCh38/hg38: chrX 15331216, GRCh38/hg38: chrX 15331216, GRCh38/hg38: chr3 146124138, GRCh38/hg38: chr22 37958471, GRCh38/hg38: chr9 125171909, GRCh38/hg38: chr6 162262525, GRCh38/hg38: chr1 151027327, GRCh38/hg38: chr14 35308995, GRCh38/hg38: chr17 42573478, GRCh38/hg38: chr20 50565069, GRCh38/hg38: chr19 8402279, GRCh38/hg38: chr4 26320815, GRCh38/hg38: chr4 26320815, GRCh38/hg38: chr16 2264573, GRCh38/hg38: chr16 2264573, GRCh38/hg38: chr16 2264573, GRCh38/hg38: chr16 2264573, GRCh38/hg38: chr1 92833660, GRCh38/hg38: chr8 56073695, GRCh38/hg38: chr9 89325676, GRCh38/hg38: chr1 151369713, GRCh38/hg38: chr1 151369713, GRCh38/hg38: chr4 76995938, GRCh38/hg38: chr19 38893819, GRCh38/hg38: chr19 38893819, GRCh38/hg38: chr19 38893819, GRCh38/hg38: chr19 38893819, GRCh38/hg38: chr19 38893819, GRCh38/hg38: chr3 66243312, GRCh38/hg38: chr3 66243312, GRCh38/hg38: chr17 40644385, GRCh38/hg38: chrX 53421895, GRCh38/hg38: chr16 18899987, GRCh38/hg38: chr13 112382507, GRCh38/hg38: chr15 38299547, GRCh38/hg38: chr14 35001020, GRCh38/hg38: chr7 105268806, GRCh38/hg38: chr2 218673765, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr2 202214357, GRCh38/hg38: chr3 177064920, GRCh38/hg38: chr3 177064920, GRCh38/hg38: chr3 177064920, GRCh38/hg38: chr3 177064920, GRCh38/hg38: chrX 12910442, GRCh38/hg38: chr22 19880856, GRCh38/hg38: chr15 25408620, GRCh38/hg38: chr15 25408620, GRCh38/hg38: chr13 38350227, GRCh38/hg38: chr10 28535757, GRCh38/hg38: chr17 1728626, GRCh38/hg38: chr10 27153227, and GRCh38/hg38: chr3 50345124.

In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of a genomic site selected from the group consisting of: GRCh38/hg38: chr14 23071125, GRCh38/hg38: chr14 68925558, GRCh38/hg38: chr1 236735720, GRCh38/hg38: chr4 99143166, GRCh38/hg38: chr4 99143166, GRCh38/hg38: chr6 32182568, GRCh38/hg38: chr10 5077977, GRCh38/hg38: chr15 58058012, GRCh38/hg38: chr11 78138710, GRCh38/hg38: chr1 109621266, GRCh38/hg38: chr3 49422104, GRCh38/hg38: chr3 49422104, GRCh38/hg38: chr3 49422104, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 89316933, GRCh38/hg38: chr16 4726659, GRCh38/hg38: chr22 36201700, GRCh38/hg38: chr3 57230767, GRCh38/hg38: chr11 118573783, GRCh38/hg38: chr3 138188530, GRCh38/hg38: chr3 138188530, GRCh38/hg38: chr11 13355296, GRCh38/hg38: chr11 13355296, GRCh38/hg38: chr3 113249899, GRCh38/hg38: chr3 113249899, GRCh38/hg38: chr3 113249899, GRCh38/hg38: chr3 113249899, GRCh38/hg38: chr17 43106456, GRCh38/hg38: chr17 43106456, GRCh38/hg38: chr1 56959529, GRCh38/hg38: chr1 56959529, GRCh38/hg38: chr14 90399087, GRCh38/hg38: chr19 46608340, GRCh38/hg38: chr11 105008807, GRCh38/hg38: chr5 96726859, GRCh38/hg38: chr3 196708528, GRCh38/hg38: chr11 95795559, GRCh38/hg38: chr11 125627830, GRCh38/hg38: chr15 78111165, GRCh38/hg38: chr15 78111165, GRCh38/hg38: chr16 85805104, GRCh38/hg38: chr10 14939810, GRCh38/hg38: chr8 90005369, GRCh38/hg38: chr8 90015666, GRCh38/hg38: chr5 80649389, GRCh38/hg38: chr4 106925799, GRCh38/hg38: chr11 83651839, GRCh38/hg38: chr8 21910673, GRCh38/hg38: chr14 72879804, GRCh38/hg38: chr14 72879804, GRCh38/hg38: chr18 34757288, GRCh38/hg38: chr18 34757288, GRCh38/hg38: chr21 37456308, GRCh38/hg38: chr11 49206778, GRCh38/hg38: chr19 49812251, GRCh38/hg38: chr11 62638983, GRCh38/hg38: chr17 749814, GRCh38/hg38: chr17 75243447, GRCh38/hg38: chr17 46924468, GRCh38/hg38: chr4 175812191, GRCh38/hg38: chr7 50710875, GRCh38/hg38: chr9 121300126, GRCh38/hg38: chrX 72572057, GRCh38/hg38: chr6 12020418, GRCh38/hg38: chr10 69300861, GRCh38/hg38: chr10 69300861, GRCh38/hg38: chr6 137215267, GRCh38/hg38: chr8 42272205, GRCh38/hg38: chr8 42272205, GRCh38/hg38: chr8 42272205, GRCh38/hg38: chr8 42272205, GRCh38/hg38: chr12 108564155, GRCh38/hg38: chr16 75644283, GRCh38/hg38: chr16 75644283, GRCh38/hg38: chr6 24600619, GRCh38/hg38: chr14 58429433, GRCh38/hg38: chr14 58429433, GRCh38/hg38: chr20 21132159, GRCh38/hg38: chr19 50963302, GRCh38/hg38: chr1 156126915, GRCh38/hg38: chr12 25552871, GRCh38/hg38: chr12 25552871, GRCh38/hg38: chr1 69716218, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr11 72089165, GRCh38/hg38: chr3 45837927, GRCh38/hg38: chr7 78554567, GRCh38/hg38: chrX 154092184, GRCh38/hg38: chrX 154092184, GRCh38/hg38: chr2 111945060, GRCh38/hg38: chr1 39965334, GRCh38/hg38: chr3 158592581, GRCh38/hg38: chr3 158592581, GRCh38/hg38: chr3 158592581, GRCh38/hg38: chr3 158592581, GRCh38/hg38: chr3 158592581, GRCh38/hg38: chr4 70950811, GRCh38/hg38: chr12 65308655, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr1 161236459, GRCh38/hg38: chr7 33035934, GRCh38/hg38: chr7 33035934, GRCh38/hg38: chr15 34346035, GRCh38/hg38: chr2 26477649, GRCh38/hg38: chr2 26477649, GRCh38/hg38: chr10 110876753, GRCh38/hg38: chr16 2566453, GRCh38/hg38: chr16 47463993, GRCh38/hg38: chr16 47463993, GRCh38/hg38: chrX 15331216, GRCh38/hg38: chrX 15331216, GRCh38/hg38: chr3 146124138, GRCh38/hg38: chr22 37958471, GRCh38/hg38: chr9 125171909, GRCh38/hg38: chr6 162262525, GRCh38/hg38: chr1 151027327, GRCh38/hg38: chr14 35308995, GRCh38/hg38: chr17 42573478, GRCh38/hg38: chr20 50565069, GRCh38/hg38: chr19 8402279, GRCh38/hg38: chr4 26320815, GRCh38/hg38: chr4 26320815, GRCh38/hg38: chr16 2264573, GRCh38/hg38: chr16 2264573, GRCh38/hg38: chr16 2264573, GRCh38/hg38: chr16 2264573, GRCh38/hg38: chr1 92833660, GRCh38/hg38: chr8 56073695, GRCh38/hg38: chr9 89325676, GRCh38/hg38: chr1 151369713, GRCh38/hg38: chr1 151369713, GRCh38/hg38: chr4 76995938, GRCh38/hg38: chr19 38893819, GRCh38/hg38: chr19 38893819, GRCh38/hg38: chr19 38893819, GRCh38/hg38: chr19 38893819, GRCh38/hg38: chr19 38893819, GRCh38/hg38: chr3 66243312, GRCh38/hg38: chr3 66243312, GRCh38/hg38: chr17 40644385, GRCh38/hg38: chrX 53421895, GRCh38/hg38: chr16 18899987, GRCh38/hg38: chr13 112382507, GRCh38/hg38: chr15 38299547, GRCh38/hg38: chr14 35001020, GRCh38/hg38: chr7 105268806, GRCh38/hg38: chr2 218673765, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr17 63714006, GRCh38/hg38: chr2 202214357, GRCh38/hg38: chr3 177064920, GRCh38/hg38: chr3 177064920, GRCh38/hg38: chr3 177064920, GRCh38/hg38: chr3 177064920, GRCh38/hg38: chrX 12910442, GRCh38/hg38: chr22 19880856, GRCh38/hg38: chr15 25408620, GRCh38/hg38: chr15 25408620, GRCh38/hg38: chr13 38350227, GRCh38/hg38: chr10 28535757, GRCh38/hg38: chr17 1728626, GRCh38/hg38: chr10 27153227, and GRCh38/hg38: chr3 50345124.

In some cases, the targeted region of the pre-mRNA is within a sequence selected from the group consisting of: SEQ ID NOs: 130-331. In some cases, the targeted region of the pre-mRNA is within a sequence between a pair of genomic sites selected from the group consisting of: GRCh38/hg38: chr14 23071186, and GRCh38/hg38: chr14 23071125; GRCh38/hg38: chr14 68925672, and GRCh38/hg38: chr14 68925558; GRCh38/hg38: chr1 236735635, and GRCh38/hg38: chr1 236735720; GRCh38/hg38: chr4 99143305, and GRCh38/hg38: chr4 99143166; GRCh38/hg38: chr4 99143305, and GRCh38/hg38: chr4 99143166; GRCh38/hg38: chr6 32182698, and GRCh38/hg38: chr6 32182568; GRCh38/hg38: chr10 5077900, and GRCh38/hg38: chr10 5077977; GRCh38/hg38: chr15 58058108, and GRCh38/hg38: chr15 58058012; GRCh38/hg38: chr11 78138767, and GRCh38/hg38: chr11 78138710; GRCh38/hg38: chr1 109620914, and GRCh38/hg38: chr1 109621266; GRCh38/hg38: chr3 49422257, and GRCh38/hg38: chr3 49422104; GRCh38/hg38: chr3 49422257, and GRCh38/hg38: chr3 49422104; GRCh38/hg38: chr3 49422257, and GRCh38/hg38: chr3 49422104; GRCh38/hg38: chr16 89317078, and GRCh38/hg38: chr16 89316933; GRCh38/hg38: chr16 89317078, and GRCh38/hg38: chr16 89316933; GRCh38/hg38: chr16 89317078, and GRCh38/hg38: chr16 89316933; GRCh38/hg38: chr16 89317075, and GRCh38/hg38: chr16 89316933; GRCh38/hg38: chr16 89317078, and GRCh38/hg38: chr16 89316933; GRCh38/hg38: chr16 89317078, and GRCh38/hg38: chr16 89316933; GRCh38/hg38: chr16 89317075, and GRCh38/hg38: chr16 89316933; GRCh38/hg38: chr16 89317078, and GRCh38/hg38: chr16 89316933; GRCh38/hg38: chr16 89317078, and GRCh38/hg38: chr16 89316933; GRCh38/hg38: chr16 89317075, and GRCh38/hg38: chr16 89316933; GRCh38/hg38: chr16 89317078, and GRCh38/hg38: chr16 89316933; GRCh38/hg38: chr16 89317075, and GRCh38/hg38: chr16 89316933; GRCh38/hg38: chr16 89317078, and GRCh38/hg38: chr16 89316933; GRCh38/hg38: chr16 89317078, and GRCh38/hg38: chr16 89316933; GRCh38/hg38: chr16 4726780, and GRCh38/hg38: chr16 4726659; GRCh38/hg38: chr22 36201796, and GRCh38/hg38: chr22 36201700; GRCh38/hg38: chr3 57230714, and GRCh38/hg38: chr3 57230767; GRCh38/hg38: chr11 118573620, and GRCh38/hg38: chr11 118573783; GRCh38/hg38: chr3 138188455, and GRCh38/hg38: chr3 138188530; GRCh38/hg38: chr3 138188455, and GRCh38/hg38: chr3 138188530; GRCh38/hg38: chr11 13355226, and GRCh38/hg38: chr11 13355296; GRCh38/hg38: chr11 13355226, and GRCh38/hg38: chr11 13355296; GRCh38/hg38: chr3 113249722, and GRCh38/hg38: chr3 113249899; GRCh38/hg38: chr3 113249722, and GRCh38/hg38: chr3 113249899; GRCh38/hg38: chr3 113249722, and GRCh38/hg38: chr3 113249899; GRCh38/hg38: chr3 113249722, and GRCh38/hg38: chr3 113249899; GRCh38/hg38: chr17 43106533, and GRCh38/hg38: chr17 43106456; GRCh38/hg38: chr17 43106533, and GRCh38/hg38: chr17 43106456; GRCh38/hg38: chr1 56959657, and GRCh38/hg38: chr1 56959529; GRCh38/hg38: chr1 56959657, and GRCh38/hg38: chr1 56959529; GRCh38/hg38: chr14 90398991, and GRCh38/hg38: chr14 90399087; GRCh38/hg38: chr19 46608205, and GRCh38/hg38: chr19 46608340; GRCh38/hg38: chr11 105009053, and GRCh38/hg38: chr11 105008807; GRCh38/hg38: chr5 96726754, and GRCh38/hg38: chr5 96726859; GRCh38/hg38: chr3 196708727, and GRCh38/hg38: chr3 196708528; GRCh38/hg38: chr11 95795516, and GRCh38/hg38: chr11 95795559; GRCh38/hg38: chr11 125627607, and GRCh38/hg38: chr11 125627830; GRCh38/hg38: chr15 78111276, and GRCh38/hg38: chr15 78111165; GRCh38/hg38: chr15 78111276, and GRCh38/hg38: chr15 78111165; GRCh38/hg38: chr16 85804941, and GRCh38/hg38: chr16 85805104; GRCh38/hg38: chr10 14939869, and GRCh38/hg38: chr10 14939810; GRCh38/hg38: chr8 90005292, and GRCh38/hg38: chr8 90005369; GRCh38/hg38: chr8 90015541, and GRCh38/hg38: chr8 90015666; GRCh38/hg38: chr5 80649494, and GRCh38/hg38: chr5 80649389; GRCh38/hg38: chr4 106925949, and GRCh38/hg38: chr4 106925799; GRCh38/hg38: chr11 83651930, and GRCh38/hg38: chr11 83651839; GRCh38/hg38: chr8 21910857, and GRCh38/hg38: chr8 21910673; GRCh38/hg38: chr14 72879947, and GRCh38/hg38: chr14 72879804; GRCh38/hg38: chr14 72879947, and GRCh38/hg38: chr14 72879804; GRCh38/hg38: chr18 34757158, and GRCh38/hg38: chr18 34757288; GRCh38/hg38: chr18 34757158, and GRCh38/hg38: chr18 34757288; GRCh38/hg38: chr21 37456130, and GRCh38/hg38: chr21 37456308; GRCh38/hg38: chr11 49206874, and GRCh38/hg38: chr11 49206778; GRCh38/hg38: chr19 49812335, and GRCh38/hg38: chr19 49812251; GRCh38/hg38: chr11 62639110, and GRCh38/hg38: chr11 62638983; GRCh38/hg38: chr17 749909, and GRCh38/hg38: chr17 749814; GRCh38/hg38: chr17 75243570, and GRCh38/hg38: chr17 75243447; GRCh38/hg38: chr17 46924261, and GRCh38/hg38: chr17 46924468; GRCh38/hg38: chr4 175812249, and GRCh38/hg38: chr4 175812191; GRCh38/hg38: chr7 50710990, and GRCh38/hg38: chr7 50710875; GRCh38/hg38: chr9 121300056, and GRCh38/hg38: chr9 121300126; GRCh38/hg38: chrX 72572109, and GRCh38/hg38: chrX 72572057; GRCh38/hg38: chr6 12020248, and GRCh38/hg38: chr6 12020418; GRCh38/hg38: chr10 69300769, and GRCh38/hg38: chr10 69300861; GRCh38/hg38: chr10 69300769, and GRCh38/hg38: chr10 69300861; GRCh38/hg38: chr6 137215377, and GRCh38/hg38: chr6 137215267; GRCh38/hg38: chr8 42272083, and GRCh38/hg38: chr8 42272205; GRCh38/hg38: chr8 42272083, and GRCh38/hg38: chr8 42272205; GRCh38/hg38: chr8 42272083, and GRCh38/hg38: chr8 42272205; GRCh38/hg38: chr8 42272083, and GRCh38/hg38: chr8 42272205; GRCh38/hg38: chr12 108564060, and GRCh38/hg38: chr12 108564155; GRCh38/hg38: chr16 75644462, and GRCh38/hg38: chr16 75644283; GRCh38/hg38: chr16 75644462, and GRCh38/hg38: chr16 75644283; GRCh38/hg38: chr6 24600709, and GRCh38/hg38: chr6 24600619; GRCh38/hg38: chr14 58429363, and GRCh38/hg38: chr14 58429433; GRCh38/hg38: chr14 58429363, and GRCh38/hg38: chr14 58429433; GRCh38/hg38: chr20 21132097, and GRCh38/hg38: chr20 21132159; GRCh38/hg38: chr19 50963549, and GRCh38/hg38: chr19 50963302; GRCh38/hg38: chr1 156126736, and GRCh38/hg38: chr1 156126915; GRCh38/hg38: chr12 25552989, and GRCh38/hg38: chr12 25552871; GRCh38/hg38: chr12 25552989, and GRCh38/hg38: chr12 25552871; GRCh38/hg38: chr1 69716142, and GRCh38/hg38: chr1 69716218; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr11 72089029, and GRCh38/hg38: chr11 72089165; GRCh38/hg38: chr3 45838051, and GRCh38/hg38: chr3 45837927; GRCh38/hg38: chr7 78554671, and GRCh38/hg38: chr7 78554567; GRCh38/hg38: chrX 154092307, and GRCh38/hg38: chrX 154092184; GRCh38/hg38: chrX 154092307, and GRCh38/hg38: chrX 154092184; GRCh38/hg38: chr2 111944960, and GRCh38/hg38: chr2 111945060; GRCh38/hg38: chr1 39965211, and GRCh38/hg38: chr1 39965334; GRCh38/hg38: chr3 158592434, and GRCh38/hg38: chr3 158592581; GRCh38/hg38: chr3 158592434, and GRCh38/hg38: chr3 158592581; GRCh38/hg38: chr3 158592434, and GRCh38/hg38: chr3 158592581; GRCh38/hg38: chr3 158592434, and GRCh38/hg38: chr3 158592581; GRCh38/hg38: chr3 158592434, and GRCh38/hg38: chr3 158592581; GRCh38/hg38: chr4 70950700, and GRCh38/hg38: chr4 70950811; GRCh38/hg38: chr12 65308529, and GRCh38/hg38: chr12 65308655; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr1 161236598, and GRCh38/hg38: chr1 161236459; GRCh38/hg38: chr7 33035988, and GRCh38/hg38: chr7 33035934; GRCh38/hg38: chr7 33035988, and GRCh38/hg38: chr7 33035934; GRCh38/hg38: chr15 34345857, and GRCh38/hg38: chr15 34346035; GRCh38/hg38: chr2 26477749, and GRCh38/hg38: chr2 26477649; GRCh38/hg38: chr2 26477749, and GRCh38/hg38: chr2 26477649; GRCh38/hg38: chr10 110876670, and GRCh38/hg38: chr10 110876753; GRCh38/hg38: chr16 2566356, and GRCh38/hg38: chr16 2566453; GRCh38/hg38: chr16 47463899, and GRCh38/hg38: chr16 47463993; GRCh38/hg38: chr16 47463882, and GRCh38/hg38: chr16 47463993; GRCh38/hg38: chrX 15331992, and GRCh38/hg38: chrX 15331216; GRCh38/hg38: chrX 15331992, and GRCh38/hg38: chrX 15331216; GRCh38/hg38: chr3 146124229, and GRCh38/hg38: chr3 146124138; GRCh38/hg38: chr22 37958374, and GRCh38/hg38: chr22 37958471; GRCh38/hg38: chr9 125172001, and GRCh38/hg38: chr9 125171909; GRCh38/hg38: chr6 162262765, and GRCh38/hg38: chr6 162262525; GRCh38/hg38: chr1 151027233, and GRCh38/hg38: chr1 151027327; GRCh38/hg38: chr14 35308914, and GRCh38/hg38: chr14 35308995; GRCh38/hg38: chr17 42573623, and GRCh38/hg38: chr17 42573478; GRCh38/hg38: chr20 50564969, and GRCh38/hg38: chr20 50565069; GRCh38/hg38: chr19 8402086, and GRCh38/hg38: chr19 8402279; GRCh38/hg38: chr4 26320700, and GRCh38/hg38: chr4 26320815; GRCh38/hg38: chr4 26320700, and GRCh38/hg38: chr4 26320815; GRCh38/hg38: chr16 2264760, and GRCh38/hg38: chr16 2264573; GRCh38/hg38: chr16 2264760, and GRCh38/hg38: chr16 2264573; GRCh38/hg38: chr16 2264760, and GRCh38/hg38: chr16 2264573; GRCh38/hg38: chr16 2264760, and GRCh38/hg38: chr16 2264573; GRCh38/hg38: chr1 92833545, and GRCh38/hg38: chr1 92833660; GRCh38/hg38: chr8 56073768, and GRCh38/hg38: chr8 56073695; GRCh38/hg38: chr9 89325427, and GRCh38/hg38: chr9 89325676; GRCh38/hg38: chr1 151369978, and GRCh38/hg38: chr1 151369713; GRCh38/hg38: chr1 151369978, and GRCh38/hg38: chr1 151369713; GRCh38/hg38: chr4 76995781, and GRCh38/hg38: chr4 76995938; GRCh38/hg38: chr19 38893867, and GRCh38/hg38: chr19 38893819; GRCh38/hg38: chr19 38893867, and GRCh38/hg38: chr19 38893819; GRCh38/hg38: chr19 38893867, and GRCh38/hg38: chr19 38893819; GRCh38/hg38: chr19 38893867, and GRCh38/hg38: chr19 38893819; GRCh38/hg38: chr19 38893867, and GRCh38/hg38: chr19 38893819; GRCh38/hg38: chr3 66243203, and GRCh38/hg38: chr3 66243312; GRCh38/hg38: chr3 66243203, and GRCh38/hg38: chr3 66243312; GRCh38/hg38: chr17 40644459, and GRCh38/hg38: chr17 40644385; GRCh38/hg38: chrX 53422040, and GRCh38/hg38: chrX 53421895; GRCh38/hg38: chr16 18900044, and GRCh38/hg38: chr16 18899987; GRCh38/hg38: chr13 112382278, and GRCh38/hg38: chr13 112382507; GRCh38/hg38: chr15 38299373, and GRCh38/hg38: chr15 38299547; GRCh38/hg38: chr14 35000936, and GRCh38/hg38: chr14 35001020; GRCh38/hg38: chr7 105268869, and GRCh38/hg38: chr7 105268806; GRCh38/hg38: chr2 218673665, and GRCh38/hg38: chr2 218673765; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr17 63714108, and GRCh38/hg38: chr17 63714006; GRCh38/hg38: chr2 202214429, and GRCh38/hg38: chr2 202214357; GRCh38/hg38: chr3 177065022, and GRCh38/hg38: chr3 177064920; GRCh38/hg38: chr3 177065022, and GRCh38/hg38: chr3 177064920; GRCh38/hg38: chr3 177065022, and GRCh38/hg38: chr3 177064920; GRCh38/hg38: chr3 177065022, and GRCh38/hg38: chr3 177064920; GRCh38/hg38: chrX 12910306, and GRCh38/hg38: chrX 12910442; GRCh38/hg38: chr22 19880945, and GRCh38/hg38: chr22 19880856; GRCh38/hg38: chr15 25408684, and GRCh38/hg38: chr15 25408620; GRCh38/hg38: chr15 25408684, and GRCh38/hg38: chr15 25408620; GRCh38/hg38: chr13 38349999, and GRCh38/hg38: chr13 38350227; GRCh38/hg38: chr10 28535562, and GRCh38/hg38: chr10 28535757; GRCh38/hg38: chr17 1727990, and GRCh38/hg38: chr17 1728626; GRCh38/hg38: chr10 27153281, and GRCh38/hg38: chr10 27153227; GRCh38/hg38: chr3 50345232, and GRCh38/hg38: chr3 50345124.

In some cases, the therapeutic agent is an antisense oligomer complementary to a sequence with at least about 80%, 85%, 90%, 95%, 96%, 97%, 98%, 99% or 100% sequence identity to a sequence selected from the group consisting of SEQ ID NOs: 130-331. In some cases, the therapeutic agent is an antisense oligomer complementary to a sequence with 100% sequence identity to a sequence selected from the group consisting of SEQ ID NOs: 130-331. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 534-8095. In some cases, the therapeutic agent is an antisense oligomer that has a sequence with 100% sequence identity to a sequence selected from the group consisting of SEQ ID NOs: 534-8095.

In some cases, the disease or the condition is selected from the group consisting of: Intellectual Disability; Colorectal Cancer; Bleeding Disorder, Platelet-Type, 15; Autosomal dominant macrothrombocytopenia; Hypertrophic Cardiomyopathy; Cardiomyopathy, Dilated, 1AA; cardiomyopathy, familial hypertrophic, 23, with or without ventricular noncompaction; Familial dilated cardiomyopathy; Charcot-Marie-Tooth Disease; Conduction disorder of the heart; Cardiomyopathy, Dilated; Alcohol Use Disorder; Alcohol abuse; Alcoholic Intoxication, Chronic; Alcohol Use Disorder; Alcohol abuse; Alcoholic Intoxication, Chronic; Schizophrenia; Bipolar Disorder; Alcoholic Intoxication, Chronic; Congenital Disorders of Glycosylation; Epileptic encephalopathy; Depressive disorder; Congenital disorder of glycosylation type 1H; pontocerebellar hypoplasia, type 9; epileptic encephalopathy; ataxias, hereditary; spastic paraplegia 63, autosomal recessive; Cerebellar Hypoplasia; Spastic Paraplegia, Hereditary; Nonketotic Hyperglycinemia; Hyperglycinemia, Transient Neonatal; Glycine encephalopathy; KBG syndrome; 16q24.3 microdeletion syndrome; Situs Inversus; Malignant neoplasm of breast; Maturity onset diabetes mellitus in young; Maturity-Onset Diabetes Of The Young, Type 14; Microcephaly; Polydactyly; Ciliopathies; Mental Depression; Seasonal Affective Disorder; Bipolar Disorder; Mood Disorders; Depressive disorder; Malignant neoplasm of breast; Cytopenia; Gastrointestinal Neoplasms; Depressive disorder; Prostate cancer, familial; Primary peritoneal carcinoma; ovarian cancer, familial, susceptibility to, 1; Breast Cancer, Familial Male; Malignant Neoplasms; Pancreatic Neoplasm; Breast Cancer, Familial; Neoplasm of uncertain or unknown behavior of breast; Ovarian Carcinoma; Adenocarcinoma of prostate; Neoplasm of uncertain or unknown behavior of ovary; Malignant neoplasm of pancreas; Pancreatic carcinoma; Malignant neoplasm of ovary; Congenital anemia; Hematologic Neoplasms; Breast adenocarcinoma; breast-ovarian cancer, familial, susceptibility to, 1; Pancreatic carcinoma, familial; Benign tumor of pancreas; Breast Carcinoma; breast cancer, familial, susceptibility to, 1; ovarian neoplasm; Hereditary Breast and Ovarian Cancer Syndrome; Mental Depression; Epithelial ovarian cancer; Malignant neoplasm of breast; Sporadic Breast Carcinoma; Breast-ovarian cancer, familial, 1; Pancreatic cancer, susceptibility to, 4; C8 deficiency, type II; ventricular tachycardia, catecholaminergic polymorphic, 4; ventricular tachycardia, catecholaminergic polymorphic, 1; Long QT Syndrome; Romano-Ward Syndrome; Long Qt Syndrome 14; peeling skin syndrome; peeling skin with leukonychia, acral punctate keratoses, cheilitis, and knuckle pads; erythrokeratoderma; palmoplantar keratosis; morbid obesity and spermatogenic failure; warburton anyane yeboa syndrome; mosaic variegated aneuploidy syndrome; Breast Cancer, Familial; Malignant neoplasm of ovary; Mitochondrial Diseases; Severe combined immunodeficiency with sensitivity to ionizing radiation; severe combined immunodeficiency, partial; Reticuloendotheliosis, familial, with eosinophilia; Omenn Syndrome; Athabaskan severe combined immunodeficiency; Severe Combined Immunodeficiency, Athabaskan-Type; Cocaine Abuse; Congenital anemia; Cytopenia; Neurodegeneration Due To Cerebral Folate Transport Deficiency; Megaloblastic Anemia due to Dihydrofolate Reductase Deficiency; Alcoholic Intoxication, Chronic; Bipolar Disorder; Unipolar Depression; Major Depressive Disorder; Schizophrenia and related disorders; Left ventricular noncompaction cardiomyopathy; Left ventricular noncompaction; Familial Meniere's disease; Charcot-Marie-Tooth Disease; noncompaction of left ventricular myocardium, familial isolated, autosomal dominant 1; Primary microcephaly; Microcephaly; Epileptic encephalopathy; Mental retardation, autosomal dominant 7; Colorectal Cancer; Depressive disorder; Mental Depression; Caudal Regression Syndrome; Arnold Chiari Malformation; Caudal dysplasia syndrome; Chiari malformation type II; Sacral Agenesis Syndrome; Sacral agenesis; Currarino triad; Spina bifida aperta of cervical spine; Neural tube defects; Polycystic Kidney, Autosomal Dominant; polycystic kidney disease 3, autosomal dominant; alcoholic intoxication, chronic; malignant neoplasm of breast; ataxias, hereditary; epilepsy, progressive myoclonic, 6; Epileptic encephalopathy; Lattice corneal dystrophy Type II; Meretoja syndrome; Malignant neoplasm of breast; Periodic Fever Syndrome; Familial Amyloid Polyneuropathy, Type IV; Cerebral Amyloid Angiopathy, Gsn-Related; Abnormality of the cornea; Amyloidosis, Finnish type; Primary microcephaly; cornelia de lange syndrome 5; radial club hand; cornelia de lange syndrome; wilson-turner x-linked mental retardation syndrome; congenital anemia; retinitis pigmentosa 79; charcot-marie-tooth disease; cytopenia; hemolytic anemia, nonspherocytic, due to hexokinase deficiency; neuropathy, hereditary motor and sensory, russe type; interferon gamma, receptor 1, deficiency; immunodeficiency 27a; immunodeficiency 27b; H. pylori infection, susceptibility to; Tuberculosis infection, protection against; Immunodeficiency 27A, mycobacteriosis, AR; Immunodeficiency 27B, mycobacteriosis, AD; Hepatitis B virus infection, susceptibility to; Tuberculosis, susceptibility to; Malignant neoplasm of breast; Lymphoma, Non-Hodgkin; immunodeficiency 15; Congenital myopathy; Arthrogryposis; Rhabdomyolysis; myopathy with exercise intolerance, Swedish type; Myopathy with lactic acidosis, hereditary; Charcot-Marie-Tooth disease, recessive intermediate B; Charcot-Marie-Tooth Disease; Deafness, Autosomal Recessive 89; Deafness, autosomal recessive 89; Dyslexia, susceptibility to, 2; Joubert Syndrome 23; Polydactyly; Familial aplasia of the vermis; Joubert syndrome with Jeune asphyxiating thoracic dystrophy; Hydrocephalus; Ciliopathies; Short-rib thoracic dysplasia 14 with polydactyly; Retinitis Pigmentosa; Retinitis pigmentosa 69; Muscular Dystrophy, Limb-Girdle, Type 1B; Hypertrophic Cardiomyopathy; Left ventricular noncompaction; Osteogenesis Imperfecta; Charcot-Marie-Tooth Disease; Cardiomyopathy, Familial Idiopathic; Familial Partial Lipodystrophy, Type 1; Left ventricular noncompaction cardiomyopathy; Mandibuloacral dysostosis; Muscular Dystrophy, Congenital, Lmna-Related; Congenital myopathy; Arthrogryposis; Arrhythmogenic Right Ventricular Dysplasia; Muscular Dystrophies, Limb-Girdle; Malouf syndrome; Najjar syndrome; Charcot-Marie-Tooth disease, Type 2B1; Emery-Dreifuss Muscular Dystrophy 3; Lethal tight skin contracture syndrome; Familial Partial Lipodystrophy, Type 2; Conduction disorder of the heart; Cardiomyopathy, Dilated; Progeria Syndrome, Childhood-Onset; Atypical Werner syndrome; Congenital muscular dystrophy; Autosomal Recessive Emery-Dreifuss Muscular Dystrophy; Heart-hand syndrome, Slovenian type; Progeria; Autosomal Dominant Emery-Dreifuss Muscular Dystrophy; Cardiomyopathy, dilated, 1A; Mandibuloacral dysplasia; Muscular dystrophy, limb-girdle, type 1B; Emery-Dreifuss muscular dystrophy 2, AD; Malouf syndrome; Restrictive dermopathy, lethal; Emery-Dreifuss muscular dystrophy 3, AR; Heart-hand syndrome, Slovenian type; Charcot-Marie-Tooth disease, type 2B1; Hutchinson-Gilford progeria; Lipodystrophy, familial partial, type 2; Muscular dystrophy, congenital; Malignant neoplasm of breast; Deafness, Autosomal Recessive 63; Bardet-Biedl Syndrome; Polydactyly; Ciliopathies; Bardet-Biedl Syndrome 17; Major Affective Disorder 2; Bipolar Disorder; Epileptic encephalopathy; Rett Syndrome, Preserved Speech Variant; Lubs X-linked mental retardation syndrome; Encephalopathy, Neonatal Severe, Due To Mecp2 Mutations; Mental Retardation with Psychosis, Pyramidal Signs, and Macroorchidism; Mental Retardation, X-Linked, With Spasticity; Trisomy Xq28; Mental Retardation, X-Linked 16; Epileptic encephalopathy; Mental Retardation, X-Linked, Syndromic 13; Mental Retardation, X-Linked 1; Rett Syndrome, Atypical; Mental Retardation, X-Linked 79; Microcephaly; Ppm-X Syndrome; Rett Syndrome, Zappella Variant; Rett Syndrome; Autism susceptibility, X-linked 3; Benign neoplasm of adrenal gland; Retinitis Pigmentosa; Conventional (Clear Cell) Renal Cell Carcinoma; Squamous cell carcinoma of the head and neck; Malignant neoplasm of aortic body and other paraganglia; Retinitis Pigmentosa 38; Malignant Adrenal Medulla Neoplasm; Benign neoplasm of aortic body and other paraganglia; Primary microcephaly; Microcephaly 15, Primary, Autosomal Recessive; Autosomal Recessive Primary Microcephaly; Microcephaly; Leukemia, acute myeloid; Deafness, Autosomal Recessive 74; Cytopenia; Uridine 5-Prime Monophosphate Hydrolase Deficiency, Hemolytic Anemia due to; Congenital anemia; NUT midline carcinoma; Malignant neoplasm of breast; Deafness, Autosomal Recessive; Deafness, Autosomal Recessive 9; Auditory Neuropathy, Nonsyndromic Recessive; Auditory neuropathy, autosomal recessive, 1; Malignant neoplasm of breast; Breast Carcinoma; Neoplasm of uncertain or unknown behavior of breast; Breast adenocarcinoma; Ketotic hypoglycemia; Rhabdomyolysis; Glycogen Storage Disease IXB; Phosphorylase kinase deficiency of liver and muscle, autosomal recessive; Congenital anemia; Congenital Disorders of Glycosylation; Cytopenia; Epileptic encephalopathy; Paroxysmal nocturnal hemoglobinuria; Paroxysmal Nocturnal Hemoglobinuria 1; West Syndrome; Multiple Congenital Anomalies-Hypotonia-Seizures Syndrome 2; Congenital anemia; Congenital Disorders of Glycosylation; Cytopenia; Epileptic encephalopathy; Paroxysmal nocturnal hemoglobinuria; Paroxysmal Nocturnal Hemoglobinuria 1; West Syndrome; Multiple Congenital Anomalies-Hypotonia-Seizures Syndrome 2; Bruck Syndrome 2; Osteogenesis Imperfecta; Bruck Syndrome 1; Arthrogryposis; Bruck Syndrome; Cutaneous Melanoma; Melanoma; Parkinson Disease 2, Autosomal Recessive Juvenile; Parkinson Disease; Young Onset Parkinson Disease; Parkinson Disease, Late-Onset; Leprosy, Susceptibility To; Adenocarcinoma, Ovarian, Somatic; Adenocarcinoma Of Lung, Somatic; Primary Microcephaly; Neurodevelopmental Disorder With Microcephaly, Hypotonia, And Variable Brain Anomalies; Myocardial infarcation, susceptibility to; Pure Gonadal Dysgenesis, 46, XX; Ovarian dysgenesis 3; Insulin resistance, susceptibility to; Leukodystrophy; Epileptic encephalopathy; Adams-Oliver Syndrome 3; Adams Oliver syndrome; Mood Disorders; Congenital anemia; Hematologic Neoplasms; Anemia, Diamond-Blackfan; Cytopenia; Aase Smith syndrome 2; Radial club hand; Precursor T-Cell Lymphoblastic Leukemia-Lymphoma; Diamond-Blackfan anemia 6; Hereditary non-polyposis colorectal cancer syndrome; Familial Colorectal Cancer Type X; Hereditary Nonpolyposis Colorectal Cancer; Hereditary Nonpolyposis Colorectal Neoplasms; Thyroid Hormone Metabolism, Abnormal; Thyroid Hormone Resistance Syndrome; Bipolar Disorder; Disturbance in mood; Combined Oxidative Phosphorylation Deficiency 28; Coffin-Siris Syndrome 5; Coffin-Siris syndrome; Meningioma; Meningiomas, Multiple; Meningioma, familial, susceptibility to; Primary microcephaly; Congenital anemia; Osteogenesis Imperfecta; Cytopenia; Epileptic encephalopathy; Radial club hand; Cornelia De Lange Syndrome; Growth Deficiency and Mental Retardation with Facial Dysmorphism; Congenital muscular hypertrophy-cerebral syndrome; Cornelia de Lange syndrome 2; Infiltrating duct carcinoma of female breast; Colorectal Cancer; Neurofibromatosis 1; Neurofibromatosis, Type 1-Like Syndrome; Legius syndrome; Shwachman syndrome; Glioblastoma Multiforme; Ovarian Serous Adenocarcinoma; Polynesian Bronchiectasis; Kartagener Syndrome; Polyhydramnios, Megalencephaly, And Symptomatic Epilepsy; Hydrocephalus; Epileptic encephalopathy; Oligodontia; Hypodontia; Orofacial cleft 10; Malignant neoplasm of urinary bladder; Acute Promyelocytic Leukemia; Adenocarcinoma of large intestine; Bladder Neoplasm; Epileptic encephalopathy; Neoplasm of uncertain or unknown behavior of bladder; Mental Retardation, Autosomal Dominant 41; Benign neoplasm of bladder; Lymphoma, Non-Hodgkin; Carcinoma in situ of bladder; Central nervous system lymphoma; Carcinoma of bladder; Plantar Lipomatosis, Unusual Facies, and Developmental Delay; Pierpont syndrome; X-linked Adrenal Hypoplasia; Depressive Symptoms; Familial dilated cardiomyopathy; Familial Glucocorticoid Deficiency Type 1; Conduction disorder of the heart; Cardiomyopathy, Dilated; Angelman Syndrome; Epileptic encephalopathy; Microcephaly; Duplication 15q11-q13 Syndrome; Leukodystrophy, Hypomyelinating, 6; Chromosome 10p12-p11 Deletion Syndrome; Colorectal Cancer; Desanto-Shinawi Syndrome; Ataxias, Hereditary; Cerebellar Ataxia, Mental Retardation, And Dysequilibrium Syndrome 2; Hydrocephalus; Cerebellar Hypoplasia; Dysequilibrium Syndrome; OPTIC ATROPHY 11; Polynesian Bronchiectasis; Ciliary Dyskinesia, Primary, 22; Kartagener Syndrome; and Ciliopathies.

In some cases, the disease or the condition is caused by a deficient amount or activity of a functional target protein, and wherein the first protein comprising the target peptide sequence is at least partially functionally equivalent to the functional target protein. In some cases, the disease or the condition is caused by a deficient amount or activity of the target peptide sequence. In some cases, the therapeutic agent increases the level of the first processed mRNA in the cell. In some cases, the level of the first processed mRNA in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the first processed mRNA in a control cell.

In some cases, the therapeutic agent increases the expression of the target peptide sequence in the cell. In some cases, the level of the target peptide sequence in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the target peptide sequence in a control cell.

In some cases, the therapeutic agent is an antisense oligonucleotide, and wherein the therapeutic agent comprises a backbone modification comprising a phosphorothioate linkage or a phosphorodiamidate linkage. In some cases, the therapeutic agent is an antisense oligonucleotide, and wherein the therapeutic agent comprises a phosphorodiamidate morpholino, a locked nucleic acid, a peptide nucleic acid, a 2′-O-methyl, a 2′-Fluoro, or a 2′-O-methoxyethyl moiety. In some cases, the therapeutic agent is an antisense oligonucleotide, and wherein the therapeutic agent comprises at least one modified sugar moiety. In some cases, each sugar moiety is a modified sugar moiety. In some cases, the therapeutic agent is an antisense oligonucleotide, and wherein the therapeutic agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some cases, the method further comprises assessing mRNA level encoding the target peptide sequence or expression level of the target peptide sequence.

In some cases, the subject is a human. In some cases, the subject is a non-human animal. In some cases, the subject is a fetus, an embryo, or a child. In some cases, the cell or the cells is ex vivo, or in a tissue, or organ ex vivo.

In some cases, the therapeutic agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

Disclosed herein, in some aspects, is a therapeutic agent for use in the method disclosed herein.

Disclosed herein, in some aspects, is a pharmaceutical composition comprising the therapeutic agent described herein and a pharmaceutically acceptable excipient.

Disclosed herein, in some aspects, is a method of treating a subject in need thereof, comprising administering the pharmaceutical composition described herein by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B depict a schematic representation of MECP2 pre-mRNA splicing isoforms (FIG. 1A) and protein domains (FIG. 1B).

FIG. 2 is a schematic depicting the targeting strategy for modulating the alternative splicing of MECP2 pre-mRNA.

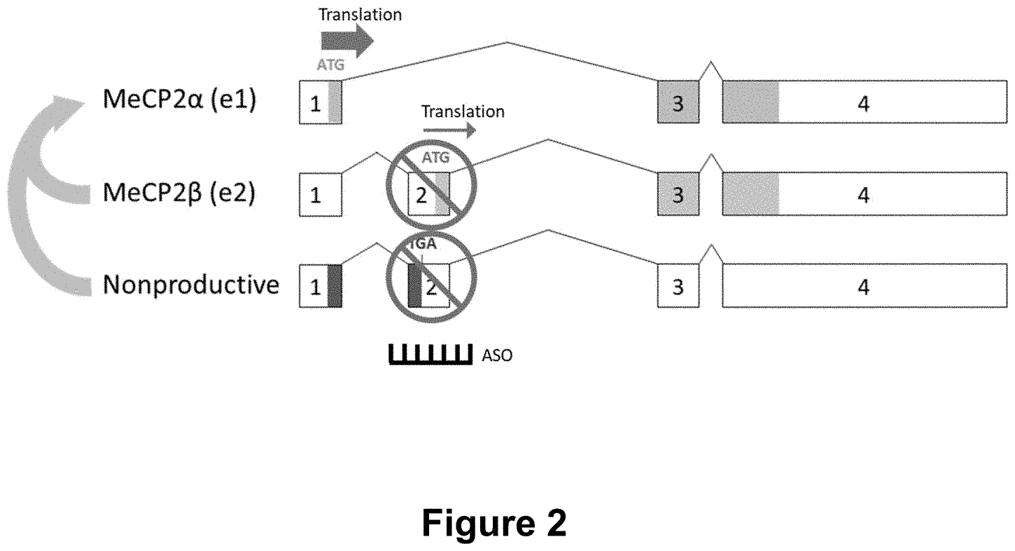

FIGS. 3A-3B show RT-PCR analysis performed to confirm exon 2 inclusion event during MECP2 mRNA processing.

FIG. 4 depicts an ASO walk for MECP2 exon 2 region.

FIG. 5 shows RT-PCR analysis performed to evaluate the ASO walk over MECP2 exon 2 region.

FIGS. 6A-6B show effects of select ASOs on MECP2 exon 2 inclusion (FIG. 6A) and MECP2 protein expression (FIG. 6B).

FIG. 7 shows the quantification of total MECP2 mRNA (e1+e2).

FIG. 8 shows a dose-dependent effect of selected ASOs Mecp2 exon 2 inclusion in mouse embryonic fibroblast (MEF) cells

FIG. 9 shows RT-PCR analysis performed to evaluate the ASO microwalk in the region of ASOs 31-33 and 37-38.

### DETAILED DESCRIPTION

Alternative splicing in certain genes can lead to non-productive or less productive mRNA transcripts which in turn can downregulate protein expression. Therapeutic agents that can target the alternative splicing events in these genes can modulate the expression level of proteins. Such therapeutic agents can be used to treat a condition caused by protein deficiency.

One of the alternative splicing events that can lead to non-productive or less productive mRNA transcripts can be the inclusion of an exon containing an inefficient translation region, which can result in inefficient translation of the mRNA transcript. In some cases, the mRNA transcript with the inefficient translation region has significantly lower translation efficiency as compared to a corresponding mRNA transcript that is otherwise identical but without the inefficient region.

In one aspect, the present disclosure provides compositions and methods for modulating alternative splicing of genes that can have a pre-mRNA that has an exon having an inefficient translation region to increase the production of mRNAs that is efficiently translated and codes for a target peptide sequence, and thus the translated target peptide sequence. The compositions and methods include antisense oligomers (ASOs) that can cause exon skipping and promote the generation of mRNA that can be efficiently translated. In various examples, the target peptide sequence can be increased using the methods of the disclosure to treat a condition caused by deficiency of the target peptide sequence or a relevant protein.

Described herein, in some embodiments, is a method of modulating expression of a target peptide sequence by cells having a pre-mRNA that encodes the target peptide sequence and comprises an inefficient translation region, the method comprising contacting the cells with a therapeutic agent that binds to a targeted region of the pre-mRNA encoding the target peptide sequence, whereby splicing of the inefficient translation region from the pre-mRNA encoding the target peptide sequence is modulated, thereby modulating a level of a first processed mRNA that is devoid of the inefficient translation region and encodes the target peptide sequence, and thereby modulating the expression of the target peptide sequence in the cells, wherein the processed mRNA has a higher translation efficiency for producing the target peptide sequence in the cells as compared to a second processed mRNA that comprises the inefficient translation region. In some cases, the second processed mRNA is otherwise identical to the first processed mRNA but comprises the inefficient translation region. In some cases, the first processed mRNA and the second processed mRNA are both splicing products of the same pre-mRNA. In some cases, the subject therapeutic agent modulates the splicing of the pre-mRNA, thereby modulating the balance between the first processed mRNA and the second processed mRNA in the cell.

In some embodiments, one of the alternative splicing events that can lead to non-productive or less productive mRNA transcript is the inclusion of an exon that contains a premature termination codon (PTC) followed by an alternative start codon. In these cases, the pre-mRNA has a first start codon, a second start codon (alternative start codon), and a PTC located downstream of the first start codon and upstream of the second start codon. Without wishing to be bound by a certain theory, the motif or the exon containing the PTC and the alternative start codon can contribute to the low productivity of the mRNA transcript. In some embodiments, the motif or the exon containing the PTC and the alternative start codon leads to inefficient translation of the downstream exon sequences. In some embodiments, the motif or the exon containing the PTC and the alternative start codon leads to less stable protein product expressed by the downstream exon sequences or deficient post-translational modification, or some other processes at the molecular or cellular level, which can result in deficient protein expression or function. Without wishing to be bound by a certain theory, the presence of the alternative start codon can contribute to the lack of nonsense-mediated decay (NMD) of the mRNA transcript, as the translating ribosome can reinitiate translation at the alternative start codon right after encountering the PTC rather than triggering the recruitment of the NMD machinery, which ultimately can lead to the degradation of the mRNA transcript.

In one aspect, the present disclosure provides compositions and methods for modulating alternative splicing of genes that can have a pre-mRNA that has an exon containing a PTC followed by an alternative start codon to increase the production of mature mRNAs that codes for a target peptide sequence, and thus the translated target peptide sequence. In some cases, the compositions include ASOs that can cause exon skipping and promote the generation of an mRNA that can be efficiently translated. In various examples, the target peptide sequence can be increased using the methods of the disclosure to treat a condition caused by deficiency of the target peptide sequence or a relevant protein.

Described herein, in some embodiments, is a method of modulating expression of a target peptide sequence by cells having a pre-mRNA that encodes the target peptide sequence and comprises a first start codon, a second start codon, and a premature termination codon (PTC) located downstream of the first start codon and upstream of the second start codon, the method comprising contacting the cells with a therapeutic agent that binds to a targeted portion of the pre-mRNA encoding the target peptide sequence, whereby splicing of the PTC and the second start codon from the pre-mRNA is modulated, thereby modulating a level of a first processed mRNA that that is devoid of the PTC and the second start codon and encodes the target peptide sequence, and thereby modulating the expression of the target peptide sequence in the cells.

### RNA Splicing

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not can be typically modeled as a stochastic rather than deterministic process, such that even the most defined splicing signals can sometimes splice incorrectly. However, under normal conditions, pre-mRNA splicing can proceed at surprisingly high fidelity. This can be attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. In some embodiments, however, an alternative start codon that follows the PTC can prevent the induction of NMD event, as exemplified in the case of MECP2 e2 mRNA isoform (discussed below). Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

In some embodiments, the compositions and methods make use of cryptic splice sites to modulate alternative splicing in order to produce desirable splicing isoform, in order to modulate the expression level of target peptide sequence. Cryptic (or pseudo-) splice sites can have the same splicing recognition sequences as genuine splice sites but are not used in splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (available at www.ncbi.nlm.nih.gov/pmc/articles/PMC2095810/pdf/gkm680.pdf)

The cryptic splice sites or splicing regulatory sequences may compete for RNA-binding proteins, such as U2AF. In some embodiments, an agent may bind to a cryptic splice site or splicing regulatory sequence to prevent binding of RNA-binding proteins and thereby favor binding of RNA-binding proteins to the desirable splice sites.

MECP2 mRNA Splicing

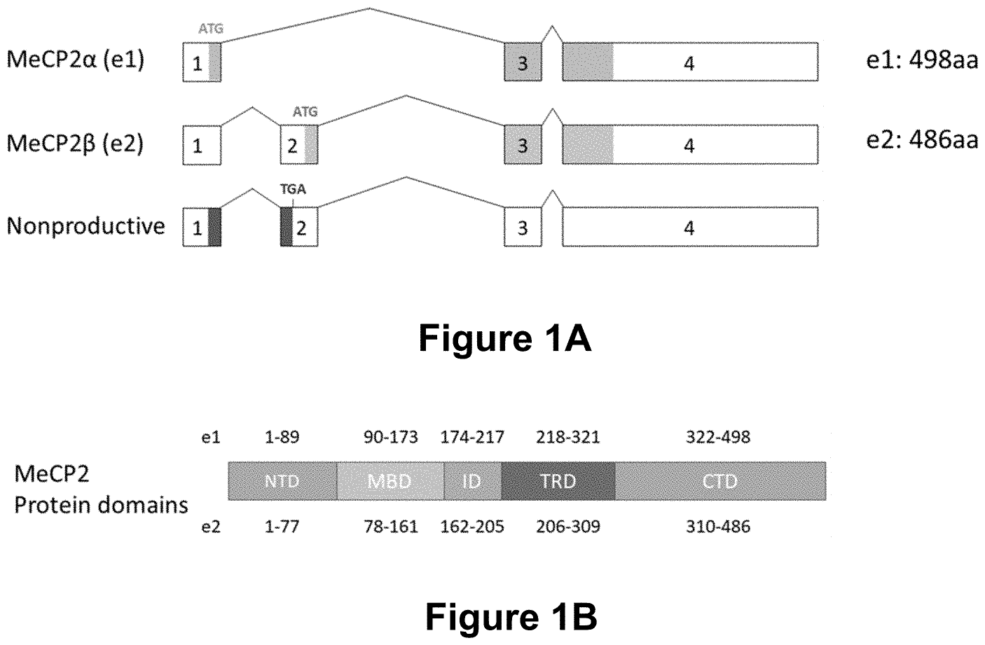

In some embodiments, the methods of the present disclosure exploit the alternative splicing of the pre-mRNA transcribed from MECP2 gene. MECP2 pre-mRNA can have 4 exons and be alternatively spliced (FIG. 1). Exemplary MECP2 pre-mRNA sequences include SEQ ID NOs: 2-27. In some embodiments, MECP2 pre-mRNA can be spliced into two isoforms, which can result in production of MeCP2a or e1 protein isoform, and MeCP20 or e2 isoform, respectively. Skipping of exon 2 can result in the production of the major MeCP2a or e1 isoform, whereas inclusion of exon 2 can give rise to MeCP20 or e2-isoform. The MeCP2-e1 and e2 isoforms can have a unique N-terminal sequence of 21aa and 9aa respectively. The relative expression level of these isoforms can vary among tissues, with MeCP2-e1 being more dominant in adult brain. MeCP2-e2 is expressed more abundantly in placenta, liver, and skeletal muscle. Furthermore, in some cases, deletion of MeCP2-e2 by disrupting exon 2 does not result in RTT-associated phenotypes in mice. Exon 2 is an out-of-frame exon. It has both a PTC as well as an alternative start codon. Translation from a downstream start codon can be very inefficient. Mutagenesis studies have shown that the presence of two start codons in the full-length (e2) transcript dramatically reduces translation efficiency of the downstream open-reading-frame. When the upstream ATG in exon 1 is eliminated, translation of MeCP2-e2 can increase significantly. In some embodiments, skipping (splicing out) of exon 2 from full length (e2) MECP2 transcripts can reduce the nonproductive transcript pool, leading to increased translation of MeCP2-e1 protein. In adult human brain, approximately 50% of the transcripts can contain exon 2 as determined by RNA-seq (Lister PMID 23828890). In some cases, the isoform containing exon 2 can be dispensable in adult brain, as a result, exon 2 skipping induced by a therapeutic agent (e.g., ASO) can be a viable therapeutic strategy to increase MeCP2 expression in patients suffering from MeCP2 protein deficiency, e.g., Rett syndrome patients.

In some embodiments, the therapeutic agent targets a sequence about 4 to about 300 nucleotides upstream (or 5′) from the 5′ end of the inefficient translation region, e.g., exon 2 region of MECP2 pre-mRNA. In some embodiments, the therapeutic agent targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the inefficient translation region. In some embodiments, the therapeutic agent may target a sequence more than 300 nucleotides upstream from the 5′ end of the inefficient translation region. In some embodiments, the therapeutic agent targets a sequence about 4 to about 300 nucleotides downstream (or 3′) from the 3′ end of the inefficient translation region. In some embodiments, the therapeutic agent targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the inefficient translation region. In some embodiments, the therapeutic agent targets a sequence more than 300 nucleotides downstream from the 3′ end of the inefficient translation region.

In some embodiments, the therapeutic agent targets a sequence about 4 to about 300 nucleotides upstream (or 5′) from the 5′ end of the exon containing the PTC and the second start codon (alternative start codon), e.g., exon 2 region of MECP2 pre-mRNA. In some embodiments, the therapeutic agent targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the exon containing the PTC and the alternative start codon. In some embodiments, the therapeutic agent may target a sequence more than 300 nucleotides upstream from the 5′ end of the exon containing the PTC and the alternative start codon. In some embodiments, the therapeutic agent targets a sequence about 4 to about 300 nucleotides downstream (or 3′) from the 3′ end of the exon containing the PTC and the alternative start codon. In some embodiments, the therapeutic agent targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the exon containing the PTC and the alternative start codon. In some embodiments, the therapeutic agent targets a sequence more than 300 nucleotides downstream from the 3′ end of the exon containing the PTC and the alternative start codon.

In some embodiments, the pre-mRNA transcript that can be targeted by a method or composition provided herein is encoded by a genetic sequence with at least about 80%, 85%, 90%, 95%, 96%, 97%, 98%, 99% or 100% sequence identity to SEQ ID NO: 1. In some embodiments, the pre-mRNA transcript that can be targeted by a method or composition provided herein comprises a sequence with at least about 80%, 85%, 90%, 95%, 96%, 97%, 98%, 99% or 100% sequence identity to any one of SEQ ID NOs: 2-27.

In some embodiments, the therapeutic agent targets intron 1, exon 2, or intron 2 of MECP2 pre-mRNA. In some embodiments, the therapeutic agent targets an exon sequence that has at least about 80%, 85%, 90%, 95%, 96%, 97%, 98%, 99% or 100% sequence identity to any selected from the group consisting of SEQ ID NOs: 28-31. In some embodiments, the therapeutic agent targets exon 2 of MECP2 pre-mRNA. In some embodiments, the therapeutic agent targets an exon sequence that has at least about 80%, 85%, 90%, 95%, 96%, 97%, 98%, 99% or 100% sequence identity to SEQ ID NO: 28.

In some embodiments, the therapeutic agent targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 2 of MECP2 pre-mRNA. In some embodiments, the therapeutic agent targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 2 of MECP2 pre-mRNA.

In some embodiments, the therapeutic agent targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 2 of MECP2 pre-mRNA. In some embodiments, the therapeutic agent targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 2 of MECP2 pre-mRNA.

In some embodiments, the therapeutic agent is an ASO and the ASO has a sequence complementary to the targeted portion of the pre-mRNA according to SEQ ID NOs: 2-27. In some embodiments, the ASO has a sequence complementary to a sequence with at least about 80%, 85%, 90%, 95%, 96%, 97%, 98%, 99% or 100% sequence identity to any one selected from the group consisting of SEQ ID NOs: 28-31. In some embodiments, the ASO has a sequence complementary to a sequence with 100% sequence identity to any one selected from the group consisting of SEQ ID NOs: 28-31. In some embodiments, the ASO has a sequence complementary to a sequence with 100% sequence identity to SEQ ID NO: 28.

In some embodiments, the ASOs target a sequence containing an exon-intron boundary (or junction).

In some embodiments, the methods and compositions of the present disclosure are used to increase the expression of MeCP2 by inducing exon skipping of exon 2 of a MECP2 pre-mRNA.

In some cases, the target peptide sequence is a portion of MECP2 protein. In some cases, the therapeutic agent increases level of a first processed mRNA encoding a protein having a sequence SEQ ID NO: 8204, and decreases level of a second processed mRNA encoding a protein having a sequence SEQ ID NO: 440. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 238. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 238. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 154092307. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 154092307. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 238. In some cases, the targeted region of the pre-mRNA is at least about 1500 nucleotides, about 1000 nucleotides, about 800 nucleotides, about some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 154092184. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 154092184. In some cases, the targeted region of the pre-mRNA is within a sequence SEQ ID NO: 238. In some cases, the targeted region of the pre-mRNA is within a sequence between a pair of genomic sites GRCh38/hg38: chrX 154092307, and GRCh38/hg38: chrX 154092184. In some cases, the therapeutic agent is an antisense oligomer complementary to a sequence with at least about 80%, 85%, 90%, 95%, 96%, 97%, 98%, 99% or 100% sequence identity to SEQ ID NO:238. In some cases, the therapeutic agent is an antisense oligomer complementary to a sequence with 100% sequence identity to SEQ ID NO:238. In some cases, the therapeutic agent increases level of a first processed mRNA encoding a protein having a sequence SEQ ID NO: 8205, and decreases level of a second processed mRNA encoding a protein having a sequence SEQ ID NO: 441. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 239. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 239. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 154092307. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 154092307. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 239. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 239. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 154092184. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 154092184. In some cases, the targeted region of the pre-mRNA is within a sequence SEQ ID NO: 239. In some cases, the targeted region of the pre-mRNA is within a sequence between a pair of genomic sites GRCh38/hg38: chrX 154092307, and GRCh38/hg38: chrX 154092184. In some cases, the therapeutic agent is an antisense oligomer complementary to a sequence with at least about 80%, 85%, 90%, 95%, 96%, 97%, 98%, 99% or 100% sequence identity to SEQ ID NO:239. In some cases, the therapeutic agent is an antisense oligomer complementary to a sequence with 100% sequence identity to SEQ ID NO:239. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4548-4611. In some cases, the therapeutic agent is an antisense oligomer that has a sequence selected from the group consisting of SEQ ID NOs: 4548-4611. In some cases, the target peptide sequence is a portion of MECP2 protein, and the method treats a disease or the condition that comprises Intellectual Disability; Rett Syndrome, Preserved Speech Variant; Lubs X-linked mental retardation syndrome; Encephalopathy, Neonatal Severe, due to MeCP2 Mutations; Mental Retardation with Psychosis, Pyramidal Signs, and Macroorchidism; Mental Retardation, X-Linked, With Spasticity; Trisomy Xq28; Mental Retardation, X-Linked 16; Epileptic encephalopathy; Mental Retardation, X-Linked, Syndromic 13; Mental Retardation, X-Linked 1; Rett Syndrome, Atypical; Mental Retardation, X-Linked 79; Microcephaly; Ppm-X Syndrome; Rett Syndrome, Zappella Variant; Rett Syndrome; or Autism susceptibility, X-linked 3.

### Protein Expression

In some embodiments, the methods described herein are used to increase the production of a functional protein, e.g., having a target peptide sequence, e.g., a MeCP2 protein. As used herein, the term “functional” refers to the amount of activity or function of a protein that is necessary to eliminate any one or more symptoms of a treated condition or disease, e.g., Rett syndrome. In some embodiments, the methods are used to increase the production of a partially functional MeCP2 protein. As used herein, the term “partially functional” refers to any amount of activity or function of the protein that is less than the amount of activity or function that is necessary to eliminate or prevent any one or more symptoms of a disease or condition, e.g., Rett syndrome. In some embodiments, a partially functional protein will have at least 10%, at least 20%, at least 30%, at least 40%, at least 50%, at least 60%, at least 70%, at least 75%, at least 80%, at least 85%, at least 90%, or at least 95% less activity relative to the fully functional protein.

In some embodiments, the method is a method of increasing the expression of a target peptide sequence by cells of a subject having a pre-mRNA encoding the target peptide sequence, wherein the subject has a disease or condition caused by a deficient amount of activity of a functional target protein that the target peptide sequence is at least functionally equivalent to. In such an embodiment, the subject has a first allele encoding a functional target protein is not produced. In another such embodiment, the subject has a first allele encoding a functional target protein, and a second allele encoding a nonfunctional target protein. In another such embodiment, the subject has a first allele encoding a functional target protein, and a second allele encoding a partially functional target protein. In some of these embodiments, the antisense oligomer binds to a targeted portion of the pre-mRNA transcribed from the second allele that codes for a target peptide sequence and comprises an exon containing an inefficient translation region, thereby inducing skipping of the inefficient translation region from the pre-mRNA, and causing an increase in the level of mature mRNA encoding the target peptide sequence, and an increase in the expression of the target peptide sequence in the cells of the subject. In some of these embodiments, the antisense oligomer binds to a targeted portion of the pre-mRNA transcribed from the second allele that codes for a target peptide sequence and comprises an exon containing a PTC followed by an alternative start codon, thereby inducing skipping of the PTC and the start codon from the pre-mRNA, and causing an increase in the level of mature mRNA encoding the target peptide sequence, and an increase in the expression of the target peptide sequence in the cells of the subject.

In some embodiments, the target peptide sequence as described herein can be a fully functional protein. In some embodiments, the target peptide sequence is a portion of a full-length protein, for instance, an N-terminal portion of a full-length protein, or a C-terminal portion of a full-length protein. In the case of MeCP2, the target peptide sequence can be a C-terminal portion of MeCP2. In some embodiments, the target peptide sequence is translated from a sequence selected from the group consisting of SEQ ID NOs: 32-34. In some embodiments, the target peptide sequence is translated from a sequence having SEQ ID NOs: 32 or 33 and 34. In some embodiments, the target peptide sequence is encoded by a portion of the pre-mRNA downstream of the inefficient translation region or the alternative start codon. In some embodiments, the target peptide sequence is encoded by a portion of the pre-mRNA upstream of the inefficient translation region or the alternative start codon. The target peptide sequence can comprise at least about 10 amino acids (aa), 20 aa, 30 aa, 40 aa, 50 aa, 60 aa, 70 aa, 80 aa, 90 aa, 100 aa, 120 aa, 150 aa, 200 aa, 300 aa, 400 aa, 500 aa, 600 aa, 700 aa, 800 aa, 100 aa, 1500 aa, or 2000 aa. The target peptide sequence can comprise about 10 amino acids (aa), 20 aa, 30 aa, 40 aa, 50 aa, 60 aa, 70 aa, 80 aa, 90 aa, 100 aa, 120 aa, 150 aa, 200 aa, 300 aa, 400 aa, 500 aa, 600 aa, 700 aa, 800 aa, 100 aa, 1500 aa, or 2000 aa.

In some embodiments, the method is a method of increasing the expression of a functional target protein having a target peptide sequence, e.g., MeCP2-e1 isoform encoded by exon 1, 3 and 4 of MECP2 gene, by cells of a subject having a pre-mRNA encoding the target peptide sequence, wherein the subject has a disease or condition caused by a deficient amount of activity of MeCP2 protein. In such an embodiment, the subject has an allele encoding a partially functional MeCP2, e.g., a hypomorphic allele. In such embodiment, the subject has a first allele encoding a functional MeCP2 protein, and a second allele encoding a partially functional MeCP2 protein. In some of these embodiments, the antisense oligomer binds to a targeted portion of the pre-mRNA transcribed from the second allele that codes for MeCP2 and comprises exon 2 of MECP2, thereby inducing skipping of at least part of MECP2 exon 2 from the pre-mRNA, and causing an increase in the level of mature mRNA encoding MeCP2-e1 isoform, and an increase in the expression of MeCP2-e1 isoform in the cells of the subject. Without wishing to be bound by a certain theory, due to X inactivation, in a subject having cells having a WT allele and a mutant allele encoding a partially functional MeCP2, e.g., a hypomorphic allele, there can be about 50% cells having the mutant allele that express the WT allele but not the mutant allele, and another about 50% cells having the mutant allele that express the mutant allele but not the WT allele. In some cases, the methods and compositions provided herein can increase expression of a functional target protein having a target peptide sequence, e.g., MeCP2-e1 isoform encoded by exon 1, 3, and 4 of MECP2 gene, from the mutant hypomorphic allele in cells having pre-mRNA transcripts from the hypomorphic allele. In some cases, the methods and compositions provided herein can increase expression of MeCP2-e1 isoform in about 50% of cells having a hypomorphic mutant allele in a subject. In some cases, the methods and compositions restore the functional of MeCP2, e.g., equivalent to the functional level as having a normal amount of WT MeCP2 protein, in about 50% of cells having a hypomorphic mutant allele in a subject.

In some embodiments, the method is a method of increasing the expression of the target protein by cells of a subject having a pre-mRNA encoding the target protein and comprising an exon that contains an inefficient translation region or a PTC followed by an alternative start codon, wherein the subject has a disease or condition caused by a deficient amount or activity of target protein is caused by autosomal recessive inheritance. In some embodiments, the method is a method of increasing the expression of the target protein by cells of a subject having a pre-mRNA encoding the target protein and comprising an exon that contains an inefficient translation region or a PTC followed by an alternative start codon, wherein the subject has a disease or condition caused by a deficient amount or activity of target protein that is caused by autosomal dominant inheritance. In some embodiments, the disease or condition is caused by or associated with haploinsufficiency of the target gene encoding the target protein. In some embodiments, the method is a method of increasing the expression of the target protein by cells of a subject having a pre-mRNA encoding the target protein and comprising an exon that contains an inefficient translation region or a PTC followed by an alternative start codon, wherein the subject has a disease or condition caused by a deficient amount or activity of target protein is caused by X-linked dominant mutation. For instance, the method can be a method of increasing the expression of MeCP2-e1 isoform by cells (e.g., brain cells, e.g., neurons or glial cells) of a subject having a pre-mRNA encoding MeCP2 and comprising exon2, wherein the subject has Rett syndrome caused by a deficient amount or activity of MeCP2 that is caused by X-linked dominant mutation.

In some embodiments, the pre-mRNA transcript that encodes the protein that is causative of the disease or condition is targeted by the ASOs described herein. In some embodiments, a pre-mRNA transcript that encodes a protein that is not causative of the disease is targeted by the ASOs. For example, a disease that is the result of a mutation or deficiency of a first protein in a particular pathway may be ameliorated by targeting a pre-mRNA that encodes a second protein (e.g., containing a target peptide sequence), thereby increasing production of the second protein. In some embodiments, the function of the second protein is able to compensate for the mutation or deficiency of the first protein (which is causative of the disease or condition).

In some embodiments, the methods and compositions are applicable to treat a subject that has:

(a) a first mutant allele from which
(i) the target protein is produced at a reduced level compared to production from a wild-type allele,
(ii) the target protein is produced in a form having reduced function compared to an equivalent wild-type protein, or
(iii) the target protein is not produced; and

(b) a second mutant allele from which
(i) the target protein is produced at a reduced level compared to production from a wild-type allele,
(ii) the target protein is produced in a form having reduced function compared to an equivalent wild-type protein, or
(iii) the target protein is not produced.

In some embodiments, the level of the first processed mRNA encoding the first protein that comprises the target peptide sequence in the cell contacted with the therapeutic agent provided herein is increased as compared to the level of the first processed mRNA in a control cell that is otherwise identical but not contacted with the therapeutic agent. In some embodiments, the level of the first processed mRNA encoding the first protein that comprises the target peptide sequence in the cell contacted with the therapeutic agent provided herein is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of first processed mRNA in a control cell. In some embodiments, the level of the second processed mRNA that comprises the inefficient translation region and encodes the second protein comprising the target peptide sequence in the cell contacted with the therapeutic agent provided herein is decreased as compared to the level of the second processed mRNA in a control cell that is otherwise identical but not contacted with the therapeutic agent. In some embodiments, the level of the second processed mRNA that comprises the inefficient translation region and encodes the second protein comprising the target peptide sequence in the cell contacted with the therapeutic agent provided herein is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of second processed mRNA in a control cell.

In some embodiments, the therapeutic agent provided herein increases the expression of the target peptide sequence in the cell. In some embodiments, the level of the target peptide sequence in the cell contacted with the therapeutic agent is increased compared to the level of the target peptide sequence in a control cell that is otherwise identical but not contacted with the therapeutic agent. In some embodiments, the level of the target peptide sequence in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the target peptide sequence in a control cell.

### Inefficient Translation Region

As described herein, an inefficient translation region can refer to any region in an mRNA transcript that contributes to inefficient translation of a target peptide sequence encoded by the mRNA transcript, in a manner that a corresponding mRNA transcript without the inefficient translation region would have a higher translation efficiency for producing the target peptide sequence as compared to the mRNA transcript with the inefficient translation region. The inefficient translation region can decrease the translation efficiency of the mRNA transcript for the target peptide sequence at the stage of translation initiation, elongation, termination, or any combination thereof. In some embodiments, an mRNA transcript having the inefficient translation region has a translation efficiency about 10%, 20%, 30%, 40%, 50%, 60%, 70%, 80%, 90%, or 100% lower than a corresponding mRNA transcript without the inefficient translation region. In some embodiments, an mRNA transcript having the inefficient translation region has a translation efficiency about 1.2, 1.5, 1.8, 2, 2.5, 3, 3.5, 4, 4.5, 5, 5.5, 6, 6.5, 7, 7.5, 8, 9, 10, 11, 12, 15, 18, 20, 22, 25, 28, 30, 35, 40, 50, 60, 70, 80, 90, 100, 120, 150, 200, 300, 400, 500, 600, 700, 800, 900, 1000, 2000, 5000, 10⁴, 10⁵, or even greater times lower than a corresponding mRNA transcript without the inefficient translation region. In some embodiments, an mRNA transcript having the inefficient translation region has a translation efficiency at least about 10%, 20%, 30%, 40%, 50%, 60%, 70%, 80%, 90%, or 100% lower than a corresponding mRNA transcript without the inefficient translation region. In some embodiments, an mRNA transcript having the inefficient translation region has a translation efficiency at least about 1.2, 1.5, 1.8, 2, 2.5, 3, 3.5, 4, 4.5, 5, 5.5, 6, 6.5, 7, 7.5, 8, 9, 10, 11, 12, 15, 18, 20, 22, 25, 28, 30, 35, 40, 50, 60, 70, 80, 90, 100, 120, 150, 200, 300, 400, 500, 600, 700, 800, 900, 1000, 2000, 5000, 10⁴, or 10⁵ times lower than a corresponding mRNA transcript without the inefficient translation region.

In some embodiments, the inefficient translation region comprises a PTC followed by an alternative start codon. Without wishing to be bound by a certain theory, the motif or the exon containing the PTC and the alternative start codon can contribute to the inefficient translation of the downstream sequences because while the PTC induces premature termination of translation of the upstream open reading frame in the mRNA, the close proximity of the alternative start codon to the PTC can prevent the induction of nonsense-mediated decay, rather re-initiation of translation of the downstream open reading frame. In this regards, the efficiency of the translation initiation of the downstream open reading frame can be relatively much lower than the canonical open reading frame starting from the canonical start codon (e.g., upstream of the downstream open reading frame). As exemplified in the case of MECP2 mRNA processing, the removal of exon 2 that contains a PTC and an alternative start codon can lead to increased production of MeCP2-e1 isoform, however, the relative mature mRNA level can remain stable, suggesting the MECP2 e1 mRNA isoform, which is devoid of exon 2 containing the PTC and alternative start codon, has a higher translation efficiency as compared to MECP2 e2 mRNA isoform with exon 2. In some embodiments, the distance between PTC and the downstream start codon in the inefficient translation region is short, for instance, at most 75 nucleotides (nt), 70 nt, 60 nt, 55 nt, 50 nt, 45 nt, 40 nt, 35 nt, 30 nt, 27 nt, 24 nt, 21 nt, 18 nt, 15 nt, 12 nt, 10 nt, 9 nt, 6 nt, 3 nt, or 2 nt. In some embodiments, the distance between PTC and the downstream start codon in the inefficient translation region is about 3 nt, 6 nt, 9 nt, 10 nt, 12 nt, 15 nt, 18 nt, 21 nt, 24 nt, 27 nt, 30 nt, 35 nt, 40 nt, 45 nt, 50 nt, 55 nt, 60 nt, 70 nt, or 75 nt. In some cases, the distance between PTC and the downstream start codon in the inefficient translation region is at least 2 nt, 3 nt, 6 nt, 9 nt, 10 nt, 12 nt, 15 nt, 18 nt, 21 nt, 24 nt, 27 nt, 30 nt, 35 nt, 40 nt, 45 nt, 50 nt, or 55 nt. In some cases, distance between PTC and the downstream start codon in the inefficient translation region is 2 nt to 75 nt, 5 nt to 70 nt, 10 nt to 65 nt, 15 nt to 60 nt, 20 nt to 55 nt, 25 nt to 50 nt, 30 nt to 45 nt, 40 nt to 50 nt, 45 nt to 55 nt, 50 nt to 60 nt, 55 nt to 70 nt, or 60 nt to 75 nt.

In some embodiments, the inefficient translation region comprises a region that codes for proline-rich peptide sequence. Without wishing to be bound by a certain theory, mRNA sequence coding for proline-rich peptide sequence can contribute to inefficient translation, for instance, inefficient translation elongation, of the mRNA transcript that contains it. In some embodiments, the inefficient translation region encodes peptide sequence having at least about 5, 8, 10, 12, 15, 16, 18, 20, 22, 24, 26, 28, 30, 35, 40, 45, 50, 55, 60, 65, 70, 75, 80, 95, or 100 prolines. In some embodiments, the inefficient translation region encodes peptide sequence having contiguous peptide sequences consisting of at least about 5, 8, 10, 12, 15, 16, 18, 20, 22, 24, 26, 28, 30, 35, 40, 45, 50, 55, 60, 65, 70, 75, 80, 95, or 100 prolines. In some embodiments, the inefficient translation region encodes peptide sequence having at least about 5%, 10%, 20%, 30%, 40%, 50%, 60%, 70%, 80%, 90%, or 100% proline.

The inefficient translation region can be in any length. In some embodiments, the inefficient translation region can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the inefficient translation region can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the inefficient translation region can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the inefficient translation region may be longer than 1,000 nucleotides in length.

### Modulation of Translation of Various Targets

In aspects, the methods, compositions, and kits are applicable to modulation of translation of various targets, for instance, for target peptide sequences whose translation is modulated by an inefficient translation region present in a processed mRNA transcript encoding the target peptide sequence. In some cases, the inefficient translation region comprises at least a portion of an exon that comprises a PTC followed by an alternative start codon. Table 1 below lists exemplary target genes and their corresponding pre-mRNA transcript that can be processed into mRNA transcript having an exon that comprises a PTC followed by an alternative start codon. The sequences of the exons (and SEQ ID NOs) and their corresponding genomic coordinates are listed in the table. In some cases, the subject methods, compositions, and kits are applicable to modulation of translation of the target peptide sequences encoded by the target genes that are listed Table 1.

TABLE 1
Exemplary Genes and Exons Containing PTC and Alternative Start Codon

| | | SEQ | Sequence of Exon containing | Corresponding |
|---|---|---|---|---|
| | Pre-mRNA | ID | PTC and alternative start | Genomic |
| Gene | transcript | NO: | codon | Coordinates |
| ACIN1 | ENST000003 | 130 | AAAGGGATAAACAGCACTTCCAGGGGG | GRCh38/hg38: |
| | 57481.6 | | AGAAAAAAAATCATGTTATCAGAAAGC | chr14:23071125: |
| | | | AAAGAAGG | 23071186:− |
| ACTN1 | ENST000005 | 131 | ACATTCACGGCATGGTGTAACTCCCAC | GRCh38/hg38: |
| | 55616.5 | | CTCCGGAAGGCGGGGACACAGATCGAG | chr14:68925558: |
| | | | AACATCGAAGAGGACTTCCGGGATGGC | 68925672:− |
| | | | CTGAAGCTCATGCTGCTGCTGGAGGTC | |
| | | | ATCTCAG | |
| ACTN2 | ENST000005 | 132 | ACATCGTGAACACCCCTAAACCCGATG | GRCh38/hg38: |
| | 46208.5 | | AAAGAGCCATCATGACGTACGTCTCTT | chr1:236735635: |
| | | | GCTTCTACCACGCTTTTGCGGGCGCGG | 236735720:+ |
| | | | AGCAG | |
| ADH4 | ENST000005 | 133 | attttgatttagtggatttgaggcagg | GRCh38/hg38: |
| | 06705.5 | | gctttcatctctaacaaattcccaagt | chr4:99143166: |
| | | | gatgtgaataaatgcttgtccgaggac | 99143305:− |
| | | | cacacttcgagttgcaGAGATGTAAAA | |
| | | | CACATCTATTCTCCAGCAATTATCTGA | |
| | | | CTCAG | |
| ADH4 | ENST000005 | 134 | attttgatttagtggatttgaggcagg | GRCh38/hg38: |
| | 05590.5 | | gctttcatctctaacaaattcccaagt | chr4:99143166: |
| | | | gatgtgaataaatgcttgtccgaggac | 99143305:− |
| | | | cacacttcgagttgcaGAGATGTAAAA | |
| | | | CACATCTATTCTCCAGCAATTATCTGA | |
| | | | CTCAG | |
| AGER | ENST000003 | 135 | AGCCTGTGCCTCTGGAGGAGGTCCAAT | GRCh38/hg38: |
| | 75065.6 | | TGGTGGTGGAGCCAGAAGGTGGAGCAG | chr6:32182568: |
| | | | TAGCTCCTGGTGGAACCGTAACCCTGA | 32182698:− |
| | | | CCTGTGAAGTCCCTGCCCAGCCCTCTC | |
| | | | CTCAAATCCACTGGATGAAGGAT | |
| AKR1C3 | ENST000006 | 136 | aatcatctcttttgaaaaatcgattca | GRCh38/hg38: |
| | 02997.5 | | tcaaatgaatcttcagccaacaactgt | chr10:5077900: |
| | | | tcaagaaggatgcaaatatcacag | 5077977:+ |
| ALDH1A | ENST000005 | 137 | GTGTGAAAATTGGACTTGCTGGCAGAA | GRCh38/hg38: |
| 2 | 37372.5 | | TCCTTTTTGGGAGAAGATGAAGAATCA | chr15:58058012: |
| | | | GTGTGAGACTGTTTGGTTAAAATCCCC | 58058108:− |
| | | | AATAAAACTGAAACTG | |
| ALG8 | ENST000005 | 138 | GGCCAAGACTGAAAGCGTGGAAACCAA | GRCh38/hg38: |
| | 25761.2 | | GAAGCAACCATGAAATGGTGGTGTGTA | chr11:78138710: |
| | | | TACA | 78138767:− |
| AMPD2 | ENST000005 | 139 | GCCCAGCCACCATCAGTCACGTCACTC | GRCh38/hg38: |
| | 28667.5 | | CTGGGACTGAGGAGGCAGGGGAGGGAT | chr1:109620914: |
| | | | AAGGGGCAGAGATGGAGGCCCCACTCC | 109621266:+ |
| | | | CCGAGGTTGCCTAGACAACATGAGAAA | |
| | | | TCGTGGCCAGGGCCTCTTCCGCCTGCG | |
| | | | GAGCCGCTGCTTCCTGCATCAGTCACT | |
| | | | CCCGCTGGGGGCGGGGCGGAGGAAGGG | |
| | | | GTTGGATGTGGCAGAGCCAGGCCCCAG | |
| | | | CCGGTGCCGCTCAGACTCCCCCGCTGT | |
| | | | CGCCGCCGTGGTCCCAGCCATGGCATC | |
| | | | CTATCCATCTGGCTCTGGCAAGCCCAA | |
| | | | GGCCAAATATCCCTTTAAGAAGCGGGC | |
| | | | CAGCCTGCAGGCCTCCACTGCAGCTCC | |
| | | | AG | |
| AMT | ENST000005 | 140 | GACACCGCTCTATGACTTCCACCTGGC | GRCh38/hg38: |
| | 38581.6 | | CCACGGCGGGAAAATGGTGGCGTTTGC | chr3:49422104: |
| | | | GGGTTGGAGTCTGCCAGTGCAGTACCG | 49422257:− |
| | | | GGACAGTCACACTGACTCGCACCTGCA | |
| | | | CACACGCCAGCACTGCTCGCTCTTTGA | |
| | | | CGTGTCTCATATGCTGCAG | |
| AMT | ENST000004 | 141 | GACACCGCTCTATGACTTCCACCTGGC | GRCh38/hg38: |
| | 27987.6 | | CCACGGCGGGAAAATGGTGGCGTTTGC | chr3:49422104: |
| | | | GGGTTGGAGTCTGCCAGTGCAGTACCG | 49422257:− |
| | | | GGACAGTCACACTGACTCGCACCTGCA | |
| | | | CACACGCCAGCACTGCTCGCTCTTTGA | |
| | | | CGTGTCTCATATGCTGCAG | |
| AMT | ENST000006 | 142 | GACACCGCTCTATGACTTCCACCTGGC | GRCh38/hg38: |
| | 36865.1 | | CCACGGCGGGAAAATGGTGGCGTTTGC | chr3:49422104: |
| | | | GGGTTGGAGTCTGCCAGTGCAGTACCG | 49422257:− |
| | | | GGACAGTCACACTGACTCGCACCTGCA | |
| | | | CACACGCCAGCACTGCTCGCTCTTTGA | |
| | | | CGTGTCTCATATGCTGCAG | |
| ANKRD1 | ENST000006 | 143 | CAGACGGTTGATGATGAAGCGCCGGCC | GRCh38/hg38: |
| 1 | 42443.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000003 | 144 | CAGACGGTTGATGATGAAGCGCCGGCC | GRCh38/hg38: |
| 1 | 78330.7 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000006 | 145 | CAGACGGTTGATGATGAAGCGCCGGCC | GRCh38/hg38: |
| 1 | 45278.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000003 | 146 | ACGGTTGATGATGAAGCGCCGGCCGTG | GRCh38/hg38: |
| 1 | 30736.10 | | TAAATGAAGATCGGGTGAGGAGCAGGA | chr16:89316933: |
| | | | CGATGCCCAAGGGTGGGTGCCCTAAAG | 89317075:− |
| | | | CACCACAGCAGGAAGAGCTTCCCCTCA | |
| | | | GCAGCGACATGGTGGAGAAGCAGACTG | |
| | | | GGAAAAAG | |
| ANKRD1 | ENST000006 | 147 | CAGACGGTTGATGATGAAGCGCCGGCC | GRCh38/hg38: |
| 1 | 13312.4 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000006 | 148 | CAGACGGTTGATGATGAAGCGCCGGCC | GRCh38/hg38: |
| 1 | 46975.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000006 | 149 | ACGGTTGATGATGAAGCGCCGGCCGTG | GRCh38/hg38: |
| 1 | 42600.1 | | TAAATGAAGATCGGGTGAGGAGCAGGA | chr16:89316933: |
| | | | CGATGCCCAAGGGTGGGTGCCCTAAAG | 89317075:− |
| | | | CACCACAGCAGGAAGAGCTTCCCCTCA | |
| | | | GCAGCGACATGGTGGAGAAGCAGACTG | |
| | | | GGAAAAAG | |
| ANKRD1 | ENST000006 | 150 | CAGACGGTTGATGATGAAGCGCCGGCC | GRCh38/hg38: |
| 1 | 43964.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000006 | 151 | CAGACGGTTGATGATGAAGCGCCGGCC | GRCh38/hg38: |
| 1 | 44045.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000006 | 152 | ACGGTTGATGATGAAGCGCCGGCCGTG | GRCh38/hg38: |
| 1 | 44784.1 | | TAAATGAAGATCGGGTGAGGAGCAGGA | chr16:89316933: |
| | | | CGATGCCCAAGGGTGGGTGCCCTAAAG | 89317075:− |
| | | | CACCACAGCAGGAAGAGCTTCCCCTCA | |
| | | | GCAGCGACATGGTGGAGAAGCAGACTG | |
| | | | GGAAAAAG | |
| ANKRD1 | ENST000006 | 153 | CAGACGGTTGATGATGAAGCGCCGGCC | GRCh38/hg38: |
| 1 | 42695.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000005 | 154 | ACGGTTGATGATGAAGCGCCGGCCGTG | GRCh38/hg38: |
| 1 | 62275.6 | | TAAATGAAGATCGGGTGAGGAGCAGGA | chr16:89316933: |
| | | | CGATGCCCAAGGGTGGGTGCCCTAAAG | 89317075:− |
| | | | CACCACAGCAGGAAGAGCTTCCCCTCA | |
| | | | GCAGCGACATGGTGGAGAAGCAGACTG | |
| | | | GGAAAAAG | |
| ANKRD1 | ENST000006 | 155 | CAGACGGTTGATGATGAAGCGCCGGCC | GRCh38/hg38: |
| 1 | 47238.1 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKRD1 | ENST000003 | 156 | CAGACGGTTGATGATGAAGCGCCGGCC | GRCh38/hg38: |
| 1 | 01030.9 | | GTGTAAATGAAGATCGGGTGAGGAGCA | chr16:89316933: |
| | | | GGACGATGCCCAAGGGTGGGTGCCCTA | 89317078:− |
| | | | AAGCACCACAGCAGGAAGAGCTTCCCC | |
| | | | TCAGCAGCGACATGGTGGAGAAGCAGA | |
| | | | CTGGGAAAAAG | |
| ANKS3 | ENST000004 | 157 | CAAGGTGCAGAGCTAGAAATGAAAGAC | GRCh38/hg38: |
| | 46014.6 | | ATCCAGGGCTGGACAGCCCTCTTCCAC | chr16:4726659: |
| | | | TGTACCAGCGCCGGGCACCAGCACATG | 4726780:− |
| | | | GTCAGGTTCCTCTTGGACAGTGGAGCC | |
| | | | AATGCCAACGTGAG | |
| APOL4 | ENST000004 | 158 | CCTCAACATTCAGCAGAGGCCCCAGAT | GRCh38/hg38: |
| | 57630.6 | | CAGCGTCTGAGCCAGGCCAACAATGAC | chr22:36201700: |
| | | | CAAGGAGGATGGGATCCTGGGTGCAGC | 36201796:− |
| | | | TCATCACAAGCGTCGG | |
| APPL1 | ENST000004 | 159 | aatagagtccaagaagcagaGCAGCAA | GRCh38/hg38: |
| | 44459.1 | | AGAATATGAAGACTTTTAAAGCTCATG | chr3:57230714: |
| | | | | 57230767:+ |
| ARCN1 | ENST000003 | 160 | ATAACTTGATTTAAAACATCGTCCAGT | GRCh38/hg38: |
| | 59415.8 | | GTACCTGAGAGATGGCAGAATGTAATT | chr11:118573620: |
| | | | TGGTGGCAATCCTAATAAGTTCTATTG | 118573783:+ |
| | | | ACAACCCTTTAGATAAGAACTTGGATA | |
| | | | ATGGGGGGAATTCATGCTTGGACTTTA | |
| | | | GGCCACTGAACAGCTTTTCTCAGCCTC | |
| | | | AG | |
| ARMC8 | ENST000004 | 161 | gaccaggaatgaaagttactgggagat | GRCh38/hg38: |
| | 81646.5 | | aacttcgaaggattttgcaacaaattc | chr3:138188455: |
| | | | gagctgtccgactctgagaatg | 138188530:+ |
| ARMC8 | ENST000004 | 162 | gaccaggaatgaaagttactgggagat | GRCh38/hg38: |
| | 71453.5 | | aacttcgaaggattttgcaacaaattc | chr3:138188455: |
| | | | gagctgtccgactctgagaatg | 138188530:+ |
| ARNTL | ENST000004 | 163 | TTCACAGGTCGAATTTGGGGAGCACAA | GRCh38/hg38: |
| | 03510.7 | | TGGCTGGAGGTCAGATGCCCACTAGGA | chr11:13355226: |
| | | | GATGCTATGATTAATAT | 13355296:+ |
| ARNTL | ENST000005 | 164 | TTCACAGGTCGAATTTGGGGAGCACAA | GRCh38/hg38: |
| | 30357.5 | | TGGCTGGAGGTCAGATGCCCACTAGGA | chr11:13355226: |
| | | | GATGCTATGATTAATAT | 13355296:+ |
| BOC | ENST000004 | 165 | AGTGTTGCCAGGGACGGCAGTATCTCT | GRCh38/hg38: |
| | 64546.5 | | TTGTGTGACCCTGGCGGCTTATGGGAC | chr3:113249722: |
| | | | GTTGGCTTCAGACCTTTGTGATACACC | 113249899:+ |
| | | | ATGCTGCGTGGGACGATGACGGCGTGG | |
| | | | AGAGGAATGAGGCCTGAGGTCACACTG | |
| | | | GCTTGCCTCCTCCTAGCCACAGCAGGC | |
| | | | TGCTTTGCTGACTTGA | |
| BOC | ENST000002 | 166 | AGTGTTGCCAGGGACGGCAGTATCTCT | GRCh38/hg38: |
| | 73395.8 | | TTGTGTGACCCTGGCGGCTTATGGGAC | chr3:113249722: |
| | | | GTTGGCTTCAGACCTTTGTGATACACC | 113249899:+ |
| | | | ATGCTGCGTGGGACGATGACGGCGTGG | |
| | | | AGAGGAATGAGGCCTGAGGTCACACTG | |
| | | | GCTTGCCTCCTCCTAGCCACAGCAGGC | |
| | | | TGCTTTGCTGACTTGA | |
| BOC | ENST000004 | 167 | AGTGTTGCCAGGGACGGCAGTATCTCT | GRCh38/hg38: |
| | 85230.5 | | TTGTGTGACCCTGGCGGCTTATGGGAC | chr3:113249722: |
| | | | GTTGGCTTCAGACCTTTGTGATACACC | 113249899:+ |
| | | | ATGCTGCGTGGGACGATGACGGCGTGG | |
| | | | AGAGGAATGAGGCCTGAGGTCACACTG | |
| | | | GCTTGCCTCCTCCTAGCCACAGCAGGC | |
| | | | TGCTTTGCTGACTTGA | |
| BOC | ENST000003 | 168 | AGTGTTGCCAGGGACGGCAGTATCTCT | GRCh38/hg38: |
| | 55385.7 | | TTGTGTGACCCTGGCGGCTTATGGGAC | chr3:113249722: |
| | | | GTTGGCTTCAGACCTTTGTGATACACC | 113249899:+ |
| | | | ATGCTGCGTGGGACGATGACGGCGTGG | |
| | | | AGAGGAATGAGGCCTGAGGTCACACTG | |
| | | | GCTTGCCTCCTCCTAGCCACAGCAGGC | |
| | | | TGCTTTGCTGACTTGA | |
| BRCA1 | ENST000004 | 169 | ATTTTGCATGCTGAAACTTCTCAACCA | GRCh38/hg38: |
| | 93795.5 | | GAAGAAAGGGCCTTCACAGTGTCCTTT | chr17:43106456: |
| | | | ATGTAAGAATGATATAACCAAAAG | 43106533:− |
| BRCA1 | ENST000004 | 170 | ATTTTGCATGCTGAAACTTCTCAACCA | GRCh38/hg38: |
| | 93919.5 | | GAAGAAAGGGCCTTCACAGTGTCCTTT | chr17:43106456: |
| | | | ATGTAAGAATGATATAACCAAAAG | 43106533:− |
| C8B | ENST000005 | 171 | GATCAAGTCCAGGATCATGGTTTTCGA | GRCh38/hg38: |
| | 35057.5 | | GGATCCCTGTGACCGAATCTAGCCTAC | chr1:56959529: |
| | | | ATTTCCAGCACAATGGACACTTGTATG | 56959657:− |
| | | | ACTCTGGCcttcactctctctgggcgt | |
| | | | ttcttcatgctgctttctcag | |
| C8B | ENST000005 | 172 | GATCAAGTCCAGGATCATGGTTTTCGA | GRCh38/hg38: |
| | 35057.5 | | GGATCCCTGTGACCGAATCTAGCCTAC | chr1:56959529: |
| | | | ATTTCCAGCACAATGGACACTTGTATG | 56959657:− |
| | | | ACTCTGGCcttcactctctctgggcgt | |
| | | | ttcttcatgctgctttctcag | |
| CALM1 | ENST000004 | 173 | GCATCCTTCTTTAAAAGAGAAATCCAC | GRCh38/hg38: |
| | 47653.7 | | GTTAGCTCTCCTTGAGGTCTCGAGTTC | chr14:90398991: |
| | | | CCTCGGCTGGAGGCACAGGTTCAGTGG | 90399087:+ |
| | | | AGACCAAATAATGCAG | |
| CALM3 | ENST000005 | 174 | GAGGCCTTCTCCCTCTTTGACAAGGAT | GRCh38/hg38: |
| | 94523.5 | | GGAGATGGCACTATCACCACCAAGGAG | chr19:46608205: |
| | | | TTGGGGACAGTGATGAGATCCCTGGGA | 46608340:+ |
| | | | CAGAACCCCACTGAAGCAGAGCTGCAG | |
| | | | GATATGATCAATGAGGTGGATGCAGAT | |
| | | | G | |
| CASP5 | ENST000004 | 175 | AGCTTTGTCTAAAGCAAGGGCAAAACT | GRCh38/hg38: |
| | 56094.1 | | CTTTAAGCTGTGCCCAGAGTTGAAGGA | chr11:105008807: |
| | | | GTCTTTATATTTCTGATAGAAGACAGT | 105009053:− |
| | | | GGCAAAAAAAAAAGGCGTAAGAATTTT | |
| | | | GAAGCTATGTTCAAAGGTATCCTTCAG | |
| | | | AGTGGATTGGATAACTTCGTGATAAAC | |
| | | | CACATGCTAAAGAACAACGTGGCTGGA | |
| | | | CAAACATCTATCCAGACCCTAGTACCT | |
| | | | AATACGGATCAAAAGTCGACCAGTGTA | |
| | | | AAAA | |
| CAST | ENST000005 | 176 | ATAAAATAAAATTATACCTGGGGCTGT | GRCh38/hg38: |
| | 11049.5 | | GCTCTTATATTAGGCCATTCCAGTCAG | chr5:96726754: |
| | | | CCAACAGATGGAAGGACCACATCTTCC | 96726859:+ |
| | | | TAACAAGAAAAAACACAAAAAACAG | |
| CEP19 | ENST000004 | 177 | CAGGAAGTCATTATGCAACTTACACAT | GRCh38/hg38: |
| | 09690.3 | | ATTCATCAGATTTCCTCTGACTTACCC | chr3:196708528: |
| | | | GGACATGTACATGGGAATGATGTGCAC | 196708727:− |
| | | | TGCCAAGAAATGTGGGATTAGGTTTCA | |
| | | | GCCTCCAGCTATTATCTTAATCTATGA | |
| | | | GAGTGAAATCAAGGGGAAAATTCGCCA | |
| | | | GCGCATTATGCCAGTTCGAAACTTTTC | |
| | | | AAAGTTTTCAG | |
| CEP57 | ENST000005 | 178 | tgtgcagactagggaaatgaagtacaa | GRCh38/hg38: |
| | 44522.5 | | tgttgaccagaattgat | chr11:95795516: |
| | | | | 95795559:+ |
| CHEK1 | ENST000005 | 179 | AGTTCAACTTGCTGTGAATAGAGTAAC | GRCh38/hg38: |
| | 31607.5 | | TGAAGAAGCAGTCGCAGTGAAGATTGT | chr11:125627607: |
| | | | AGATATGAAGCGTGCCGTAGACTGTCC | 125627830:+ |
| | | | AGAAAATATTAAGAAAGAGATCTGTAT | |
| | | | CAATAAAATGCTAAATCATGAAAATGT | |
| | | | AGTAAAATTCTATGGTCACAGGAGAGA | |
| | | | AGGCAATATCCAATATTTATTTCTGGA | |
| | | | GTACTGTAGTGGAGGAGAGCTTTTTGA | |
| | | | CAGAATAG | |
| CIB2 | ENST000005 | 180 | GCTGCATTCGCGATTCTATGAGCTGGC | GRCh38/hg38: |
| | 39011.5 | | CCCCAACCTCGTCCCAATGGACTACAG | chr15:78111165: |
| | | | GAAGAGCCCCATCGTCCACGTGCCCAT | 78111276:− |
| | | | GAGCCTCATCATCCAGATGCCAGAGCT | |
| | | | CCGG | |
| CIB2 | ENST000005 | 181 | GCTGCATTCGCGATTCTATGAGCTGGC | GRCh38/hg38: |
| | 60618.5 | | CCCCAACCTCGTCCCAATGGACTACAG | chr15:78111165: |
| | | | GAAGAGCCCCATCGTCCACGTGCCCAT | 78111276:− |
| | | | GAGCCTCATCATCCAGATGCCAGAGCT | |
| | | | CCGG | |
| COX4I1 | ENST000005 | 182 | TGTTGTGAAGAGCGAAGACTTTTCGCT | GRCh38/hg38: |
| | 66405.5 | | CCCAGCTTATATGGATCGGCGTGACCA | chr16:85804941: |
| | | | CCCCTTGCCGGAGGTGGCCCATGTCAA | 85805104:+ |
| | | | GCACCTGTCTGCCAGCCAGAAGGCATT | |
| | | | GAAGGAGAAGGAGAAGGCCTCCTGGAG | |
| | | | CAGCCTCTCCATGGATGAGAAAGTCGA | |
| | | | GT | |
| DCLRE1 | ENST000003 | 183 | ATATCTATTGAAATCGAGACTCCTACC | GRCh38/hg38: |
| C | 57717.6 | | CAGATATCTTTAGTGGATGAAGCATCA | chr10:14939810: |
| | | | GGAGAG | 14939869:− |
| DECR1 | ENST000005 | 184 | gcaccttactagggatatgaaggtgag | GRCh38/hg38: |
| | 19410.5 | | aagaagagctgattcctgctgtcaaga | chr8:90005292: |
| | | | agcatagagtccaatggCATTATG | 90005369:+ |
| DECR1 | ENST000005 | 185 | gtgatgaacagcctccttctaaatcat | GRCh38/hg38: |
| | 17761.5 | | tgaccacatttcttattctttcctgag | chr8:90015541: |
| | | | gataaatttcaagaTATCCTGAACCAA | 90015666:+ |
| | | | CTGATGACAGCCTATTTAGCTACAGGA | |
| | | | ATACTCTGGAAGGAAATG | |
| DHFR | ENST000005 | 186 | GTAAACAGAATCTGGTGATTATGGGTA | GRCh38/hg38: |
| | 04396.1 | | AGAAGACCTGGTTCTCCATTCCTGAGA | chr5:80649389: |
| | | | AGAATCGACCTTTAAAGGGTAGAATTA | 80649494:− |
| | | | ATTTAGTTCTCAGCAGAGAACTCAA | |
| DKK2 | ENST000005 | 187 | GCCTACCCTTGTAGCAGTGATAAGGAG | GRCh38/hg38: |
| | 13208.5 | | TGTGAAGTTGGGAGGTATTGCCACAGT | chr4:106925799: |
| | | | CCCCACCAAGGATCATCGGCCTGCATG | 106925949:− |
| | | | GTGTGTCGGAGAAAAAAGAAGCGCTGC | |
| | | | CACCGAGATGGCATGTGCTGCCCCAGT | |
| | | | ACCCGCTGCAATAATG | |
| DLG2 | ENST000004 | 188 | GTGCCGATATCACATAAGATTTCACCA | GRCh38/hg38: |
| | 34967.1 | | GGGCTTCTTCTAGCCAGAAGACATGTC | chr11:83651839: |
| | | | CTTTGACAGCCAATGTGACCTTCGTAG | 83651930:− |
| | | | CTCTGAGCCAG | |
| DOK2 | ENST000005 | 189 | TCGGCCCCCACAAGGAATTTGCTGTGA | GRCh38/hg38: |
| | 18197.1 | | CCATGAGACCTACAGAAGCCAGTGAGA | chr8:21910673: |
| | | | GGTGCCACCTGCGGGGGTCCTATACCC | 21910857:− |
| | | | TCCGGGCTGGGGAGAGTGCCCTGGAGC | |
| | | | TGTGGGGTGGGCCCGAGCCAGGGACCC | |
| | | | AGCTGTACGACTGGCCCTACAGGTTTC | |
| | | | TGCGGCGCTTTGGGCGGGACAAG | |
| DPF3 | ENST000005 | 190 | gccctttcaagaatcctatgaaagttg | GRCh38/hg38: |
| | 46183.1 | | tggatcatctccccggaaaacacgcat | chr14:72879804: |
| | | | atagatgtgaacatctgcctatggttt | 72879947:− |
| | | | tatggggttcacagacctggaagagcc | |
| | | | catctctggatgccCTGGAGGCCCATG | |
| | | | GGCTCTAGG | |
| DPF3 | ENST000006 | 191 | gccctttcaagaatcctatgaaagttg | GRCh38/hg38: |
| | 14862.4 | | tggatcatctccccggaaaacacgcat | chr14:72879804: |
| | | | atagatgtgaacatctgcctatggttt | 72879947:− |
| | | | tatggggttcacagacctggaagagcc | |
| | | | catctctggatgccCTGGAGGCCCATG | |
| | | | GGCTCTAGG | |
| DTNA | ENST000005 | 192 | AGATGTGCTTAGAGTGTTTGGGAGAGT | GRCh38/hg38: |
| | 88949.5 | | TTCAGCCAGGCCTTCCAATTTCCTAGA | chr18:34757158: |
| | | | CCCCATGTGTGGTACTGTGTCTTCACC | 34757288:+ |
| | | | TGGAATTGTGACCTTTGTGCAGTTACA | |
| | | | AATCCAACAGAGCAAGGAGCCTA | |
| DTNA | ENST000005 | 193 | AGATGTGCTTAGAGTGTTTGGGAGAGT | GRCh38/hg38: |
| | 88949.5 | | TTCAGCCAGGCCTTCCAATTTCCTAGA | chr18:34757158: |
| | | | CCCCATGTGTGGTACTGTGTCTTCACC | 34757288:+ |
| | | | TGGAATTGTGACCTTTGTGCAGTTACA | |
| | | | AATCCAACAGAGCAAGGAGCCTA | |
| DYRK1A | ENST000006 | 194 | CCAAATATGAGGTGACGTTTAAGCTGC | GRCh38/hg38: |
| | 45774.1 | | TACTTGAAAGAGAAGTGGGAGTTAGGC | chr21:37456130: |
| | | | AGAGCAGTAGGGGAATCATGTTTGGGG | 37456308:+ |
| | | | AAGAGTGAAGAGTGTACTTGAGAGAGT | |
| | | | GTGGAGGTGCCTTGGAGGAGCTGGAGC | |
| | | | CCAGAGGTGCCCCATGAGAACAACACA | |
| | | | GGAGGCTGCAGGTGGAG | |
| FOLH1 | ENST000003 | 195 | gttaaaagccaactgcaaggtctaatg | GRCh38/hg38: |
| | 40334.11 | | actgcaggatctagctatccattgttt | chr11:49206778: |
| | | | ctggccGCCTATGCGTGCACTGGGTGT | 49206874:− |
| | | | CTGGCAGAGAGGCTGG | |
| FUZ | ENST000005 | 196 | CATCACCCTCATTGTTCTGTCATCTGA | GRCh38/hg38: |
| | 29302.1 | | GGTGGGCATCTCTGAGCTGAGGCTGGA | chr19:49812251: |
| | | | GAGACTACTCCAAATGGTGTTTGGAGC | 49812335:− |
| | | | CATG | |
| GANAB | ENST000005 | 197 | GTGTTGCTGGTGCTAGAGCTTCAGGGG | GRCh38/hg38: |
| | 40933.5 | | CTTCAAAAGAACATGACTCGGTTCAGG | chr11:62638983: |
| | | | ATTGATGAGCTGGAGCCTCGGCGACCC | 62639110:− |
| | | | CGATACCGTGTACCAGATGTTTTGGTG | |
| | | | GCTGATCCACCAATAGCCCG | |
| GEMIN4 | ENST000005 | 198 | AGCTAGGGCCATGAGTACCTTGTTTTT | GRCh38/hg38: |
| | 73482.5 | | GACTGGAAGGAGCTGTGGGGTGGACCG | chr17:749814: |
| | | | TTTCCCTGAAAGCTAGAAGAATGTTTG | 749909:− |
| | | | AAGCCTGTTCCCAAG | |
| GGA3 | ENST000005 | 199 | TACCTGGGGGACAGGGTGTCTGAGAAA | GRCh38/hg38: |
| | 80799.2 | | GTGAAGACCAAGGTTATTGAGCTGCTG | chr17:75243447: |
| | | | TACAGCTGGACCATGGCCCTGCCAGAA | 75243570:− |
| | | | GAAGCAAAGATCAAAGACGCCTACCAC | |
| | | | ATGCTGAAGAGACAGG | |
| GOSR2 | ENST000005 | 200 | ggttcatctatattgtagcctgttacc | GRCh38/hg38: |
| | 70879.2 | | agaatttcattctgttttatgacggaa | chr17:46924261: |
| | | | taatattccgttttatgtgtctaccac | 46924468:+ |
| | | | attttgttatccattcatctgttgatg | |
| | | | gacacttgagttgtttccaccattggg | |
| | | | ctgttgagaataatactgctatgaact | |
| | | | ctggtgtagaagtatctgttttagttt | |
| | | | ctcttctcaattttttgag | |
| GPM6A | ENST000002 | 201 | ATTCTACTGAAGAAAGGTAGCCATGGA | GRCh38/hg38: |
| | 80187.11 | | AGAGAATATGGAAGAGGGACAGACACA | chr4:175812191: |
| | | | AAAAG | 175812249:− |
| GRB10 | ENST000006 | 202 | gttctgaaaatattgtgcacagggcag | GRCh38/hg38: |
| | 45075.1 | | gctgaggacacagccacgtgataccca | chr7:50710875: |
| | | | ctgtagagaGAGGGAGAGAGAGACCTC | 50710990:− |
| | | | CTATGCAAGCTGCCGGCCCTCTGTTCC | |
| | | | GTAGTAAG | |
| GSN | ENST000004 | 203 | TTCTTGCAGATACAGCGCTAGGAAAAG | GRCh38/hg38: |
| | 77104.2 | | GGGAGTAATTCAGGTCTAGAATGGAAA | chr9:121300056: |
| | | | AACTGTTTTGTTGCTTT | 121300126:+ |
| HDAC8 | ENST000004 | 204 | GCCAGTATGGTGCATTCTTTGATTGAA | GRCh38/hg38: |
| | 21523.6 | | GCATATGCACTGCATAAGCAGATGAG | chrX:72572057: |
| | | | | 72572109:− |
| HIVEP1 | ENST000004 | 205 | GAAACGTGGTGGGTGCCGTTTTGACAC | GRCh38/hg38: |
| | 42081.6 | | CGACCTATCCTTTGTGAGGTTGAAGAA | chr6:12020248: |
| | | | TTTTGGAAGAGCATGAGAGCAGCACTT | 12020418:+ |
| | | | GGTGTTCAGAAGAAAGGCTTAGCATGT | |
| | | | TGTCCTGGGTTCCGTGGAGAAGGGTCA | |
| | | | CTGGACTGCACGGGGTGGCTCTTCCGC | |
| | | | ATGGTCTTG | |
| HK1 | ENST000004 | 206 | CATTGTTGATGCTCTTGAGAGCATCAG | GRCh38/hg38: |
| | 21088.5 | | CCAGGACATTAATGTGCACCACTGTGG | chr10:69300769: |
| | | | TGGCGTGGAAAGATGGCAAAAAGAGCC | 69300861:+ |
| | | | CTGcatgatttt | |
| HK1 | ENST000003 | 207 | CATTGTTGATGCTCTTGAGAGCATCAG | GRCh38/hg38: |
| | 60289.6 | | CCAGGACATTAATGTGCACCACTGTGG | chr10:69300769: |
| | | | TGGCGTGGAAAGATGGCAAAAAGAGCC | 69300861:+ |
| | | | CTGcatgatttt | |
| IFNGR1 | ENST000004 | 208 | TGGCCATAATATAATTGTGATAATGTA | GRCh38/hg38: |
| | 14770.5 | | ATAAAATCAGGAAACATTACCTGAAGC | chr6:137215267: |
| | | | AGATGCTTTTGAAGTCACCAGAAAATA | 137215377:− |
| | | | GTCTTCTCCAATTCCAATTTAAATATG | |
| | | | GAG | |
| IKBKB | ENST000003 | 209 | AGTTAGCACGACATCAGTATGAGCTGG | GRCh38/hg38: |
| | 42222.6 | | TCACCTTCCCTGACAACGCAGACATGT | chr8:42272083: |
| | | | GGGGCCTGGGAAATGAAAGAGCGCCTT | 42272205:+ |
| | | | GGGACAGGGGGATTTGGAAATGTCATC | |
| | | | CGATGGCACAATCAG | |
| IKBKB | ENST000006 | 210 | AGTTAGCACGACATCAGTATGAGCTGG | GRCh38/hg38: |
| | 49612.1 | | TCACCTTCCCTGACAACGCAGACATGT | chr8:42272083: |
| | | | GGGGCCTGGGAAATGAAAGAGCGCCTT | 42272205:+ |
| | | | GGGACAGGGGGATTTGGAAATGTCATC | |
| | | | CGATGGCACAATCAG | |
| IKBKB | ENST000005 | 211 | AGTTAGCACGACATCAGTATGAGCTGG | GRCh38/hg38: |
| | 20810.6 | | TCACCTTCCCTGACAACGCAGACATGT | chr8:42272083: |
| | | | GGGGCCTGGGAAATGAAAGAGCGCCTT | 42272205:+ |
| | | | GGGACAGGGGGATTTGGAAATGTCATC | |
| | | | CGATGGCACAATCAG | |
| IKBKB | ENST000006 | 212 | AGTTAGCACGACATCAGTATGAGCTGG | GRCh38/hg38: |
| | 29753.1 | | TCACCTTCCCTGACAACGCAGACATGT | chr8:42272083: |
| | | | GGGGCCTGGGAAATGAAAGAGCGCCTT | 42272205:+ |
| | | | GGGACAGGGGGATTTGGAAATGTCATC | |
| | | | CGATGGCACAATCAG | |
| ISCU | ENST000003 | 213 | GTATCTCAAATCTGTGAAGTATTGTAG | GRCh38/hg38: |
| | 92807.8 | | AGGAGACACAAAAGGAATTGGGGGTCA | chr12:108564060: |
| | | | CAAATGGTTCTCATTGACATGAGTGTA | 108564155:+ |
| | | | GACCTTTCTACTCAG | |
| KARS | ENST000005 | 214 | GTGGTGGTAATCATTAGTTCCAGGGTG | GRCh38/hg38: |
| | 64578.5 | | CTCTGCCATGTTGACGCAAGCTGCTGT | chr16:75644283: |
| | | | AAGGCTTGTTAGGGGGTCCCTGCGCAA | 75644462:− |
| | | | AACCTCCTGGGCAGAGTGGGGTCACAG | |
| | | | GGAACTGCGACTGGGTCAACTTGCTCC | |
| | | | TTTCACAGCGCCTCACAAGGACAAGTC | |
| | | | ATTTTCTGATCAAAGAAG | |
| KARS | ENST000003 | 215 | GTGGTGGTAATCATTAGTTCCAGGGTG | GRCh38/hg38: |
| | 19410.9 | | CTCTGCCATGTTGACGCAAGCTGCTGT | chr16:75644283: |
| | | | AAGGCTTGTTAGGGGGTCCCTGCGCAA | 75644462:− |
| | | | AACCTCCTGGGCAGAGTGGGGTCACAG | |
| | | | GGAACTGCGACTGGGTCAACTTGCTCC | |
| | | | TTTCACAGCGCCTCACAAGGACAAGTC | |
| | | | ATTTTCTGATCAAAGAAG | |
| KIAA03 | ENST000005 | 216 | TTTGCTTATGGTGGATGCACTCATGGC | GRCh38/hg38: |
| 19 | 35378.5 | | AAAAAAATCACTGGTGAGCATCATTTA | chr6:24600619: |
| | | | AGAAGACCCATGACTAGACTGGGCTGG | 24600709:− |
| | | | CCGAGCCCAT | |
| KIAA05 | ENST000006 | 217 | GTTCATCAGACTTAACTTCTGCTAGAA | GRCh38/hg38: |
| 86 | 19722.4 | | ATTGTTACCAGCCTCTATTAGAAAATC | chr14:58429363: |
| | | | CCATGGTGTCAGAAAGT | 58429433:+ |
| KIAA05 | ENST000004 | 218 | GTTCATCAGACTTAACTTCTGCTAGAA | GRCh38/hg38: |
| 86 | 23743.7 | | ATTGTTACCAGCCTCTATTAGAAAATC | chr14:58429363: |
| | | | CCATGGTGTCAGAAAGT | 58429433:+ |
| KIZ | ENST000006 | 219 | TGAAAAGAAGAGATTGGACCTGGAAAA | GRCh38/hg38: |
| | 20891.4 | | GAAACTTTATGAATATAATCAGTCTGA | chr20:21132097: |
| | | | TACATGCAG | 21132159:+ |
| KLK6 | ENST000003 | 220 | GAATCTTCAGGTCTTCCTGGGGAAGCA | GRCh38/hg38: |
| | 91808.5 | | TAACCTTCGGCAAAGGGAGAGTTCCCA | chr19:50963302: |
| | | | GGAGCAGAGTTCTGTTGTCCGGGCTGT | 50963549:− |
| | | | GATCCACCCTGACTATGATGCCGCCAG | |
| | | | CCATGACCAGGACATCATGCTGTTGCG | |
| | | | CCTGGCACGCCCAGCCAAACTCTCTGA | |
| | | | ACTCATCCAGCCCCTTCCCCTGGAGAG | |
| | | | GGACTGCTCAGCCAACACCACCAGCTG | |
| | | | CCACATCCTGGGCTGGGGCAAGACAGC | |
| | | | AGATG | |
| LMNA | ENST000003 | 221 | ATTTGCCAGTGATGGGAAGAGTTAGAA | GRCh38/hg38: |
| | 68297.5 | | ACAGGATGCCCAGCCCTTCTCGCCTCA | chr1:156126736: |
| | | | AGAGGCCACTGGGATGCAGCCACTCCT | 156126915:+ |
| | | | GTGCTTGGGGAACCTGGAGGATGCAAG | |
| | | | GGAAAGGACTGGCACTCTGCTGGCACA | |
| | | | GCACCCGGCCTGGGGCAGGACACGGGC | |
| | | | GAAGCCAGGGTCTCCCCT | |
| LMNTD1 | ENST000004 | 222 | GAAAGAAAAGAGACTTCTTTTCTAGCC | GRCh38/hg38: |
| | 58174.6 | | AAGATGAAAGATACACAAGACATTCAG | chr12:25552871: |
| | | | GAAGCTTCGAAGGCAATGCAGAATAAA | 25552989:− |
| | | | GTCCATGAGCAGGAAGATAAGAATGAG | |
| | | | AAACAAAAACA | |
| LMNTD1 | ENST000004 | 223 | GAAAGAAAAGAGACTTCTTTTCTAGCC | GRCh38/hg38: |
| | 13632.6 | | AAGATGAAAGATACACAAGACATTCAG | chr12:25552871: |
| | | | GAAGCTTCGAAGGCAATGCAGAATAAA | 25552989:− |
| | | | GTCCATGAGCAGGAAGATAAGAATGAG | |
| | | | AAACAAAAACA | |
| LRRC7 | ENST000003 | 224 | GAGCCCGCTACATGAAAAAAAATGCAA | GRCh38/hg38: |
| | 10961.9 | | GTCAAAGTGGTCACAACCTAGAAGTCT | chr1:69716142: |
| | | | GCAGAGACAGGAATAACTAGCAA | 69716218:+ |
| LRTOMT | ENST000005 | 225 | GCTGAACCCAGACTCCCAGGGCACCTG | GRCh38/hg38: |
| | 41614.5 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000006 | 226 | GCTGAACCCAGACTCCCAGGGCACCTG | GRCh38/hg38: |
| | 47530.1 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000005 | 227 | GCTGAACCCAGACTCCCAGGGCACCTG | GRCh38/hg38: |
| | 42846.5 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000005 | 228 | GCTGAACCCAGACTCCCAGGGCACCTG | GRCh38/hg38: |
| | 35883.6 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000005 | 229 | GCTGAACCCAGACTCCCAGGGCACCTG | GRCh38/hg38: |
| | 36917.2 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000002 | 230 | GCTGAACCCAGACTCCCAGGGCACCTG | GRCh38/hg38: |
| | 89488.7 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000006 | 231 | GCTGAACCCAGACTCCCAGGGCACCTG | GRCh38/hg38: |
| | 42648.1 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000006 | 232 | GCTGAACCCAGACTCCCAGGGCACCTG | GRCh38/hg38: |
| | 42478.1 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000003 | 233 | GCTGAACCCAGACTCCCAGGGCACCTG | GRCh38/hg38: |
| | 24866.11 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000006 | 234 | GCTGAACCCAGACTCCCAGGGCACCTG | GRCh38/hg38: |
| | 45358.1 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LRTOMT | ENST000005 | 235 | GCTGAACCCAGACTCCCAGGGCACCTG | GRCh38/hg38: |
| | 38478.5 | | CTTGCACCTTTGAATGATGGCCTGAAC | chr11:72089029: |
| | | | TATGAACAAACGGGACTATATGAACAC | 72089165:+ |
| | | | TTCGGTACAGGAGCCCCCTCTTGACTA | |
| | | | CTCCTTCAGAAGCATCCACGTCATTCA | |
| | | | AG | |
| LZTFL1 | ENST000005 | 236 | GCAGAGTTGGGCCTAAATGAGCACCAT | GRCh38/hg38: |
| | 36047.5 | | CAAAATGAAGTTATTAATTATATGCGT | chr3:45837927: |
| | | | TTTGCTCGTTCAAAGAGAGGCTTGAGA | 45838051:− |
| | | | CTCAAAACTGTAGATTCCTGCTTCCAA | |
| | | | GACCTCAAGGAGAGCAG | |
| MAGI2 | ENST000006 | 237 | ggagggtacctcttgaatgagggtcct | GRCh38/hg38: |
| | 36717.1 | | atgacctgcttcaggggaaggttagaa | chr7:78554567: |
| | | | aattcttcctgggatttatggccgcct | 78554671:− |
| | | | tccggggagatgggcgagaagaag | |
| MECP2 | ENST000006 | 238 | GCTCCATAAAAATACAGACTCACCAGT | GRCh38/hg38: |
| | 28176.2 | | TCCTGCTTTGATGTGACATGTGACTCC | chrX:154092184: |
| | | | CCAGAATACACCTTGCTTCTGTAGACC | 154092307:− |
| | | | AGCTCCAACAGGATTCCATGGTAGCTG | |
| | | | GGATGTTAGGGCTCAG | |
| MECP2 | ENST000003 | 239 | GCTCCATAAAAATACAGACTCACCAGT | GRCh38/hg38: |
| | 03391.10 | | TCCTGCTTTGATGTGACATGTGACTCC | chrX:154092184: |
| | | | CCAGAATACACCTTGCTTCTGTAGACC | 154092307:− |
| | | | AGCTCCAACAGGATTCCATGGTAGCTG | |
| | | | GGATGTTAGGGCTCAG | |
| MERTK | ENST000004 | 240 | CATAACCAGTGTGCAGCGTTCAGACAA | GRCh38/hg38: |
| | 09780.5 | | TGGGTCGTATATCTGTAAGATGAAAAT | chr2:111944960: |
| | | | AAACAATGAAGAGATCGTGTCTGATCC | 111945060:+ |
| | | | CATCTACATCGAAGTACAAG | |
| MFSD2A | ENST000004 | 241 | GATCATCTTCTCCACGCCCCTGGCCGT | GRCh38/hg38: |
| | 20632.6 | | CATTGCCTACTTCCTCATCTGGTTCGT | chr1:39965211: |
| | | | GCCCGACTTCCCACACGGCCAGACCTA | 39965334:+ |
| | | | TTGGTACCTGCTTTTCTATTGCCTCTT | |
| | | | TGAAACAATGGTCACG | |
| MLF1 | ENST000004 | 242 | TGAGTCCATTCTTGCACACCGAGAAAA | GRCh38/hg38: |
| | 82628.5 | | TATGCGACAGATGATAAGAAGTTTTTC | chr3:158592434: |
| | | | TGAACCCTTTGGAAGAGACTTGCTCAG | 158592581:+ |
| | | | TATCTCTGATGGTAGAGGGAGAGCTCA | |
| | | | TAATCGTAGAGGACATAATGATGGTGA | |
| | | | AGATTCTTTGACT | |
| MLF1 | ENST000006 | 243 | TGAGTCCATTCTTGCACACCGAGAAAA | GRCh38/hg38: |
| | 19577.4 | | TATGCGACAGATGATAAGAAGTTTTTC | chr3:158592434: |
| | | | TGAACCCTTTGGAAGAGACTTGCTCAG | 158592581:+ |
| | | | TATCTCTGATGGTAGAGGGAGAGCTCA | |
| | | | TAATCGTAGAGGACATAATGATGGTGA | |
| | | | AGATTCTTTGACT | |
| MLF1 | ENST000003 | 244 | TGAGTCCATTCTTGCACACCGAGAAAA | GRCh38/hg38: |
| | 59117.9 | | TATGCGACAGATGATAAGAAGTTTTTC | chr3:158592434: |
| | | | TGAACCCTTTGGAAGAGACTTGCTCAG | 158592581:+ |
| | | | TATCTCTGATGGTAGAGGGAGAGCTCA | |
| | | | TAATCGTAGAGGACATAATGATGGTGA | |
| | | | AGATTCTTTGACT | |
| MLF1 | ENST000006 | 245 | TGAGTCCATTCTTGCACACCGAGAAAA | GRCh38/hg38: |
| | 18075.4 | | TATGCGACAGATGATAAGAAGTTTTTC | chr3:158592434: |
| | | | TGAACCCTTTGGAAGAGACTTGCTCAG | 158592581:+ |
| | | | TATCTCTGATGGTAGAGGGAGAGCTCA | |
| | | | TAATCGTAGAGGACATAATGATGGTGA | |
| | | | AGATTCTTTGACT | |
| MLF1 | ENST000004 | 246 | TGAGTCCATTCTTGCACACCGAGAAAA | GRCh38/hg38: |
| | 77042.5 | | TATGCGACAGATGATAAGAAGTTTTTC | chr3:158592434: |
| | | | TGAACCCTTTGGAAGAGACTTGCTCAG | 158592581:+ |
| | | | TATCTCTGATGGTAGAGGGAGAGCTCA | |
| | | | TAATCGTAGAGGACATAATGATGGTGA | |
| | | | AGATTCTTTGACT | |
| MOB1B | ENST000003 | 247 | GAGTTCTCAGCCTGAAGTTGACTGGAA | GRCh38/hg38: |
| | 96051.2 | | CTTTCAGTTAACAAGTATTTATCGAAT | chr4:70950700: |
| | | | ACCTGATCTGTAGTGTTGGACTTAGAC | 70950811:+ |
| | | | CTATGGAAGGAGCTACTGATGTGAATG | |
| | | | AAAG | |
| MSRB3 | ENST000003 | 248 | CTCTTGCCCCTGTTCTTTGCTTCTCGT | GRCh38/hg38: |
| | 08259.9 | | TTTGTTGGTGAAGATATCACAGTGATG | chr12:65308529: |
| | | | TCTGCATTCAACCTGCTGCATTTGGTG | 65308655:+ |
| | | | ACAAAGAGCCAGCCAGTAGCCCTTCGA | |
| | | | GCCTGTGGGCTTCCCTCAG | |
| NR1I3 | ENST000005 | 249 | GAGTCTGTGACAGCCACCCCAACACGT | GRCh38/hg38: |
| | 02848.5 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000005 | 250 | GAGTCTGTGACAGCCACCCCAACACGT | GRCh38/hg38: |
| | 15452.1 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000003 | 251 | GAGTCTGTGACAGCCACCCCAACACGT | GRCh38/hg38: |
| | 67983.8 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000003 | 252 | GAGTCTGTGACAGCCACCCCAACACGT | GRCh38/hg38: |
| | 67982.8 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000005 | 253 | GAGTCTGTGACAGCCACCCCAACACGT | GRCh38/hg38: |
| | 11944.5 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000003 | 254 | GAGTCTGTGACAGCCACCCCAACACGT | GRCh38/hg38: |
| | 67980.6 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000003 | 255 | GAGTCTGTGACAGCCACCCCAACACGT | GRCh38/hg38: |
| | 67984.8 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000005 | 256 | GAGTCTGTGACAGCCACCCCAACACGT | GRCh38/hg38: |
| | 05005.5 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000005 | 257 | GAGTCTGTGACAGCCACCCCAACACGT | GRCh38/hg38: |
| | 02985.5 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000004 | 258 | GAGTCTGTGACAGCCACCCCAACACGT | GRCh38/hg38: |
| | 28574.6 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000003 | 259 | GAGTCTGTGACAGCCACCCCAACACGT | GRCh38/hg38: |
| | 67985.7 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000006 | 260 | GAGTCTGTGACAGCCACCCCAACACGT | GRCh38/hg38: |
| | 28566.2 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NR1I3 | ENST000004 | 261 | GAGTCTGTGACAGCCACCCCAACACGT | GRCh38/hg38: |
| | 42691.6 | | GACGTCATGGCCAGTAGGGAAGATGAG | chr1:161236459: |
| | | | CTGAGGAACTGTGTGGTATGTGGGGAC | 161236598:− |
| | | | CAAGCCACAGGCTACCACTTTAATGCG | |
| | | | CTGACTTGTGAGGGCTGCAAGGGTTTC | |
| | | | TTCAG | |
| NT5C3A | ENST000004 | 262 | AAGATTGGATAACCAAGAAATGACTAA | GRCh38/hg38: |
| | 05342.5 | | TCAAGAGTCTGCCGTACATGTGAAAAT | chr7:33035934: |
| | | | G | 33035988:− |
| NT5C3A | ENST000004 | 263 | AAGATTGGATAACCAAGAAATGACTAA | GRCh38/hg38: |
| | 05342.5 | | TCAAGAGTCTGCCGTACATGTGAAAAT | chr7:33035934: |
| | | | G | 33035988:− |
| NUTM1 | ENST000006 | 264 | AATGCCTTGAGTTCCGTATTCTAGTTC | GRCh38/hg38: |
| | 14490.4 | | TGTGTGATCTGATCTTTACCTTCCCTT | chr15:34345857: |
| | | | CCTTGGATCCCTGTGCACCTACTGGAG | 34346035:+ |
| | | | CCAGGTTACTCTGGGTCCTGGACCTGA | |
| | | | CTGCCTCATTCTGGAGGCTTCCAGACA | |
| | | | GCCACAGTTAGTGCCCAAACCTGAGAG | |
| | | | GATGGCTTCAGATGGAG | |
| OTOF | ENST000003 | 265 | GAAGAAGGCCTGAACGACATACAGGAG | GRCh38/hg38: |
| | 38581.10 | | ATGATCAAAACGGAGAAGTCCTACCCT | chr2:26477649: |
| | | | GAGCGTCGCCTGCGGGGCGTCCTGGAG | 26477749:− |
| | | | GAGCTGAGCTGTGGCTGCTG | |
| OTOF | ENST000003 | 266 | GAAGAAGGCCTGAACGACATACAGGAG | GRCh38/hg38: |
| | 39598.7 | | ATGATCAAAACGGAGAAGTCCTACCCT | chr2:26477649: |
| | | | GAGCGTCGCCTGCGGGGCGTCCTGGAG | 26477749:− |
| | | | GAGCTGAGCTGTGGCTGCTG | |
| PDCD4 | ENST000003 | 267 | GGTATTTTCCCTAATTCTCCATGGTGC | GRCh38/hg38: |
| | 93104.6 | | TTCAATAGCATGTTATTATCATAAAAA | chr10:110876670: |
| | | | TGAACAGTTTTGTGGAATAGATGACCA | 110876753:+ |
| | | | AAT | |
| PDPK1 | ENST000005 | 268 | GGACCTTAAACCGGAAAACATTTTGTT | GRCh38/hg38: |
| | 66659.1 | | AAATGAAGATATGCACATCCAGATCAC | chr16:2566356: |
| | | | AGATTTTGGAACAGCAAAAGTCTTATC | 2566453:+ |
| | | | CCCAGAGAGCAAACAAG | |
| PHKB | ENST000005 | 269 | CTTAGCCTGCGACGCTTATGATTAGAG | GRCh38/hg38: |
| | 66037.6 | | CCAACAATTTGAAATGGCCTGCTCACC | chr16:47463899: |
| | | | TGATGCAGTCGTCTCTCCGTCTTCCGC | 47463993:+ |
| | | | TTTCTTAAGGTCTG | |
| PHKB | ENST000005 | 270 | TTTTAAAATTGCTATAGCTTAGCCTGC | GRCh38/hg38: |
| | 66044.5 | | GACGCTTATGATTAGAGCCAACAATTT | chr16:47463882: |
| | | | GAAATGGCCTGCTCACCTGATGCAGTC | 47463993:+ |
| | | | GTCTCTCCGTCTTCCGCTTTCTTAAGG | |
| | | | TCTG | |
| PIGA | ENST000003 | 271 | GTAATAGAGGACACATCTCTTAACTGG | GRCh38/hg38: |
| | 33590.5 | | GTTGCTCTAAGAACTGATGTCTAAACC | chrX:15331216: |
| | | | GTCTCAGCATGGCCTGTAGAGGAGGAG | 15331992:− |
| | | | CTGGGAATGGCCACCGTGCCTCAGCTA | |
| | | | CACTCTCTCGGGTTAGCCCTGGAAGTC | |
| | | | TTTACACATGTAGAACCCGTACCCATA | |
| | | | ATATATGCATGGTATCTGACTTTTTCT | |
| | | | ACCCAAATATGGGAGGCGTGGAAAGCC | |
| | | | ACATTTACCAGCTCTCTCAGTGCCTGA | |
| | | | TTGAAAGAGGGCATAAGGTTATAATTG | |
| | | | TCACCCATGCTTATGGAAATCGAAAAG | |
| | | | GCATCCGTTACCTCACCAGTGGCCTCA | |
| | | | AAGTCTATTACTTGCCTCTGAAAGTCA | |
| | | | TGTACAACCAGTCTACAGCCACGACCC | |
| | | | TCTTTCACAGTCTGCCATTGCTCAGGT | |
| | | | ACATATTTGTTCGGGAGAGAGTCACGA | |
| | | | TAATCCATTCACATAGTTCTTTTTCTG | |
| | | | CTATGGCCCATGATGCTCTCTTCCACG | |
| | | | CCAAGACAATGGGGCTTCAGACAGTCT | |
| | | | TCACGGACCATTCCCTTTTTGGATTTG | |
| | | | CTGATGTCAGCTCGGTGCTTACAAACA | |
| | | | AGCTTCTAACCGTGTCTCTTTGTGATA | |
| | | | CAAACCACATCATTTGTGTGTCTTATA | |
| | | | CTAGTAAGGAAAATACTGTACTAAGAG | |
| | | | CAGCACTGAATCCTGAAATAGTGTCCG | |
| | | | TCATTCCTAATGCTGTAGATCCTACTG | |
| | | | ACTTCACTCCAGACCCATTTAGAAGGC | |
| | | | ATGATAGTATAACTATTGTTGTTGTCA | |
| | | | GCAGACTTGTTTACAGAAAAG | |
| PIGA | ENST000006 | 272 | GTAATAGAGGACACATCTCTTAACTGG | GRCh38/hg38: |
| | 37626.1 | | GTTGCTCTAAGAACTGATGTCTAAACC | chrX:15331216: |
| | | | GTCTCAGCATGGCCTGTAGAGGAGGAG | 15331992:− |
| | | | CTGGGAATGGCCACCGTGCCTCAGCTA | |
| | | | CACTCTCTCGGGTTAGCCCTGGAAGTC | |
| | | | TTTACACATGTAGAACCCGTACCCATA | |
| | | | ATATATGCATGGTATCTGACTTTTTCT | |
| | | | ACCCAAATATGGGAGGCGTGGAAAGCC | |
| | | | ACATTTACCAGCTCTCTCAGTGCCTGA | |
| | | | TTGAAAGAGGGCATAAGGTTATAATTG | |
| | | | TCACCCATGCTTATGGAAATCGAAAAG | |
| | | | GCATCCGTTACCTCACCAGTGGCCTCA | |
| | | | AAGTCTATTACTTGCCTCTGAAAGTCA | |
| | | | TGTACAACCAGTCTACAGCCACGACCC | |
| | | | TCTTTCACAGTCTGCCATTGCTCAGGT | |
| | | | ACATATTTGTTCGGGAGAGAGTCACGA | |
| | | | TAATCCATTCACATAGTTCTTTTTCTG | |
| | | | CTATGGCCCATGATGCTCTCTTCCACG | |
| | | | CCAAGACAATGGGGCTTCAGACAGTCT | |
| | | | TCACGGACCATTCCCTTTTTGGATTTG | |
| | | | CTGATGTCAGCTCGGTGCTTACAAACA | |
| | | | AGCTTCTAACCGTGTCTCTTTGTGATA | |
| | | | CAAACCACATCATTTGTGTGTCTTATA | |
| | | | CTAGTAAGGAAAATACTGTACTAAGAG | |
| | | | CAGCACTGAATCCTGAAATAGTGTCCG | |
| | | | TCATTCCTAATGCTGTAGATCCTACTG | |
| | | | ACTTCACTCCAGACCCATTTAGAAGGC | |
| | | | ATGATAGTATAACTATTGTTGTTGTCA | |
| | | | GCAGACTTGTTTACAGAAAAG | |
| PLOD2 | ENST000004 | 273 | ATAAATTATTAGTCATAACTGTAGCAA | GRCh38/hg38: |
| | 94950.5 | | CAAAAGAAAGTGATGGATTCCATCGAT | chr3:146124138: |
| | | | TTATGCAGTCAGCCAAATATTTCAATT | 146124229:− |
| | | | ATACTGTGAAG | |
| POLR2F | ENST000004 | 274 | ttttagtagtgacggggttcaccgtgt | GRCh38/hg38: |
| | 92213.5 | | tagccaggatggtctcgatctcctgcc | chr22:37958374: |
| | | | ttgtgatccgcccgcctcggcctccca | 37958471:+ |
| | | | aagtgctgggattacag | |
| PPP6C | ENST000004 | 275 | ttttgggctatgatgcattaggcttct | GRCh38/hg38: |
| | 56642.1 | | gcgagtatttatatacagattttgaac | chr9:125171909: |
| | | | atgtttttattgccaagactacagttg | 125172001:− |
| | | | ctgggtcaaatg | |
| PRKN | ENST000004 | 276 | AATTGTGACCTGGATCAGCAGAGCATT | GRCh38/hg38: |
| | 79615.5 | | GTTCACATTGTGCAGAGACCGTGGAGA | chr6:162262525: |
| | | | AAAGGTCAAGAAATGAATGCAACTGGA | 162262765:− |
| | | | GGCGACGACCCCAGAAACGCGGCGGGA | |
| | | | GGCTGTGAGCGGGAGCCCCAGAGCTTG | |
| | | | ACTCGGGTGGACCTCAGCAGCTCAGTC | |
| | | | CTCCCAGGAGACTCTGTGGGGCTGGCT | |
| | | | GTCATTCTGCACACTGACAGCAGGAAG | |
| | | | GACTCACCACCAGCTGGAAGTCCAG | |
| PRUNE1 | ENST000003 | 277 | GACTGACCACTGAGCAGATGCTGAGAA | GRCh38/hg38: |
| | 68935.1 | | AAGACCAGAAGACTATCTATAGACAAG | chr1:151027233: |
| | | | GCGTCAAGGTGGCCATTAGTGCAATAT | 151027327:+ |
| | | | ATATGGATTTGGAG | |
| PSMA6 | ENST000006 | 278 | GACAAATTATTGGATTCCAGCACAGTG | GRCh38/hg38: |
| | 22405.4 | | ACTCACTTATTCAAGATAACTGAAAAC | chr14:35308914: |
| | | | ATTGGTTGTGTGATGACCGGAATGACA | 35308995:+ |
| | | | G | |
| PSMC3I | ENST000005 | 279 | AGCTCAAGGAATTATCTAGTGCCCTGA | GRCh38/hg38: |
| P | 90760.5 | | CCACACCAGAGATGCAGAAAGAAATCC | chr17:42573478: |
| | | | AGGAGTTAAAGAAGGAATGCGCTGGCT | 42573623:− |
| | | | ACAGAGAGAGATTGAAGAACATTAAAG | |
| | | | CAGCTACCAATCATGTGACTCCAGAAG | |
| | | | AGAAAGAGCAG | |
| PTPN1 | ENST000005 | 280 | TTGACCATAGTCGGATTAAACTACATC | GRCh38/hg38: |
| | 41713.5 | | AAGAAGATAATGACTATATCAACGCTA | chr20:50564969: |
| | | | GTTTGATAAAAATGGAAGAAGCCCAAA | 50565069:+ |
| | | | GGAGTTACATTCTTACCCAG | |
| RAB11B | ENST000006 | 281 | GTACTACCGTGGTGCAGTGGGCGCCCT | GRCh38/hg38: |
| | 01897.1 | | GCTGGTGTACGACATCGCCAAGCACCT | chr19:8402086: |
| | | | GACCTATGAGAACGTGGAGCGCTGGCT | 8402279:+ |
| | | | GAAGGAGCTGCGGGACCACGCAGACAG | |
| | | | CAACATCGTCATCATGCTGGTGGGCAA | |
| | | | CAAGAGTGACCTGCGCCACCTGCGGGC | |
| | | | TGTGCCCACTGACGAGGCCCGCGCCTT | |
| | | | CGCAG | |
| RBPJ | ENST000005 | 282 | TCTCCACGTACGTCCCTCAAAGCGCGT | GRCh38/hg38: |
| | 12671.5 | | CCTAAAACCCGGATAACCGGAGCGCTC | chr4:26320700: |
| | | | CCCATGGACCACACGGAGGGCTCGCCC | 26320815:+ |
| | | | GCGGAGGAGCCGCCTGCGCATGCTCCA | |
| | | | TCGCCTGG | |
| RBPJ | ENST000003 | 283 | TCTCCACGTACGTCCCTCAAAGCGCGT | GRCh38/hg38: |
| | 42295.5 | | CCTAAAACCCGGATAACCGGAGCGCTC | chr4:26320700: |
| | | | CCCATGGACCACACGGAGGGCTCGCCC | 26320815:+ |
| | | | GCGGAGGAGCCGCCTGCGCATGCTCCA | |
| | | | TCGCCTGG | |
| RNPS1 | ENST000003 | 284 | GCTGCTCTTTGGCGTAAATTGCAATCG | GRCh38/hg38: |
| | 01730.12 | | ATTAGGGATCGTTTCTCAGAATCAAGT | chr16:2264573: |
| | | | TAGAAGTGAGAGTTCAGATAAGTGAGG | 2264760:− |
| | | | CCGCCATTGCTGCTTTGAACACCTCAG | |
| | | | AAGGGGAGAATGGATTTATCAGGAGTG | |
| | | | AAAAAGAAGAGCTTGCTAGGAGTCAAA | |
| | | | GAAAATAATAAAAAGTCCAGCACTAG | |
| RNPS1 | ENST000003 | 285 | GCTGCTCTTTGGCGTAAATTGCAATCG | GRCh38/hg38: |
| | 20225.9 | | ATTAGGGATCGTTTCTCAGAATCAAGT | chr16:2264573: |
| | | | TAGAAGTGAGAGTTCAGATAAGTGAGG | 2264760:− |
| | | | CCGCCATTGCTGCTTTGAACACCTCAG | |
| | | | AAGGGGAGAATGGATTTATCAGGAGTG | |
| | | | AAAAAGAAGAGCTTGCTAGGAGTCAAA | |
| | | | GAAAATAATAAAAAGTCCAGCACTAG | |
| RNPS1 | ENST000005 | 286 | GCTGCTCTTTGGCGTAAATTGCAATCG | GRCh38/hg38: |
| | 64311.5 | | ATTAGGGATCGTTTCTCAGAATCAAGT | chr16:2264573: |
| | | | TAGAAGTGAGAGTTCAGATAAGTGAGG | 2264760:− |
| | | | CCGCCATTGCTGCTTTGAACACCTCAG | |
| | | | AAGGGGAGAATGGATTTATCAGGAGTG | |
| | | | AAAAAGAAGAGCTTGCTAGGAGTCAAA | |
| | | | GAAAATAATAAAAAGTCCAGCACTAG | |
| RNPS1 | ENST000005 | 287 | GCTGCTCTTTGGCGTAAATTGCAATCG | GRCh38/hg38: |
| | 69598.6 | | ATTAGGGATCGTTTCTCAGAATCAAGT | chr16:2264573: |
| | | | TAGAAGTGAGAGTTCAGATAAGTGAGG | 2264760:− |
| | | | CCGCCATTGCTGCTTTGAACACCTCAG | |
| | | | AAGGGGAGAATGGATTTATCAGGAGTG | |
| | | | AAAAAGAAGAGCTTGCTAGGAGTCAAA | |
| | | | GAAAATAATAAAAAGTCCAGCACTAG | |
| RPL5 | ENST000006 | 288 | AGGGTAAAACTGATTATTATGCTCGGA | GRCh38/hg38: |
| | 45300.1 | | AACGCTTGGTGATACAAGATAAAAATA | chr1:92833545: |
| | | | AATACAACACACCCAAATACAGGATGA | 92833660:+ |
| | | | TAGTTCGTGTGACAAACAGAGATATCA | |
| | | | TTTGTCAG | |
| RPS20 | ENST000005 | 289 | TGTGTGCTGACTTGATAAGAGGCGCAA | GRCh38/hg38: |
| | 20627.1 | | AAGAAAAGAATCTCAAAGTGAAAGGAC | chr8:56073695: |
| | | | CAGTTCGAATGCCTACCAAG | 56073768:− |
| SECISB | ENST000005 | 290 | GCAGAAAATATATACTGAAGACATGGC | GRCh38/hg38: |
| P2 | 34113.6 | | CTTTGGAGCTTCAACTTTTCCACCTCA | chr9:89325427: |
| | | | GTATTTATCTTCTGAGATAACTCTTCA | 89325676:+ |
| | | | TCCATATGCCTATTCTCCTTATACCCT | |
| | | | TGACTCCACACAGAATGTTTACTCAGT | |
| | | | GCCTGGCTCCCAGTATCTTTATAACCA | |
| | | | ACCCAGTTGTTACCGAGGTTTTCAAAC | |
| | | | AGTGAAGCATCGAAATGAGAACACATG | |
| | | | CCCTCTCCCACAAGAAATGAAAGCTCT | |
| | | | GTTTAAG | |
| SELENB | ENST000004 | 291 | GTCATGTGTCTCCAGCTCTTCCCAGCA | GRCh38/hg38: |
| P1 | 26705.6 | | CAACCTCACCCTCCTCAGGGCTCTGAG | chr1:151369713: |
| | | | GGGGCGGAGAGGACAGGCCAGAGCGAT | 151369978:− |
| | | | GAGGCTGGAGTGGGGACCTAGGCCAGC | |
| | | | CGCACTGCCGTGGCCCGCTGGGATGTG | |
| | | | TGCTGCAGAACGTGCGGAGGGAGCCTT | |
| | | | CACCCTCCAGAGCGTGGCCCAGCCAAT | |
| | | | GCGCCCCATTGCTTCCACAGCTACGAA | |
| | | | ATGTGGGAATTGTGGACCCGGCTACTC | |
| | | | CACCCCTCTGGAGGCCATGAAAG | |
| SELENB | ENST000004 | 292 | GTCATGTGTCTCCAGCTCTTCCCAGCA | GRCh38/hg38: |
| P1 | 23070.5 | | CAACCTCACCCTCCTCAGGGCTCTGAG | chr1:151369713: |
| | | | GGGGCGGAGAGGACAGGCCAGAGCGAT | 151369978:− |
| | | | GAGGCTGGAGTGGGGACCTAGGCCAGC | |
| | | | CGCACTGCCGTGGCCCGCTGGGATGTG | |
| | | | TGCTGCAGAACGTGCGGAGGGAGCCTT | |
| | | | CACCCTCCAGAGCGTGGCCCAGCCAAT | |
| | | | GCGCCCCATTGCTTCCACAGCTACGAA | |
| | | | ATGTGGGAATTGTGGACCCGGCTACTC | |
| | | | CACCCCTCTGGAGGCCATGAAAG | |
| SEPT11 | ENST000005 | 293 | TCTACATCGGTAAACTCTGCAAAACTC | GRCh38/hg38: |
| | 04637.5 | | CAGCAGTTACAGAGCCTGTGCTAGGAG | chr4:76995781: |
| | | | CTACAGGGTGAAGAGGGGGAAGAAGAC | 76995938:+ |
| | | | TCAAGAAACCTGTAAAACTAATGGAGG | |
| | | | AGAGGAAACCAGCTCATGTGCTGAGAA | |
| | | | GTTTTAAATATGCTGCATTTATG | |
| SIRT2 | ENST000004 | 294 | GACTCAGATTCAGACTCTGAGGGAGGA | GRCh38/hg38: |
| | 23526.5 | | GCCGCTGGTGGAGAAGCAGACA | chr19:38893819: |
| | | | | 38893867:− |
| SIRT2 | ENST000003 | 295 | GACTCAGATTCAGACTCTGAGGGAGGA | GRCh38/hg38: |
| | 92081.6 | | GCCGCTGGTGGAGAAGCAGACA | chr19:38893819: |
| | | | | 38893867:− |
| SIRT2 | ENST000003 | 296 | GACTCAGATTCAGACTCTGAGGGAGGA | GRCh38/hg38: |
| | 58931.9 | | GCCGCTGGTGGAGAAGCAGACA | chr19:38893819: |
| | | | | 38893867:− |
| SIRT2 | ENST000004 | 297 | GACTCAGATTCAGACTCTGAGGGAGGA | GRCh38/hg38: |
| | 51193.5 | | GCCGCTGGTGGAGAAGCAGACA | chr19:38893819: |
| | | | | 38893867:− |
| SIRT2 | ENST000004 | 298 | GACTCAGATTCAGACTCTGAGGGAGGA | GRCh38/hg38: |
| | 20440.5 | | GCCGCTGGTGGAGAAGCAGACA | chr19:38893819: |
| | | | | 38893867:− |
| SLC25A | ENST000004 | 299 | CTGCTGCATTTTTTATCACCTATGAAT | GRCh38/hg38: |
| 26 | 64350.6 | | ATGTGAAGTGGTTTTTGCATGCTGATT | chr3:66243203: |
| | | | CATCTTCATATTTGACACCTATGAAAC | 66243312:+ |
| | | | ATATGTTGGCTGCCTCTGCTGGAGAAG | |
| | | | TG | |
| SLC25A | ENST000003 | 300 | CTGCTGCATTTTTTATCACCTATGAAT | GRCh38/hg38: |
| 26 | 36733.10 | | ATGTGAAGTGGTTTTTGCATGCTGATT | chr3:66243203: |
| | | | CATCTTCATATTTGACACCTATGAAAC | 66243312:+ |
| | | | ATATGTTGGCTGCCTCTGCTGGAGAAG | |
| | | | TG | |
| SMARCE | ENST000004 | 301 | TGGCTATACAGGTGGAGCTGAACAATT | GRCh38/hg38: |
| 1 | 78349.7 | | GAGATTCTGACCTCAAAGAGCTAACCA | chr17:40644385: |
| | | | TGTACTTCAGCTTTGGAAATG | 40644459:− |
| SMC1A | ENST000003 | 302 | CCCAGCGTCCTCTCTCGCCTCGCATTG | GRCh38/hg38: |
| | 75340.10 | | GTACACTCCAACTCCTTTCTACTCTTC | chrX:53421895: |
| | | | TCTGGAGGCGGCACTTTTGAATTTCAG | 53422040:− |
| | | | ACCCAGCGCTCCAGTCTTTAGAATGCT | |
| | | | TGAGGTGTCGATCCCCACCCCCCACCG | |
| | | | CTATGTCAGAG | |
| SMG1 | ENST000005 | 303 | ATTGACTTGATGCAAAACATCACAAAG | GRCh38/hg38: |
| | 32700.6 | | GCTCCATATTATACAGTATGCGATTTT | chr16:18899987: |
| | | | TGAG | 18900044:− |
| SPACA7 | ENST000004 | 304 | agtctcactctgttgccagactggagt | GRCh38/hg38: |
| | 14180.5 | | gcagaggcgcctctcgatttcttgacc | chr13:112382278: |
| | | | tcatggtccgcccgcctcagcctccca | 112382507:+ |
| | | | aagtgctgggactacaggcatgagcca | |
| | | | ccgtgcctggccaataacaccttaatc | |
| | | | ttcaggtgcaggctgtgaagatgggaa | |
| | | | aaggctgtcaatgctctggcttcttcc | |
| | | | cactgacaggggacgtagtgggaatgg | |
| | | | gaatgaaccccaag | |
| SPRED1 | ENST000005 | 305 | TAATAGTTATGCACGAGTGCGAGCTGT | GRCh38/hg38: |
| | 61317.1 | | GGTGATGACCCGAGATGACTCAAGTGG | chr15:38299373: |
| | | | TGGATGGTTACCACTTGGAGGGAGTGG | 38299547:+ |
| | | | ACTAAGCAGCGTCACTGTCTTCAAAGT | |
| | | | CCCTCATCAGGAAGAGAATGGCTGTGC | |
| | | | TGACTTTTTTATCCGTGGAGAGCGACT | |
| | | | CAGGGACAAAATG | |
| SRP54 | ENST000005 | 306 | GTCTGCTATTGATCTTGAAGAGATGGC | GRCh38/hg38: |
| | 55557.5 | | ATCTGGTCTTAACAAAAGAAAAATGAT | chr14:35000936: |
| | | | TCAGCATGCTGTATTTAAAGAACTTGT | 35001020:+ |
| | | | GAAG | |
| SRPK2 | ENST000004 | 307 | AAATCAAGCAGAAGATTGAGCTGCTGA | GRCh38/hg38: |
| | 60391.5 | | TGTCAGTTAACTCTGAGAAGTCGTCCT | chr7:105268806: |
| | | | CTTCAGAAAG | 105268869:− |
| STK36 | ENST000004 | 308 | AGAAGGAGCTGAGGAATTTGCAACGAG | GRCh38/hg38: |
| | 55724.5 | | AGATTGAAATAATGCGGGGTCTGCGGC | chr2:218673665: |
| | | | ATCCCAACATTGTGCATATGCTTGACA | 218673765:+ |
| | | | GCTTTGAAACTGATAAAGAG | |
| STRADA | ENST000006 | 309 | ACCAATGATGCGAGCTCAGAGTCAATA | GRCh38/hg38: |
| | 38718.1 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000005 | 310 | ACCAATGATGCGAGCTCAGAGTCAATA | GRCh38/hg38: |
| | 80338.5 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000005 | 311 | ACCAATGATGCGAGCTCAGAGTCAATA | GRCh38/hg38: |
| | 79340.5 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000006 | 312 | ACCAATGATGCGAGCTCAGAGTCAATA | GRCh38/hg38: |
| | 40979.1 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000005 | 313 | ACCAATGATGCGAGCTCAGAGTCAATA | GRCh38/hg38: |
| | 82030.5 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000006 | 314 | ACCAATGATGCGAGCTCAGAGTCAATA | GRCh38/hg38: |
| | 38276.1 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000003 | 315 | ACCAATGATGCGAGCTCAGAGTCAATA | GRCh38/hg38: |
| | 75840.9 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000006 | 316 | ACCAATGATGCGAGCTCAGAGTCAATA | GRCh38/hg38: |
| | 38702.1 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| STRADA | ENST000002 | 317 | ACCAATGATGCGAGCTCAGAGTCAATA | GRCh38/hg38: |
| | 45865.10 | | GCATCCTTCTCTAAACAGGAGGTCATG | chr17:63714006: |
| | | | AGTAGCTTTCTGCCAGAGGGAGGGTGT | 63714108:− |
| | | | TACGAGCTGCTCACTGTGATAG | |
| SUMO1 | ENST000004 | 318 | CAGTGAGATTCACTTCAAAGTGAAAAT | GRCh38/hg38: |
| | 09205.5 | | GACAACACATCTCAAGAAACTCAAAGA | chr2:202214357: |
| | | | ATCATACTGTCAAAGACAG | 202214429:− |
| TBL1XR | ENST000004 | 319 | GGTTATATCCTGTGTTGTGACCTCATG | GRCh38/hg38: |
| 1 | 22442.5 | | GTTTAAGTGGGAATAAAGATGAGTATA | chr3:177064920: |
| | | | AGCAGTGATGAGGTCAACTTCTTGGTA | 177065022:− |
| | | | TATAGATACTTGCAAGAGTCAG | |
| TBL1XR | ENST000004 | 320 | GGTTATATCCTGTGTTGTGACCTCATG | GRCh38/hg38: |
| 1 | 50267.5 | | GTTTAAGTGGGAATAAAGATGAGTATA | chr3:177064920: |
| | | | AGCAGTGATGAGGTCAACTTCTTGGTA | 177065022:− |
| | | | TATAGATACTTGCAAGAGTCAG | |
| TBL1XR | ENST000006 | 321 | GGTTATATCCTGTGTTGTGACCTCATG | GRCh38/hg38: |
| 1 | 35794.1 | | GTTTAAGTGGGAATAAAGATGAGTATA | chr3:177064920: |
| | | | AGCAGTGATGAGGTCAACTTCTTGGTA | 177065022:− |
| | | | TATAGATACTTGCAAGAGTCAG | |
| TBL1XR | ENST000004 | 322 | GGTTATATCCTGTGTTGTGACCTCATG | GRCh38/hg38: |
| 1 | 43315.5 | | GTTTAAGTGGGAATAAAGATGAGTATA | chr3:177064920: |
| | | | AGCAGTGATGAGGTCAACTTCTTGGTA | 177065022:− |
| | | | TATAGATACTTGCAAGAGTCAG | |
| TLR8 | ENST000003 | 323 | GTTCTCTTGACACTTCAGTGTTAGGGA | GRCh38/hg38: |
| | 11912.5 | | ACATCAGCAAGACCCATCCCAGGAGAC | chrX:12910306: |
| | | | CTTGAAGGAAGCCTTTGAAAGGGAGAA | 12910442:+ |
| | | | TGAAGGAGTCATCTTTGCAAAATAGCT | |
| | | | CCTGCAGCCTGGGAAAGGAGACTAAAA | |
| | | | AG | |
| TXNRD2 | ENST000004 | 324 | GGCAGAGTGGAGGGCAGCAAACAGGAG | GRCh38/hg38: |
| | 62843.2 | | AATGAGCAACCTACCTAGGTCCTTGGG | chr22:19880856: |
| | | | ATGCCCCTCAGGAGGACGAGCTCTAGG | 19880945:− |
| | | | GGGAATGCT | |
| UBE3A | ENST000006 | 325 | AGAGTTACAGTGGAGGTAAAAGGAGTG | GRCh38/hg38: |
| | 50110.1 | | GCTTGCAGGATGGAGAAGCTGCACCAG | chr15:25408620: |
| | | | TGTTATTGGAA | 25408684:− |
| UBE3A | ENST000003 | 326 | AGAGTTACAGTGGAGGTAAAAGGAGTG | GRCh38/hg38: |
| | 97954.6 | | GCTTGCAGGATGGAGAAGCTGCACCAG | chr15:25408620: |
| | | | TGTTATTGGAA | 25408684:− |
| UFM1 | ENST000003 | 327 | GTCGAAGGTTTCCTTTAAGATCACGCT | GRCh38/hg38: |
| | 79649.5 | | GACGTCGGACCCACGGCTGCCGTACAA | chr13:38349999: |
| | | | AGTGTGAGTAGCTCGGCCGAGATGGGC | 38350227:+ |
| | | | CTTTTGGGGCCGGACAAGACGGGGCTG | |
| | | | GGTTGGGGATGATCCGAGCTTTTCCAA | |
| | | | CAACTACCCCACGCAGTCTTCATCTCT | |
| | | | TCACTTCATCTACTTTCCTGGCTCGCG | |
| | | | CCCTTCCAGGAGCCTTTCCCACCGGAG | |
| | | | CCTGCGAGGAGAG | |
| WAC | ENST000004 | 328 | GCACTTAAGTATTCATCGAAGAGTCAC | GRCh38/hg38: |
| | 20266.5 | | CCCAGTAGCGGTGATCACAGACATGAA | chr10:28535562: |
| | | | AAGATGCGAGACGCCGGAGATCCTTCA | 28535757:+ |
| | | | CCACCAAATAAAATGTTGCGGAGATCT | |
| | | | GATAGTCCTGAAAACAAATACAGTGAC | |
| | | | AGCACAGGTCACAGTAAGGCCAAAAAT | |
| | | | GTGCATACTCACAGAGTTAGAGAGAGG | |
| | | | GATGGTG | |
| WDR81 | ENST000003 | 329 | GTGCTGGCGGGCGCAGAGGCCTCCCAG | GRCh38/hg38: |
| | 09182.9 | | GAGGAGAGCAAGGACCTGGCAGGGGCT | chr17:1727990: |
| | | | GCTGAGGAGGAGGAGAGCGGGCTGCCC | 1728626:+ |
| | | | GGGGCCGGGCCTGGCTCCTGTGCTTTT | |
| | | | GGGGAGGAGATTCCCATGGATGGGGAG | |
| | | | CCTCCTGCCTCCTCGGGCCTGGGGCTC | |
| | | | CCAGACTACACGTCTGGCGTCAGCTTC | |
| | | | CACGACCAGGCTGACCTCCCTGAGACA | |
| | | | GAGGACTTCCAAGCCGGGCTCTATGTG | |
| | | | ACTGAGTCTCCCCAGCCCCAGGAGGCT | |
| | | | GAGGCTGTGAGCCTGGGCCGGCTGAGT | |
| | | | GACAAGAGCAGCACCAGCGAGACCTCC | |
| | | | CTGGGTGAGGAGCGGGCTCCAGACGAG | |
| | | | GGGGGTGCCCCCGTGGACAAGAGCAGC | |
| | | | CTTCGATCAGGTGACAGCAGCCAGGAC | |
| | | | TTGAAGCAAAGCGAGGGCTCCgaggag | |
| | | | gaagaggaggaggaggacagctgcgtg | |
| | | | gtgctagaggaggaggagggggagcag | |
| | | | gaggaggTCACCGGGGCATCTGAGCTC | |
| | | | ACTCTGTCTGACACGGTGCTGTCCATG | |
| | | | GAGACGGTTGTGGCCGGCGGCAGTGGG | |
| | | | GGAGATGGAGAAGAAGAGGAGGAGGCA | |
| | | | CTGCCTGAGCAGTCAGAAGGCAAAGAA | |
| | | | CAGAAGATCCTCCTTG | |
| YME1L1 | ENST000003 | 330 | TACTGGCTAACAAACTGAAGCAGCTGC | GRCh38/hg38: |
| | 96296.7 | | TTGTTATTGGGCATCAGTTATGTACCA | chr10:27153227: |
| | | | G | 27153281:− |
| ZMYND1 | ENST000004 | 331 | GTGGAACCAGCAGCATGAGAACCTGGA | GRCh38/hg38: |
| 0 | 42887.1 | | GAAGCTGAACATGCAAGCCATCCTCGA | chr3:50345124: |
| | | | TGCCACAGTCAGCCAGGGCGAGCCCAT | 50345232:− |
| | | | TCAGGAGCTGCTGGTCACCCATGGGAA | |
| | | | G | |

ACIN1

In some cases, the target peptide sequence is a portion of ACIN1 (apoptotic chromatin condensation inducer 1) protein. In some cases, the therapeutic agent increases level of a first processed mRNA encoding a protein having a sequence SEQ ID NO: 8096, and decreases level of a second processed mRNA encoding a protein having a sequence SEQ ID NO: 332. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 130. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 130. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 23071186. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 23071186. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 130. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 130. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 23071125. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 23071125. In some cases, the targeted region of the pre-mRNA is within a sequence SEQ ID NO: 130. In some cases, the targeted region of the pre-mRNA is within a sequence between a pair of genomic sites GRCh38/hg38: chr14 23071186, and GRCh38/hg38: chr14 23071125. In some cases, the therapeutic agent is an antisense oligomer complementary to a sequence with at least about 80%, 85%, 90%, 95%, 96%, 97%, 98%, 99% or 100% sequence identity to SEQ ID NO:130. In some cases, the therapeutic agent is an antisense oligomer complementary to a sequence with 100% sequence identity to SEQ ID NO:130. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 534-584. In some cases, the therapeutic agent is an antisense oligomer that has a sequence selected from the group consisting of SEQ ID NOs: 534-584. In some cases, the target peptide sequence is a portion of ACIN1 protein, and the method treats a disease or the condition that comprises Intellectual Disability; or Colorectal Cancer.

ACTN1

In some cases, the target peptide sequence is a portion of ACTN1 protein. In some cases, the therapeutic agent increases level of a first processed mRNA encoding a protein having a sequence SEQ ID NO: 8097, and decreases level of a second processed mRNA encoding a protein having a sequence SEQ ID NO: 333. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 131. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 131. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 68925672. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 68925672. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 131. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 131. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 68925558. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 68925558. In some cases, the targeted region of the pre-mRNA is within a sequence SEQ ID NO: 131. In some cases, the targeted region of the pre-mRNA is within a sequence between a pair of genomic sites GRCh38/hg38: chr14 68925672, and GRCh38/hg38: chr14 68925558. In some cases, the therapeutic agent is an antisense oligomer complementary to a sequence with at least about 80%, 85%, 90%, 95%, 96%, 97%, 98%, 99% or 100% sequence identity to SEQ ID NO:131. In some cases, the therapeutic agent is an antisense oligomer complementary to a sequence with 100% sequence identity to SEQ ID NO:131. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 585-646. In some cases, the therapeutic agent is an antisense oligomer that has a sequence selected from the group consisting of SEQ ID NOs: 585-646. In some cases, the target peptide sequence is a portion of ACTN1 protein, and the method treats a disease or the condition that comprises Bleeding Disorder, Platelet-Type, 15; or Autosomal dominant macrothrombocytopenia.

ACTN2

In some cases, the target peptide sequence is a portion of ACTN2 protein. In some cases, the therapeutic agent increases level of a first processed mRNA encoding a protein having a sequence SEQ ID NO: 8098, and decreases level of a second processed mRNA encoding a protein having a sequence SEQ ID NO: 334. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 132. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 132. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 236735635. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 236735635. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 132. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 132. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 236735720. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 236735720. In some cases, the targeted region of the pre-mRNA is within a sequence SEQ ID NO: 132. In some cases, the targeted region of the pre-mRNA is within a sequence between a pair of genomic sites GRCh38/hg38: chr1 236735635, and GRCh38/hg38: chr1 236735720. In some cases, the therapeutic agent is an antisense oligomer complementary to a sequence with at least about 80%, 85%, 90%, 95%, 96%, 97%, 98%, 99% or 100% sequence identity to SEQ ID NO:132. In some cases, the therapeutic agent is an antisense oligomer complementary to a sequence with 100% sequence identity to SEQ ID NO:132. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 647-702. In some cases, the therapeutic agent is an antisense oligomer that has a sequence selected from the group consisting of SEQ ID NOs: 647-702. In some cases, the target peptide sequence is a portion of ACTN2 protein, and the method treats a disease or the condition that comprises Hypertrophic Cardiomyopathy; Cardiomyopathy, Dilated, 1AA; Cardiomyopathy, Familial Hypertrophic, 23, with or without Ventricular Noncompaction; Familial dilated cardiomyopathy; Charcot-Marie-Tooth Disease; Conduction disorder of the heart; or Cardiomyopathy, Dilated.

ADH4

In some cases, the target peptide sequence is a portion of ADH4 protein. In some cases, the therapeutic agent increases level of a first processed mRNA encoding a protein having a sequence SEQ ID NO: 8099, and decreases level of a second processed mRNA encoding a protein having a sequence SEQ ID NO: 335. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 133. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 133. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 99143305. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 99143305. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 133. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 133. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 99143166. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 99143166. In some cases, the targeted region of the pre-mRNA is within a sequence SEQ ID NO: 133. In some cases, the targeted region of the pre-mRNA is within a sequence between a pair of genomic sites GRCh38/hg38: chr4 99143305, and GRCh38/hg38: chr4 99143166. In some cases, the therapeutic agent is an antisense oligomer complementary to a sequence with at least about 80%, 85%, 90%, 95%, 96%, 97%, 98%, 99% or 100% sequence identity to SEQ ID NO:133. In some cases, the therapeutic agent is an antisense oligomer complementary to a sequence with 100% sequence identity to SEQ ID NO:133. In some cases, the therapeutic agent increases level of a first processed mRNA encoding a protein having a sequence SEQ ID NO: 8100, and decreases level of a second processed mRNA encoding a protein having a sequence SEQ ID NO: 336. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 134. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 134. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 99143305. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 99143305. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 134. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 134. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 99143166. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 99143166. In some cases, the targeted region of the pre-mRNA is within a sequence SEQ ID NO: 134. In some cases, the targeted region of the pre-mRNA is within a sequence between a pair of genomic sites GRCh38/hg38: chr4 99143305, and GRCh38/hg38: chr4 99143166. In some cases, the therapeutic agent is an antisense oligomer complementary to a sequence with at least about 80%, 85%, 90%, 95%, 96%, 97%, 98%, 99% or 100% sequence identity to SEQ ID NO:134. In some cases, the therapeutic agent is an antisense oligomer complementary to a sequence with 100% sequence identity to SEQ ID NO:134. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 703-769. In some cases, the therapeutic agent is an antisense oligomer that has a sequence selected from the group consisting of SEQ ID NOs: 703-769. In some cases, the target peptide sequence is a portion of ADH4 protein, and the method treats a disease or the condition that comprises Alcohol Use Disorder; Alcohol abuse; or Alcoholic Intoxication, Chronic.

AGER

In some cases, the target peptide sequence is a portion of AGER protein. In some cases, the therapeutic agent increases level of a first processed mRNA encoding a protein having a sequence SEQ ID NO: 8101, and decreases level of a second processed mRNA encoding a protein having a sequence SEQ ID NO: 337. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 135. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 135. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 32182698. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 32182698. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 135. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 135. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 32182568. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 32182568. In some cases, the targeted region of the pre-mRNA is within a sequence SEQ ID NO: 135. In some cases, the targeted region of the pre-mRNA is within a sequence between a pair of genomic sites GRCh38/hg38: chr6 32182698, and GRCh38/hg38: chr6 32182568. In some cases, the therapeutic agent is an antisense oligomer complementary to a sequence with at least about 80%, 85%, 90%, 95%, 96%, 97%, 98%, 99% or 100% sequence identity to SEQ ID NO:135. In some cases, the therapeutic agent is an antisense oligomer complementary to a sequence with 100% sequence identity to SEQ ID NO:135. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 8031-8095. In some cases, the therapeutic agent is an antisense oligomer that has a sequence selected from the group consisting of SEQ ID NOs: 8031-8095. In some cases, the target peptide sequence is a portion of AGER protein, and the method treats a disease or the condition that comprises Schizophrenia.

AKR1C3

In some cases, the target peptide sequence is a portion of AKR1C3 protein. In some cases, the therapeutic agent increases level of a first processed mRNA encoding a protein having a sequence SEQ ID NO: 8102, and decreases level of a second processed mRNA encoding a protein having a sequence SEQ ID NO: 338. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 136. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 136. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 5077900. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 5077900. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 136. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 136. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 5077977. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 5077977. In some cases, the targeted region of the pre-mRNA is within a sequence SEQ ID NO: 136. In some cases, the targeted region of the pre-mRNA is within a sequence between a pair of genomic sites GRCh38/hg38: chr10 5077900, and GRCh38/hg38: chr10 5077977. In some cases, the therapeutic agent is an antisense oligomer complementary to a sequence with at least about 80%, 85%, 90%, 95%, 96%, 97%, 98%, 99% or 100% sequence identity to SEQ ID NO:136. In some cases, the therapeutic agent is an antisense oligomer complementary to a sequence with 100% sequence identity to SEQ ID NO:136. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 770-824. In some cases, the therapeutic agent is an antisense oligomer that has a sequence selected from the group consisting of SEQ ID NOs: 770-824. In some cases, the target peptide sequence is a portion of AKR1C3 protein, and the method treats a disease or the condition that comprises Bipolar Disorder; or Alcoholic Intoxication, Chronic.

ALDH1A2

In some cases, the target peptide sequence is a portion of ALDH1A2 protein. In some cases, the therapeutic agent increases level of a first processed mRNA encoding a protein having a sequence SEQ ID NO: 8103, and decreases level of a second processed mRNA encoding a protein having a sequence SEQ ID NO: 339. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 137. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 137. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 58058108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 58058108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 137. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 137. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 58058012. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 58058012. In some cases, the targeted region of the pre-mRNA is within a sequence SEQ ID NO: 137. In some cases, the targeted region of the pre-mRNA is within a sequence between a pair of genomic sites GRCh38/hg38: chr15 58058108, and GRCh38/hg38: chr15 58058012. In some cases, the therapeutic agent is an antisense oligomer complementary to a sequence with at least about 80%, 85%, 90%, 95%, 96%, 97%, 98%, 99% or 100% sequence identity to SEQ ID NO:137. In some cases, the therapeutic agent is an antisense oligomer complementary to a sequence with 100% sequence identity to SEQ ID NO:137. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 825-882. In some cases, the therapeutic agent is an antisense oligomer that has a sequence selected from the group consisting of SEQ ID NOs: 825-882. In some cases, the target peptide sequence is a portion of ALDH1A2 protein, and the method treats a disease or the condition that comprises Schizophrenia.

ALG8

In some cases, the target peptide sequence is a portion of ALG8 protein. In some cases, the therapeutic agent increases level of a first processed mRNA encoding a protein having a sequence SEQ ID NO: 8104, and decreases level of a second processed mRNA encoding a protein having a sequence SEQ ID NO: 340. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 138. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 138. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 78138767. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 78138767. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 138. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 138. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 78138710. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 78138710. In some cases, the targeted region of the pre-mRNA is within a sequence SEQ ID NO: 138. In some cases, the targeted region of the pre-mRNA is within a sequence between a pair of genomic sites GRCh38/hg38: chr11 78138767, and GRCh38/hg38: chr11 78138710. In some cases, the therapeutic agent is an antisense oligomer complementary to a sequence with at least about 80%, 85%, 90%, 95%, 96%, 97%, 98%, 99% or 100% sequence identity to SEQ ID NO:138. In some cases, the therapeutic agent is an antisense oligomer complementary to a sequence with 100% sequence identity to SEQ ID NO:138. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 883-933. In some cases, the therapeutic agent is an antisense oligomer that has a sequence selected from the group consisting of SEQ ID NOs: 883-933. In some cases, the target peptide sequence is a portion of ALG8 protein, and the method treats a disease or the condition that comprises Intellectual Disability; Congenital Disorders of Glycosylation; Epileptic encephalopathy; Depressive disorder; or Congenital disorder of glycosylation type 1H.

AMPD2

In some cases, the target peptide sequence is a portion of AMPD2 protein. In some cases, the therapeutic agent increases level of a first processed mRNA encoding a protein having a sequence SEQ ID NO: 8105, and decreases level of a second processed mRNA encoding a protein having a sequence SEQ ID NO: 341. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 139. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 139. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 109620914. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 109620914. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 139. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 139. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 109621266. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 109621266. In some cases, the targeted region of the pre-mRNA is within a sequence SEQ ID NO: 139. In some cases, the targeted region of the pre-mRNA is within a sequence between a pair of genomic sites GRCh38/hg38: chr1 109620914, and GRCh38/hg38: chr1 109621266. In some cases, the therapeutic agent is an antisense oligomer complementary to a sequence with at least about 80%, 85%, 90%, 95%, 96%, 97%, 98%, 99% or 100% sequence identity to SEQ ID NO:139. In some cases, the therapeutic agent is an antisense oligomer complementary to a sequence with 100% sequence identity to SEQ ID NO:139. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 934-1043. In some cases, the therapeutic agent is an antisense oligomer that has a sequence selected from the group consisting of SEQ ID NOs: 934-1043. In some cases, the target peptide sequence is a portion of AMPD2 protein, and the method treats a disease or the condition that comprises Intellectual Disability; Pontocerebellar Hypoplasia, Type 9; Epileptic encephalopathy; Ataxias, Hereditary; Spastic Paraplegia 63, Autosomal Recessive; Cerebellar Hypoplasia; or Spastic Paraplegia, Hereditary.

AMT

In some cases, the target peptide sequence is a portion of AMT protein. In some cases, the therapeutic agent increases level of a first processed mRNA encoding a protein having a sequence SEQ ID NO: 8106, and decreases level of a second processed mRNA encoding a protein having a sequence SEQ ID NO: 342. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 140. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 140. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 49422257. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 49422257. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 140. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 140. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 49422104. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 49422104. In some cases, the targeted region of the pre-mRNA is within a sequence SEQ ID NO: 140. In some cases, the targeted region of the pre-mRNA is within a sequence between a pair of genomic sites GRCh38/hg38: chr3 49422257, and GRCh38/hg38: chr3 49422104. In some cases, the therapeutic agent is an antisense oligomer complementary to a sequence with at least about 80%, 85%, 90%, 95%, 96%, 97%, 98%, 99% or 100% sequence identity to SEQ ID NO:140. In some cases, the therapeutic agent is an antisense oligomer complementary to a sequence with 100% sequence identity to SEQ ID NO:140. In some cases, the therapeutic agent increases level of a first processed mRNA encoding a protein having a sequence SEQ ID NO: 8107, and decreases level of a second processed mRNA encoding a protein having a sequence SEQ ID NO: 343. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 141. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 141. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 49422257. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 49422257. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 141. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 141. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 49422104. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 49422104. In some cases, the targeted region of the pre-mRNA is within a sequence SEQ ID NO: 141. In some cases, the targeted region of the pre-mRNA is within a sequence between a pair of genomic sites GRCh38/hg38: chr3 49422257, and GRCh38/hg38: chr3 49422104. In some cases, the therapeutic agent is an antisense oligomer complementary to a sequence with at least about 80%, 85%, 90%, 95%, 96%, 97%, 98%, 99% or 100% sequence identity to SEQ ID NO:141. In some cases, the therapeutic agent is an antisense oligomer complementary to a sequence with 100% sequence identity to SEQ ID NO:141. In some cases, the therapeutic agent increases level of a first processed mRNA encoding a protein having a sequence SEQ ID NO: 8108, and decreases level of a second processed mRNA encoding a protein having a sequence SEQ ID NO: 344. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 142. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 142. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 49422257. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 49422257. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 142. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 142. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 49422104. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 49422104. In some cases, the targeted region of the pre-mRNA is within a sequence SEQ ID NO: 142. In some cases, the targeted region of the pre-mRNA is within a sequence between a pair of genomic sites GRCh38/hg38: chr3 49422257, and GRCh38/hg38: chr3 49422104. In some cases, the therapeutic agent is an antisense oligomer complementary to a sequence with at least about 80%, 85%, 90%, 95%, 96%, 97%, 98%, 99% or 100% sequence identity to SEQ ID NO:142. In some cases, the therapeutic agent is an antisense oligomer complementary to a sequence with 100% sequence identity to SEQ ID NO:142. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1044-1113. In some cases, the therapeutic agent is an antisense oligomer that has a sequence selected from the group consisting of SEQ ID NOs: 1044-1113. In some cases, the target peptide sequence is a portion of AMT protein, and the method treats a disease or the condition that comprises Nonketotic Hyperglycinemia; Hyperglycinemia, Transient Neonatal; or Glycine encephalopathy.

ANKRD11

In some cases, the target peptide sequence is a portion of ANKRD1 1 protein. In some cases, the therapeutic agent increases level of a first processed mRNA encoding a protein having a sequence SEQ ID NO: 8109, and decreases level of a second processed mRNA encoding a protein having a sequence SEQ ID NO: 345. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 143. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 143. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 143. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 143. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 143. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:143. In some cases, the therapeutic agent is an antisense oligomer complementary to a sequence with 100% sequence identity to SEQ ID NO:143. In some cases, the therapeutic agent increases level of a first processed mRNA encoding a protein having a sequence SEQ ID NO: 8110, and decreases level of a second processed mRNA encoding a protein having a sequence SEQ ID NO: 346. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 144. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 144. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 144. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 144. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 144. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:144. In some cases, the therapeutic agent is an antisense oligomer complementary to a sequence with 100% sequence identity to SEQ ID NO:144. In some cases, the therapeutic agent increases level of a first processed mRNA encoding a protein having a sequence SEQ ID NO: 8111, and decreases level of a second processed mRNA encoding a protein having a sequence SEQ ID NO: 347. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 145. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 145. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 145. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 145. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 145. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:145. In some cases, the therapeutic agent is an antisense oligomer complementary to a sequence with 100% sequence identity to SEQ ID NO:145. In some cases, the therapeutic agent increases level of a first processed mRNA encoding a protein having a sequence SEQ ID NO: 8112, and decreases level of a second processed mRNA encoding a protein having a sequence SEQ ID NO: 348. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 146. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 146. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 146. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 146. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 146. In some cases, the targeted region of the pre-mRNA is within a sequence between a pair of genomic sites GRCh38/hg38: chr16 89317075, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:146. In some cases, the therapeutic agent is an antisense oligomer complementary to a sequence with 100% sequence identity to SEQ ID NO:146. In some cases, the therapeutic agent increases level of a first processed mRNA encoding a protein having a sequence SEQ ID NO: 8113, and decreases level of a second processed mRNA encoding a protein having a sequence SEQ ID NO: 349. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 147. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 147. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 147. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 147. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 147. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:147. In some cases, the therapeutic agent is an antisense oligomer complementary to a sequence with 100% sequence identity to SEQ ID NO:147. In some cases, the therapeutic agent increases level of a first processed mRNA encoding a protein having a sequence SEQ ID NO: 8114, and decreases level of a second processed mRNA encoding a protein having a sequence SEQ ID NO: 350. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 148. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 148. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 148. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 148. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 148. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:148. In some cases, the therapeutic agent is an antisense oligomer complementary to a sequence with 100% sequence identity to SEQ ID NO:148. In some cases, the therapeutic agent increases level of a first processed mRNA encoding a protein having a sequence SEQ ID NO: 8115, and decreases level of a second processed mRNA encoding a protein having a sequence SEQ ID NO: 351. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 149. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 149. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 149. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 149. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 149. In some cases, the targeted region of the pre-mRNA is within a sequence between a pair of genomic sites GRCh38/hg38: chr16 89317075, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:149. In some cases, the therapeutic agent is an antisense oligomer complementary to a sequence with 100% sequence identity to SEQ ID NO:149. In some cases, the therapeutic agent increases level of a first processed mRNA encoding a protein having a sequence SEQ ID NO: 8116, and decreases level of a second processed mRNA encoding a protein having a sequence SEQ ID NO: 352. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 150. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 150. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 150. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 150. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 150. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:150. In some cases, the therapeutic agent is an antisense oligomer complementary to a sequence with 100% sequence identity to SEQ ID NO:150. In some cases, the therapeutic agent increases level of a first processed mRNA encoding a protein having a sequence SEQ ID NO: 8117, and decreases level of a second processed mRNA encoding a protein having a sequence SEQ ID NO: 353. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 151. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 151. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 151. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 151. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 151. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:151. In some cases, the therapeutic agent is an antisense oligomer complementary to a sequence with 100% sequence identity to SEQ ID NO:151. In some cases, the therapeutic agent increases level of a first processed mRNA encoding a protein having a sequence SEQ ID NO: 8118, and decreases level of a second processed mRNA encoding a protein having a sequence SEQ ID NO: 354. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 152. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 152. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 152. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 152. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 152. In some cases, the targeted region of the pre-mRNA is within a sequence between a pair of genomic sites GRCh38/hg38: chr16 89317075, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:152. In some cases, the therapeutic agent is an antisense oligomer complementary to a sequence with 100% sequence identity to SEQ ID NO:152. In some cases, the therapeutic agent increases level of a first processed mRNA encoding a protein having a sequence SEQ ID NO: 8119, and decreases level of a second processed mRNA encoding a protein having a sequence SEQ ID NO: 355. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 153. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 153. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 153. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 153. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 153. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:153. In some cases, the therapeutic agent is an antisense oligomer complementary to a sequence with 100% sequence identity to SEQ ID NO:153. In some cases, the therapeutic agent increases level of a first processed mRNA encoding a protein having a sequence SEQ ID NO: 8120, and decreases level of a second processed mRNA encoding a protein having a sequence SEQ ID NO: 356. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 154. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 154. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317075. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 154. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 154. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 154. In some cases, the targeted region of the pre-mRNA is within a sequence between a pair of genomic sites GRCh38/hg38: chr16 89317075, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:154. In some cases, the therapeutic agent is an antisense oligomer complementary to a sequence with 100% sequence identity to SEQ ID NO:154. In some cases, the therapeutic agent increases level of a first processed mRNA encoding a protein having a sequence SEQ ID NO: 8121, and decreases level of a second processed mRNA encoding a protein having a sequence SEQ ID NO: 357. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 155. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 155. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 155. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 155. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 155. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:155. In some cases, the therapeutic agent is an antisense oligomer complementary to a sequence with 100% sequence identity to SEQ ID NO:155. In some cases, the therapeutic agent increases level of a first processed mRNA encoding a protein having a sequence SEQ ID NO: 8122, and decreases level of a second processed mRNA encoding a protein having a sequence SEQ ID NO: 358. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 156. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 156. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 89317078. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 156. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 156. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 89316933. In some cases, the targeted region of the pre-mRNA is within a sequence SEQ ID NO: 156. In some cases, the targeted region of the pre-mRNA is within a sequence between a pair of genomic sites GRCh38/hg38: chr16 89317078, and GRCh38/hg38: chr16 89316933. In some cases, the therapeutic agent is an antisense oligomer complementary to a sequence with at least about 80%, 85%, 90%, 95%, 96%, 97%, 98%, 99% or 100% sequence identity to SEQ ID NO:156. In some cases, the therapeutic agent is an antisense oligomer complementary to a sequence with 100% sequence identity to SEQ ID NO:156. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1114-1181. In some cases, the therapeutic agent is an antisense oligomer that has a sequence selected from the group consisting of SEQ ID NOs: 1114-1181. In some cases, the target peptide sequence is a portion of ANKRD1 1 protein, and the method treats a disease or the condition that comprises Intellectual Disability; KBG syndrome; or 16924.3 microdeletion syndrome.

ANKS3

In some cases, the target peptide sequence is a portion of ANKS3 protein. In some cases, the therapeutic agent increases level of a first processed mRNA encoding a protein having a sequence SEQ ID NO: 8123, and decreases level of a second processed mRNA encoding a protein having a sequence SEQ ID NO: 359. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 157. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 157. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 4726780. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 4726780. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 157. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 157. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 4726659. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 4726659. In some cases, the targeted region of the pre-mRNA is within a sequence SEQ ID NO: 157. In some cases, the targeted region of the pre-mRNA is within a sequence between a pair of genomic sites GRCh38/hg38: chr16 4726780, and GRCh38/hg38: chr16 4726659. In some cases, the therapeutic agent is an antisense oligomer complementary to a sequence with at least about 80%, 85%, 90%, 95%, 96%, 97%, 98%, 99% or 100% sequence identity to SEQ ID NO:157. In some cases, the therapeutic agent is an antisense oligomer complementary to a sequence with 100% sequence identity to SEQ ID NO:157. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1182-1244. In some cases, the therapeutic agent is an antisense oligomer that has a sequence selected from the group consisting of SEQ ID NOs: 1182-1244. In some cases, the target peptide sequence is a portion of ANKS3 protein, and the method treats a disease or the condition that comprises Situs Inversus.

APOL4

In some cases, the target peptide sequence is a portion of APOL4 protein. In some cases, the therapeutic agent increases level of a first processed mRNA encoding a protein having a sequence SEQ ID NO: 8124, and decreases level of a second processed mRNA encoding a protein having a sequence SEQ ID NO: 360. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 158. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 158. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr22 36201796. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr22 36201796. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 158. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 158. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr22 36201700. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr22 36201700. In some cases, the targeted region of the pre-mRNA is within a sequence SEQ ID NO: 158. In some cases, the targeted region of the pre-mRNA is within a sequence between a pair of genomic sites GRCh38/hg38: chr22 36201796, and GRCh38/hg38: chr22 36201700. In some cases, the therapeutic agent is an antisense oligomer complementary to a sequence with at least about 80%, 85%, 90%, 95%, 96%, 97%, 98%, 99% or 100% sequence identity to SEQ ID NO:158. In some cases, the therapeutic agent is an antisense oligomer complementary to a sequence with 100% sequence identity to SEQ ID NO:158. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1245-1302. In some cases, the therapeutic agent is an antisense oligomer that has a sequence selected from the group consisting of SEQ ID NOs: 1245-1302. In some cases, the target peptide sequence is a portion of APOL4 protein, and the method treats a disease or the condition that comprises Schizophrenia.

APPL1

In some cases, the target peptide sequence is a portion of APPL1 protein. In some cases, the therapeutic agent increases level of a first processed mRNA encoding a protein having a sequence SEQ ID NO: 8125, and decreases level of a second processed mRNA encoding a protein having a sequence SEQ ID NO: 361. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 159. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 159. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 57230714. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 57230714. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 159. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 159. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 57230767. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 57230767. In some cases, the targeted region of the pre-mRNA is within a sequence SEQ ID NO: 159. In some cases, the targeted region of the pre-mRNA is within a sequence between a pair of genomic sites GRCh38/hg38: chr3 57230714, and GRCh38/hg38: chr3 57230767. In some cases, the therapeutic agent is an antisense oligomer complementary to a sequence with at least about 80%, 85%, 90%, 95%, 96%, 97%, 98%, 99% or 100% sequence identity to SEQ ID NO:159. In some cases, the therapeutic agent is an antisense oligomer complementary to a sequence with 100% sequence identity to SEQ ID NO:159. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1303-1352. In some cases, the therapeutic agent is an antisense oligomer that has a sequence selected from the group consisting of SEQ ID NOs: 1303-1352. In some cases, the target peptide sequence is a portion of APPL1 protein, and the method treats a disease or the condition that comprises Malignant neoplasm of breast; Maturity onset diabetes mellitus in young; or Maturity onset diabetes mellitus of the young, Type 14.

ARCN1

In some cases, the target peptide sequence is a portion of ARCN1 protein. In some cases, the therapeutic agent increases level of a first processed mRNA encoding a protein having a sequence SEQ ID NO: 8126, and decreases level of a second processed mRNA encoding a protein having a sequence SEQ ID NO: 362. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 160. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 160. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 118573620. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 118573620. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 160. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 160. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 118573783. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 118573783. In some cases, the targeted region of the pre-mRNA is within a sequence SEQ ID NO: 160. In some cases, the targeted region of the pre-mRNA is within a sequence between a pair of genomic sites GRCh38/hg38: chr11 118573620, and GRCh38/hg38: chr11 118573783. In some cases, the therapeutic agent is an antisense oligomer complementary to a sequence with at least about 80%, 85%, 90%, 95%, 96%, 97%, 98%, 99% or 100% sequence identity to SEQ ID NO:160. In some cases, the therapeutic agent is an antisense oligomer complementary to a sequence with 100% sequence identity to SEQ ID NO:160. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1353-1424. In some cases, the therapeutic agent is an antisense oligomer that has a sequence selected from the group consisting of SEQ ID NOs: 1353-1424. In some cases, the target peptide sequence is a portion of ARCN1 protein, and the method treats a disease or the condition that comprises Intellectual Disability; or Microcephaly.

ARMC8

In some cases, the target peptide sequence is a portion of ARMC8 protein. In some cases, the therapeutic agent increases level of a first processed mRNA encoding a protein having a sequence SEQ ID NO: 8127, and decreases level of a second processed mRNA encoding a protein having a sequence SEQ ID NO: 363. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 161. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 161. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 138188455. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 138188455. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 161. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 161. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 138188530. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 138188530. In some cases, the targeted region of the pre-mRNA is within a sequence SEQ ID NO: 161. In some cases, the targeted region of the pre-mRNA is within a sequence between a pair of genomic sites GRCh38/hg38: chr3 138188455, and GRCh38/hg38: chr3 138188530. In some cases, the therapeutic agent is an antisense oligomer complementary to a sequence with at least about 80%, 85%, 90%, 95%, 96%, 97%, 98%, 99% or 100% sequence identity to SEQ ID NO:161. In some cases, the therapeutic agent is an antisense oligomer complementary to a sequence with 100% sequence identity to SEQ ID NO:161. In some cases, the therapeutic agent increases level of a first processed mRNA encoding a protein having a sequence SEQ ID NO: 8128, and decreases level of a second processed mRNA encoding a protein having a sequence SEQ ID NO: 364. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 162. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 162. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 138188455. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 138188455. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 162. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 162. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 138188530. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 138188530. In some cases, the targeted region of the pre-mRNA is within a sequence SEQ ID NO: 162. In some cases, the targeted region of the pre-mRNA is within a sequence between a pair of genomic sites GRCh38/hg38: chr3 138188455, and GRCh38/hg38: chr3 138188530. In some cases, the therapeutic agent is an antisense oligomer complementary to a sequence with at least about 80%, 85%, 90%, 95%, 96%, 97%, 98%, 99% or 100% sequence identity to SEQ ID NO:162. In some cases, the therapeutic agent is an antisense oligomer complementary to a sequence with 100% sequence identity to SEQ ID NO:162. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1425-1478. In some cases, the therapeutic agent is an antisense oligomer that has a sequence selected from the group consisting of SEQ ID NOs: 1425-1478. In some cases, the target peptide sequence is a portion of ARMC8 protein, and the method treats a disease or the condition that comprises Polydactyly; or Ciliopathies.

ARNTL

In some cases, the target peptide sequence is a portion of ARNTL protein. In some cases, the therapeutic agent increases level of a first processed mRNA encoding a protein having a sequence SEQ ID NO: 8129, and decreases level of a second processed mRNA encoding a protein having a sequence SEQ ID NO: 365. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 163. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 163. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 13355226. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 13355226. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 163. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 163. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 13355296. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 13355296. In some cases, the targeted region of the pre-mRNA is within a sequence SEQ ID NO: 163. In some cases, the targeted region of the pre-mRNA is within a sequence between a pair of genomic sites GRCh38/hg38: chr11 13355226, and GRCh38/hg38: chr11 13355296. In some cases, the therapeutic agent is an antisense oligomer complementary to a sequence with at least about 80%, 85%, 90%, 95%, 96%, 97%, 98%, 99% or 100% sequence identity to SEQ ID NO:163. In some cases, the therapeutic agent is an antisense oligomer complementary to a sequence with 100% sequence identity to SEQ ID NO:163. In some cases, the therapeutic agent increases level of a first processed mRNA encoding a protein having a sequence SEQ ID NO: 8130, and decreases level of a second processed mRNA encoding a protein having a sequence SEQ ID NO: 366. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 164. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 164. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 13355226. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 13355226. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 164. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 164. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 13355296. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 13355296. In some cases, the targeted region of the pre-mRNA is within a sequence SEQ ID NO: 164. In some cases, the targeted region of the pre-mRNA is within a sequence between a pair of genomic sites GRCh38/hg38: chr11 13355226, and GRCh38/hg38: chr11 13355296. In some cases, the therapeutic agent is an antisense oligomer complementary to a sequence with at least about 80%, 85%, 90%, 95%, 96%, 97%, 98%, 99% or 100% sequence identity to SEQ ID NO:164. In some cases, the therapeutic agent is an antisense oligomer complementary to a sequence with 100% sequence identity to SEQ ID NO:164. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1479-1531. In some cases, the therapeutic agent is an antisense oligomer that has a sequence selected from the group consisting of SEQ ID NOs: 1479-1531. In some cases, the target peptide sequence is a portion of ARNTL protein, and the method treats a disease or the condition that comprises Mental Depression; Seasonal Affective Disorder; Bipolar Disorder; Mood Disorders; or Depressive disorder. In some cases, the method treats a cancer that is associated with ARNTL protein.

BOC

In some cases, the target peptide sequence is a portion of BOC protein. In some cases, the therapeutic agent increases level of a first processed mRNA encoding a protein having a sequence SEQ ID NO: 8131, and decreases level of a second processed mRNA encoding a protein having a sequence SEQ ID NO: 367. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 165. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 165. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 165. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 165. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases, the targeted region of the pre-mRNA is within a sequence SEQ ID NO: 165. In some cases, the targeted region of the pre-mRNA is within a sequence between a pair of genomic sites GRCh38/hg38: chr3 113249722, and GRCh38/hg38: chr3 113249899. In some cases, the therapeutic agent is an antisense oligomer complementary to a sequence with at least about 80%, 85%, 90%, 95%, 96%, 97%, 98%, 99% or 100% sequence identity to SEQ ID NO:165. In some cases, the therapeutic agent is an antisense oligomer complementary to a sequence with 100% sequence identity to SEQ ID NO:165. In some cases, the therapeutic agent increases level of a first processed mRNA encoding a protein having a sequence SEQ ID NO: 8132, and decreases level of a second processed mRNA encoding a protein having a sequence SEQ ID NO: 368. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 166. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 166. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 166. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 166. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases, the targeted region of the pre-mRNA is within a sequence SEQ ID NO: 166. In some cases, the targeted region of the pre-mRNA is within a sequence between a pair of genomic sites GRCh38/hg38: chr3 113249722, and GRCh38/hg38: chr3 113249899. In some cases, the therapeutic agent is an antisense oligomer complementary to a sequence with at least about 80%, 85%, 90%, 95%, 96%, 97%, 98%, 99% or 100% sequence identity to SEQ ID NO:166. In some cases, the therapeutic agent is an antisense oligomer complementary to a sequence with 100% sequence identity to SEQ ID NO:166. In some cases, the therapeutic agent increases level of a first processed mRNA encoding a protein having a sequence SEQ ID NO: 8133, and decreases level of a second processed mRNA encoding a protein having a sequence SEQ ID NO: 369. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 167. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 167. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 167. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 167. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases, the targeted region of the pre-mRNA is within a sequence SEQ ID NO: 167. In some cases, the targeted region of the pre-mRNA is within a sequence between a pair of genomic sites GRCh38/hg38: chr3 113249722, and GRCh38/hg38: chr3 113249899. In some cases, the therapeutic agent is an antisense oligomer complementary to a sequence with at least about 80%, 85%, 90%, 95%, 96%, 97%, 98%, 99% or 100% sequence identity to SEQ ID NO:167. In some cases, the therapeutic agent is an antisense oligomer complementary to a sequence with 100% sequence identity to SEQ ID NO:167. In some cases, the therapeutic agent increases level of a first processed mRNA encoding a protein having a sequence SEQ ID NO: 8134, and decreases level of a second processed mRNA encoding a protein having a sequence SEQ ID NO: 370. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 168. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 168. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 113249722. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 168. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 168. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 113249899. In some cases, the targeted region of the pre-mRNA is within a sequence SEQ ID NO: 168. In some cases, the targeted region of the pre-mRNA is within a sequence between a pair of genomic sites GRCh38/hg38: chr3 113249722, and GRCh38/hg38: chr3 113249899. In some cases, the therapeutic agent is an antisense oligomer complementary to a sequence with at least about 80%, 85%, 90%, 95%, 96%, 97%, 98%, 99% or 100% sequence identity to SEQ ID NO:168. In some cases, the therapeutic agent is an antisense oligomer complementary to a sequence with 100% sequence identity to SEQ ID NO:168. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1532-1606. In some cases, the therapeutic agent is an antisense oligomer that has a sequence selected from the group consisting of SEQ ID NOs: 1532-1606. In some cases, the target peptide sequence is a portion of BOC protein, and the method treats a disease or the condition that comprises Malignant neoplasm of breast.

BRCA1

In some cases, the target peptide sequence is a portion of BRCA1 protein. In some cases, the therapeutic agent increases level of a first processed mRNA encoding a protein having a sequence SEQ ID NO: 8135, and decreases level of a second processed mRNA encoding a protein having a sequence SEQ ID NO: 371. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 169. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 169. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 43106533. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 43106533. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 169. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 169. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 43106456. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 43106456. In some cases, the targeted region of the pre-mRNA is within a sequence SEQ ID NO: 169. In some cases, the targeted region of the pre-mRNA is within a sequence between a pair of genomic sites GRCh38/hg38: chr17 43106533, and GRCh38/hg38: chr17 43106456. In some cases, the therapeutic agent is an antisense oligomer complementary to a sequence with at least about 80%, 85%, 90%, 95%, 96%, 97%, 98%, 99% or 100% sequence identity to SEQ ID NO:169. In some cases, the therapeutic agent is an antisense oligomer complementary to a sequence with 100% sequence identity to SEQ ID NO:169. In some cases, the therapeutic agent increases level of a first processed mRNA encoding a protein having a sequence SEQ ID NO: 8136, and decreases level of a second processed mRNA encoding a protein having a sequence SEQ ID NO: 372. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 170. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 170. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 43106533. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 43106533. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 170. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 170. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 43106456. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 43106456. In some cases, the targeted region of the pre-mRNA is within a sequence SEQ ID NO: 170. In some cases, the targeted region of the pre-mRNA is within a sequence between a pair of genomic sites GRCh38/hg38: chr17 43106533, and GRCh38/hg38: chr17 43106456. In some cases, the therapeutic agent is an antisense oligomer complementary to a sequence with at least about 80%, 85%, 90%, 95%, 96%, 97%, 98%, 99% or 100% sequence identity to SEQ ID NO:170. In some cases, the therapeutic agent is an antisense oligomer complementary to a sequence with 100% sequence identity to SEQ ID NO:170. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1607-1661. In some cases, the therapeutic agent is an antisense oligomer that has a sequence selected from the group consisting of SEQ ID NOs: 1607-1661. In some cases, the target peptide sequence is a portion of BRCA1 protein, and the method treats a disease or the condition that comprises Cytopenia; Gastrointestinal Neoplasms; Depressive disorder; Prostate cancer, familial; Primary peritoneal carcinoma; Ovarian Cancer, Familial, Susceptibility to, 1; Breast Cancer, Familial Male; Malignant Neoplasms; Pancreatic Neoplasm; Breast Cancer, Familial; Neoplasm of uncertain or unknown behavior of breast; Ovarian Carcinoma; Adenocarcinoma of prostate; Neoplasm of uncertain or unknown behavior of ovary; Malignant neoplasm of pancreas; Pancreatic carcinoma; Malignant neoplasm of ovary; Congenital anemia; Hematologic Neoplasms; Breast adenocarcinoma; Breast-Ovarian Cancer, Familial, Susceptibility to, 1; Pancreatic carcinoma, familial; Benign tumor of pancreas; Breast Carcinoma; Ovarian neoplasm; Hereditary Breast and Ovarian Cancer Syndrome; Mental Depression; Epithelial ovarian cancer; Malignant neoplasm of breast; Sporadic Breast Carcinoma; Schizophrenia; Breast-ovarian cancer, familial, 1; or Pancreatic cancer, susceptibility to, 4.

C8B

In some cases, the target peptide sequence is a portion of C8B protein. In some cases, the therapeutic agent increases level of a first processed mRNA encoding a protein having a sequence SEQ ID NO: 8137, and decreases level of a second processed mRNA encoding a protein having a sequence SEQ ID NO: 373. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 171. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 171. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 56959657. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 56959657. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 171. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 171. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 56959529. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 56959529. In some cases, the targeted region of the pre-mRNA is within a sequence SEQ ID NO: 171. In some cases, the targeted region of the pre-mRNA is within a sequence between a pair of genomic sites GRCh38/hg38: chr1 56959657, and GRCh38/hg38: chr1 56959529. In some cases, the therapeutic agent is an antisense oligomer complementary to a sequence with at least about 80%, 85%, 90%, 95%, 96%, 97%, 98%, 99% or 100% sequence identity to SEQ ID NO:171. In some cases, the therapeutic agent is an antisense oligomer complementary to a sequence with 100% sequence identity to SEQ ID NO:171. In some cases, the therapeutic agent increases level of a first processed mRNA encoding a protein having a sequence SEQ ID NO: 8138, and decreases level of a second processed mRNA encoding a protein having a sequence SEQ ID NO: 374. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 172. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 172. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 56959657. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 56959657. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 172. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 172. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 56959529. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 56959529. In some cases, the targeted region of the pre-mRNA is within a sequence SEQ ID NO: 172. In some cases, the targeted region of the pre-mRNA is within a sequence between a pair of genomic sites GRCh38/hg38: chr1 56959657, and GRCh38/hg38: chr1 56959529. In some cases, the therapeutic agent is an antisense oligomer complementary to a sequence with at least about 80%, 85%, 90%, 95%, 96%, 97%, 98%, 99% or 100% sequence identity to SEQ ID NO:172. In some cases, the therapeutic agent is an antisense oligomer complementary to a sequence with 100% sequence identity to SEQ ID NO:172. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1662-1726. In some cases, the therapeutic agent is an antisense oligomer that has a sequence selected from the group consisting of SEQ ID NOs: 1662-1726. In some cases, the target peptide sequence is a portion of C8B protein, and the method treats a disease or the condition that comprises C8 deficiency, type II.

CALM1

In some cases, the target peptide sequence is a portion of CALM1 protein. In some cases, the therapeutic agent increases level of a first processed mRNA encoding a protein having a sequence SEQ ID NO: 8139, and decreases level of a second processed mRNA encoding a protein having a sequence SEQ ID NO: 375. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 173. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 173. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 90398991. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 90398991. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 173. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 173. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 90399087. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 90399087. In some cases, the targeted region of the pre-mRNA is within a sequence SEQ ID NO: 173. In some cases, the targeted region of the pre-mRNA is within a sequence between a pair of genomic sites GRCh38/hg38: chr14 90398991, and GRCh38/hg38: chr14 90399087. In some cases, the therapeutic agent is an antisense oligomer complementary to a sequence with at least about 80%, 85%, 90%, 95%, 96%, 97%, 98%, 99% or 100% sequence identity to SEQ ID NO:173. In some cases, the therapeutic agent is an antisense oligomer complementary to a sequence with 100% sequence identity to SEQ ID NO:173. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1727-1784. In some cases, the therapeutic agent is an antisense oligomer that has a sequence selected from the group consisting of SEQ ID NOs: 1727-1784. In some cases, the target peptide sequence is a portion of CALM1 protein, and the method treats a disease or the condition that comprises Ventricular Tachycardia, Catecholaminergic Polymorphic, 4; Ventricular Tachycardia, Catecholaminergic Polymorphic, 1; Long QT Syndrome; Romano-Ward Syndrome; Long QT Syndrome 14; or Schizophrenia.

CALM3

In some cases, the target peptide sequence is a portion of CALM3 protein. In some cases, the therapeutic agent increases level of a first processed mRNA encoding a protein having a sequence SEQ ID NO: 8140, and decreases level of a second processed mRNA encoding a protein having a sequence SEQ ID NO: 376. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 174. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 174. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 46608205. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 46608205. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 174. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 174. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 46608340. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 46608340. In some cases, the targeted region of the pre-mRNA is within a sequence SEQ ID NO: 174. In some cases, the targeted region of the pre-mRNA is within a sequence between a pair of genomic sites GRCh38/hg38: chr19 46608205, and GRCh38/hg38: chr19 46608340. In some cases, the therapeutic agent is an antisense oligomer complementary to a sequence with at least about 80%, 85%, 90%, 95%, 96%, 97%, 98%, 99% or 100% sequence identity to SEQ ID NO:174. In some cases, the therapeutic agent is an antisense oligomer complementary to a sequence with 100% sequence identity to SEQ ID NO:174. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1785-1850. In some cases, the therapeutic agent is an antisense oligomer that has a sequence selected from the group consisting of SEQ ID NOs: 1785-1850. In some cases, the target peptide sequence is a portion of CALM3 protein, and the method treats a disease or the condition that comprises Ventricular Tachycardia, Catecholaminergic Polymorphic, 1; or Long QT Syndrome.

CASP5

In some cases, the target peptide sequence is a portion of CASP5 protein. In some cases, the therapeutic agent increases level of a first processed mRNA encoding a protein having a sequence SEQ ID NO: 8141, and decreases level of a second processed mRNA encoding a protein having a sequence SEQ ID NO: 377. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 175. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 175. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 105009053. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 105009053. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 175. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 175. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 105008807. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 105008807. In some cases, the targeted region of the pre-mRNA is within a sequence SEQ ID NO: 175. In some cases, the targeted region of the pre-mRNA is within a sequence between a pair of genomic sites GRCh38/hg38: chr11 105009053, and GRCh38/hg38: chr11 105008807. In some cases, the therapeutic agent is an antisense oligomer complementary to a sequence with at least about 80%, 85%, 90%, 95%, 96%, 97%, 98%, 99% or 100% sequence identity to SEQ ID NO:175. In some cases, the therapeutic agent is an antisense oligomer complementary to a sequence with 100% sequence identity to SEQ ID NO:175. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1851-1938. In some cases, the therapeutic agent is an antisense oligomer that has a sequence selected from the group consisting of SEQ ID NOs: 1851-1938. In some cases, the target peptide sequence is a portion of CASP5 protein, and the method treats a cancer that is associated with CASP5 protein

CAST

In some cases, the target peptide sequence is a portion of CAST protein. In some cases, the therapeutic agent increases level of a first processed mRNA encoding a protein having a sequence SEQ ID NO: 8142, and decreases level of a second processed mRNA encoding a protein having a sequence SEQ ID NO: 378. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 176. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 176. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr5 96726754. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr5 96726754. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 176. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 176. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr5 96726859. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr5 96726859. In some cases, the targeted region of the pre-mRNA is within a sequence SEQ ID NO: 176. In some cases, the targeted region of the pre-mRNA is within a sequence between a pair of genomic sites GRCh38/hg38: chr5 96726754, and GRCh38/hg38: chr5 96726859. In some cases, the therapeutic agent is an antisense oligomer complementary to a sequence with at least about 80%, 85%, 90%, 95%, 96%, 97%, 98%, 99% or 100% sequence identity to SEQ ID NO:176. In some cases, the therapeutic agent is an antisense oligomer complementary to a sequence with 100% sequence identity to SEQ ID NO:176. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1939-1998. In some cases, the therapeutic agent is an antisense oligomer that has a sequence selected from the group consisting of SEQ ID NOs: 1939-1998. In some cases, the target peptide sequence is a portion of CAST protein, and the method treats a disease or the condition that comprises Peeling Skin Syndrome; Peeling Skin with Leukonychia, Acral Punctate Keratoses, Cheilitis, and Knuckle Pads; Erythrokeratoderma; or Palmoplantar Keratosis.

CEP19

In some cases, the target peptide sequence is a portion of CEP19 protein. In some cases, the therapeutic agent increases level of a first processed mRNA encoding a protein having a sequence SEQ ID NO: 8143, and decreases level of a second processed mRNA encoding a protein having a sequence SEQ ID NO: 379. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 177. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 177. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 196708727. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 196708727. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 177. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 177. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 196708528. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 196708528. In some cases, the targeted region of the pre-mRNA is within a sequence SEQ ID NO: 177. In some cases, the targeted region of the pre-mRNA is within a sequence between a pair of genomic sites GRCh38/hg38: chr3 196708727, and GRCh38/hg38: chr3 196708528. In some cases, the therapeutic agent is an antisense oligomer complementary to a sequence with at least about 80%, 85%, 90%, 95%, 96%, 97%, 98%, 99% or 100% sequence identity to SEQ ID NO:177. In some cases, the therapeutic agent is an antisense oligomer complementary to a sequence with 100% sequence identity to SEQ ID NO:177. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 1999-2077. In some cases, the therapeutic agent is an antisense oligomer that has a sequence selected from the group consisting of SEQ ID NOs: 1999-2077. In some cases, the target peptide sequence is a portion of CEP19 protein, and the method treats a disease or the condition that comprises Morbid Obesity and Spermatogenic Failure.

CEP57

In some cases, the target peptide sequence is a portion of CEP57 protein. In some cases, the therapeutic agent increases level of a first processed mRNA encoding a protein having a sequence SEQ ID NO: 8144, and decreases level of a second processed mRNA encoding a protein having a sequence SEQ ID NO: 380. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 178. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 178. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 95795516. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 95795516. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 178. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 178. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 95795559. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 95795559. In some cases, the targeted region of the pre-mRNA is within a sequence SEQ ID NO: 178. In some cases, the targeted region of the pre-mRNA is within a sequence between a pair of genomic sites GRCh38/hg38: chr11 95795516, and GRCh38/hg38: chr1l 95795559. In some cases, the therapeutic agent is an antisense oligomer complementary to a sequence with at least about 80%, 85%, 90%, 95%, 96%, 97%, 98%, 99% or 100% sequence identity to SEQ ID NO:178. In some cases, the therapeutic agent is an antisense oligomer complementary to a sequence with 100% sequence identity to SEQ ID NO:178. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2078-2125. In some cases, the therapeutic agent is an antisense oligomer that has a sequence selected from the group consisting of SEQ ID NOs: 2078-2125. In some cases, the target peptide sequence is a portion of CEP57 protein, and the method treats a disease or the condition that comprises Intellectual Disability; Warburton Anyane Yeboa syndrome; or Mosaic Variegated Aneuploidy Syndrome.

CHEK1

In some cases, the target peptide sequence is a portion of CHEK1 protein. In some cases, the therapeutic agent increases level of a first processed mRNA encoding a protein having a sequence SEQ ID NO: 8145, and decreases level of a second processed mRNA encoding a protein having a sequence SEQ ID NO: 381. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 179. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 179. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 125627607. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 125627607. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 179. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 179. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 125627830. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 125627830. In some cases, the targeted region of the pre-mRNA is within a sequence SEQ ID NO: 179. In some cases, the targeted region of the pre-mRNA is within a sequence between a pair of genomic sites GRCh38/hg38: chr11 125627607, and GRCh38/hg38: chr11 125627830. In some cases, the therapeutic agent is an antisense oligomer complementary to a sequence with at least about 80%, 85%, 90%, 95%, 96%, 97%, 98%, 99% or 100% sequence identity to SEQ ID NO:179. In some cases, the therapeutic agent is an antisense oligomer complementary to a sequence with 100% sequence identity to SEQ ID NO:179. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2126-2209. In some cases, the therapeutic agent is an antisense oligomer that has a sequence selected from the group consisting of SEQ ID NOs: 2126-2209. In some cases, the target peptide sequence is a portion of CHEK1 protein, and the method treats a disease or the condition that comprises Breast Cancer, Familial; Malignant neoplasm of ovary; or Schizophrenia.

CIB2

In some cases, the target peptide sequence is a portion of CIB2 protein. In some cases, the therapeutic agent increases level of a first processed mRNA encoding a protein having a sequence SEQ ID NO: 8146, and decreases level of a second processed mRNA encoding a protein having a sequence SEQ ID NO: 382. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 180. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 180. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 78111276. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 78111276. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 180. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 180. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 78111165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 78111165. In some cases, the targeted region of the pre-mRNA is within a sequence SEQ ID NO: 180. In some cases, the targeted region of the pre-mRNA is within a sequence between a pair of genomic sites GRCh38/hg38: chr15 78111276, and GRCh38/hg38: chr15 78111165. In some cases, the therapeutic agent is an antisense oligomer complementary to a sequence with at least about 80%, 85%, 90%, 95%, 96%, 97%, 98%, 99% or 100% sequence identity to SEQ ID NO:180. In some cases, the therapeutic agent is an antisense oligomer complementary to a sequence with 100% sequence identity to SEQ ID NO:180. In some cases, the therapeutic agent increases level of a first processed mRNA encoding a protein having a sequence SEQ ID NO: 8147, and decreases level of a second processed mRNA encoding a protein having a sequence SEQ ID NO: 383. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 181. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 181. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 78111276. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 78111276. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 181. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 181. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 78111165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 78111165. In some cases, the targeted region of the pre-mRNA is within a sequence SEQ ID NO: 181. In some cases, the targeted region of the pre-mRNA is within a sequence between a pair of genomic sites GRCh38/hg38: chr15 78111276, and GRCh38/hg38: chr15 78111165. In some cases, the therapeutic agent is an antisense oligomer complementary to a sequence with at least about 80%, 85%, 90%, 95%, 96%, 97%, 98%, 99% or 100% sequence identity to SEQ ID NO:181. In some cases, the therapeutic agent is an antisense oligomer complementary to a sequence with 100% sequence identity to SEQ ID NO:181. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2210-2270. In some cases, the therapeutic agent is an antisense oligomer that has a sequence selected from the group consisting of SEQ ID NOs: 2210-2270. In some cases, the target peptide sequence is a portion of CIB2 protein, and the method treats a disease or the condition that comprises Usher Syndrome, Type I; Intellectual Disability; Deafness, Autosomal Recessive 48; or Usher Syndrome, Type IJ.

COX4I1

In some cases, the target peptide sequence is a portion of COX4I1 protein. In some cases, the therapeutic agent increases level of a first processed mRNA encoding a protein having a sequence SEQ ID NO: 8148, and decreases level of a second processed mRNA encoding a protein having a sequence SEQ ID NO: 384. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 182. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 182. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 85804941. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 85804941. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 182. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 182. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 85805104. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 85805104. In some cases, the targeted region of the pre-mRNA is within a sequence SEQ ID NO: 182. In some cases, the targeted region of the pre-mRNA is within a sequence between a pair of genomic sites GRCh38/hg38: chr16 85804941, and GRCh38/hg38: chr16 85805104. In some cases, the therapeutic agent is an antisense oligomer complementary to a sequence with at least about 80%, 85%, 90%, 95%, 96%, 97%, 98%, 99% or 100% sequence identity to SEQ ID NO:182. In some cases, the therapeutic agent is an antisense oligomer complementary to a sequence with 100% sequence identity to SEQ ID NO:182. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2271-2342. In some cases, the therapeutic agent is an antisense oligomer that has a sequence selected from the group consisting of SEQ ID NOs: 2271-2342. In some cases, the target peptide sequence is a portion of COX4I1 protein, and the method treats a disease or the condition that comprises Mitochondrial Diseases.

DCLRE1C

In some cases, the target peptide sequence is a portion of DCLRE1C protein. In some cases, the therapeutic agent increases level of a first processed mRNA encoding a protein having a sequence SEQ ID NO: 8149, and decreases level of a second processed mRNA encoding a protein having a sequence SEQ ID NO: 385. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 183. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 183. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 14939869. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 14939869. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 183. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 183. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 14939810. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 14939810. In some cases, the targeted region of the pre-mRNA is within a sequence SEQ ID NO: 183. In some cases, the targeted region of the pre-mRNA is within a sequence between a pair of genomic sites GRCh38/hg38: chr10 14939869, and GRCh38/hg38: chr10 14939810. In some cases, the therapeutic agent is an antisense oligomer complementary to a sequence with at least about 80%, 85%, 90%, 95%, 96%, 97%, 98%, 99% or 100% sequence identity to SEQ ID NO:183. In some cases, the therapeutic agent is an antisense oligomer complementary to a sequence with 100% sequence identity to SEQ ID NO:183. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2343-2393. In some cases, the therapeutic agent is an antisense oligomer that has a sequence selected from the group consisting of SEQ ID NOs: 2343-2393. In some cases, the target peptide sequence is a portion of DCLRE1C protein, and the method treats a disease or the condition that comprises Severe combined immunodeficiency with sensitivity to ionizing radiation; Severe Combined Immunodeficiency, Partial; Reticuloendotheliosis, familial, with eosinophilia; Omenn Syndrome; Athabaskan severe combined immunodeficiency; or Severe Combined Immunodeficiency, Athabaskan-Type.

DECR1

In some cases, the target peptide sequence is a portion of DECR1 protein. In some cases, the therapeutic agent increases level of a first processed mRNA encoding a protein having a sequence SEQ ID NO: 8150, and decreases level of a second processed mRNA encoding a protein having a sequence SEQ ID NO: 386. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 184. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 184. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 90005292. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 90005292. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 184. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 184. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 90005369. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 90005369. In some cases, the targeted region of the pre-mRNA is within a sequence SEQ ID NO: 184. In some cases, the targeted region of the pre-mRNA is within a sequence between a pair of genomic sites GRCh38/hg38: chr8 90005292, and GRCh38/hg38: chr8 90005369. In some cases, the therapeutic agent is an antisense oligomer complementary to a sequence with at least about 80%, 85%, 90%, 95%, 96%, 97%, 98%, 99% or 100% sequence identity to SEQ ID NO:184. In some cases, the therapeutic agent is an antisense oligomer complementary to a sequence with 100% sequence identity to SEQ ID NO:184. In some cases, the therapeutic agent increases level of a first processed mRNA encoding a protein having a sequence SEQ ID NO: 8151, and decreases level of a second processed mRNA encoding a protein having a sequence SEQ ID NO: 387. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 185. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 185. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 90015541. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 90015541. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 185. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 185. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 90015666. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 90015666. In some cases, the targeted region of the pre-mRNA is within a sequence SEQ ID NO: 185. In some cases, the targeted region of the pre-mRNA is within a sequence between a pair of genomic sites GRCh38/hg38: chr8 90015541, and GRCh38/hg38: chr8 90015666. In some cases, the therapeutic agent is an antisense oligomer complementary to a sequence with at least about 80%, 85%, 90%, 95%, 96%, 97%, 98%, 99% or 100% sequence identity to SEQ ID NO:185. In some cases, the therapeutic agent is an antisense oligomer complementary to a sequence with 100% sequence identity to SEQ ID NO:185. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2394-2457. In some cases, the therapeutic agent is an antisense oligomer that has a sequence selected from the group consisting of SEQ ID NOs: 2394-2457. In some cases, the target peptide sequence is a portion of DECR1 protein, and the method treats a disease or the condition that comprises Intellectual Disability; or Cocaine Abuse.

DHFR

In some cases, the target peptide sequence is a portion of DHFR protein. In some cases, the therapeutic agent increases level of a first processed mRNA encoding a protein having a sequence SEQ ID NO: 8152, and decreases level of a second processed mRNA encoding a protein having a sequence SEQ ID NO: 388. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 186. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 186. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr5 80649494. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr5 80649494. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 186. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 186. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr5 80649389. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr5 80649389. In some cases, the targeted region of the pre-mRNA is within a sequence SEQ ID NO: 186. In some cases, the targeted region of the pre-mRNA is within a sequence between a pair of genomic sites GRCh38/hg38: chr5 80649494, and GRCh38/hg38: chr5 80649389. In some cases, the therapeutic agent is an antisense oligomer complementary to a sequence with at least about 80%, 85%, 90%, 95%, 96%, 97%, 98%, 99% or 100% sequence identity to SEQ ID NO:186. In some cases, the therapeutic agent is an antisense oligomer complementary to a sequence with 100% sequence identity to SEQ ID NO:186. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2458-2517. In some cases, the therapeutic agent is an antisense oligomer that has a sequence selected from the group consisting of SEQ ID NOs: 2458-2517. In some cases, the target peptide sequence is a portion of DHFR protein, and the method treats a disease or the condition that comprises Intellectual Disability; Congenital anemia; Cytopenia; Neurodegeneration Due To Cerebral Folate Transport Deficiency; or Megaloblastic Anemia due to Dihydrofolate Reductase Deficiency.

DKK2

In some cases, the target peptide sequence is a portion of DKK2 protein. In some cases, the therapeutic agent increases level of a first processed mRNA encoding a protein having a sequence SEQ ID NO: 8153, and decreases level of a second processed mRNA encoding a protein having a sequence SEQ ID NO: 389. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 187. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 187. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 106925949. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 106925949. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 187. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 187. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 106925799. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 106925799. In some cases, the targeted region of the pre-mRNA is within a sequence SEQ ID NO: 187. In some cases, the targeted region of the pre-mRNA is within a sequence between a pair of genomic sites GRCh38/hg38: chr4 106925949, and GRCh38/hg38: chr4 106925799. In some cases, the therapeutic agent is an antisense oligomer complementary to a sequence with at least about 80%, 85%, 90%, 95%, 96%, 97%, 98%, 99% or 100% sequence identity to SEQ ID NO:187. In some cases, the therapeutic agent is an antisense oligomer complementary to a sequence with 100% sequence identity to SEQ ID NO:187. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2518-2586. In some cases, the therapeutic agent is an antisense oligomer that has a sequence selected from the group consisting of SEQ ID NOs: 2518-2586. In some cases, the target peptide sequence is a portion of DKK2 protein, and the method treats a disease or the condition that comprises Alcoholic Intoxication, Chronic.

DLG2

In some cases, the target peptide sequence is a portion of DLG2 protein. In some cases, the therapeutic agent increases level of a first processed mRNA encoding a protein having a sequence SEQ ID NO: 8154, and decreases level of a second processed mRNA encoding a protein having a sequence SEQ ID NO: 390. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 188. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 188. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 83651930. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 83651930. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 188. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 188. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 83651839. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 83651839. In some cases, the targeted region of the pre-mRNA is within a sequence SEQ ID NO: 188. In some cases, the targeted region of the pre-mRNA is within a sequence between a pair of genomic sites GRCh38/hg38: chr11 83651930, and GRCh38/hg38: chr11 83651839. In some cases, the therapeutic agent is an antisense oligomer complementary to a sequence with at least about 80%, 85%, 90%, 95%, 96%, 97%, 98%, 99% or 100% sequence identity to SEQ ID NO:188. In some cases, the therapeutic agent is an antisense oligomer complementary to a sequence with 100% sequence identity to SEQ ID NO:188. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2587-2643. In some cases, the therapeutic agent is an antisense oligomer that has a sequence selected from the group consisting of SEQ ID NOs: 2587-2643. In some cases, the target peptide sequence is a portion of DLG2 protein, and the method treats a disease or the condition that comprises Intellectual Disability; Bipolar Disorder; Unipolar Depression; Major Depressive Disorder; Schizophrenia related disorders; or Schizophrenia.

DOK2

In some cases, the target peptide sequence is a portion of DOK2 protein. In some cases, the therapeutic agent increases level of a first processed mRNA encoding a protein having a sequence SEQ ID NO: 8155, and decreases level of a second processed mRNA encoding a protein having a sequence SEQ ID NO: 391. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 189. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 189. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 21910857. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 21910857. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 189. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 189. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 21910673. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 21910673. In some cases, the targeted region of the pre-mRNA is within a sequence SEQ ID NO: 189. In some cases, the targeted region of the pre-mRNA is within a sequence between a pair of genomic sites GRCh38/hg38: chr8 21910857, and GRCh38/hg38: chr8 21910673. In some cases, the therapeutic agent is an antisense oligomer complementary to a sequence with at least about 80%, 85%, 90%, 95%, 96%, 97%, 98%, 99% or 100% sequence identity to SEQ ID NO:189. In some cases, the therapeutic agent is an antisense oligomer complementary to a sequence with 100% sequence identity to SEQ ID NO:189. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2644-2719. In some cases, the therapeutic agent is an antisense oligomer that has a sequence selected from the group consisting of SEQ ID NOs: 2644-2719. In some cases, the target peptide sequence is a portion of DOK2 protein, and the method treats a cancer that is associated with DOK2 protein.

DPF3

In some cases, the target peptide sequence is a portion of DPF3 protein. In some cases, the therapeutic agent increases level of a first processed mRNA encoding a protein having a sequence SEQ ID NO: 8156, and decreases level of a second processed mRNA encoding a protein having a sequence SEQ ID NO: 392. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 190. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 190. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 72879947. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 72879947. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 190. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 190. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 72879804. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 72879804. In some cases, the targeted region of the pre-mRNA is within a sequence SEQ ID NO: 190. In some cases, the targeted region of the pre-mRNA is within a sequence between a pair of genomic sites GRCh38/hg38: chr14 72879947, and GRCh38/hg38: chr14 72879804. In some cases, the therapeutic agent is an antisense oligomer complementary to a sequence with at least about 80%, 85%, 90%, 95%, 96%, 97%, 98%, 99% or 100% sequence identity to SEQ ID NO:190. In some cases, the therapeutic agent is an antisense oligomer complementary to a sequence with 100% sequence identity to SEQ ID NO:190. In some cases, the therapeutic agent increases level of a first processed mRNA encoding a protein having a sequence SEQ ID NO: 8157, and decreases level of a second processed mRNA encoding a protein having a sequence SEQ ID NO: 393. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 191. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 191. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 72879947. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 72879947. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 191. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 191. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 72879804. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 72879804. In some cases, the targeted region of the pre-mRNA is within a sequence SEQ ID NO: 191. In some cases, the targeted region of the pre-mRNA is within a sequence between a pair of genomic sites GRCh38/hg38: chr14 72879947, and GRCh38/hg38: chr14 72879804. In some cases, the therapeutic agent is an antisense oligomer complementary to a sequence with at least about 80%, 85%, 90%, 95%, 96%, 97%, 98%, 99% or 100% sequence identity to SEQ ID NO:191. In some cases, the therapeutic agent is an antisense oligomer complementary to a sequence with 100% sequence identity to SEQ ID NO:191. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2720-2787. In some cases, the therapeutic agent is an antisense oligomer that has a sequence selected from the group consisting of SEQ ID NOs: 2720-2787. In some cases, the target peptide sequence is a portion of DPF3 protein, and the method treats a disease or the condition that comprises Intellectual Disability.

DTNA

In some cases, the target peptide sequence is a portion of DTNA protein. In some cases, the therapeutic agent increases level of a first processed mRNA encoding a protein having a sequence SEQ ID NO: 8158, and decreases level of a second processed mRNA encoding a protein having a sequence SEQ ID NO: 394. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 192. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 192. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr18 34757158. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr18 34757158. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 192. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 192. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr18 34757288. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr18 34757288. In some cases, the targeted region of the pre-mRNA is within a sequence SEQ ID NO: 192. In some cases, the targeted region of the pre-mRNA is within a sequence between a pair of genomic sites GRCh38/hg38: chr18 34757158, and GRCh38/hg38: chr18 34757288. In some cases, the therapeutic agent is an antisense oligomer complementary to a sequence with at least about 80%, 85%, 90%, 95%, 96%, 97%, 98%, 99% or 100% sequence identity to SEQ ID NO:192. In some cases, the therapeutic agent is an antisense oligomer complementary to a sequence with 100% sequence identity to SEQ ID NO:192. In some cases, the therapeutic agent increases level of a first processed mRNA encoding a protein having a sequence SEQ ID NO: 8159, and decreases level of a second processed mRNA encoding a protein having a sequence SEQ ID NO: 395. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 193. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 193. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr18 34757158. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr18 34757158. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 193. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 193. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr18 34757288. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr18 34757288. In some cases, the targeted region of the pre-mRNA is within a sequence SEQ ID NO: 193. In some cases, the targeted region of the pre-mRNA is within a sequence between a pair of genomic sites GRCh38/hg38: chr18 34757158, and GRCh38/hg38: chr18 34757288. In some cases, the therapeutic agent is an antisense oligomer complementary to a sequence with at least about 80%, 85%, 90%, 95%, 96%, 97%, 98%, 99% or 100% sequence identity to SEQ ID NO:193. In some cases, the therapeutic agent is an antisense oligomer complementary to a sequence with 100% sequence identity to SEQ ID NO:193. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2788-2852. In some cases, the therapeutic agent is an antisense oligomer that has a sequence selected from the group consisting of SEQ ID NOs: 2788-2852. In some cases, the target peptide sequence is a portion of DTNA protein, and the method treats a disease or the condition that comprises Left ventricular noncompaction cardiomyopathy; Left ventricular noncompaction; Familial Meniere's disease; Charcot-Marie-Tooth Disease; or Noncompaction of Left Ventricular Myocardium, Familial Isolated, Autosomal Dominant 1.

DYRK1A

In some cases, the target peptide sequence is a portion of DYRK1A protein. In some cases, the therapeutic agent increases level of a first processed mRNA encoding a protein having a sequence SEQ ID NO: 8160, and decreases level of a second processed mRNA encoding a protein having a sequence SEQ ID NO: 396. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 194. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 194. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr21 37456130. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr21 37456130. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 194. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 194. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr21 37456308. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr21 37456308. In some cases, the targeted region of the pre-mRNA is within a sequence SEQ ID NO: 194. In some cases, the targeted region of the pre-mRNA is within a sequence between a pair of genomic sites GRCh38/hg38: chr21 37456130, and GRCh38/hg38: chr21 37456308. In some cases, the therapeutic agent is an antisense oligomer complementary to a sequence with at least about 80%, 85%, 90%, 95%, 96%, 97%, 98%, 99% or 100% sequence identity to SEQ ID NO:194. In some cases, the therapeutic agent is an antisense oligomer complementary to a sequence with 100% sequence identity to SEQ ID NO:194. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2853-2927. In some cases, the therapeutic agent is an antisense oligomer that has a sequence selected from the group consisting of SEQ ID NOs: 2853-2927. In some cases, the target peptide sequence is a portion of DYRK1A protein, and the method treats a disease or the condition that comprises Intellectual Disability; Primary microcephaly; Microcephaly; Epileptic encephalopathy; or Mental retardation, autosomal dominant 7.

FOLH1

In some cases, the target peptide sequence is a portion of FOLH1 protein. In some cases, the therapeutic agent increases level of a first processed mRNA encoding a protein having a sequence SEQ ID NO: 8161, and decreases level of a second processed mRNA encoding a protein having a sequence SEQ ID NO: 397. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 195. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 195. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 49206874. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 49206874. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 195. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 195. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 49206778. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 49206778. In some cases, the targeted region of the pre-mRNA is within a sequence SEQ ID NO: 195. In some cases, the targeted region of the pre-mRNA is within a sequence between a pair of genomic sites GRCh38/hg38: chr11 49206874, and GRCh38/hg38: chr11 49206778. In some cases, the therapeutic agent is an antisense oligomer complementary to a sequence with at least about 80%, 85%, 90%, 95%, 96%, 97%, 98%, 99% or 100% sequence identity to SEQ ID NO:195. In some cases, the therapeutic agent is an antisense oligomer complementary to a sequence with 100% sequence identity to SEQ ID NO:195. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2928-2985. In some cases, the therapeutic agent is an antisense oligomer that has a sequence selected from the group consisting of SEQ ID NOs: 2928-2985. In some cases, the target peptide sequence is a portion of FOLH1 protein, and the method treats a disease or the condition that comprises Colorectal Cancer; Depressive disorder; Mental Depression; or Schizophrenia.

FUZ

In some cases, the target peptide sequence is a portion of FUZ protein. In some cases, the therapeutic agent increases level of a first processed mRNA encoding a protein having a sequence SEQ ID NO: 8162, and decreases level of a second processed mRNA encoding a protein having a sequence SEQ ID NO: 398. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 196. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 196. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 49812335. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 49812335. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 196. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 196. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 49812251. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 49812251. In some cases, the targeted region of the pre-mRNA is within a sequence SEQ ID NO: 196. In some cases, the targeted region of the pre-mRNA is within a sequence between a pair of genomic sites GRCh38/hg38: chr19 49812335, and GRCh38/hg38: chr19 49812251. In some cases, the therapeutic agent is an antisense oligomer complementary to a sequence with at least about 80%, 85%, 90%, 95%, 96%, 97%, 98%, 99% or 100% sequence identity to SEQ ID NO:196. In some cases, the therapeutic agent is an antisense oligomer complementary to a sequence with 100% sequence identity to SEQ ID NO:196. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 2986-3041. In some cases, the therapeutic agent is an antisense oligomer that has a sequence selected from the group consisting of SEQ ID NOs: 2986-3041. In some cases, the target peptide sequence is a portion of FUZ protein, and the method treats a disease or the condition that comprises Caudal Regression Syndrome; Arnold Chiari Malformation; Caudal dysplasia syndrome; Chiari malformation type II; Sacral Agenesis Syndrome; Sacral agenesis; Currarino triad; Spina bifida aperta of cervical spine; or Neural tube defects.

GANAB

In some cases, the target peptide sequence is a portion of GANAB protein. In some cases, the therapeutic agent increases level of a first processed mRNA encoding a protein having a sequence SEQ ID NO: 8163, and decreases level of a second processed mRNA encoding a protein having a sequence SEQ ID NO: 399. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 197. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 197. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 62639110. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 62639110. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 197. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 197. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 62638983. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 62638983. In some cases, the targeted region of the pre-mRNA is within a sequence SEQ ID NO: 197. In some cases, the targeted region of the pre-mRNA is within a sequence between a pair of genomic sites GRCh38/hg38: chr11 62639110, and GRCh38/hg38: chr11 62638983. In some cases, the therapeutic agent is an antisense oligomer complementary to a sequence with at least about 80%, 85%, 90%, 95%, 96%, 97%, 98%, 99% or 100% sequence identity to SEQ ID NO:197. In some cases, the therapeutic agent is an antisense oligomer complementary to a sequence with 100% sequence identity to SEQ ID NO:197. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3042-3106. In some cases, the therapeutic agent is an antisense oligomer that has a sequence selected from the group consisting of SEQ ID NOs: 3042-3106. In some cases, the target peptide sequence is a portion of GANAB protein, and the method treats a disease or the condition that comprises Polycystic Kidney, Autosomal Dominant; or Polycystic Kidney Disease 3, Autosomal Dominant. In some cases, the method treats a cancer that is associated with GANAB protein.

GEMIN4

In some cases, the target peptide sequence is a portion of GEMIN4 protein. In some cases, the therapeutic agent increases level of a first processed mRNA encoding a protein having a sequence SEQ ID NO: 8164, and decreases level of a second processed mRNA encoding a protein having a sequence SEQ ID NO: 400. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 198. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 198. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 749909. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 749909. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 198. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 198. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 749814. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 749814. In some cases, the targeted region of the pre-mRNA is within a sequence SEQ ID NO: 198. In some cases, the targeted region of the pre-mRNA is within a sequence between a pair of genomic sites GRCh38/hg38: chr17 749909, and GRCh38/hg38: chr17 749814. In some cases, the therapeutic agent is an antisense oligomer complementary to a sequence with at least about 80%, 85%, 90%, 95%, 96%, 97%, 98%, 99% or 100% sequence identity to SEQ ID NO:198. In some cases, the therapeutic agent is an antisense oligomer complementary to a sequence with 100% sequence identity to SEQ ID NO:198. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3107-3164. In some cases, the therapeutic agent is an antisense oligomer that has a sequence selected from the group consisting of SEQ ID NOs: 3107-3164. In some cases, the target peptide sequence is a portion of GEMIN4 protein, and the method treats a disease or the condition that comprises Intellectual Disability; Alcoholic Intoxication, Chronic; or Schizophrenia.

GGA3

In some cases, the target peptide sequence is a portion of GGA3 protein. In some cases, the therapeutic agent increases level of a first processed mRNA encoding a protein having a sequence SEQ ID NO: 8165, and decreases level of a second processed mRNA encoding a protein having a sequence SEQ ID NO: 401. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 199. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 199. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 75243570. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 75243570. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 199. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 199. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 75243447. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 75243447. In some cases, the targeted region of the pre-mRNA is within a sequence SEQ ID NO: 199. In some cases, the targeted region of the pre-mRNA is within a sequence between a pair of genomic sites GRCh38/hg38: chr17 75243570, and GRCh38/hg38: chr17 75243447. In some cases, the therapeutic agent is an antisense oligomer complementary to a sequence with at least about 80%, 85%, 90%, 95%, 96%, 97%, 98%, 99% or 100% sequence identity to SEQ ID NO:199. In some cases, the therapeutic agent is an antisense oligomer complementary to a sequence with 100% sequence identity to SEQ ID NO:199. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3165-3228. In some cases, the therapeutic agent is an antisense oligomer that has a sequence selected from the group consisting of SEQ ID NOs: 3165-3228. In some cases, the target peptide sequence is a portion of GGA3 protein, and the method treats a disease or the condition that comprises Malignant neoplasm of breast.

GOSR2

In some cases, the target peptide sequence is a portion of GOSR2 protein. In some cases, the therapeutic agent increases level of a first processed mRNA encoding a protein having a sequence SEQ ID NO: 8166, and decreases level of a second processed mRNA encoding a protein having a sequence SEQ ID NO: 402. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 200. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 200. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 46924261. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 46924261. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 200. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 200. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 46924468. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 46924468. In some cases, the targeted region of the pre-mRNA is within a sequence SEQ ID NO: 200. In some cases, the targeted region of the pre-mRNA is within a sequence between a pair of genomic sites GRCh38/hg38: chr17 46924261, and GRCh38/hg38: chr17 46924468. In some cases, the therapeutic agent is an antisense oligomer complementary to a sequence with at least about 80%, 85%, 90%, 95%, 96%, 97%, 98%, 99% or 100% sequence identity to SEQ ID NO:200. In some cases, the therapeutic agent is an antisense oligomer complementary to a sequence with 100% sequence identity to SEQ ID NO:200. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3229-3309. In some cases, the therapeutic agent is an antisense oligomer that has a sequence selected from the group consisting of SEQ ID NOs: 3229-3309. In some cases, the target peptide sequence is a portion of GOSR2 protein, and the method treats a disease or the condition that comprises Ataxias, Hereditary; Intellectual Disability; Epilepsy, Progressive Myoclonic, 6; or Epileptic encephalopathy.

GPM6A

In some cases, the target peptide sequence is a portion of GPM6A protein. In some cases, the therapeutic agent increases level of a first processed mRNA encoding a protein having a sequence SEQ ID NO: 8167, and decreases level of a second processed mRNA encoding a protein having a sequence SEQ ID NO: 403. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 201. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 201. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 175812249. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 175812249. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 201. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 201. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 175812191. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 175812191. In some cases, the targeted region of the pre-mRNA is within a sequence SEQ ID NO: 201. In some cases, the targeted region of the pre-mRNA is within a sequence between a pair of genomic sites GRCh38/hg38: chr4 175812249, and GRCh38/hg38: chr4 175812191. In some cases, the therapeutic agent is an antisense oligomer complementary to a sequence with at least about 80%, 85%, 90%, 95%, 96%, 97%, 98%, 99% or 100% sequence identity to SEQ ID NO:201. In some cases, the therapeutic agent is an antisense oligomer complementary to a sequence with 100% sequence identity to SEQ ID NO:201. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3310-3360. In some cases, the therapeutic agent is an antisense oligomer that has a sequence selected from the group consisting of SEQ ID NOs: 3310-3360. In some cases, the target peptide sequence is a portion of GPM6A protein, and the method treats a disease or the condition that comprises Schizophrenia.

GRB10

In some cases, the target peptide sequence is a portion of GRB10 protein. In some cases, the therapeutic agent increases level of a first processed mRNA encoding a protein having a sequence SEQ ID NO: 8168, and decreases level of a second processed mRNA encoding a protein having a sequence SEQ ID NO: 404. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 202. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 202. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 50710990. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 50710990. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 202. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 202. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 50710875. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 50710875. In some cases, the targeted region of the pre-mRNA is within a sequence SEQ ID NO: 202. In some cases, the targeted region of the pre-mRNA is within a sequence between a pair of genomic sites GRCh38/hg38: chr7 50710990, and GRCh38/hg38: chr7 50710875. In some cases, the therapeutic agent is an antisense oligomer complementary to a sequence with at least about 80%, 85%, 90%, 95%, 96%, 97%, 98%, 99% or 100% sequence identity to SEQ ID NO:202. In some cases, the therapeutic agent is an antisense oligomer complementary to a sequence with 100% sequence identity to SEQ ID NO:202. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3361-3422. In some cases, the therapeutic agent is an antisense oligomer that has a sequence selected from the group consisting of SEQ ID NOs: 3361-3422. In some cases, the target peptide sequence is a portion of GRB10 protein, and the method treats a disease or the condition that comprises Schizophrenia.

GSN

In some cases, the target peptide sequence is a portion of GSN protein. In some cases, the therapeutic agent increases level of a first processed mRNA encoding a protein having a sequence SEQ ID NO: 8169, and decreases level of a second processed mRNA encoding a protein having a sequence SEQ ID NO: 405. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 203. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 203. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr9 121300056. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr9 121300056. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 203. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 203. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr9 121300126. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr9 121300126. In some cases, the targeted region of the pre-mRNA is within a sequence SEQ ID NO: 203. In some cases, the targeted region of the pre-mRNA is within a sequence between a pair of genomic sites GRCh38/hg38: chr9 121300056, and GRCh38/hg38: chr9 121300126. In some cases, the therapeutic agent is an antisense oligomer complementary to a sequence with at least about 80%, 85%, 90%, 95%, 96%, 97%, 98%, 99% or 100% sequence identity to SEQ ID NO:203. In some cases, the therapeutic agent is an antisense oligomer complementary to a sequence with 100% sequence identity to SEQ ID NO:203. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3423-3475. In some cases, the therapeutic agent is an antisense oligomer that has a sequence selected from the group consisting of SEQ ID NOs: 3423-3475. In some cases, the target peptide sequence is a portion of GSN protein, and the method treats a disease or the condition that comprises Lattice corneal dystrophy Type II; Meretoja syndrome; Malignant neoplasm of breast; Periodic Fever Syndrome; Familial Amyloid Polyneuropathy, Type IV; Cerebral Amyloid Angiopathy, Gsn-Related; Abnormality of the cornea; Schizophrenia; or Amyloidosis, Finnish type. In some cases, the method treats a cancer that is associated with GSN protein.

HDAC8

In some cases, the target peptide sequence is a portion of HDAC8 protein. In some cases, the therapeutic agent increases level of a first processed mRNA encoding a protein having a sequence SEQ ID NO: 8170, and decreases level of a second processed mRNA encoding a protein having a sequence SEQ ID NO: 406. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 204. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 204. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 72572109. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 72572109. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 204. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 204. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 72572057. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 72572057. In some cases, the targeted region of the pre-mRNA is within a sequence SEQ ID NO: 204. In some cases, the targeted region of the pre-mRNA is within a sequence between a pair of genomic sites GRCh38/hg38: chrX 72572109, and GRCh38/hg38: chrX 72572057. In some cases, the therapeutic agent is an antisense oligomer complementary to a sequence with at least about 80%, 85%, 90%, 95%, 96%, 97%, 98%, 99% or 100% sequence identity to SEQ ID NO:204. In some cases, the therapeutic agent is an antisense oligomer complementary to a sequence with 100% sequence identity to SEQ ID NO:204. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3476-3525. In some cases, the therapeutic agent is an antisense oligomer that has a sequence selected from the group consisting of SEQ ID NOs: 3476-3525. In some cases, the target peptide sequence is a portion of HDAC8 protein, and the method treats a disease or the condition that comprises Intellectual Disability; Primary microcephaly; CORNELIA DE LANGE SYNDROME 5; Radial club hand; Cornelia De Lange Syndrome; or Wilson-Turner X-linked Mental Retardation Syndrome.

HIVEP1

In some cases, the target peptide sequence is a portion of HIVEP1 protein. In some cases, the therapeutic agent increases level of a first processed mRNA encoding a protein having a sequence SEQ ID NO: 8171, and decreases level of a second processed mRNA encoding a protein having a sequence SEQ ID NO: 407. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 205. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 205. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 12020248. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 12020248. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 205. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 205. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 12020418. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 12020418. In some cases, the targeted region of the pre-mRNA is within a sequence SEQ ID NO: 205. In some cases, the targeted region of the pre-mRNA is within a sequence between a pair of genomic sites GRCh38/hg38: chr6 12020248, and GRCh38/hg38: chr6 12020418. In some cases, the therapeutic agent is an antisense oligomer complementary to a sequence with at least about 80%, 85%, 90%, 95%, 96%, 97%, 98%, 99% or 100% sequence identity to SEQ ID NO:205. In some cases, the therapeutic agent is an antisense oligomer complementary to a sequence with 100% sequence identity to SEQ ID NO:205. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3526-3598. In some cases, the therapeutic agent is an antisense oligomer that has a sequence selected from the group consisting of SEQ ID NOs: 3526-3598. In some cases, the target peptide sequence is a portion of HIVEP1 protein, and the method treats a cancer that is associated with HIVEP1 protein.

HK1

In some cases, the target peptide sequence is a portion of HK1 protein. In some cases, the therapeutic agent increases level of a first processed mRNA encoding a protein having a sequence SEQ ID NO: 8172, and decreases level of a second processed mRNA encoding a protein having a sequence SEQ ID NO: 408. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 206. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 206. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 69300769. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 69300769. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 206. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 206. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 69300861. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 69300861. In some cases, the targeted region of the pre-mRNA is within a sequence SEQ ID NO: 206. In some cases, the targeted region of the pre-mRNA is within a sequence between a pair of genomic sites GRCh38/hg38: chr10 69300769, and GRCh38/hg38: chr10 69300861. In some cases, the therapeutic agent is an antisense oligomer complementary to a sequence with at least about 80%, 85%, 90%, 95%, 96%, 97%, 98%, 99% or 100% sequence identity to SEQ ID NO:206. In some cases, the therapeutic agent is an antisense oligomer complementary to a sequence with 100% sequence identity to SEQ ID NO:206. In some cases, the therapeutic agent increases level of a first processed mRNA encoding a protein having a sequence SEQ ID NO: 8173, and decreases level of a second processed mRNA encoding a protein having a sequence SEQ ID NO: 409. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 207. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 207. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 69300769. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 69300769. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 207. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 207. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 69300861. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 69300861. In some cases, the targeted region of the pre-mRNA is within a sequence SEQ ID NO: 207. In some cases, the targeted region of the pre-mRNA is within a sequence between a pair of genomic sites GRCh38/hg38: chr10 69300769, and GRCh38/hg38: chr10 69300861. In some cases, the therapeutic agent is an antisense oligomer complementary to a sequence with at least about 80%, 85%, 90%, 95%, 96%, 97%, 98%, 99% or 100% sequence identity to SEQ ID NO:207. In some cases, the therapeutic agent is an antisense oligomer complementary to a sequence with 100% sequence identity to SEQ ID NO:207. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3599-3656. In some cases, the therapeutic agent is an antisense oligomer that has a sequence selected from the group consisting of SEQ ID NOs: 3599-3656. In some cases, the target peptide sequence is a portion of HK1 protein, and the method treats a disease or the condition that comprises Intellectual Disability; Congenital anemia; Retinitis Pigmentosa 79; Charcot-Marie-Tooth Disease; Cytopenia; Hemolytic Anemia, Nonspherocytic, due to Hexokinase Deficiency; Neuropathy, hereditary motor and sensory, Russe type; or Schizophrenia.

IFNGR1

In some cases, the target peptide sequence is a portion of IFNGR1 protein. In some cases, the therapeutic agent increases level of a first processed mRNA encoding a protein having a sequence SEQ ID NO: 8174, and decreases level of a second processed mRNA encoding a protein having a sequence SEQ ID NO: 410. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 208. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 208. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 137215377. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 137215377. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 208. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 208. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 137215267. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 137215267. In some cases, the targeted region of the pre-mRNA is within a sequence SEQ ID NO: 208. In some cases, the targeted region of the pre-mRNA is within a sequence between a pair of genomic sites GRCh38/hg38: chr6 137215377, and GRCh38/hg38: chr6 137215267. In some cases, the therapeutic agent is an antisense oligomer complementary to a sequence with at least about 80%, 85%, 90%, 95%, 96%, 97%, 98%, 99% or 100% sequence identity to SEQ ID NO:208. In some cases, the therapeutic agent is an antisense oligomer complementary to a sequence with 100% sequence identity to SEQ ID NO:208. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3657-3717. In some cases, the therapeutic agent is an antisense oligomer that has a sequence selected from the group consisting of SEQ ID NOs: 3657-3717. In some cases, the target peptide sequence is a portion of IFNGR1 protein, and the method treats a disease or the condition that comprises Interferon gamma, receptor 1, deficiency; Immunodeficiency 27A; Immunodeficiency 27B; H. pylori infection, susceptibility to; Tuberculosis infection, protection against; Immunodeficiency 27A, mycobacteriosis, AR; Immunodeficiency 27B, mycobacteriosis, AD; Hepatitis B virus infection, susceptibility to; or Tuberculosis, susceptibility to.

IKBKB

In some cases, the target peptide sequence is a portion of IKBKB protein. In some cases, the therapeutic agent increases level of a first processed mRNA encoding a protein having a sequence SEQ ID NO: 8175, and decreases level of a second processed mRNA encoding a protein having a sequence SEQ ID NO: 411. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 209. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 209. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 209. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 209. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases, the targeted region of the pre-mRNA is within a sequence SEQ ID NO: 209. In some cases, the targeted region of the pre-mRNA is within a sequence between a pair of genomic sites GRCh38/hg38: chr8 42272083, and GRCh38/hg38: chr8 42272205. In some cases, the therapeutic agent is an antisense oligomer complementary to a sequence with at least about 80%, 85%, 90%, 95%, 96%, 97%, 98%, 99% or 100% sequence identity to SEQ ID NO:209. In some cases, the therapeutic agent is an antisense oligomer complementary to a sequence with 100% sequence identity to SEQ ID NO:209. In some cases, the therapeutic agent increases level of a first processed mRNA encoding a protein having a sequence SEQ ID NO: 8176, and decreases level of a second processed mRNA encoding a protein having a sequence SEQ ID NO: 412. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 210. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 210. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 210. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 210. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases, the targeted region of the pre-mRNA is within a sequence SEQ ID NO: 210. In some cases, the targeted region of the pre-mRNA is within a sequence between a pair of genomic sites GRCh38/hg38: chr8 42272083, and GRCh38/hg38: chr8 42272205. In some cases, the therapeutic agent is an antisense oligomer complementary to a sequence with at least about 80%, 85%, 90%, 95%, 96%, 97%, 98%, 99% or 100% sequence identity to SEQ ID NO:210. In some cases, the therapeutic agent is an antisense oligomer complementary to a sequence with 100% sequence identity to SEQ ID NO:210. In some cases, the therapeutic agent increases level of a first processed mRNA encoding a protein having a sequence SEQ ID NO: 8177, and decreases level of a second processed mRNA encoding a protein having a sequence SEQ ID NO: 413. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 211. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 211. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 211. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 211. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases, the targeted region of the pre-mRNA is within a sequence SEQ ID NO: 211. In some cases, the targeted region of the pre-mRNA is within a sequence between a pair of genomic sites GRCh38/hg38: chr8 42272083, and GRCh38/hg38: chr8 42272205. In some cases, the therapeutic agent is an antisense oligomer complementary to a sequence with at least about 80%, 85%, 90%, 95%, 96%, 97%, 98%, 99% or 100% sequence identity to SEQ ID NO:211. In some cases, the therapeutic agent is an antisense oligomer complementary to a sequence with 100% sequence identity to SEQ ID NO:211. In some cases, the therapeutic agent increases level of a first processed mRNA encoding a protein having a sequence SEQ ID NO: 8178, and decreases level of a second processed mRNA encoding a protein having a sequence SEQ ID NO: 414. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 212. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 212. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 42272083. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 212. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 212. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 42272205. In some cases, the targeted region of the pre-mRNA is within a sequence SEQ ID NO: 212. In some cases, the targeted region of the pre-mRNA is within a sequence between a pair of genomic sites GRCh38/hg38: chr8 42272083, and GRCh38/hg38: chr8 42272205. In some cases, the therapeutic agent is an antisense oligomer complementary to a sequence with at least about 80%, 85%, 90%, 95%, 96%, 97%, 98%, 99% or 100% sequence identity to SEQ ID NO:212. In some cases, the therapeutic agent is an antisense oligomer complementary to a sequence with 100% sequence identity to SEQ ID NO:212. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3718-3781. In some cases, the therapeutic agent is an antisense oligomer that has a sequence selected from the group consisting of SEQ ID NOs: 3718-3781. In some cases, the target peptide sequence is a portion of IKBKB protein, and the method treats a disease or the condition that comprises Malignant neoplasm of breast; Lymphoma, Non-Hodgkin; or Immunodeficiency 18.

ISCU

In some cases, the target peptide sequence is a portion of ISCU protein. In some cases, the therapeutic agent increases level of a first processed mRNA encoding a protein having a sequence SEQ ID NO: 8179, and decreases level of a second processed mRNA encoding a protein having a sequence SEQ ID NO: 415. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 213. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 213. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 108564060. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 108564060. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 213. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 213. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 108564155. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 108564155. In some cases, the targeted region of the pre-mRNA is within a sequence SEQ ID NO: 213. In some cases, the targeted region of the pre-mRNA is within a sequence between a pair of genomic sites GRCh38/hg38: chr12 108564060, and GRCh38/hg38: chr12 108564155. In some cases, the therapeutic agent is an antisense oligomer complementary to a sequence with at least about 80%, 85%, 90%, 95%, 96%, 97%, 98%, 99% or 100% sequence identity to SEQ ID NO:213. In some cases, the therapeutic agent is an antisense oligomer complementary to a sequence with 100% sequence identity to SEQ ID NO:213. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3782-3839. In some cases, the therapeutic agent is an antisense oligomer that has a sequence selected from the group consisting of SEQ ID NOs: 3782-3839. In some cases, the target peptide sequence is a portion of ISCU protein, and the method treats a disease or the condition that comprises Mitochondrial Diseases; Congenital myopathy (disorder); Arthrogryposis; Rhabdomyolysis; Myopathy with Exercise Intolerance, Swedish Type; or Myopathy with lactic acidosis, hereditary.

KARS

In some cases, the target peptide sequence is a portion of KARS protein. In some cases, the therapeutic agent increases level of a first processed mRNA encoding a protein having a sequence SEQ ID NO: 8180, and decreases level of a second processed mRNA encoding a protein having a sequence SEQ ID NO: 416. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 214. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 214. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 75644462. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 75644462. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 214. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 214. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 75644283. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 75644283. In some cases, the targeted region of the pre-mRNA is within a sequence SEQ ID NO: 214. In some cases, the targeted region of the pre-mRNA is within a sequence between a pair of genomic sites GRCh38/hg38: chr16 75644462, and GRCh38/hg38: chr16 75644283. In some cases, the therapeutic agent is an antisense oligomer complementary to a sequence with at least about 80%, 85%, 90%, 95%, 96%, 97%, 98%, 99% or 100% sequence identity to SEQ ID NO:214. In some cases, the therapeutic agent is an antisense oligomer complementary to a sequence with 100% sequence identity to SEQ ID NO:214. In some cases, the therapeutic agent increases level of a first processed mRNA encoding a protein having a sequence SEQ ID NO: 8181, and decreases level of a second processed mRNA encoding a protein having a sequence SEQ ID NO: 417. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 215. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 215. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 75644462. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 75644462. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 215. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 215. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 75644283. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 75644283. In some cases, the targeted region of the pre-mRNA is within a sequence SEQ ID NO: 215. In some cases, the targeted region of the pre-mRNA is within a sequence between a pair of genomic sites GRCh38/hg38: chr16 75644462, and GRCh38/hg38: chr16 75644283. In some cases, the therapeutic agent is an antisense oligomer complementary to a sequence with at least about 80%, 85%, 90%, 95%, 96%, 97%, 98%, 99% or 100% sequence identity to SEQ ID NO:215. In some cases, the therapeutic agent is an antisense oligomer complementary to a sequence with 100% sequence identity to SEQ ID NO:215. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3840-3914. In some cases, the therapeutic agent is an antisense oligomer that has a sequence selected from the group consisting of SEQ ID NOs: 3840-3914. In some cases, the target peptide sequence is a portion of KARS protein, and the method treats a disease or the condition that comprises Intellectual Disability; Mitochondrial Diseases; Charcot-Marie-Tooth Disease, Recessive Intermediate B; Charcot-Marie-Tooth Disease; or Deafness, autosomal recessive 89.

KIAA0319

In some cases, the target peptide sequence is a portion of KIAA0319 protein. In some cases, the therapeutic agent increases level of a first processed mRNA encoding a protein having a sequence SEQ ID NO: 8182, and decreases level of a second processed mRNA encoding a protein having a sequence SEQ ID NO: 418. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 216. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 216. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 24600709. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 24600709. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 216. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 216. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 24600619. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 24600619. In some cases, the targeted region of the pre-mRNA is within a sequence SEQ ID NO: 216. In some cases, the targeted region of the pre-mRNA is within a sequence between a pair of genomic sites GRCh38/hg38: chr6 24600709, and GRCh38/hg38: chr6 24600619. In some cases, the therapeutic agent is an antisense oligomer complementary to a sequence with at least about 80%, 85%, 90%, 95%, 96%, 97%, 98%, 99% or 100% sequence identity to SEQ ID NO:216. In some cases, the therapeutic agent is an antisense oligomer complementary to a sequence with 100% sequence identity to SEQ ID NO:216. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3915-3971. In some cases, the therapeutic agent is an antisense oligomer that has a sequence selected from the group consisting of SEQ ID NOs: 3915-3971. In some cases, the target peptide sequence is a portion of KIAA0319 protein, and the method treats a disease or the condition that comprises; Dyslexia, susceptibility to, 2.

KIAA0586

In some cases, the target peptide sequence is a portion of KIAA0586 protein. In some cases, the therapeutic agent increases level of a first processed mRNA encoding a protein having a sequence SEQ ID NO: 8183, and decreases level of a second processed mRNA encoding a protein having a sequence SEQ ID NO: 419. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 217. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 217. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 58429363. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 58429363. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 217. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 217. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 58429433. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 58429433. In some cases, the targeted region of the pre-mRNA is within a sequence SEQ ID NO: 217. In some cases, the targeted region of the pre-mRNA is within a sequence between a pair of genomic sites GRCh38/hg38: chr14 58429363, and GRCh38/hg38: chr14 58429433. In some cases, the therapeutic agent is an antisense oligomer complementary to a sequence with at least about 80%, 85%, 90%, 95%, 96%, 97%, 98%, 99% or 100% sequence identity to SEQ ID NO:217. In some cases, the therapeutic agent is an antisense oligomer complementary to a sequence with 100% sequence identity to SEQ ID NO:217. In some cases, the therapeutic agent increases level of a first processed mRNA encoding a protein having a sequence SEQ ID NO: 8184, and decreases level of a second processed mRNA encoding a protein having a sequence SEQ ID NO: 420. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 218. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 218. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 58429363. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 58429363. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 218. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 218. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 58429433. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 58429433. In some cases, the targeted region of the pre-mRNA is within a sequence SEQ ID NO: 218. In some cases, the targeted region of the pre-mRNA is within a sequence between a pair of genomic sites GRCh38/hg38: chr14 58429363, and GRCh38/hg38: chr14 58429433. In some cases, the therapeutic agent is an antisense oligomer complementary to a sequence with at least about 80%, 85%, 90%, 95%, 96%, 97%, 98%, 99% or 100% sequence identity to SEQ ID NO:218. In some cases, the therapeutic agent is an antisense oligomer complementary to a sequence with 100% sequence identity to SEQ ID NO:218. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 3972-4024. In some cases, the therapeutic agent is an antisense oligomer that has a sequence selected from the group consisting of SEQ ID NOs: 3972-4024. In some cases, the target peptide sequence is a portion of KIAA0586 protein, and the method treats a disease or the condition that comprises Intellectual Disability; Joubert Syndrome 23; Polydactyly; Familial aplasia of the vermis; Joubert syndrome with Jeune asphyxiating thoracic dystrophy; Hydrocephalus; Ciliopathies; or Short-rib thoracic dysplasia 14 with polydactyly.

KIZ

In some cases, the target peptide sequence is a portion of KIZ protein. In some cases, the therapeutic agent increases level of a first processed mRNA encoding a protein having a sequence SEQ ID NO: 8185, and decreases level of a second processed mRNA encoding a protein having a sequence SEQ ID NO: 421. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 219. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 219. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr20 21132097. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr20 21132097. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 219. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 219. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr20 21132159. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr20 21132159. In some cases, the targeted region of the pre-mRNA is within a sequence SEQ ID NO: 219. In some cases, the targeted region of the pre-mRNA is within a sequence between a pair of genomic sites GRCh38/hg38: chr20 21132097, and GRCh38/hg38: chr20 21132159. In some cases, the therapeutic agent is an antisense oligomer complementary to a sequence with at least about 80%, 85%, 90%, 95%, 96%, 97%, 98%, 99% or 100% sequence identity to SEQ ID NO:219. In some cases, the therapeutic agent is an antisense oligomer complementary to a sequence with 100% sequence identity to SEQ ID NO:219. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4025-4076. In some cases, the therapeutic agent is an antisense oligomer that has a sequence selected from the group consisting of SEQ ID NOs: 4025-4076. In some cases, the target peptide sequence is a portion of KIZ protein, and the method treats a disease or the condition that comprises Retinitis Pigmentosa; or Retinitis pigmentosa 69.

KLK6

In some cases, the target peptide sequence is a portion of KLK6 protein. In some cases, the therapeutic agent increases level of a first processed mRNA encoding a protein having a sequence SEQ ID NO: 8186, and decreases level of a second processed mRNA encoding a protein having a sequence SEQ ID NO: 422. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 220. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 220. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 50963549. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 50963549. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 220. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 220. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 50963302. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 50963302. In some cases, the targeted region of the pre-mRNA is within a sequence SEQ ID NO: 220. In some cases, the targeted region of the pre-mRNA is within a sequence between a pair of genomic sites GRCh38/hg38: chr19 50963549, and GRCh38/hg38: chr19 50963302. In some cases, the therapeutic agent is an antisense oligomer complementary to a sequence with at least about 80%, 85%, 90%, 95%, 96%, 97%, 98%, 99% or 100% sequence identity to SEQ ID NO:220. In some cases, the therapeutic agent is an antisense oligomer complementary to a sequence with 100% sequence identity to SEQ ID NO:220. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4077-4165. In some cases, the therapeutic agent is an antisense oligomer that has a sequence selected from the group consisting of SEQ ID NOs: 4077-4165. In some cases, the target peptide sequence is a portion of KLK6 protein, and the method treats a cancer that is associated with KLK6 protein.

LMNA

In some cases, the target peptide sequence is a portion of LMNA protein. In some cases, the therapeutic agent increases level of a first processed mRNA encoding a protein having a sequence SEQ ID NO: 8187, and decreases level of a second processed mRNA encoding a protein having a sequence SEQ ID NO: 423. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 221. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 221. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 156126736. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 156126736. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 221. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 221. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 156126915. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 156126915. In some cases, the targeted region of the pre-mRNA is within a sequence SEQ ID NO: 221. In some cases, the targeted region of the pre-mRNA is within a sequence between a pair of genomic sites GRCh38/hg38: chr1 156126736, and GRCh38/hg38: chr1 156126915. In some cases, the therapeutic agent is an antisense oligomer complementary to a sequence with at least about 80%, 85%, 90%, 95%, 96%, 97%, 98%, 99% or 100% sequence identity to SEQ ID NO:221. In some cases, the therapeutic agent is an antisense oligomer complementary to a sequence with 100% sequence identity to SEQ ID NO:221. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4166-4240. In some cases, the therapeutic agent is an antisense oligomer that has a sequence selected from the group consisting of SEQ ID NOs: 4166-4240. In some cases, the target peptide sequence is a portion of LMNA protein, and the method treats a disease or the condition that comprises Intellectual Disability; Muscular Dystrophy, Limb-Girdle, Type 1B; Hypertrophic Cardiomyopathy; Left ventricular noncompaction; Osteogenesis Imperfecta; Charcot-Marie-Tooth Disease; Cardiomyopathy, Familial Idiopathic; Familial Partial Lipodystrophy, Type 1; Left ventricular noncompaction cardiomyopathy; Mandibuloacral dysostosis; MUSCULAR DYSTROPHY, CONGENITAL, LMNA-RELATED (disorder); Congenital myopathy (disorder); Arthrogryposis; Arrhythmogenic Right Ventricular Dysplasia; Muscular Dystrophies, Limb-Girdle; Malouf syndrome; Najjar syndrome; Charcot-Marie-Tooth disease, Type 2B1; Emery-Dreifuss Muscular Dystrophy 3; Lethal tight skin contracture syndrome (disorder); Familial Partial Lipodystrophy, Type 2; Conduction disorder of the heart; Cardiomyopathy, Dilated; Progeria Syndrome, Childhood-Onset; Atypical Werner syndrome; Congenital muscular dystrophy (disorder); Autosomal Recessive Emery-Dreifuss Muscular Dystrophy; Heart-hand syndrome, Slovenian type; Progeria; Autosomal Dominant Emery-Dreifuss Muscular Dystrophy; Cardiomyopathy, dilated, 1A; Mandibuloacral dysplasia; Muscular dystrophy, limb-girdle, type 1B; Emery-Dreifuss muscular dystrophy 2, AD; Malouf syndrome; Restrictive dermopathy, lethal; Emery-Dreifuss muscular dystrophy 3, AR; Heart-hand syndrome, Slovenian type; Charcot-Marie-Tooth disease, type 2B1; Hutchinson-Gilford progeria; Lipodystrophy, familial partial, type 2; or Muscular dystrophy, congenital.

LMNTD1

In some cases, the target peptide sequence is a portion of LMNTD1 protein. In some cases, the therapeutic agent increases level of a first processed mRNA encoding a protein having a sequence SEQ ID NO: 8188, and decreases level of a second processed mRNA encoding a protein having a sequence SEQ ID NO: 424. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 222. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 222. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 25552989. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 25552989. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 222. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 222. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 25552871. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 25552871. In some cases, the targeted region of the pre-mRNA is within a sequence SEQ ID NO: 222. In some cases, the targeted region of the pre-mRNA is within a sequence between a pair of genomic sites GRCh38/hg38: chr12 25552989, and GRCh38/hg38: chr12 25552871. In some cases, the therapeutic agent is an antisense oligomer complementary to a sequence with at least about 80%, 85%, 90%, 95%, 96%, 97%, 98%, 99% or 100% sequence identity to SEQ ID NO:222. In some cases, the therapeutic agent is an antisense oligomer complementary to a sequence with 100% sequence identity to SEQ ID NO:222. In some cases, the therapeutic agent increases level of a first processed mRNA encoding a protein having a sequence SEQ ID NO: 8189, and decreases level of a second processed mRNA encoding a protein having a sequence SEQ ID NO: 425. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 223. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 223. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 25552989. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 25552989. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 223. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 223. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 25552871. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 25552871. In some cases, the targeted region of the pre-mRNA is within a sequence SEQ ID NO: 223. In some cases, the targeted region of the pre-mRNA is within a sequence between a pair of genomic sites GRCh38/hg38: chr12 25552989, and GRCh38/hg38: chr12 25552871. In some cases, the therapeutic agent is an antisense oligomer complementary to a sequence with at least about 80%, 85%, 90%, 95%, 96%, 97%, 98%, 99% or 100% sequence identity to SEQ ID NO:223. In some cases, the therapeutic agent is an antisense oligomer complementary to a sequence with 100% sequence identity to SEQ ID NO:223. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4241-4303. In some cases, the therapeutic agent is an antisense oligomer that has a sequence selected from the group consisting of SEQ ID NOs: 4241-4303. In some cases, the target peptide sequence is a portion of LMNTD1 protein, and the method treats a cancer that is associated with LMNTD1 protein.

LRRC7

In some cases, the target peptide sequence is a portion of LRRC7 protein. In some cases, the therapeutic agent increases level of a first processed mRNA encoding a protein having a sequence SEQ ID NO: 8190, and decreases level of a second processed mRNA encoding a protein having a sequence SEQ ID NO: 426. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 224. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 224. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 69716142. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 69716142. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 224. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 224. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 69716218. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 69716218. In some cases, the targeted region of the pre-mRNA is within a sequence SEQ ID NO: 224. In some cases, the targeted region of the pre-mRNA is within a sequence between a pair of genomic sites GRCh38/hg38: chr1 69716142, and GRCh38/hg38: chr1 69716218. In some cases, the therapeutic agent is an antisense oligomer complementary to a sequence with at least about 80%, 85%, 90%, 95%, 96%, 97%, 98%, 99% or 100% sequence identity to SEQ ID NO:224. In some cases, the therapeutic agent is an antisense oligomer complementary to a sequence with 100% sequence identity to SEQ ID NO:224. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4304-4357. In some cases, the therapeutic agent is an antisense oligomer that has a sequence selected from the group consisting of SEQ ID NOs: 4304-4357. In some cases, the target peptide sequence is a portion of LRRC7 protein, and the method treats a disease or the condition that comprises Malignant neoplasm of breast.

LRTOMT

In some cases, the target peptide sequence is a portion of LRTOMT protein. In some cases, the therapeutic agent increases level of a first processed mRNA encoding a protein having a sequence SEQ ID NO: 8191, and decreases level of a second processed mRNA encoding a protein having a sequence SEQ ID NO: 427. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 225. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 225. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 225. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 225. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 225. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:225. In some cases, the therapeutic agent is an antisense oligomer complementary to a sequence with 100% sequence identity to SEQ ID NO:225. In some cases, the therapeutic agent increases level of a first processed mRNA encoding a protein having a sequence SEQ ID NO: 8192, and decreases level of a second processed mRNA encoding a protein having a sequence SEQ ID NO: 428. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 226. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 226. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 226. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 226. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 226. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:226. In some cases, the therapeutic agent is an antisense oligomer complementary to a sequence with 100% sequence identity to SEQ ID NO:226. In some cases, the therapeutic agent increases level of a first processed mRNA encoding a protein having a sequence SEQ ID NO: 8193, and decreases level of a second processed mRNA encoding a protein having a sequence SEQ ID NO: 429. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 227. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 227. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 227. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 227. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 227. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:227. In some cases, the therapeutic agent is an antisense oligomer complementary to a sequence with 100% sequence identity to SEQ ID NO:227. In some cases, the therapeutic agent increases level of a first processed mRNA encoding a protein having a sequence SEQ ID NO: 8194, and decreases level of a second processed mRNA encoding a protein having a sequence SEQ ID NO: 430. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 228. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 228. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 228. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 228. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 228. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:228. In some cases, the therapeutic agent is an antisense oligomer complementary to a sequence with 100% sequence identity to SEQ ID NO:228. In some cases, the therapeutic agent increases level of a first processed mRNA encoding a protein having a sequence SEQ ID NO: 8195, and decreases level of a second processed mRNA encoding a protein having a sequence SEQ ID NO: 431. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 229. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 229. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 229. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 229. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 229. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:229. In some cases, the therapeutic agent is an antisense oligomer complementary to a sequence with 100% sequence identity to SEQ ID NO:229. In some cases, the therapeutic agent increases level of a first processed mRNA encoding a protein having a sequence SEQ ID NO: 8196, and decreases level of a second processed mRNA encoding a protein having a sequence SEQ ID NO: 432. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 230. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 230. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 230. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 230. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 230. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:230. In some cases, the therapeutic agent is an antisense oligomer complementary to a sequence with 100% sequence identity to SEQ ID NO:230. In some cases, the therapeutic agent increases level of a first processed mRNA encoding a protein having a sequence SEQ ID NO: 8197, and decreases level of a second processed mRNA encoding a protein having a sequence SEQ ID NO: 433. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 231. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 231. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 231. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 231. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 231. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:231. In some cases, the therapeutic agent is an antisense oligomer complementary to a sequence with 100% sequence identity to SEQ ID NO:231. In some cases, the therapeutic agent increases level of a first processed mRNA encoding a protein having a sequence SEQ ID NO: 8198, and decreases level of a second processed mRNA encoding a protein having a sequence SEQ ID NO: 434. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 232. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 232. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 232. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 232. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 232. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:232. In some cases, the therapeutic agent is an antisense oligomer complementary to a sequence with 100% sequence identity to SEQ ID NO:232. In some cases, the therapeutic agent increases level of a first processed mRNA encoding a protein having a sequence SEQ ID NO: 8199, and decreases level of a second processed mRNA encoding a protein having a sequence SEQ ID NO: 435. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 233. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 233. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 233. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 233. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 233. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:233. In some cases, the therapeutic agent is an antisense oligomer complementary to a sequence with 100% sequence identity to SEQ ID NO:233. In some cases, the therapeutic agent increases level of a first processed mRNA encoding a protein having a sequence SEQ ID NO: 8200, and decreases level of a second processed mRNA encoding a protein having a sequence SEQ ID NO: 436. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 234. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 234. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 234. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 234. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 234. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:234. In some cases, the therapeutic agent is an antisense oligomer complementary to a sequence with 100% sequence identity to SEQ ID NO:234. In some cases, the therapeutic agent increases level of a first processed mRNA encoding a protein having a sequence SEQ ID NO: 8201, and decreases level of a second processed mRNA encoding a protein having a sequence SEQ ID NO: 437. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 235. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 235. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1l 72089029. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr11 72089029. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 235. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 235. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr11 72089165. In some cases, the targeted region of the pre-mRNA is within a sequence SEQ ID NO: 235. In some cases, the targeted region of the pre-mRNA is within a sequence between a pair of genomic sites GRCh38/hg38: chr11 72089029, and GRCh38/hg38: chr11 72089165. In some cases, the therapeutic agent is an antisense oligomer complementary to a sequence with at least about 80%, 85%, 90%, 95%, 96%, 97%, 98%, 99% or 100% sequence identity to SEQ ID NO:235. In some cases, the therapeutic agent is an antisense oligomer complementary to a sequence with 100% sequence identity to SEQ ID NO:235. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4358-4423. In some cases, the therapeutic agent is an antisense oligomer that has a sequence selected from the group consisting of SEQ ID NOs: 4358-4423. In some cases, the target peptide sequence is a portion of LRTOMT protein, and the method treats a disease or the condition that comprises Deafness, Automal Recessive 71.

LZTFL1

In some cases, the target peptide sequence is a portion of LZTFL1 protein. In some cases, the therapeutic agent increases level of a first processed mRNA encoding a protein having a sequence SEQ ID NO: 8202, and decreases level of a second processed mRNA encoding a protein having a sequence SEQ ID NO: 438. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 236. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 236. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 45838051. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 45838051. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 236. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 236. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 45837927. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 45837927. In some cases, the targeted region of the pre-mRNA is within a sequence SEQ ID NO: 236. In some cases, the targeted region of the pre-mRNA is within a sequence between a pair of genomic sites GRCh38/hg38: chr3 45838051, and GRCh38/hg38: chr3 45837927. In some cases, the therapeutic agent is an antisense oligomer complementary to a sequence with at least about 80%, 85%, 90%, 95%, 96%, 97%, 98%, 99% or 100% sequence identity to SEQ ID NO:236. In some cases, the therapeutic agent is an antisense oligomer complementary to a sequence with 100% sequence identity to SEQ ID NO:236. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4424-4487. In some cases, the therapeutic agent is an antisense oligomer that has a sequence selected from the group consisting of SEQ ID NOs: 4424-4487. In some cases, the target peptide sequence is a portion of LZTFL1 protein, and the method treats a disease or the condition that comprises Bardet-Biedl Syndrome; Polydactyly; Ciliopathies; or Bardet-Biedl Syndrome 17.

MAGI2

In some cases, the target peptide sequence is a portion of MAGI2 protein. In some cases, the therapeutic agent increases level of a first processed mRNA encoding a protein having a sequence SEQ ID NO: 8203, and decreases level of a second processed mRNA encoding a protein having a sequence SEQ ID NO: 439. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 237. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 237. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 78554671. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 78554671. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 237. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 237. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 78554567. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 78554567. In some cases, the targeted region of the pre-mRNA is within a sequence SEQ ID NO: 237. In some cases, the targeted region of the pre-mRNA is within a sequence between a pair of genomic sites GRCh38/hg38: chr7 78554671, and GRCh38/hg38: chr7 78554567. In some cases, the therapeutic agent is an antisense oligomer complementary to a sequence with at least about 80%, 85%, 90%, 95%, 96%, 97%, 98%, 99% or 100% sequence identity to SEQ ID NO:237. In some cases, the therapeutic agent is an antisense oligomer complementary to a sequence with 100% sequence identity to SEQ ID NO:237. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4488-4547. In some cases, the therapeutic agent is an antisense oligomer that has a sequence selected from the group consisting of SEQ ID NOs: 4488-4547. In some cases, the target peptide sequence is a portion of MAGI2 protein, and the method treats a disease or the condition that comprises Intellectual Disability; Major Affective Disorder 2; Bipolar Disorder; Epileptic encephalopathy; or Schizophrenia.

MERTK

In some cases, the target peptide sequence is a portion of MERTK protein. In some cases, the therapeutic agent increases level of a first processed mRNA encoding a protein having a sequence SEQ ID NO: 8206, and decreases level of a second processed mRNA encoding a protein having a sequence SEQ ID NO: 442. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 240. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 240. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 111944960. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 111944960. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 240. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 240. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 111945060. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 111945060. In some cases, the targeted region of the pre-mRNA is within a sequence SEQ ID NO: 240. In some cases, the targeted region of the pre-mRNA is within a sequence between a pair of genomic sites GRCh38/hg38: chr2 111944960, and GRCh38/hg38: chr2 111945060. In some cases, the therapeutic agent is an antisense oligomer complementary to a sequence with at least about 80%, 85%, 90%, 95%, 96%, 97%, 98%, 99% or 100% sequence identity to SEQ ID NO:240. In some cases, the therapeutic agent is an antisense oligomer complementary to a sequence with 100% sequence identity to SEQ ID NO:240. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4612-4670. In some cases, the therapeutic agent is an antisense oligomer that has a sequence selected from the group consisting of SEQ ID NOs: 4612-4670. In some cases, the target peptide sequence is a portion of MERTK protein, and the method treats a disease or the condition that comprises Benign neoplasm of adrenal gland; Retinitis Pigmentosa; Conventional (Clear Cell) Renal Cell Carcinoma; Squamous cell carcinoma of the head and neck; Malignant neoplasm of aortic body and other paraganglia; Retinitis Pigmentosa 38; Malignant Adrenal Medulla Neoplasm; or Benign neoplasm of aortic body and other paraganglia.

MFSD2A

In some cases, the target peptide sequence is a portion of MFSD2A protein. In some cases, the therapeutic agent increases level of a first processed mRNA encoding a protein having a sequence SEQ ID NO: 8207, and decreases level of a second processed mRNA encoding a protein having a sequence SEQ ID NO: 443. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 241. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 241. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 39965211. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 39965211. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 241. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 241. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 39965334. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 39965334. In some cases, the targeted region of the pre-mRNA is within a sequence SEQ ID NO: 241. In some cases, the targeted region of the pre-mRNA is within a sequence between a pair of genomic sites GRCh38/hg38: chr1 39965211, and GRCh38/hg38: chr1 39965334. In some cases, the therapeutic agent is an antisense oligomer complementary to a sequence with at least about 80%, 85%, 90%, 95%, 96%, 97%, 98%, 99% or 100% sequence identity to SEQ ID NO:241. In some cases, the therapeutic agent is an antisense oligomer complementary to a sequence with 100% sequence identity to SEQ ID NO:241. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4671-4734. In some cases, the therapeutic agent is an antisense oligomer that has a sequence selected from the group consisting of SEQ ID NOs: 4671-4734. In some cases, the target peptide sequence is a portion of MFSD2A protein, and the method treats a disease or the condition that comprises Intellectual Disability; Primary microcephaly; Microcephaly 15, Primary, Autosomal Recessive; Autosomal Recessive Primary Microcephaly; or Microcephaly. In some cases, the method treats a cancer that is associated with MFSD2A protein.

MLF1

In some cases, the target peptide sequence is a portion of MLF1 protein. In some cases, the therapeutic agent increases level of a first processed mRNA encoding a protein having a sequence SEQ ID NO: 8208, and decreases level of a second processed mRNA encoding a protein having a sequence SEQ ID NO: 444. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 242. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 242. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 242. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 242. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases, the targeted region of the pre-mRNA is within a sequence SEQ ID NO: 242. In some cases, the targeted region of the pre-mRNA is within a sequence between a pair of genomic sites GRCh38/hg38: chr3 158592434, and GRCh38/hg38: chr3 158592581. In some cases, the therapeutic agent is an antisense oligomer complementary to a sequence with at least about 80%, 85%, 90%, 95%, 96%, 97%, 98%, 99% or 100% sequence identity to SEQ ID NO:242. In some cases, the therapeutic agent is an antisense oligomer complementary to a sequence with 100% sequence identity to SEQ ID NO:242. In some cases, the therapeutic agent increases level of a first processed mRNA encoding a protein having a sequence SEQ ID NO: 8209, and decreases level of a second processed mRNA encoding a protein having a sequence SEQ ID NO: 445. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 243. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 243. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 243. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 243. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases, the targeted region of the pre-mRNA is within a sequence SEQ ID NO: 243. In some cases, the targeted region of the pre-mRNA is within a sequence between a pair of genomic sites GRCh38/hg38: chr3 158592434, and GRCh38/hg38: chr3 158592581. In some cases, the therapeutic agent is an antisense oligomer complementary to a sequence with at least about 80%, 85%, 90%, 95%, 96%, 97%, 98%, 99% or 100% sequence identity to SEQ ID NO:243. In some cases, the therapeutic agent is an antisense oligomer complementary to a sequence with 100% sequence identity to SEQ ID NO:243. In some cases, the therapeutic agent increases level of a first processed mRNA encoding a protein having a sequence SEQ ID NO: 8210, and decreases level of a second processed mRNA encoding a protein having a sequence SEQ ID NO: 446. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 244. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 244. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 244. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 244. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases, the targeted region of the pre-mRNA is within a sequence SEQ ID NO: 244. In some cases, the targeted region of the pre-mRNA is within a sequence between a pair of genomic sites GRCh38/hg38: chr3 158592434, and GRCh38/hg38: chr3 158592581. In some cases, the therapeutic agent is an antisense oligomer complementary to a sequence with at least about 80%, 85%, 90%, 95%, 96%, 97%, 98%, 99% or 100% sequence identity to SEQ ID NO:244. In some cases, the therapeutic agent is an antisense oligomer complementary to a sequence with 100% sequence identity to SEQ ID NO:244. In some cases, the therapeutic agent increases level of a first processed mRNA encoding a protein having a sequence SEQ ID NO: 8211, and decreases level of a second processed mRNA encoding a protein having a sequence SEQ ID NO: 447. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 245. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 245. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 245. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 245. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases, the targeted region of the pre-mRNA is within a sequence SEQ ID NO: 245. In some cases, the targeted region of the pre-mRNA is within a sequence between a pair of genomic sites GRCh38/hg38: chr3 158592434, and GRCh38/hg38: chr3 158592581. In some cases, the therapeutic agent is an antisense oligomer complementary to a sequence with at least about 80%, 85%, 90%, 95%, 96%, 97%, 98%, 99% or 100% sequence identity to SEQ ID NO:245. In some cases, the therapeutic agent is an antisense oligomer complementary to a sequence with 100% sequence identity to SEQ ID NO:245. In some cases, the therapeutic agent increases level of a first processed mRNA encoding a protein having a sequence SEQ ID NO: 8212, and decreases level of a second processed mRNA encoding a protein having a sequence SEQ ID NO: 448. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 246. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 246. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 158592434. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 246. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 246. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 158592581. In some cases, the targeted region of the pre-mRNA is within a sequence SEQ ID NO: 246. In some cases, the targeted region of the pre-mRNA is within a sequence between a pair of genomic sites GRCh38/hg38: chr3 158592434, and GRCh38/hg38: chr3 158592581. In some cases, the therapeutic agent is an antisense oligomer complementary to a sequence with at least about 80%, 85%, 90%, 95%, 96%, 97%, 98%, 99% or 100% sequence identity to SEQ ID NO:246. In some cases, the therapeutic agent is an antisense oligomer complementary to a sequence with 100% sequence identity to SEQ ID NO:246. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4735-4803. In some cases, the therapeutic agent is an antisense oligomer that has a sequence selected from the group consisting of SEQ ID NOs: 4735-4803. In some cases, the target peptide sequence is a portion of MLF1 protein, and the method treats a disease or the condition that comprises Leukemia, acute myeloid.

MOB1B

In some cases, the target peptide sequence is a portion of MOB1B protein. In some cases, the therapeutic agent increases level of a first processed mRNA encoding a protein having a sequence SEQ ID NO: 8213, and decreases level of a second processed mRNA encoding a protein having a sequence SEQ ID NO: 449. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 247. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 247. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 70950700. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 70950700. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 247. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 247. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 70950811. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 70950811. In some cases, the targeted region of the pre-mRNA is within a sequence SEQ ID NO: 247. In some cases, the targeted region of the pre-mRNA is within a sequence between a pair of genomic sites GRCh38/hg38: chr4 70950700, and GRCh38/hg38: chr4 70950811. In some cases, the therapeutic agent is an antisense oligomer complementary to a sequence with at least about 80%, 85%, 90%, 95%, 96%, 97%, 98%, 99% or 100% sequence identity to SEQ ID NO:247. In some cases, the therapeutic agent is an antisense oligomer complementary to a sequence with 100% sequence identity to SEQ ID NO:247. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4804-4864. In some cases, the therapeutic agent is an antisense oligomer that has a sequence selected from the group consisting of SEQ ID NOs: 4804-4864. In some cases, the target peptide sequence is a portion of MOB1B protein, and the method treats a cancer that is associated with MOB1B protein.

MSRB3

In some cases, the target peptide sequence is a portion of MSRB3 protein. In some cases, the therapeutic agent increases level of a first processed mRNA encoding a protein having a sequence SEQ ID NO: 8214, and decreases level of a second processed mRNA encoding a protein having a sequence SEQ ID NO: 450. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 248. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 248. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 65308529. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr12 65308529. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 248. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 248. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 65308655. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr12 65308655. In some cases, the targeted region of the pre-mRNA is within a sequence SEQ ID NO: 248. In some cases, the targeted region of the pre-mRNA is within a sequence between a pair of genomic sites GRCh38/hg38: chr12 65308529, and GRCh38/hg38: chr12 65308655. In some cases, the therapeutic agent is an antisense oligomer complementary to a sequence with at least about 80%, 85%, 90%, 95%, 96%, 97%, 98%, 99% or 100% sequence identity to SEQ ID NO:248. In some cases, the therapeutic agent is an antisense oligomer complementary to a sequence with 100% sequence identity to SEQ ID NO:248. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4865-4928. In some cases, the therapeutic agent is an antisense oligomer that has a sequence selected from the group consisting of SEQ ID NOs: 4865-4928. In some cases, the target peptide sequence is a portion of MSRB3 protein, and the method treats a disease or the condition that comprises Deafness, Autosomal Recessive 74.

NR1I3

In some cases, the target peptide sequence is a portion of NR1I3 protein. In some cases, the therapeutic agent increases level of a first processed mRNA encoding a protein having a sequence SEQ ID NO: 8215, and decreases level of a second processed mRNA encoding a protein having a sequence SEQ ID NO: 451. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 249. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 249. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 249. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 249. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 249. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:249. In some cases, the therapeutic agent is an antisense oligomer complementary to a sequence with 100% sequence identity to SEQ ID NO:249. In some cases, the therapeutic agent increases level of a first processed mRNA encoding a protein having a sequence SEQ ID NO: 8216, and decreases level of a second processed mRNA encoding a protein having a sequence SEQ ID NO: 452. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 250. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 250. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 250. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 250. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 250. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:250. In some cases, the therapeutic agent is an antisense oligomer complementary to a sequence with 100% sequence identity to SEQ ID NO:250. In some cases, the therapeutic agent increases level of a first processed mRNA encoding a protein having a sequence SEQ ID NO: 8217, and decreases level of a second processed mRNA encoding a protein having a sequence SEQ ID NO: 453. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 251. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 251. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 251. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 251. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 251. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:251. In some cases, the therapeutic agent is an antisense oligomer complementary to a sequence with 100% sequence identity to SEQ ID NO:251. In some cases, the therapeutic agent increases level of a first processed mRNA encoding a protein having a sequence SEQ ID NO: 8218, and decreases level of a second processed mRNA encoding a protein having a sequence SEQ ID NO: 454. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 252. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 252. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 252. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 252. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 252. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:252. In some cases, the therapeutic agent is an antisense oligomer complementary to a sequence with 100% sequence identity to SEQ ID NO:252. In some cases, the therapeutic agent increases level of a first processed mRNA encoding a protein having a sequence SEQ ID NO: 8219, and decreases level of a second processed mRNA encoding a protein having a sequence SEQ ID NO: 455. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 253. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 253. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 253. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 253. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 253. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:253. In some cases, the therapeutic agent is an antisense oligomer complementary to a sequence with 100% sequence identity to SEQ ID NO:253. In some cases, the therapeutic agent increases level of a first processed mRNA encoding a protein having a sequence SEQ ID NO: 8220, and decreases level of a second processed mRNA encoding a protein having a sequence SEQ ID NO: 456. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 254. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 254. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 254. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 254. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 254. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:254. In some cases, the therapeutic agent is an antisense oligomer complementary to a sequence with 100% sequence identity to SEQ ID NO:254. In some cases, the therapeutic agent increases level of a first processed mRNA encoding a protein having a sequence SEQ ID NO: 8221, and decreases level of a second processed mRNA encoding a protein having a sequence SEQ ID NO: 457. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 255. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 255. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 255. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 255. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 255. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:255. In some cases, the therapeutic agent is an antisense oligomer complementary to a sequence with 100% sequence identity to SEQ ID NO:255. In some cases, the therapeutic agent increases level of a first processed mRNA encoding a protein having a sequence SEQ ID NO: 8222, and decreases level of a second processed mRNA encoding a protein having a sequence SEQ ID NO: 458. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 256. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 256. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 256. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 256. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 256. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:256. In some cases, the therapeutic agent is an antisense oligomer complementary to a sequence with 100% sequence identity to SEQ ID NO:256. In some cases, the therapeutic agent increases level of a first processed mRNA encoding a protein having a sequence SEQ ID NO: 8223, and decreases level of a second processed mRNA encoding a protein having a sequence SEQ ID NO: 459. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 257. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 257. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 257. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 257. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 257. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:257. In some cases, the therapeutic agent is an antisense oligomer complementary to a sequence with 100% sequence identity to SEQ ID NO:257. In some cases, the therapeutic agent increases level of a first processed mRNA encoding a protein having a sequence SEQ ID NO: 8224, and decreases level of a second processed mRNA encoding a protein having a sequence SEQ ID NO: 460. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 258. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 258. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 258. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 258. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 258. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:258. In some cases, the therapeutic agent is an antisense oligomer complementary to a sequence with 100% sequence identity to SEQ ID NO:258. In some cases, the therapeutic agent increases level of a first processed mRNA encoding a protein having a sequence SEQ ID NO: 8225, and decreases level of a second processed mRNA encoding a protein having a sequence SEQ ID NO: 461. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 259. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 259. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 259. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 259. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 259. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:259. In some cases, the therapeutic agent is an antisense oligomer complementary to a sequence with 100% sequence identity to SEQ ID NO:259. In some cases, the therapeutic agent increases level of a first processed mRNA encoding a protein having a sequence SEQ ID NO: 8226, and decreases level of a second processed mRNA encoding a protein having a sequence SEQ ID NO: 462. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 260. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 260. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 260. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 260. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 260. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:260. In some cases, the therapeutic agent is an antisense oligomer complementary to a sequence with 100% sequence identity to SEQ ID NO:260. In some cases, the therapeutic agent increases level of a first processed mRNA encoding a protein having a sequence SEQ ID NO: 8227, and decreases level of a second processed mRNA encoding a protein having a sequence SEQ ID NO: 463. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 261. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 261. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 161236598. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 261. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 261. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 161236459. In some cases, the targeted region of the pre-mRNA is within a sequence SEQ ID NO: 261. In some cases, the targeted region of the pre-mRNA is within a sequence between a pair of genomic sites GRCh38/hg38: chr1 161236598, and GRCh38/hg38: chr1 161236459. In some cases, the therapeutic agent is an antisense oligomer complementary to a sequence with at least about 80%, 85%, 90%, 95%, 96%, 97%, 98%, 99% or 100% sequence identity to SEQ ID NO:261. In some cases, the therapeutic agent is an antisense oligomer complementary to a sequence with 100% sequence identity to SEQ ID NO:261. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4929-4995. In some cases, the therapeutic agent is an antisense oligomer that has a sequence selected from the group consisting of SEQ ID NOs: 4929-4995. In some cases, the target peptide sequence is a portion of NR1I3 protein, and the method treats a disease or the condition that comprises Intellectual Disability.

NT5C3A

In some cases, the target peptide sequence is a portion of NT5C3A protein. In some cases, the therapeutic agent increases level of a first processed mRNA encoding a protein having a sequence SEQ ID NO: 8228, and decreases level of a second processed mRNA encoding a protein having a sequence SEQ ID NO: 464. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 262. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 262. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 33035988. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 33035988. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 262. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 262. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 33035934. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 33035934. In some cases, the targeted region of the pre-mRNA is within a sequence SEQ ID NO: 262. In some cases, the targeted region of the pre-mRNA is within a sequence between a pair of genomic sites GRCh38/hg38: chr7 33035988, and GRCh38/hg38: chr7 33035934. In some cases, the therapeutic agent is an antisense oligomer complementary to a sequence with at least about 80%, 85%, 90%, 95%, 96%, 97%, 98%, 99% or 100% sequence identity to SEQ ID NO:262. In some cases, the therapeutic agent is an antisense oligomer complementary to a sequence with 100% sequence identity to SEQ ID NO:262. In some cases, the therapeutic agent increases level of a first processed mRNA encoding a protein having a sequence SEQ ID NO: 8229, and decreases level of a second processed mRNA encoding a protein having a sequence SEQ ID NO: 465. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 263. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 263. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 33035988. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 33035988. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 263. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 263. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 33035934. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 33035934. In some cases, the targeted region of the pre-mRNA is within a sequence SEQ ID NO: 263. In some cases, the targeted region of the pre-mRNA is within a sequence between a pair of genomic sites GRCh38/hg38: chr7 33035988, and GRCh38/hg38: chr7 33035934. In some cases, the therapeutic agent is an antisense oligomer complementary to a sequence with at least about 80%, 85%, 90%, 95%, 96%, 97%, 98%, 99% or 100% sequence identity to SEQ ID NO:263. In some cases, the therapeutic agent is an antisense oligomer complementary to a sequence with 100% sequence identity to SEQ ID NO:263. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 4996-5045. In some cases, the therapeutic agent is an antisense oligomer that has a sequence selected from the group consisting of SEQ ID NOs: 4996-5045. In some cases, the target peptide sequence is a portion of NT5C3A protein, and the method treats a disease or the condition that comprises Intellectual Disability; Cytopenia; Uridine 5-Prime Monophosphate Hydrolase Deficiency, Hemolytic Anemia due to; or Congenital anemia.

NUTM1

In some cases, the target peptide sequence is a portion of NUTM1 protein. In some cases, the therapeutic agent increases level of a first processed mRNA encoding a protein having a sequence SEQ ID NO: 8230, and decreases level of a second processed mRNA encoding a protein having a sequence SEQ ID NO: 466. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 264. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 264. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 34345857. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 34345857. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 264. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 264. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 34346035. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 34346035. In some cases, the targeted region of the pre-mRNA is within a sequence SEQ ID NO: 264. In some cases, the targeted region of the pre-mRNA is within a sequence between a pair of genomic sites GRCh38/hg38: chr15 34345857, and GRCh38/hg38: chr15 34346035. In some cases, the therapeutic agent is an antisense oligomer complementary to a sequence with at least about 80%, 85%, 90%, 95%, 96%, 97%, 98%, 99% or 100% sequence identity to SEQ ID NO:264. In some cases, the therapeutic agent is an antisense oligomer complementary to a sequence with 100% sequence identity to SEQ ID NO:264. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5046-5120. In some cases, the therapeutic agent is an antisense oligomer that has a sequence selected from the group consisting of SEQ ID NOs: 5046-5120. In some cases, the target peptide sequence is a portion of NUTM1 protein, and the method treats a disease or the condition that comprises NUT midline carcinoma.

OTOF

In some cases, the target peptide sequence is a portion of OTOF protein. In some cases, the therapeutic agent increases level of a first processed mRNA encoding a protein having a sequence SEQ ID NO: 8231, and decreases level of a second processed mRNA encoding a protein having a sequence SEQ ID NO: 467. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 265. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 265. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 26477749. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 26477749. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 265. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 265. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 26477649. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 26477649. In some cases, the targeted region of the pre-mRNA is within a sequence SEQ ID NO: 265. In some cases, the targeted region of the pre-mRNA is within a sequence between a pair of genomic sites GRCh38/hg38: chr2 26477749, and GRCh38/hg38: chr2 26477649. In some cases, the therapeutic agent is an antisense oligomer complementary to a sequence with at least about 80%, 85%, 90%, 95%, 96%, 97%, 98%, 99% or 100% sequence identity to SEQ ID NO:265. In some cases, the therapeutic agent is an antisense oligomer complementary to a sequence with 100% sequence identity to SEQ ID NO:265. In some cases, the therapeutic agent increases level of a first processed mRNA encoding a protein having a sequence SEQ ID NO: 8232, and decreases level of a second processed mRNA encoding a protein having a sequence SEQ ID NO: 468. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 266. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 266. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 26477749. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 26477749. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 266. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 266. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 26477649. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 26477649. In some cases, the targeted region of the pre-mRNA is within a sequence SEQ ID NO: 266. In some cases, the targeted region of the pre-mRNA is within a sequence between a pair of genomic sites GRCh38/hg38: chr2 26477749, and GRCh38/hg38: chr2 26477649. In some cases, the therapeutic agent is an antisense oligomer complementary to a sequence with at least about 80%, 85%, 90%, 95%, 96%, 97%, 98%, 99% or 100% sequence identity to SEQ ID NO:266. In some cases, the therapeutic agent is an antisense oligomer complementary to a sequence with 100% sequence identity to SEQ ID NO:266. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5121-5179. In some cases, the therapeutic agent is an antisense oligomer that has a sequence selected from the group consisting of SEQ ID NOs: 5121-5179. In some cases, the target peptide sequence is a portion of OTOF protein, and the method treats a disease or the condition that comprises Malignant neoplasm of breast; Deafness, Autosomal Recessive; Deafness, Autosomal Recessive 9; Auditory Neuropathy, Nonsyndromic Recessive; or Auditory neuropathy, autosomal recessive, 1.

PDCD4

In some cases, the target peptide sequence is a portion of PDCD4 protein. In some cases, the therapeutic agent increases level of a first processed mRNA encoding a protein having a sequence SEQ ID NO: 8233, and decreases level of a second processed mRNA encoding a protein having a sequence SEQ ID NO: 469. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 267. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 267. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 110876670. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 110876670. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 267. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 267. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 110876753. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 110876753. In some cases, the targeted region of the pre-mRNA is within a sequence SEQ ID NO: 267. In some cases, the targeted region of the pre-mRNA is within a sequence between a pair of genomic sites GRCh38/hg38: chr10 110876670, and GRCh38/hg38: chr10 110876753. In some cases, the therapeutic agent is an antisense oligomer complementary to a sequence with at least about 80%, 85%, 90%, 95%, 96%, 97%, 98%, 99% or 100% sequence identity to SEQ ID NO:267. In some cases, the therapeutic agent is an antisense oligomer complementary to a sequence with 100% sequence identity to SEQ ID NO:267. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5180-5235. In some cases, the therapeutic agent is an antisense oligomer that has a sequence selected from the group consisting of SEQ ID NOs: 5180-5235. In some cases, the target peptide sequence is a portion of PDCD4 protein, and the method treats a disease or the condition that comprises Malignant neoplasm of breast. In some cases, the method treats a cancer that is associated with PDCD4 protein.

PDPK1

In some cases, the target peptide sequence is a portion of PDPK1 protein. In some cases, the therapeutic agent increases level of a first processed mRNA encoding a protein having a sequence SEQ ID NO: 8234, and decreases level of a second processed mRNA encoding a protein having a sequence SEQ ID NO: 470. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 268. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 268. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2566356. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2566356. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 268. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 268. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2566453. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2566453. In some cases, the targeted region of the pre-mRNA is within a sequence SEQ ID NO: 268. In some cases, the targeted region of the pre-mRNA is within a sequence between a pair of genomic sites GRCh38/hg38: chr16 2566356, and GRCh38/hg38: chr16 2566453. In some cases, the therapeutic agent is an antisense oligomer complementary to a sequence with at least about 80%, 85%, 90%, 95%, 96%, 97%, 98%, 99% or 100% sequence identity to SEQ ID NO:268. In some cases, the therapeutic agent is an antisense oligomer complementary to a sequence with 100% sequence identity to SEQ ID NO:268. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5236-5294. In some cases, the therapeutic agent is an antisense oligomer that has a sequence selected from the group consisting of SEQ ID NOs: 5236-5294. In some cases, the target peptide sequence is a portion of PDPK1 protein, and the method treats a disease or the condition that comprises Malignant neoplasm of breast; Breast Carcinoma; Neoplasm of uncertain or unknown behavior of breast; or Breast adenocarcinoma.

PHKB

In some cases, the target peptide sequence is a portion of PHKB protein. In some cases, the therapeutic agent increases level of a first processed mRNA encoding a protein having a sequence SEQ ID NO: 8235, and decreases level of a second processed mRNA encoding a protein having a sequence SEQ ID NO: 471. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 269. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 269. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 47463899. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 47463899. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 269. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 269. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 47463993. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 47463993. In some cases, the targeted region of the pre-mRNA is within a sequence SEQ ID NO: 269. In some cases, the targeted region of the pre-mRNA is within a sequence between a pair of genomic sites GRCh38/hg38: chr16 47463899, and GRCh38/hg38: chr16 47463993. In some cases, the therapeutic agent is an antisense oligomer complementary to a sequence with at least about 80%, 85%, 90%, 95%, 96%, 97%, 98%, 99% or 100% sequence identity to SEQ ID NO:269. In some cases, the therapeutic agent is an antisense oligomer complementary to a sequence with 100% sequence identity to SEQ ID NO:269. In some cases, the therapeutic agent increases level of a first processed mRNA encoding a protein having a sequence SEQ ID NO: 8236, and decreases level of a second processed mRNA encoding a protein having a sequence SEQ ID NO: 472. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 270. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 270. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 47463882. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 47463882. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 270. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 270. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 47463993. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 47463993. In some cases, the targeted region of the pre-mRNA is within a sequence SEQ ID NO: 270. In some cases, the targeted region of the pre-mRNA is within a sequence between a pair of genomic sites GRCh38/hg38: chr16 47463882, and GRCh38/hg38: chr16 47463993. In some cases, the therapeutic agent is an antisense oligomer complementary to a sequence with at least about 80%, 85%, 90%, 95%, 96%, 97%, 98%, 99% or 100% sequence identity to SEQ ID NO:270. In some cases, the therapeutic agent is an antisense oligomer complementary to a sequence with 100% sequence identity to SEQ ID NO:270. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5295-5352. In some cases, the therapeutic agent is an antisense oligomer that has a sequence selected from the group consisting of SEQ ID NOs: 5295-5352. In some cases, the target peptide sequence is a portion of PHKB protein, and the method treats a disease or the condition that comprises Malignant neoplasm of breast; Ketotic hypoglycemia; Rhabdomyolysis; Glycogen Storage Disease IXB; or Phosphorylase kinase deficiency of liver and muscle, autosomal recessive.

PIGA

In some cases, the target peptide sequence is a portion of PIGA protein. In some cases, the therapeutic agent increases level of a first processed mRNA encoding a protein having a sequence SEQ ID NO: 8237, and decreases level of a second processed mRNA encoding a protein having a sequence SEQ ID NO: 473. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 271. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 271. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 15331992. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 15331992. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 271. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 271. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 15331216. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 15331216. In some cases, the targeted region of the pre-mRNA is within a sequence SEQ ID NO: 271. In some cases, the targeted region of the pre-mRNA is within a sequence between a pair of genomic sites GRCh38/hg38: chrX 15331992, and GRCh38/hg38: chrX 15331216. In some cases, the therapeutic agent is an antisense oligomer complementary to a sequence with at least about 80%, 85%, 90%, 95%, 96%, 97%, 98%, 99% or 100% sequence identity to SEQ ID NO:271. In some cases, the therapeutic agent is an antisense oligomer complementary to a sequence with 100% sequence identity to SEQ ID NO:271. In some cases, the therapeutic agent increases level of a first processed mRNA encoding a protein having a sequence SEQ ID NO: 8238, and decreases level of a second processed mRNA encoding a protein having a sequence SEQ ID NO: 474. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 272. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 272. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 15331992. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 15331992. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 272. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 272. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 15331216. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 15331216. In some cases, the targeted region of the pre-mRNA is within a sequence SEQ ID NO: 272. In some cases, the targeted region of the pre-mRNA is within a sequence between a pair of genomic sites GRCh38/hg38: chrX 15331992, and GRCh38/hg38: chrX 15331216. In some cases, the therapeutic agent is an antisense oligomer complementary to a sequence with at least about 80%, 85%, 90%, 95%, 96%, 97%, 98%, 99% or 100% sequence identity to SEQ ID NO:272. In some cases, the therapeutic agent is an antisense oligomer complementary to a sequence with 100% sequence identity to SEQ ID NO:272. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5353-5546. In some cases, the therapeutic agent is an antisense oligomer that has a sequence selected from the group consisting of SEQ ID NOs: 5353-5546. In some cases, the target peptide sequence is a portion of PIGA protein, and the method treats a disease or the condition that comprises Intellectual Disability; Congenital anemia; Congenital Disorders of Glycosylation; Cytopenia; Epileptic encephalopathy; Paroxysmal nocturnal hemoglobinuria; Paroxysmal Nocturnal Hemoglobinuria 1; West Syndrome; or Multiple Congenital Anomalies-Hypotonia-Seizures Syndome 2.

PLOD2

In some cases, the target peptide sequence is a portion of PLOD2 protein. In some cases, the therapeutic agent increases level of a first processed mRNA encoding a protein having a sequence SEQ ID NO: 8239, and decreases level of a second processed mRNA encoding a protein having a sequence SEQ ID NO: 475. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 273. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 273. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 146124229. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 146124229. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 273. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 273. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 146124138. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 146124138. In some cases, the targeted region of the pre-mRNA is within a sequence SEQ ID NO: 273. In some cases, the targeted region of the pre-mRNA is within a sequence between a pair of genomic sites GRCh38/hg38: chr3 146124229, and GRCh38/hg38: chr3 146124138. In some cases, the therapeutic agent is an antisense oligomer complementary to a sequence with at least about 80%, 85%, 90%, 95%, 96%, 97%, 98%, 99% or 100% sequence identity to SEQ ID NO:273. In some cases, the therapeutic agent is an antisense oligomer complementary to a sequence with 100% sequence identity to SEQ ID NO:273. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5547-5603. In some cases, the therapeutic agent is an antisense oligomer that has a sequence selected from the group consisting of SEQ ID NOs: 5547-5603. In some cases, the target peptide sequence is a portion of PLOD2 protein, and the method treats a disease or the condition that comprises Intellectual Disability; Bruck syndrome 2; Osteogenesis Imperfecta; Bruck syndrome 1; Arthrogryposis; or Bruck syndrome.

POLR2F

In some cases, the target peptide sequence is a portion of POLR2F protein. In some cases, the therapeutic agent increases level of a first processed mRNA encoding a protein having a sequence SEQ ID NO: 8240, and decreases level of a second processed mRNA encoding a protein having a sequence SEQ ID NO: 476. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 274. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 274. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr22 37958374. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr22 37958374. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 274. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 274. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr22 37958471. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr22 37958471. In some cases, the targeted region of the pre-mRNA is within a sequence SEQ ID NO: 274. In some cases, the targeted region of the pre-mRNA is within a sequence between a pair of genomic sites GRCh38/hg38: chr22 37958374, and GRCh38/hg38: chr22 37958471. In some cases, the therapeutic agent is an antisense oligomer complementary to a sequence with at least about 80%, 85%, 90%, 95%, 96%, 97%, 98%, 99% or 100% sequence identity to SEQ ID NO:274. In some cases, the therapeutic agent is an antisense oligomer complementary to a sequence with 100% sequence identity to SEQ ID NO:274. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5604-5662. In some cases, the therapeutic agent is an antisense oligomer that has a sequence selected from the group consisting of SEQ ID NOs: 5604-5662. In some cases, the target peptide sequence is a portion of POLR2F protein, and the method treats a disease or the condition that comprises Malignant neoplasm of breast.

PPP6C

In some cases, the target peptide sequence is a portion of PPP6C protein. In some cases, the therapeutic agent increases level of a first processed mRNA encoding a protein having a sequence SEQ ID NO: 8241, and decreases level of a second processed mRNA encoding a protein having a sequence SEQ ID NO: 477. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 275. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 275. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr9 125172001. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr9 125172001. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 275. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 275. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr9 125171909. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr9 125171909. In some cases, the targeted region of the pre-mRNA is within a sequence SEQ ID NO: 275. In some cases, the targeted region of the pre-mRNA is within a sequence between a pair of genomic sites GRCh38/hg38: chr9 125172001, and GRCh38/hg38: chr9 125171909. In some cases, the therapeutic agent is an antisense oligomer complementary to a sequence with at least about 80%, 85%, 90%, 95%, 96%, 97%, 98%, 99% or 100% sequence identity to SEQ ID NO:275. In some cases, the therapeutic agent is an antisense oligomer complementary to a sequence with 100% sequence identity to SEQ ID NO:275. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5663-5720. In some cases, the therapeutic agent is an antisense oligomer that has a sequence selected from the group consisting of SEQ ID NOs: 5663-5720. In some cases, the target peptide sequence is a portion of PPP6C protein, and the method treats a disease or the condition that comprises Cutaneous Melanoma; or melanoma.

PRKN

In some cases, the target peptide sequence is a portion of PRKN protein. In some cases, the therapeutic agent increases level of a first processed mRNA encoding a protein having a sequence SEQ ID NO: 8242, and decreases level of a second processed mRNA encoding a protein having a sequence SEQ ID NO: 478. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 276. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 276. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 162262765. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr6 162262765. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 276. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 276. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 162262525. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr6 162262525. In some cases, the targeted region of the pre-mRNA is within a sequence SEQ ID NO: 276. In some cases, the targeted region of the pre-mRNA is within a sequence between a pair of genomic sites GRCh38/hg38: chr6 162262765, and GRCh38/hg38: chr6 162262525. In some cases, the therapeutic agent is an antisense oligomer complementary to a sequence with at least about 80%, 85%, 90%, 95%, 96%, 97%, 98%, 99% or 100% sequence identity to SEQ ID NO:276. In some cases, the therapeutic agent is an antisense oligomer complementary to a sequence with 100% sequence identity to SEQ ID NO:276. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5721-5807. In some cases, the therapeutic agent is an antisense oligomer that has a sequence selected from the group consisting of SEQ ID NOs: 5721-5807. In some cases, the target peptide sequence is a portion of PRKN protein, and the method treats a disease or the condition that comprises Parkinson Disease 2, Autosomal Recessive Juvenile; Parkinson Disease; Young onset Parkinson disease; Parkinson Disease, Late-onset; Leprosy, susceptibility to; Adenocarcinoma, ovarian, somatic; or Adenocarcinoma of lung, somatic.

PRUNE1

In some cases, the target peptide sequence is a portion of PRUNE1 protein. In some cases, the therapeutic agent increases level of a first processed mRNA encoding a protein having a sequence SEQ ID NO: 8243, and decreases level of a second processed mRNA encoding a protein having a sequence SEQ ID NO: 479. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 277. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 277. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 151027233. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 151027233. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 277. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 277. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 151027327. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 151027327. In some cases, the targeted region of the pre-mRNA is within a sequence SEQ ID NO: 277. In some cases, the targeted region of the pre-mRNA is within a sequence between a pair of genomic sites GRCh38/hg38: chr1 151027233, and GRCh38/hg38: chr1 151027327. In some cases, the therapeutic agent is an antisense oligomer complementary to a sequence with at least about 80%, 85%, 90%, 95%, 96%, 97%, 98%, 99% or 100% sequence identity to SEQ ID NO:277. In some cases, the therapeutic agent is an antisense oligomer complementary to a sequence with 100% sequence identity to SEQ ID NO:277. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5808-5865. In some cases, the therapeutic agent is an antisense oligomer that has a sequence selected from the group consisting of SEQ ID NOs: 5808-5865. In some cases, the target peptide sequence is a portion of PRUNE1 protein, and the method treats a disease or the condition that comprises Intellectual Disability; Primary microcephaly; or Neurodevelopmental Disorder with Microcephaly, Hypotonia, and Variable Brain Anomalies.

PSMA6

In some cases, the target peptide sequence is a portion of PSMA6 protein. In some cases, the therapeutic agent increases level of a first processed mRNA encoding a protein having a sequence SEQ ID NO: 8244, and decreases level of a second processed mRNA encoding a protein having a sequence SEQ ID NO: 480. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 278. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 278. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 35308914. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 35308914. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 278. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 278. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 35308995. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 35308995. In some cases, the targeted region of the pre-mRNA is within a sequence SEQ ID NO: 278. In some cases, the targeted region of the pre-mRNA is within a sequence between a pair of genomic sites GRCh38/hg38: chr14 35308914, and GRCh38/hg38: chr14 35308995. In some cases, the therapeutic agent is an antisense oligomer complementary to a sequence with at least about 80%, 85%, 90%, 95%, 96%, 97%, 98%, 99% or 100% sequence identity to SEQ ID NO:278. In some cases, the therapeutic agent is an antisense oligomer complementary to a sequence with 100% sequence identity to SEQ ID NO:278. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5866-5920. In some cases, the therapeutic agent is an antisense oligomer that has a sequence selected from the group consisting of SEQ ID NOs: 5866-5920. In some cases, the target peptide sequence is a portion of PSMA6 protein, and the method treats a disease or the condition that comprises Myocardial infarcation, susceptibility to.

PSMC3IP

In some cases, the target peptide sequence is a portion of PSMC3IP protein. In some cases, the therapeutic agent increases level of a first processed mRNA encoding a protein having a sequence SEQ ID NO: 8245, and decreases level of a second processed mRNA encoding a protein having a sequence SEQ ID NO: 481. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 279. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 279. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 42573623. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 42573623. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 279. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 279. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 42573478. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 42573478. In some cases, the targeted region of the pre-mRNA is within a sequence SEQ ID NO: 279. In some cases, the targeted region of the pre-mRNA is within a sequence between a pair of genomic sites GRCh38/hg38: chr17 42573623, and GRCh38/hg38: chr17 42573478. In some cases, the therapeutic agent is an antisense oligomer complementary to a sequence with at least about 80%, 85%, 90%, 95%, 96%, 97%, 98%, 99% or 100% sequence identity to SEQ ID NO:279. In some cases, the therapeutic agent is an antisense oligomer complementary to a sequence with 100% sequence identity to SEQ ID NO:279. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5921-5988. In some cases, the therapeutic agent is an antisense oligomer that has a sequence selected from the group consisting of SEQ ID NOs: 5921-5988. In some cases, the target peptide sequence is a portion of PSMC3IP protein, and the method treats a disease or the condition that comprises Pure Gonadal Dysgenesis, 46, XX; or Ovarian dysgenesis 3.

PTPN1

In some cases, the target peptide sequence is a portion of PTPN1 protein. In some cases, the therapeutic agent increases level of a first processed mRNA encoding a protein having a sequence SEQ ID NO: 8246, and decreases level of a second processed mRNA encoding a protein having a sequence SEQ ID NO: 482. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 280. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 280. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr20 50564969. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr20 50564969. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 280. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 280. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr20 50565069. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr20 50565069. In some cases, the targeted region of the pre-mRNA is within a sequence SEQ ID NO: 280. In some cases, the targeted region of the pre-mRNA is within a sequence between a pair of genomic sites GRCh38/hg38: chr20 50564969, and GRCh38/hg38: chr20 50565069. In some cases, the therapeutic agent is an antisense oligomer complementary to a sequence with at least about 80%, 85%, 90%, 95%, 96%, 97%, 98%, 99% or 100% sequence identity to SEQ ID NO:280. In some cases, the therapeutic agent is an antisense oligomer complementary to a sequence with 100% sequence identity to SEQ ID NO:280. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 5989-6047. In some cases, the therapeutic agent is an antisense oligomer that has a sequence selected from the group consisting of SEQ ID NOs: 5989-6047. In some cases, the target peptide sequence is a portion of PTPN1 protein, and the method treats a disease or the condition that comprises Schizophrenia; or Insulin resistance, susceptibility to. In some cases, the method treats a cancer that is associated with PTPN1 protein.

RAB11B

In some cases, the target peptide sequence is a portion of RAB11B protein. In some cases, the therapeutic agent increases level of a first processed mRNA encoding a protein having a sequence SEQ ID NO: 8247, and decreases level of a second processed mRNA encoding a protein having a sequence SEQ ID NO: 483. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 281. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 281. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 8402086. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 8402086. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 281. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 281. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 8402279. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 8402279. In some cases, the targeted region of the pre-mRNA is within a sequence SEQ ID NO: 281. In some cases, the targeted region of the pre-mRNA is within a sequence between a pair of genomic sites GRCh38/hg38: chr19 8402086, and GRCh38/hg38: chr19 8402279. In some cases, the therapeutic agent is an antisense oligomer complementary to a sequence with at least about 80%, 85%, 90%, 95%, 96%, 97%, 98%, 99% or 100% sequence identity to SEQ ID NO:281. In some cases, the therapeutic agent is an antisense oligomer complementary to a sequence with 100% sequence identity to SEQ ID NO:281. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6048-6125. In some cases, the therapeutic agent is an antisense oligomer that has a sequence selected from the group consisting of SEQ ID NOs: 6048-6125. In some cases, the target peptide sequence is a portion of RAB11B protein, and the method treats a disease or the condition that comprises Intellectual Disability; Leukodystrophy; or Epileptic encephalopathy.

RBPJ

In some cases, the target peptide sequence is a portion of RBPJ protein. In some cases, the therapeutic agent increases level of a first processed mRNA encoding a protein having a sequence SEQ ID NO: 8248, and decreases level of a second processed mRNA encoding a protein having a sequence SEQ ID NO: 484. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 282. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 282. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 26320700. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 26320700. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 282. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 282. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 26320815. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 26320815. In some cases, the targeted region of the pre-mRNA is within a sequence SEQ ID NO: 282. In some cases, the targeted region of the pre-mRNA is within a sequence between a pair of genomic sites GRCh38/hg38: chr4 26320700, and GRCh38/hg38: chr4 26320815. In some cases, the therapeutic agent is an antisense oligomer complementary to a sequence with at least about 80%, 85%, 90%, 95%, 96%, 97%, 98%, 99% or 100% sequence identity to SEQ ID NO:282. In some cases, the therapeutic agent is an antisense oligomer complementary to a sequence with 100% sequence identity to SEQ ID NO:282. In some cases, the therapeutic agent increases level of a first processed mRNA encoding a protein having a sequence SEQ ID NO: 8249, and decreases level of a second processed mRNA encoding a protein having a sequence SEQ ID NO: 485. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 283. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 283. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 26320700. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 26320700. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 283. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 283. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 26320815. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 26320815. In some cases, the targeted region of the pre-mRNA is within a sequence SEQ ID NO: 283. In some cases, the targeted region of the pre-mRNA is within a sequence between a pair of genomic sites GRCh38/hg38: chr4 26320700, and GRCh38/hg38: chr4 26320815. In some cases, the therapeutic agent is an antisense oligomer complementary to a sequence with at least about 80%, 85%, 90%, 95%, 96%, 97%, 98%, 99% or 100% sequence identity to SEQ ID NO:283. In some cases, the therapeutic agent is an antisense oligomer complementary to a sequence with 100% sequence identity to SEQ ID NO:283. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6126-6187. In some cases, the therapeutic agent is an antisense oligomer that has a sequence selected from the group consisting of SEQ ID NOs: 6126-6187. In some cases, the target peptide sequence is a portion of RBPJ protein, and the method treats a disease or the condition that comprises Intellectual Disability; ADAMS-OLIVER SYNDROME 3; or Adams Oliver syndrome.

RNPS1

In some cases, the target peptide sequence is a portion of RNPS1 protein. In some cases, the therapeutic agent increases level of a first processed mRNA encoding a protein having a sequence SEQ ID NO: 8250, and decreases level of a second processed mRNA encoding a protein having a sequence SEQ ID NO: 486. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 284. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 284. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 284. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 284. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases, the targeted region of the pre-mRNA is within a sequence SEQ ID NO: 284. In some cases, the targeted region of the pre-mRNA is within a sequence between a pair of genomic sites GRCh38/hg38: chr16 2264760, and GRCh38/hg38: chr16 2264573. In some cases, the therapeutic agent is an antisense oligomer complementary to a sequence with at least about 80%, 85%, 90%, 95%, 96%, 97%, 98%, 99% or 100% sequence identity to SEQ ID NO:284. In some cases, the therapeutic agent is an antisense oligomer complementary to a sequence with 100% sequence identity to SEQ ID NO:284. In some cases, the therapeutic agent increases level of a first processed mRNA encoding a protein having a sequence SEQ ID NO: 8251, and decreases level of a second processed mRNA encoding a protein having a sequence SEQ ID NO: 487. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 285. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 285. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 285. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 285. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases, the targeted region of the pre-mRNA is within a sequence SEQ ID NO: 285. In some cases, the targeted region of the pre-mRNA is within a sequence between a pair of genomic sites GRCh38/hg38: chr16 2264760, and GRCh38/hg38: chr16 2264573. In some cases, the therapeutic agent is an antisense oligomer complementary to a sequence with at least about 80%, 85%, 90%, 95%, 96%, 97%, 98%, 99% or 100% sequence identity to SEQ ID NO:285. In some cases, the therapeutic agent is an antisense oligomer complementary to a sequence with 100% sequence identity to SEQ ID NO:285. In some cases, the therapeutic agent increases level of a first processed mRNA encoding a protein having a sequence SEQ ID NO: 8252, and decreases level of a second processed mRNA encoding a protein having a sequence SEQ ID NO: 488. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 286. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 286. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 286. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 286. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases, the targeted region of the pre-mRNA is within a sequence SEQ ID NO: 286. In some cases, the targeted region of the pre-mRNA is within a sequence between a pair of genomic sites GRCh38/hg38: chr16 2264760, and GRCh38/hg38: chr16 2264573. In some cases, the therapeutic agent is an antisense oligomer complementary to a sequence with at least about 80%, 85%, 90%, 95%, 96%, 97%, 98%, 99% or 100% sequence identity to SEQ ID NO:286. In some cases, the therapeutic agent is an antisense oligomer complementary to a sequence with 100% sequence identity to SEQ ID NO:286. In some cases, the therapeutic agent increases level of a first processed mRNA encoding a protein having a sequence SEQ ID NO: 8253, and decreases level of a second processed mRNA encoding a protein having a sequence SEQ ID NO: 489. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 287. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 287. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 2264760. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 287. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 287. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 2264573. In some cases, the targeted region of the pre-mRNA is within a sequence SEQ ID NO: 287. In some cases, the targeted region of the pre-mRNA is within a sequence between a pair of genomic sites GRCh38/hg38: chr16 2264760, and GRCh38/hg38: chr16 2264573. In some cases, the therapeutic agent is an antisense oligomer complementary to a sequence with at least about 80%, 85%, 90%, 95%, 96%, 97%, 98%, 99% or 100% sequence identity to SEQ ID NO:287. In some cases, the therapeutic agent is an antisense oligomer complementary to a sequence with 100% sequence identity to SEQ ID NO:287. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6188-6264. In some cases, the therapeutic agent is an antisense oligomer that has a sequence selected from the group consisting of SEQ ID NOs: 6188-6264. In some cases, the target peptide sequence is a portion of RNPS1 protein, and the method treats a disease or the condition that comprises Mood Disorders.

RPL5

In some cases, the target peptide sequence is a portion of RPL5 protein. In some cases, the therapeutic agent increases level of a first processed mRNA encoding a protein having a sequence SEQ ID NO: 8254, and decreases level of a second processed mRNA encoding a protein having a sequence SEQ ID NO: 490. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 288. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 288. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 92833545. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 92833545. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 288. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 288. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 92833660. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 92833660. In some cases, the targeted region of the pre-mRNA is within a sequence SEQ ID NO: 288. In some cases, the targeted region of the pre-mRNA is within a sequence between a pair of genomic sites GRCh38/hg38: chr1 92833545, and GRCh38/hg38: chr1 92833660. In some cases, the therapeutic agent is an antisense oligomer complementary to a sequence with at least about 80%, 85%, 90%, 95%, 96%, 97%, 98%, 99% or 100% sequence identity to SEQ ID NO:288. In some cases, the therapeutic agent is an antisense oligomer complementary to a sequence with 100% sequence identity to SEQ ID NO:288. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6265-6326. In some cases, the therapeutic agent is an antisense oligomer that has a sequence selected from the group consisting of SEQ ID NOs: 6265-6326. In some cases, the target peptide sequence is a portion of RPL5 protein, and the method treats a disease or the condition that comprises Congenital anemia; Hematologic Neoplasms; Anemia, Diamond-Blackfan; Cytopenia; Aase Smith syndrome 2; Radial club hand; Precursor T-Cell Lymphoblastic Leukemia-Lymphoma; or Diamond-Blackfan anemia 6. In some cases, the method treats a cancer that is associated with RPL5 protein.

RPS20

In some cases, the target peptide sequence is a portion of RPS20 protein. In some cases, the therapeutic agent increases level of a first processed mRNA encoding a protein having a sequence SEQ ID NO: 8255, and decreases level of a second processed mRNA encoding a protein having a sequence SEQ ID NO: 491. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 289. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 289. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 56073768. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr8 56073768. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 289. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 289. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 56073695. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr8 56073695. In some cases, the targeted region of the pre-mRNA is within a sequence SEQ ID NO: 289. In some cases, the targeted region of the pre-mRNA is within a sequence between a pair of genomic sites GRCh38/hg38: chr8 56073768, and GRCh38/hg38: chr8 56073695. In some cases, the therapeutic agent is an antisense oligomer complementary to a sequence with at least about 80%, 85%, 90%, 95%, 96%, 97%, 98%, 99% or 100% sequence identity to SEQ ID NO:289. In some cases, the therapeutic agent is an antisense oligomer complementary to a sequence with 100% sequence identity to SEQ ID NO:289. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6327-6380. In some cases, the therapeutic agent is an antisense oligomer that has a sequence selected from the group consisting of SEQ ID NOs: 6327-6380. In some cases, the target peptide sequence is a portion of RPS20 protein, and the method treats a disease or the condition that comprises Hereditary non-polyposis colorectal cancer syndrome; Familial Colorectal Cancer Type X; Hereditary Nonpolyposis Colorectal Cancer; or Hereditary Nonpolyposis Colorectal Neoplasms.

SECISBP2

In some cases, the target peptide sequence is a portion of SECISBP2 protein. In some cases, the therapeutic agent increases level of a first processed mRNA encoding a protein having a sequence SEQ ID NO: 8256, and decreases level of a second processed mRNA encoding a protein having a sequence SEQ ID NO: 492. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 290. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 290. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr9 89325427. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr9 89325427. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 290. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 290. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr9 89325676. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr9 89325676. In some cases, the targeted region of the pre-mRNA is within a sequence SEQ ID NO: 290. In some cases, the targeted region of the pre-mRNA is within a sequence between a pair of genomic sites GRCh38/hg38: chr9 89325427, and GRCh38/hg38: chr9 89325676. In some cases, the therapeutic agent is an antisense oligomer complementary to a sequence with at least about 80%, 85%, 90%, 95%, 96%, 97%, 98%, 99% or 100% sequence identity to SEQ ID NO:290. In some cases, the therapeutic agent is an antisense oligomer complementary to a sequence with 100% sequence identity to SEQ ID NO:290. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6381-6469. In some cases, the therapeutic agent is an antisense oligomer that has a sequence selected from the group consisting of SEQ ID NOs: 6381-6469. In some cases, the target peptide sequence is a portion of SECISBP2 protein, and the method treats a disease or the condition that comprises Thyroid Hormone Metabolism, Abnormal; or Thyroid Hormone Resistance Syndrome.

SELENBP1

In some cases, the target peptide sequence is a portion of SELENBP1 protein. In some cases, the therapeutic agent increases level of a first processed mRNA encoding a protein having a sequence SEQ ID NO: 8257, and decreases level of a second processed mRNA encoding a protein having a sequence SEQ ID NO: 493. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 291. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 291. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 151369978. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 151369978. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 291. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 291. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 151369713. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 151369713. In some cases, the targeted region of the pre-mRNA is within a sequence SEQ ID NO: 291. In some cases, the targeted region of the pre-mRNA is within a sequence between a pair of genomic sites GRCh38/hg38: chr1 151369978, and GRCh38/hg38: chr1 151369713. In some cases, the therapeutic agent is an antisense oligomer complementary to a sequence with at least about 80%, 85%, 90%, 95%, 96%, 97%, 98%, 99% or 100% sequence identity to SEQ ID NO:291. In some cases, the therapeutic agent is an antisense oligomer complementary to a sequence with 100% sequence identity to SEQ ID NO:291. In some cases, the therapeutic agent increases level of a first processed mRNA encoding a protein having a sequence SEQ ID NO: 8258, and decreases level of a second processed mRNA encoding a protein having a sequence SEQ ID NO: 494. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 292. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 292. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 151369978. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr1 151369978. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 292. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 292. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 151369713. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr1 151369713. In some cases, the targeted region of the pre-mRNA is within a sequence SEQ ID NO: 292. In some cases, the targeted region of the pre-mRNA is within a sequence between a pair of genomic sites GRCh38/hg38: chr1 151369978, and GRCh38/hg38: chr1 151369713. In some cases, the therapeutic agent is an antisense oligomer complementary to a sequence with at least about 80%, 85%, 90%, 95%, 96%, 97%, 98%, 99% or 100% sequence identity to SEQ ID NO:292. In some cases, the therapeutic agent is an antisense oligomer complementary to a sequence with 100% sequence identity to SEQ ID NO:292. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6470-6561. In some cases, the therapeutic agent is an antisense oligomer that has a sequence selected from the group consisting of SEQ ID NOs: 6470-6561. In some cases, the target peptide sequence is a portion of SELENBP1 protein, and the method treats a cancer that is associated with SELENBP1 protein.

SEPT11

In some cases, the target peptide sequence is a portion of SEPT11 protein. In some cases, the therapeutic agent increases level of a first processed mRNA encoding a protein having a sequence SEQ ID NO: 8259, and decreases level of a second processed mRNA encoding a protein having a sequence SEQ ID NO: 495. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 293. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 293. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 76995781. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr4 76995781. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 293. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 293. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 76995938. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr4 76995938. In some cases, the targeted region of the pre-mRNA is within a sequence SEQ ID NO: 293. In some cases, the targeted region of the pre-mRNA is within a sequence between a pair of genomic sites GRCh38/hg38: chr4 76995781, and GRCh38/hg38: chr4 76995938. In some cases, the therapeutic agent is an antisense oligomer complementary to a sequence with at least about 80%, 85%, 90%, 95%, 96%, 97%, 98%, 99% or 100% sequence identity to SEQ ID NO:293. In some cases, the therapeutic agent is an antisense oligomer complementary to a sequence with 100% sequence identity to SEQ ID NO:293. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6562-6632. In some cases, the therapeutic agent is an antisense oligomer that has a sequence selected from the group consisting of SEQ ID NOs: 6562-6632. In some cases, the target peptide sequence is a portion of SEPT11 protein, and the method treats a disease or the condition that comprises Bipolar Disorder; or Schizophrenia.

SIRT2

In some cases, the target peptide sequence is a portion of SIRT2 protein. In some cases, the therapeutic agent increases level of a first processed mRNA encoding a protein having a sequence SEQ ID NO: 8260, and decreases level of a second processed mRNA encoding a protein having a sequence SEQ ID NO: 496. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 294. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 294. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 294. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 294. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases, the targeted region of the pre-mRNA is within a sequence SEQ ID NO: 294. In some cases, the targeted region of the pre-mRNA is within a sequence between a pair of genomic sites GRCh38/hg38: chr19 38893867, and GRCh38/hg38: chr19 38893819. In some cases, the therapeutic agent is an antisense oligomer complementary to a sequence with at least about 80%, 85%, 90%, 95%, 96%, 97%, 98%, 99% or 100% sequence identity to SEQ ID NO:294. In some cases, the therapeutic agent is an antisense oligomer complementary to a sequence with 100% sequence identity to SEQ ID NO:294. In some cases, the therapeutic agent increases level of a first processed mRNA encoding a protein having a sequence SEQ ID NO: 8261, and decreases level of a second processed mRNA encoding a protein having a sequence SEQ ID NO: 497. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 295. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 295. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 295. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 295. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases, the targeted region of the pre-mRNA is within a sequence SEQ ID NO: 295. In some cases, the targeted region of the pre-mRNA is within a sequence between a pair of genomic sites GRCh38/hg38: chr19 38893867, and GRCh38/hg38: chr19 38893819. In some cases, the therapeutic agent is an antisense oligomer complementary to a sequence with at least about 80%, 85%, 90%, 95%, 96%, 97%, 98%, 99% or 100% sequence identity to SEQ ID NO:295. In some cases, the therapeutic agent is an antisense oligomer complementary to a sequence with 100% sequence identity to SEQ ID NO:295. In some cases, the therapeutic agent increases level of a first processed mRNA encoding a protein having a sequence SEQ ID NO: 8262, and decreases level of a second processed mRNA encoding a protein having a sequence SEQ ID NO: 498. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 296. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 296. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 296. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 296. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases, the targeted region of the pre-mRNA is within a sequence SEQ ID NO: 296. In some cases, the targeted region of the pre-mRNA is within a sequence between a pair of genomic sites GRCh38/hg38: chr19 38893867, and GRCh38/hg38: chr19 38893819. In some cases, the therapeutic agent is an antisense oligomer complementary to a sequence with at least about 80%, 85%, 90%, 95%, 96%, 97%, 98%, 99% or 100% sequence identity to SEQ ID NO:296. In some cases, the therapeutic agent is an antisense oligomer complementary to a sequence with 100% sequence identity to SEQ ID NO:296. In some cases, the therapeutic agent increases level of a first processed mRNA encoding a protein having a sequence SEQ ID NO: 8263, and decreases level of a second processed mRNA encoding a protein having a sequence SEQ ID NO: 499. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 297. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 297. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 297. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 297. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases, the targeted region of the pre-mRNA is within a sequence SEQ ID NO: 297. In some cases, the targeted region of the pre-mRNA is within a sequence between a pair of genomic sites GRCh38/hg38: chr19 38893867, and GRCh38/hg38: chr19 38893819. In some cases, the therapeutic agent is an antisense oligomer complementary to a sequence with at least about 80%, 85%, 90%, 95%, 96%, 97%, 98%, 99% or 100% sequence identity to SEQ ID NO:297. In some cases, the therapeutic agent is an antisense oligomer complementary to a sequence with 100% sequence identity to SEQ ID NO:297. In some cases, the therapeutic agent increases level of a first processed mRNA encoding a protein having a sequence SEQ ID NO: 8264, and decreases level of a second processed mRNA encoding a protein having a sequence SEQ ID NO: 500. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 298. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 298. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr19 38893867. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 298. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 298. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr19 38893819. In some cases, the targeted region of the pre-mRNA is within a sequence SEQ ID NO: 298. In some cases, the targeted region of the pre-mRNA is within a sequence between a pair of genomic sites GRCh38/hg38: chr19 38893867, and GRCh38/hg38: chr19 38893819. In some cases, the therapeutic agent is an antisense oligomer complementary to a sequence with at least about 80%, 85%, 90%, 95%, 96%, 97%, 98%, 99% or 100% sequence identity to SEQ ID NO:298. In some cases, the therapeutic agent is an antisense oligomer complementary to a sequence with 100% sequence identity to SEQ ID NO:298. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6633-6681. In some cases, the therapeutic agent is an antisense oligomer that has a sequence selected from the group consisting of SEQ ID NOs: 6633-6681. In some cases, the target peptide sequence is a portion of SIRT2 protein, and the method treats a disease or the condition that comprises Disturbance in mood; or Mood Disorders. In some cases, the method treats a cancer that is associated with SIRT2 protein.

SLC25A26

In some cases, the target peptide sequence is a portion of SLC25A26 protein. In some cases, the therapeutic agent increases level of a first processed mRNA encoding a protein having a sequence SEQ ID NO: 8265, and decreases level of a second processed mRNA encoding a protein having a sequence SEQ ID NO: 501. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 299. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 299. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 66243203. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 66243203. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 299. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 299. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 66243312. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 66243312. In some cases, the targeted region of the pre-mRNA is within a sequence SEQ ID NO: 299. In some cases, the targeted region of the pre-mRNA is within a sequence between a pair of genomic sites GRCh38/hg38: chr3 66243203, and GRCh38/hg38: chr3 66243312. In some cases, the therapeutic agent is an antisense oligomer complementary to a sequence with at least about 80%, 85%, 90%, 95%, 96%, 97%, 98%, 99% or 100% sequence identity to SEQ ID NO:299. In some cases, the therapeutic agent is an antisense oligomer complementary to a sequence with 100% sequence identity to SEQ ID NO:299. In some cases, the therapeutic agent increases level of a first processed mRNA encoding a protein having a sequence SEQ ID NO: 8266, and decreases level of a second processed mRNA encoding a protein having a sequence SEQ ID NO: 502. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 300. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 300. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 66243203. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 66243203. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 300. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 300. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 66243312. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 66243312. In some cases, the targeted region of the pre-mRNA is within a sequence SEQ ID NO: 300. In some cases, the targeted region of the pre-mRNA is within a sequence between a pair of genomic sites GRCh38/hg38: chr3 66243203, and GRCh38/hg38: chr3 66243312. In some cases, the therapeutic agent is an antisense oligomer complementary to a sequence with at least about 80%, 85%, 90%, 95%, 96%, 97%, 98%, 99% or 100% sequence identity to SEQ ID NO:300. In some cases, the therapeutic agent is an antisense oligomer complementary to a sequence with 100% sequence identity to SEQ ID NO:300. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6682-6742. In some cases, the therapeutic agent is an antisense oligomer that has a sequence selected from the group consisting of SEQ ID NOs: 6682-6742. In some cases, the target peptide sequence is a portion of SLC25A26 protein, and the method treats a disease or the condition that comprises Intellectual Disability; Combined Oxidative Phosphorylation Deficiency 28; or Mitochondrial Diseases.

SMARCE1

In some cases, the target peptide sequence is a portion of SMARCE1 protein. In some cases, the therapeutic agent increases level of a first processed mRNA encoding a protein having a sequence SEQ ID NO: 8267, and decreases level of a second processed mRNA encoding a protein having a sequence SEQ ID NO: 503. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 301. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 301. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 40644459. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 40644459. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 301. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 301. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 40644385. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 40644385. In some cases, the targeted region of the pre-mRNA is within a sequence SEQ ID NO: 301. In some cases, the targeted region of the pre-mRNA is within a sequence between a pair of genomic sites GRCh38/hg38: chr17 40644459, and GRCh38/hg38: chr17 40644385. In some cases, the therapeutic agent is an antisense oligomer complementary to a sequence with at least about 80%, 85%, 90%, 95%, 96%, 97%, 98%, 99% or 100% sequence identity to SEQ ID NO:301. In some cases, the therapeutic agent is an antisense oligomer complementary to a sequence with 100% sequence identity to SEQ ID NO:301. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6743-6796. In some cases, the therapeutic agent is an antisense oligomer that has a sequence selected from the group consisting of SEQ ID NOs: 6743-6796. In some cases, the target peptide sequence is a portion of SMARCE1 protein, and the method treats a disease or the condition that comprises Intellectual Disability; Coffin-Siris Syndrome 5; Coffin-Siris syndrome; Meningioma; Meningiomas, Multiple; or Meningioma, familial, susceptibility to.

SMC1A

In some cases, the target peptide sequence is a portion of SMC1A protein. In some cases, the therapeutic agent increases level of a first processed mRNA encoding a protein having a sequence SEQ ID NO: 8268, and decreases level of a second processed mRNA encoding a protein having a sequence SEQ ID NO: 504. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 302. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 302. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 53422040. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 53422040. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 302. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 302. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 53421895. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 53421895. In some cases, the targeted region of the pre-mRNA is within a sequence SEQ ID NO: 302. In some cases, the targeted region of the pre-mRNA is within a sequence between a pair of genomic sites GRCh38/hg38: chrX 53422040, and GRCh38/hg38: chrX 53421895. In some cases, the therapeutic agent is an antisense oligomer complementary to a sequence with at least about 80%, 85%, 90%, 95%, 96%, 97%, 98%, 99% or 100% sequence identity to SEQ ID NO:302. In some cases, the therapeutic agent is an antisense oligomer complementary to a sequence with 100% sequence identity to SEQ ID NO:302. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6797-6864. In some cases, the therapeutic agent is an antisense oligomer that has a sequence selected from the group consisting of SEQ ID NOs: 6797-6864. In some cases, the target peptide sequence is a portion of SMC1A protein, and the method treats a disease or the condition that comprises Intellectual Disability; Primary microcephaly; Congenital anemia; Osteogenesis Imperfecta; Cytopenia; Epileptic encephalopathy; Radial club hand; Cornelia De Lange Syndrome; Growth Deficiency and Mental Retardation with Facial Dysmorphism; Congenital muscular hypertrophy-cerebral syndrome; or Cornelia de Lange syndrome 2.

SMG1

In some cases, the target peptide sequence is a portion of SMG1 protein. In some cases, the therapeutic agent increases level of a first processed mRNA encoding a protein having a sequence SEQ ID NO: 8269, and decreases level of a second processed mRNA encoding a protein having a sequence SEQ ID NO: 505. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 303. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 303. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 18900044. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr16 18900044. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 303. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 303. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 18899987. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr16 18899987. In some cases, the targeted region of the pre-mRNA is within a sequence SEQ ID NO: 303. In some cases, the targeted region of the pre-mRNA is within a sequence between a pair of genomic sites GRCh38/hg38: chr16 18900044, and GRCh38/hg38: chr16 18899987. In some cases, the therapeutic agent is an antisense oligomer complementary to a sequence with at least about 80%, 85%, 90%, 95%, 96%, 97%, 98%, 99% or 100% sequence identity to SEQ ID NO:303. In some cases, the therapeutic agent is an antisense oligomer complementary to a sequence with 100% sequence identity to SEQ ID NO:303. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6865-6915. In some cases, the therapeutic agent is an antisense oligomer that has a sequence selected from the group consisting of SEQ ID NOs: 6865-6915. In some cases, the target peptide sequence is a portion of SMG1 protein, and the method treats a disease or the condition that comprises Infiltrating duct carcinoma of female breast.

SPACA7

In some cases, the target peptide sequence is a portion of SPACA7 protein. In some cases, the therapeutic agent increases level of a first processed mRNA encoding a protein having a sequence SEQ ID NO: 8270, and decreases level of a second processed mRNA encoding a protein having a sequence SEQ ID NO: 506. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 304. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 304. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr13 112382278. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr13 112382278. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 304. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 304. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr13 112382507. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr13 112382507. In some cases, the targeted region of the pre-mRNA is within a sequence SEQ ID NO: 304. In some cases, the targeted region of the pre-mRNA is within a sequence between a pair of genomic sites GRCh38/hg38: chr13 112382278, and GRCh38/hg38: chr13 112382507. In some cases, the therapeutic agent is an antisense oligomer complementary to a sequence with at least about 80%, 85%, 90%, 95%, 96%, 97%, 98%, 99% or 100% sequence identity to SEQ ID NO:304. In some cases, the therapeutic agent is an antisense oligomer complementary to a sequence with 100% sequence identity to SEQ ID NO:304. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 6916-7000. In some cases, the therapeutic agent is an antisense oligomer that has a sequence selected from the group consisting of SEQ ID NOs: 6916-7000. In some cases, the target peptide sequence is a portion of SPACA7 protein, and the method treats a disease or the condition that comprises Colorectal Cancer.

SPRED1

In some cases, the target peptide sequence is a portion of SPRED1 protein. In some cases, the therapeutic agent increases level of a first processed mRNA encoding a protein having a sequence SEQ ID NO: 8271, and decreases level of a second processed mRNA encoding a protein having a sequence SEQ ID NO: 507. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 305. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 305. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 38299373. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 38299373. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 305. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 305. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 38299547. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 38299547. In some cases, the targeted region of the pre-mRNA is within a sequence SEQ ID NO: 305. In some cases, the targeted region of the pre-mRNA is within a sequence between a pair of genomic sites GRCh38/hg38: chr15 38299373, and GRCh38/hg38: chr15 38299547. In some cases, the therapeutic agent is an antisense oligomer complementary to a sequence with at least about 80%, 85%, 90%, 95%, 96%, 97%, 98%, 99% or 100% sequence identity to SEQ ID NO:305. In some cases, the therapeutic agent is an antisense oligomer complementary to a sequence with 100% sequence identity to SEQ ID NO:305. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7001-7074. In some cases, the therapeutic agent is an antisense oligomer that has a sequence selected from the group consisting of SEQ ID NOs: 7001-7074. In some cases, the target peptide sequence is a portion of SPRED1 protein, and the method treats a disease or the condition that comprises Intellectual Disability; Neurofibromatosis 1; Neurofibromatosis, Type 1-like Syndrome; or Legius syndrome.

SRP54

In some cases, the target peptide sequence is a portion of SRP54 protein. In some cases, the therapeutic agent increases level of a first processed mRNA encoding a protein having a sequence SEQ ID NO: 8272, and decreases level of a second processed mRNA encoding a protein having a sequence SEQ ID NO: 508. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 306. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 306. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 35000936. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr14 35000936. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 306. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 306. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 35001020. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr14 35001020. In some cases, the targeted region of the pre-mRNA is within a sequence SEQ ID NO: 306. In some cases, the targeted region of the pre-mRNA is within a sequence between a pair of genomic sites GRCh38/hg38: chr14 35000936, and GRCh38/hg38: chr14 35001020. In some cases, the therapeutic agent is an antisense oligomer complementary to a sequence with at least about 80%, 85%, 90%, 95%, 96%, 97%, 98%, 99% or 100% sequence identity to SEQ ID NO:306. In some cases, the therapeutic agent is an antisense oligomer complementary to a sequence with 100% sequence identity to SEQ ID NO:306. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7075-7130. In some cases, the therapeutic agent is an antisense oligomer that has a sequence selected from the group consisting of SEQ ID NOs: 7075-7130. In some cases, the target peptide sequence is a portion of SRP54 protein, and the method treats a disease or the condition that comprises Shwachman syndrome.

SRPK2

In some cases, the target peptide sequence is a portion of SRPK2 protein. In some cases, the therapeutic agent increases level of a first processed mRNA encoding a protein having a sequence SEQ ID NO: 8273, and decreases level of a second processed mRNA encoding a protein having a sequence SEQ ID NO: 509. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 307. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 307. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 105268869. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr7 105268869. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 307. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 307. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 105268806. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr7 105268806. In some cases, the targeted region of the pre-mRNA is within a sequence SEQ ID NO: 307. In some cases, the targeted region of the pre-mRNA is within a sequence between a pair of genomic sites GRCh38/hg38: chr7 105268869, and GRCh38/hg38: chr7 105268806. In some cases, the therapeutic agent is an antisense oligomer complementary to a sequence with at least about 80%, 85%, 90%, 95%, 96%, 97%, 98%, 99% or 100% sequence identity to SEQ ID NO:307. In some cases, the therapeutic agent is an antisense oligomer complementary to a sequence with 100% sequence identity to SEQ ID NO:307. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7131-7182. In some cases, the therapeutic agent is an antisense oligomer that has a sequence selected from the group consisting of SEQ ID NOs: 7131-7182. In some cases, the target peptide sequence is a portion of SRPK2 protein, and the method treats a disease or the condition that comprises Glioblastoma Multiforme.

STK36

In some cases, the target peptide sequence is a portion of STK36 protein. In some cases, the therapeutic agent increases level of a first processed mRNA encoding a protein having a sequence SEQ ID NO: 8274, and decreases level of a second processed mRNA encoding a protein having a sequence SEQ ID NO: 510. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 308. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 308. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 218673665. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 218673665. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 308. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 308. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 218673765. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 218673765. In some cases, the targeted region of the pre-mRNA is within a sequence SEQ ID NO: 308. In some cases, the targeted region of the pre-mRNA is within a sequence between a pair of genomic sites GRCh38/hg38: chr2 218673665, and GRCh38/hg38: chr2 218673765. In some cases, the therapeutic agent is an antisense oligomer complementary to a sequence with at least about 80%, 85%, 90%, 95%, 96%, 97%, 98%, 99% or 100% sequence identity to SEQ ID NO:308. In some cases, the therapeutic agent is an antisense oligomer complementary to a sequence with 100% sequence identity to SEQ ID NO:308. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7183-7241. In some cases, the therapeutic agent is an antisense oligomer that has a sequence selected from the group consisting of SEQ ID NOs: 7183-7241. In some cases, the target peptide sequence is a portion of STK36 protein, and the method treats a disease or the condition that comprises Ovarian Serous Adenocarcinoma; Polynesian Bronchiectasis; or Kartagener Syndrome.

STRADA

In some cases, the target peptide sequence is a portion of STRADA protein. In some cases, the therapeutic agent increases level of a first processed mRNA encoding a protein having a sequence SEQ ID NO: 8275, and decreases level of a second processed mRNA encoding a protein having a sequence SEQ ID NO: 511. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 309. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 309. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 309. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 309. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 309. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:309. In some cases, the therapeutic agent is an antisense oligomer complementary to a sequence with 100% sequence identity to SEQ ID NO:309. In some cases, the therapeutic agent increases level of a first processed mRNA encoding a protein having a sequence SEQ ID NO: 8276, and decreases level of a second processed mRNA encoding a protein having a sequence SEQ ID NO: 512. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 310. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 310. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 310. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 310. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 310. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:310. In some cases, the therapeutic agent is an antisense oligomer complementary to a sequence with 100% sequence identity to SEQ ID NO:310. In some cases, the therapeutic agent increases level of a first processed mRNA encoding a protein having a sequence SEQ ID NO: 8277, and decreases level of a second processed mRNA encoding a protein having a sequence SEQ ID NO: 513. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 311. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 311. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 311. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 311. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 311. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:311. In some cases, the therapeutic agent is an antisense oligomer complementary to a sequence with 100% sequence identity to SEQ ID NO:311. In some cases, the therapeutic agent increases level of a first processed mRNA encoding a protein having a sequence SEQ ID NO: 8278, and decreases level of a second processed mRNA encoding a protein having a sequence SEQ ID NO: 514. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 312. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 312. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 312. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 312. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 312. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:312. In some cases, the therapeutic agent is an antisense oligomer complementary to a sequence with 100% sequence identity to SEQ ID NO:312. In some cases, the therapeutic agent increases level of a first processed mRNA encoding a protein having a sequence SEQ ID NO: 8279, and decreases level of a second processed mRNA encoding a protein having a sequence SEQ ID NO: 515. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 313. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 313. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 313. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 313. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 313. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:313. In some cases, the therapeutic agent is an antisense oligomer complementary to a sequence with 100% sequence identity to SEQ ID NO:313. In some cases, the therapeutic agent increases level of a first processed mRNA encoding a protein having a sequence SEQ ID NO: 8280, and decreases level of a second processed mRNA encoding a protein having a sequence SEQ ID NO: 516. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 314. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 314. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 314. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 314. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 314. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:314. In some cases, the therapeutic agent is an antisense oligomer complementary to a sequence with 100% sequence identity to SEQ ID NO:314. In some cases, the therapeutic agent increases level of a first processed mRNA encoding a protein having a sequence SEQ ID NO: 8281, and decreases level of a second processed mRNA encoding a protein having a sequence SEQ ID NO: 517. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 315. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 315. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 315. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 315. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 315. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:315. In some cases, the therapeutic agent is an antisense oligomer complementary to a sequence with 100% sequence identity to SEQ ID NO:315. In some cases, the therapeutic agent increases level of a first processed mRNA encoding a protein having a sequence SEQ ID NO: 8282, and decreases level of a second processed mRNA encoding a protein having a sequence SEQ ID NO: 518. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 316. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 316. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 316. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 316. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 316. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:316. In some cases, the therapeutic agent is an antisense oligomer complementary to a sequence with 100% sequence identity to SEQ ID NO:316. In some cases, the therapeutic agent increases level of a first processed mRNA encoding a protein having a sequence SEQ ID NO: 8283, and decreases level of a second processed mRNA encoding a protein having a sequence SEQ ID NO: 519. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 317. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 317. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 63714108. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 317. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 317. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 63714006. In some cases, the targeted region of the pre-mRNA is within a sequence SEQ ID NO: 317. In some cases, the targeted region of the pre-mRNA is within a sequence between a pair of genomic sites GRCh38/hg38: chr17 63714108, and GRCh38/hg38: chr17 63714006. In some cases, the therapeutic agent is an antisense oligomer complementary to a sequence with at least about 80%, 85%, 90%, 95%, 96%, 97%, 98%, 99% or 100% sequence identity to SEQ ID NO:317. In some cases, the therapeutic agent is an antisense oligomer complementary to a sequence with 100% sequence identity to SEQ ID NO:317. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7242-7301. In some cases, the therapeutic agent is an antisense oligomer that has a sequence selected from the group consisting of SEQ ID NOs: 7242-7301. In some cases, the target peptide sequence is a portion of STRADA protein, and the method treats a disease or the condition that comprises Polyhydramnios, Megalencephaly, and Symptomatic Epilepsy; Hydrocephalus; Intellectual Disability; or Epileptic encephalopathy. In some cases, the method treats a cancer that is associated with STRADA protein.

SUMO1

In some cases, the target peptide sequence is a portion of SUMO1 protein. In some cases, the therapeutic agent increases level of a first processed mRNA encoding a protein having a sequence SEQ ID NO: 8284, and decreases level of a second processed mRNA encoding a protein having a sequence SEQ ID NO: 520. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 318. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 318. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 202214429. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr2 202214429. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 318. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 318. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 202214357. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr2 202214357. In some cases, the targeted region of the pre-mRNA is within a sequence SEQ ID NO: 318. In some cases, the targeted region of the pre-mRNA is within a sequence between a pair of genomic sites GRCh38/hg38: chr2 202214429, and GRCh38/hg38: chr2 202214357. In some cases, the therapeutic agent is an antisense oligomer complementary to a sequence with at least about 80%, 85%, 90%, 95%, 96%, 97%, 98%, 99% or 100% sequence identity to SEQ ID NO:318. In some cases, the therapeutic agent is an antisense oligomer complementary to a sequence with 100% sequence identity to SEQ ID NO:318. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7302-7355. In some cases, the therapeutic agent is an antisense oligomer that has a sequence selected from the group consisting of SEQ ID NOs: 7302-7355. In some cases, the target peptide sequence is a portion of SUMO1 protein, and the method treats a disease or the condition that comprises Oligodontia; Hypodontia; or Orofacial cleft 10.

TBL1XR1

In some cases, the target peptide sequence is a portion of TBL1XR1 protein. In some cases, the therapeutic agent increases level of a first processed mRNA encoding a protein having a sequence SEQ ID NO: 8285, and decreases level of a second processed mRNA encoding a protein having a sequence SEQ ID NO: 521. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 319. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 319. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 319. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 319. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases, the targeted region of the pre-mRNA is within a sequence SEQ ID NO: 319. In some cases, the targeted region of the pre-mRNA is within a sequence between a pair of genomic sites GRCh38/hg38: chr3 177065022, and GRCh38/hg38: chr3 177064920. In some cases, the therapeutic agent is an antisense oligomer complementary to a sequence with at least about 80%, 85%, 90%, 95%, 96%, 97%, 98%, 99% or 100% sequence identity to SEQ ID NO:319. In some cases, the therapeutic agent is an antisense oligomer complementary to a sequence with 100% sequence identity to SEQ ID NO:319. In some cases, the therapeutic agent increases level of a first processed mRNA encoding a protein having a sequence SEQ ID NO: 8286, and decreases level of a second processed mRNA encoding a protein having a sequence SEQ ID NO: 522. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 320. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 320. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 320. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 320. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases, the targeted region of the pre-mRNA is within a sequence SEQ ID NO: 320. In some cases, the targeted region of the pre-mRNA is within a sequence between a pair of genomic sites GRCh38/hg38: chr3 177065022, and GRCh38/hg38: chr3 177064920. In some cases, the therapeutic agent is an antisense oligomer complementary to a sequence with at least about 80%, 85%, 90%, 95%, 96%, 97%, 98%, 99% or 100% sequence identity to SEQ ID NO:320. In some cases, the therapeutic agent is an antisense oligomer complementary to a sequence with 100% sequence identity to SEQ ID NO:320. In some cases, the therapeutic agent increases level of a first processed mRNA encoding a protein having a sequence SEQ ID NO: 8287, and decreases level of a second processed mRNA encoding a protein having a sequence SEQ ID NO: 523. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 321. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 321. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 321. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 321. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases, the targeted region of the pre-mRNA is within a sequence SEQ ID NO: 321. In some cases, the targeted region of the pre-mRNA is within a sequence between a pair of genomic sites GRCh38/hg38: chr3 177065022, and GRCh38/hg38: chr3 177064920. In some cases, the therapeutic agent is an antisense oligomer complementary to a sequence with at least about 80%, 85%, 90%, 95%, 96%, 97%, 98%, 99% or 100% sequence identity to SEQ ID NO:321. In some cases, the therapeutic agent is an antisense oligomer complementary to a sequence with 100% sequence identity to SEQ ID NO:321. In some cases, the therapeutic agent increases level of a first processed mRNA encoding a protein having a sequence SEQ ID NO: 8288, and decreases level of a second processed mRNA encoding a protein having a sequence SEQ ID NO: 524. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 322. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 322. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 177065022. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 322. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 322. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 177064920. In some cases, the targeted region of the pre-mRNA is within a sequence SEQ ID NO: 322. In some cases, the targeted region of the pre-mRNA is within a sequence between a pair of genomic sites GRCh38/hg38: chr3 177065022, and GRCh38/hg38: chr3 177064920. In some cases, the therapeutic agent is an antisense oligomer complementary to a sequence with at least about 80%, 85%, 90%, 95%, 96%, 97%, 98%, 99% or 100% sequence identity to SEQ ID NO:322. In some cases, the therapeutic agent is an antisense oligomer complementary to a sequence with 100% sequence identity to SEQ ID NO:322. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7356-7415. In some cases, the therapeutic agent is an antisense oligomer that has a sequence selected from the group consisting of SEQ ID NOs: 7356-7415. In some cases, the target peptide sequence is a portion of TBL1XR1 protein, and the method treats a disease or the condition that comprises Malignant neoplasm of urinary bladder; Intellectual Disability; Acute Promyelocytic Leukemia; Adenocarcinoma of large intestine; Bladder Neoplasm; Epileptic encephalopathy; Neoplasm of uncertain or unknown behavior of bladder; Mental Retardation, Autosomal Dominant 41; Benign neoplasm of bladder; Lymphoma, Non-Hodgkin; Carcinoma in situ of bladder; Central nervous system lymphoma; Carcinoma of bladder; Plantar Lipomatosis, Unusual Facies, and Developmental Delay; or Pierpont syndrome.

TLR8

In some cases, the target peptide sequence is a portion of TLR8 protein. In some cases, the therapeutic agent increases level of a first processed mRNA encoding a protein having a sequence SEQ ID NO: 8289, and decreases level of a second processed mRNA encoding a protein having a sequence SEQ ID NO: 525. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 323. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 323. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 12910306. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chrX 12910306. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 323. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 323. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 12910442. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chrX 12910442. In some cases, the targeted region of the pre-mRNA is within a sequence SEQ ID NO: 323. In some cases, the targeted region of the pre-mRNA is within a sequence between a pair of genomic sites GRCh38/hg38: chrX 12910306, and GRCh38/hg38: chrX 12910442. In some cases, the therapeutic agent is an antisense oligomer complementary to a sequence with at least about 80%, 85%, 90%, 95%, 96%, 97%, 98%, 99% or 100% sequence identity to SEQ ID NO:323. In some cases, the therapeutic agent is an antisense oligomer complementary to a sequence with 100% sequence identity to SEQ ID NO:323. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7416-7481. In some cases, the therapeutic agent is an antisense oligomer that has a sequence selected from the group consisting of SEQ ID NOs: 7416-7481. In some cases, the target peptide sequence is a portion of TLR8 protein, and the method treats a disease or the condition that comprises Intellectual Disability; or Familial Meniere's disease.

TXNRD2

In some cases, the target peptide sequence is a portion of TXNRD2 protein. In some cases, the therapeutic agent increases level of a first processed mRNA encoding a protein having a sequence SEQ ID NO: 8290, and decreases level of a second processed mRNA encoding a protein having a sequence SEQ ID NO: 526. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 324. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 324. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr22 19880945. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr22 19880945. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 324. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 324. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr22 19880856. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr22 19880856. In some cases, the targeted region of the pre-mRNA is within a sequence SEQ ID NO: 324. In some cases, the targeted region of the pre-mRNA is within a sequence between a pair of genomic sites GRCh38/hg38: chr22 19880945, and GRCh38/hg38: chr22 19880856. In some cases, the therapeutic agent is an antisense oligomer complementary to a sequence with at least about 80%, 85%, 90%, 95%, 96%, 97%, 98%, 99% or 100% sequence identity to SEQ ID NO:324. In some cases, the therapeutic agent is an antisense oligomer complementary to a sequence with 100% sequence identity to SEQ ID NO:324. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7482-7538. In some cases, the therapeutic agent is an antisense oligomer that has a sequence selected from the group consisting of SEQ ID NOs: 7482-7538. In some cases, the target peptide sequence is a portion of TXNRD2 protein, and the method treats a disease or the condition that comprises X-linked Adrenal Hypoplasia; Depressive Symptoms; Familial dilated cardiomyopathy; Familial Glucocorticoid Deficiency Type 1; Conduction disorder of the heart; or Cardiomyopathy, Dilated.

UBE3A

In some cases, the target peptide sequence is a portion of UBE3A protein. In some cases, the therapeutic agent increases level of a first processed mRNA encoding a protein having a sequence SEQ ID NO: 8291, and decreases level of a second processed mRNA encoding a protein having a sequence SEQ ID NO: 527. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 325. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 325. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 25408684. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 25408684. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 325. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 325. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 25408620. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 25408620. In some cases, the targeted region of the pre-mRNA is within a sequence SEQ ID NO: 325. In some cases, the targeted region of the pre-mRNA is within a sequence between a pair of genomic sites GRCh38/hg38: chr15 25408684, and GRCh38/hg38: chr15 25408620. In some cases, the therapeutic agent is an antisense oligomer complementary to a sequence with at least about 80%, 85%, 90%, 95%, 96%, 97%, 98%, 99% or 100% sequence identity to SEQ ID NO:325. In some cases, the therapeutic agent is an antisense oligomer complementary to a sequence with 100% sequence identity to SEQ ID NO:325. In some cases, the therapeutic agent increases level of a first processed mRNA encoding a protein having a sequence SEQ ID NO: 8292, and decreases level of a second processed mRNA encoding a protein having a sequence SEQ ID NO: 528. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 326. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 326. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 25408684. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr15 25408684. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 326. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 326. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 25408620. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr15 25408620. In some cases, the targeted region of the pre-mRNA is within a sequence SEQ ID NO: 326. In some cases, the targeted region of the pre-mRNA is within a sequence between a pair of genomic sites GRCh38/hg38: chr15 25408684, and GRCh38/hg38: chr15 25408620. In some cases, the therapeutic agent is an antisense oligomer complementary to a sequence with at least about 80%, 85%, 90%, 95%, 96%, 97%, 98%, 99% or 100% sequence identity to SEQ ID NO:326. In some cases, the therapeutic agent is an antisense oligomer complementary to a sequence with 100% sequence identity to SEQ ID NO:326. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7539-7590. In some cases, the therapeutic agent is an antisense oligomer that has a sequence selected from the group consisting of SEQ ID NOs: 7539-7590. In some cases, the target peptide sequence is a portion of UBE3A protein, and the method treats a disease or the condition that comprises Intellectual Disability; Angelman Syndrome; Epileptic encephalopathy; Microcephaly; or Duplication 15q11-q13 Syndrome.

UFM1

In some cases, the target peptide sequence is a portion of UFM1 protein. In some cases, the therapeutic agent increases level of a first processed mRNA encoding a protein having a sequence SEQ ID NO: 8293, and decreases level of a second processed mRNA encoding a protein having a sequence SEQ ID NO: 529. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 327. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 327. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr13 38349999. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr13 38349999. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 327. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 327. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr13 38350227. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr13 38350227. In some cases, the targeted region of the pre-mRNA is within a sequence SEQ ID NO: 327. In some cases, the targeted region of the pre-mRNA is within a sequence between a pair of genomic sites GRCh38/hg38: chr13 38349999, and GRCh38/hg38: chr13 38350227. In some cases, the therapeutic agent is an antisense oligomer complementary to a sequence with at least about 80%, 85%, 90%, 95%, 96%, 97%, 98%, 99% or 100% sequence identity to SEQ ID NO:327. In some cases, the therapeutic agent is an antisense oligomer complementary to a sequence with 100% sequence identity to SEQ ID NO:327. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7591-7675. In some cases, the therapeutic agent is an antisense oligomer that has a sequence selected from the group consisting of SEQ ID NOs: 7591-7675. In some cases, the target peptide sequence is a portion of UFM1 protein, and the method treats a disease or the condition that comprises Leukodystrophy, Hypomyelinating, 6.

WAC

In some cases, the target peptide sequence is a portion of WAC protein. In some cases, the therapeutic agent increases level of a first processed mRNA encoding a protein having a sequence SEQ ID NO: 8294, and decreases level of a second processed mRNA encoding a protein having a sequence SEQ ID NO: 530. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 328. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 328. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 28535562. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 28535562. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 328. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 328. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 28535757. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 28535757. In some cases, the targeted region of the pre-mRNA is within a sequence SEQ ID NO: 328. In some cases, the targeted region of the pre-mRNA is within a sequence between a pair of genomic sites GRCh38/hg38: chr10 28535562, and GRCh38/hg38: chr10 28535757. In some cases, the therapeutic agent is an antisense oligomer complementary to a sequence with at least about 80%, 85%, 90%, 95%, 96%, 97%, 98%, 99% or 100% sequence identity to SEQ ID NO:328. In some cases, the therapeutic agent is an antisense oligomer complementary to a sequence with 100% sequence identity to SEQ ID NO:328. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7676-7753. In some cases, the therapeutic agent is an antisense oligomer that has a sequence selected from the group consisting of SEQ ID NOs: 7676-7753. In some cases, the target peptide sequence is a portion of WAC protein, and the method treats a disease or the condition that comprises Intellectual Disability; Chromosome 10p12-p11 Deletion Syndrome; Colorectal Cancer; or Desanto-Shinawi Syndrome.

WDR81

In some cases, the target peptide sequence is a portion of WDR81 protein. In some cases, the therapeutic agent increases level of a first processed mRNA encoding a protein having a sequence SEQ ID NO: 8295, and decreases level of a second processed mRNA encoding a protein having a sequence SEQ ID NO: 531. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 329. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 329. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 1727990. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr17 1727990. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 329. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 329. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 1728626. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr17 1728626. In some cases, the targeted region of the pre-mRNA is within a sequence SEQ ID NO: 329. In some cases, the targeted region of the pre-mRNA is within a sequence between a pair of genomic sites GRCh38/hg38: chr17 1727990, and GRCh38/hg38: chr17 1728626. In some cases, the therapeutic agent is an antisense oligomer complementary to a sequence with at least about 80%, 85%, 90%, 95%, 96%, 97%, 98%, 99% or 100% sequence identity to SEQ ID NO:329. In some cases, the therapeutic agent is an antisense oligomer complementary to a sequence with 100% sequence identity to SEQ ID NO:329. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7754-7919. In some cases, the therapeutic agent is an antisense oligomer that has a sequence selected from the group consisting of SEQ ID NOs: 7754-7919. In some cases, the target peptide sequence is a portion of WDR81 protein, and the method treats a disease or the condition that comprises Intellectual Disability; Ataxias, Hereditary; Cerebellar Ataxia, Mental Retardation, And Dysequilibrium Syndrome 2; Hydrocephalus; Cerebellar Hypoplasia; or Dysequilibrium syndrome.

YME1L1

In some cases, the target peptide sequence is a portion of YME1L1 protein. In some cases, the therapeutic agent increases level of a first processed mRNA encoding a protein having a sequence SEQ ID NO: 8296, and decreases level of a second processed mRNA encoding a protein having a sequence SEQ ID NO: 532. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 330. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 330. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 27153281. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr10 27153281. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 330. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 330. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 27153227. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr10 27153227. In some cases, the targeted region of the pre-mRNA is within a sequence SEQ ID NO: 330. In some cases, the targeted region of the pre-mRNA is within a sequence between a pair of genomic sites GRCh38/hg38: chr10 27153281, and GRCh38/hg38: chr10 27153227. In some cases, the therapeutic agent is an antisense oligomer complementary to a sequence with at least about 80%, 85%, 90%, 95%, 96%, 97%, 98%, 99% or 100% sequence identity to SEQ ID NO:330. In some cases, the therapeutic agent is an antisense oligomer complementary to a sequence with 100% sequence identity to SEQ ID NO:330. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7920-7969. In some cases, the therapeutic agent is an antisense oligomer that has a sequence selected from the group consisting of SEQ ID NOs: 7920-7969. In some cases, the target peptide sequence is a portion of YME1L1 protein, and the method treats a disease or the condition that comprises Optic Atrophy 11.

ZMYND10

In some cases, the target peptide sequence is a portion of ZMYND10 protein. In some cases, the therapeutic agent increases level of a first processed mRNA encoding a protein having a sequence SEQ ID NO: 8297, and decreases level of a second processed mRNA encoding a protein having a sequence SEQ ID NO: 533. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 331. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of SEQ ID NO: 331. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 50345232. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 50345232. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 331. In some cases, the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of SEQ ID NO: 331. In some cases, the targeted region of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 50345124. In some cases. the targeted region of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 50345124. In some cases, the targeted region of the pre-mRNA is within a sequence SEQ ID NO: 331. In some cases, the targeted region of the pre-mRNA is within a sequence between a pair of genomic sites GRCh38/hg38: chr3 50345232, and GRCh38/hg38: chr3 50345124. In some cases, the therapeutic agent is an antisense oligomer complementary to a sequence with at least about 80%, 85%, 90%, 95%, 96%, 97%, 98%, 99% or 100% sequence identity to SEQ ID NO:331. In some cases, the therapeutic agent is an antisense oligomer complementary to a sequence with 100% sequence identity to SEQ ID NO:331. In some cases, the therapeutic agent is an antisense oligomer that has a sequence with at least about 80%, 85%, 90%, 95%, 96%, 97%, 98%, 99% or 100% sequence identity to a sequence selected from the group consisting of SEQ ID NOs: 7970-8030. In some cases, the therapeutic agent is an antisense oligomer that has a sequence selected from the group consisting of SEQ ID NOs: 7970-8030. In some cases, the target peptide sequence is a portion of ZMYND10 protein, and the method treats a disease or the condition that comprises Polynesian Bronchiectasis; Ciliary Dyskinesia, Primary, 22; Kartagener Syndrome; or Ciliopathies. In some cases, the method treats a cancer that is associated with ZMYND10 protein.

### Therapeutic Agents

In various embodiments of the present disclosure, compositions and methods comprising a therapeutic agent are provided to modulate protein expression level. In some embodiments, provided herein are compositions and methods to modulate alternative splicing of a pre-mRNA that encodes a target peptide sequence. In some embodiments, provided herein are compositions and methods to induce exon skipping in the splicing of the pre-mRNA that encodes the target peptide sequence. In other embodiments, therapeutic agents may be used to induce the inclusion of an exon in order to decrease the protein expression level.

In some cases, a therapeutic agent comprises a polynucleic acid polymer. In some cases, a therapeutic agent comprises a viral vector expressing a polynucleic acid polymer that binds to the targeted region of a pre-mRNA the encodes the target peptide sequence. In some cases, the viral vector comprises an adenoviral vector, adeno-associated viral (AAV) vector, lentiviral vector, Herpes Simplex Virus (HSV) viral vector, retroviral vector, or any applicable viral vector. In some cases, a therapeutic agent comprises a gene editing tool that is configured to modify a gene encoding the target peptide sequence such that a gene region that encodes the inefficient translation region is deleted. In some cases, a gene editing tool comprises vector, e.g., viral vector, for gene editing based on CRISPR-Cas9, TALEN, Zinc Finger, or other applicable technologies.

According to one aspect of the present disclosure, provided herein is a method of treating a disease or a condition in a subject in need thereof by modulating expression of a target peptide sequence in a cell of the subject, the cell having a pre-mRNA that encodes the target peptide sequence and comprises an inefficient translation region, the method comprising: contacting the cell of the subject with a therapeutic agent that modulates splicing of the inefficient translation region from the pre-mRNA encoding the target peptide sequence, wherein the therapeutic agent binds to a targeted region of the pre-mRNA, whereby splicing of the inefficient translation region from the pre-mRNA is modulated, thereby modulating a level of a first processed mRNA that is devoid of the inefficient translation region and encodes the target peptide sequence, and thereby modulating the expression of the target peptide sequence in the cell of the subject, wherein the first processed mRNA has a higher translation efficiency for producing the target peptide sequence in the cells as compared to a second processed mRNA that comprises the inefficient translation region. In some cases, the second processed mRNA is otherwise identical to the first processed mRNA but comprises the inefficient translation region.

In some other aspect, provided herein is a method of treating a disease or a condition in a subject in need thereof by modulating expression of a target peptide sequence in a cell of the subject, the cell having a pre-mRNA that encodes the target peptide sequence and comprises a first start codon, a second start codon, and a premature termination codon (PTC) located downstream of the first start codon and upstream of the second start codon, the method comprising: contacting the cell of the subject with a therapeutic agent that modulates splicing of the PTC and the second start codon from the pre-mRNA encoding the target peptide sequence, wherein the therapeutic agent binds to a targeted region of the pre-mRNA, whereby splicing of the PTC and the second start codon from the pre-mRNA is modulated, thereby modulating a level of first processed mRNA that is devoid of the PTC and the second start codon and encodes the target peptide sequence, and thereby modulating the expression of the target peptide sequence in the cell of the subject.

Where reference is made to reducing inclusion of the inefficient translation region or the PTC followed by the alternative region in the mature mRNA, the reduction may be complete, e.g., 100%, or may be partial. The reduction may be clinically significant. The reduction/correction may be relative to the level of inclusion of the inefficient translation region or the PTC followed by the alternative region in the subject without treatment, or relative to the amount of inclusion of the inefficient translation region or the PTC followed by the alternative region in a population of similar subjects. The reduction/correction may be at least 10% less inclusion relative to the average subject, or the subject prior to treatment. The reduction may be at least 20% less inclusion relative to an average subject, or the subject prior to treatment. The reduction may be at least 40% less inclusion relative to an average subject, or the subject prior to treatment. The reduction may be at least 50% less inclusion relative to an average subject, or the subject prior to treatment. The reduction may be at least 60% less inclusion relative to an average subject, or the subject prior to treatment. The reduction may be at least 80% less inclusion relative to an average subject, or the subject prior to treatment. The reduction may be at least 90% less inclusion relative to an average subject, or the subject prior to treatment.

Where reference is made to increasing MeCP2-e1 protein levels, the increase may be clinically significant. The increase may be relative to the level of MeCP2-e1 protein in the subject without treatment, or relative to the amount of active MeCP2-e1 protein in a population of similar subjects. The increase may be at least 10% more active MeCP2-e1 protein relative to the average subject, or the subject prior to treatment. The increase may be at least 20% more active MeCP2-e1 protein relative to the average subject, or the subject prior to treatment. The increase may be at least 40% more active MeCP2-e1 protein relative to the average subject, or the subject prior to treatment. The increase may be at least 50% more active MeCP2-e1 protein relative to the average subject, or the subject prior to treatment. The increase may be at least 80% more active MeCP2-e1 protein relative to the average subject, or the subject prior to treatment. The increase may be at least 100% more active MeCP2-e1 protein relative to the average subject, or the subject prior to treatment. The increase may be at least 200% more active MeCP2-e1 protein relative to the average subject, or the subject prior to treatment. The increase may be at least 500% more active MeCP2-e1 protein relative to the average subject, or the subject prior to treatment.

In embodiments wherein the agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer comprising a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 35-129, or 534-8095. The polynucleic acid polymer may comprise a sequence with 100% sequence identity to a sequence selected from the group consisting of SEQ ID NOs: 35-129, or 534-8095. Table 2 below lists exemplary sequences that the polynucleic acid polymer can comprise.

TABLE 2
Exemplary sequences of polynucleic
acid polymers for translation modulation

| | SEQ ID NOs of |
|---|---|
| | Exemplary Sequences of |
| Target Gene | Polynucleic Acid Polymers |
| ACIN1 | 534-584 |
| ACTN1 | 585-646 |
| ACTN2 | 647-702 |
| ADH4 | 703-769 |
| AGER | 8031-8095 |
| AKR1C3 | 770-824 |
| ALDH1A2 | 825-882 |
| ALG8 | 883-933 |
| AMPD2 | 934-1043 |
| AMT | 1044-1113 |
| ANKRD11 | 1114-1181 |
| ANKS3 | 1182-1244 |
| APOL4 | 1245-1302 |
| APPL1 | 1303-1352 |
| ARCN1 | 1353-1424 |
| ARMC8 | 1425-1478 |
| ARNTL | 1479-1531 |
| BOC | 1532-1606 |
| BRCA1 | 1607-1661 |
| C8B | 1662-1726 |
| CALM1 | 1727-1784 |
| CALM3 | 1785-1850 |
| CASP5 | 1851-1938 |
| CAST | 1939-1998 |
| CEP19 | 1999-2077 |
| CEP57 | 2078-2125 |
| CHEK1 | 2126-2209 |
| CIB2 | 2210-2270 |
| COX4I1 | 2271-2342 |
| DCLRE1C | 2343-2393 |
| DECR1 | 2394-2457 |
| DHFR | 2458-2517 |
| DKK2 | 2518-2586 |
| DLG2 | 2587-2643 |
| DOK2 | 2644-2719 |
| DPF3 | 2720-2787 |
| DTNA | 2788-2852 |
| DYRK1A | 2853-2927 |
| FOLH1 | 2928-2985 |
| FUZ | 2986-3041 |
| GANAB | 3042-3106 |
| GEMIN4 | 3107-3164 |
| GGA3 | 3165-3228 |
| GOSR2 | 3229-3309 |
| GPM6A | 3310-3360 |
| GRB10 | 3361-3422 |
| GSN | 3423-3475 |
| HDAC8 | 3476-3525 |
| HIVEP1 | 3526-3598 |
| HK1 | 3599-3656 |
| IFNGR1 | 3657-3717 |
| IKBKB | 3718-3781 |
| ISCU | 3782-3839 |
| KARS | 3840-3914 |
| KIAA0319 | 3915-3971 |
| KIAA0586 | 3972-4024 |
| KIZ | 4025-4076 |
| KLK6 | 4077-4165 |
| LMNA | 4166-4240 |
| LMNTD1 | 4241-4303 |
| LRRC7 | 4304-4357 |
| LRTOMT | 4358-4423 |
| LZTFL1 | 4424-4487 |
| MAGI2 | 4488-4547 |
| MECP2 | 35-129, 4548-4611 |
| MERTK | 4612-4670 |
| MFSD2A | 4671-4734 |
| MLF1 | 4735-4803 |
| MOB1B | 4804-4864 |
| MSRB3 | 4865-4928 |
| NR1I3 | 4929-4995 |
| NT5C3A | 4996-5045 |
| NUTM1 | 5046-5120 |
| OTOF | 5121-5179 |
| PDCD4 | 5180-5235 |
| PDPK1 | 5236-5294 |
| PHKB | 5295-5352 |
| PIGA | 5353-5546 |
| PLOD2 | 5547-5603 |
| POLR2F | 5604-5662 |
| PPP6C | 5663-5720 |
| PRKN | 5721-5807 |
| PRUNE1 | 5808-5865 |
| PSMA6 | 5866-5920 |
| PSMC3IP | 5921-5988 |
| PTPN1 | 5989-6047 |
| RAB11B | 6048-6125 |
| RBPJ | 6126-6187 |
| RNPS1 | 6188-6264 |
| RPL5 | 6265-6326 |
| RPS20 | 6327-6380 |
| SECISBP2 | 6381-6469 |
| SELENBP1 | 6470-6561 |
| SEPT11 | 6562-6632 |
| SIRT2 | 6633-6681 |
| SLC25A26 | 6682-6742 |
| SMARCE1 | 6743-6796 |
| SMC1A | 6797-6864 |
| SMG1 | 6865-6915 |
| SPACA7 | 6916-7000 |
| SPRED1 | 7001-7074 |
| SRP54 | 7075-7130 |
| SRPK2 | 7131-7182 |
| STK36 | 7183-7241 |
| STRADA | 7242-7301 |
| SUMO1 | 7302-7355 |
| TBL1XR1 | 7356-7415 |
| TLR8 | 7416-7481 |
| TXNRD2 | 7482-7538 |
| UBE3A | 7539-7590 |
| UFM1 | 7591-7675 |
| WAC | 7676-7753 |
| WDR81 | 7754-7919 |
| YME1L1 | 7920-7969 |
| ZMYND10 | 7970-8030 |

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a pre-mRNA that comprises an exon containing an inefficient translation region or a PTC followed by an alternative start codon. As used herein, the terms “ASO” and “antisense oligomer” are used interchangeably and refer to an oligomer such as a polynucleotide, comprising nucleobases that hybridizes to a target nucleic acid (e.g., MECP2 pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a pre-mRNA. Typically such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of a pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the disclosure, including, but not limited to, any of the ASOs set forth herein in Tables 5 and 6, comprises an ASO having phosphorus internucleotide linkages that are not random. In some embodiments, a composition used in the methods of the disclosure comprises a pure diastereomeric ASO. In some embodiments, a composition used in the methods of the disclosure comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second generation antisense oligonucleotides containing chiral phosphorothioate linkages,” Nucleic Acids Research 42(22): 13456-13468, incorporated herein by reference). In some embodiments, an ASO used in the methods of the disclosure, including, but not limited to, any of the ASOs set forth herein in SEQ ID NOs: 35-129, or 534-8095, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 35-129, or 534-8095, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 35-129, or 534-8095, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 35-129, or 534-8095, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a pre-mRNA that that comprises an exon containing an inefficient translation region or a PTC followed by an alternative start codon, and the targeted portion is downstream (in the 3′ direction) of the 5′ splice site of the exon. In some embodiments, the target portion is within the region about +1 to about +500 relative to the 5′ splice site (or 3′ end) of the exon. In some embodiments, the targeted portion is within the region between nucleotides +6 and +40,000 relative to the 5′ splice site (or 3′ end) of the exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the exon.

In some embodiments, the ASOs are complementary to (and bind to) a targeted portion of a pre-mRNA that that comprises an exon containing an inefficient translation region or a PTC followed by an alternative start codon, and the targeted portion is upstream (in the 5′ direction) of the 5′ splice site (or 3′ end) of the exon. In some embodiments, the targeted portion is within the region about −4 to about −270 relative to the 5′ splice site (or 3′end) of the exon. In some embodiments, the targeted portion is within the region between nucleotides −1 and −40,000 relative to the 5′ splice site (or 3′ end) of the exon. In some aspects, the ASOs are complementary to a targeted portion that is within the region about −1 to about −40,000, about −1 to about −30,000, about −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the exon.

In some embodiments, the ASOs are complementary to (and bind to) a targeted portion of a pre-mRNA that that comprises an exon containing an inefficient translation region or a PTC followed by an alternative start codon, and the targeted portion is that is upstream (in the 5′ direction) of the 3′ splice site (or 5′ end) of the exon. In some embodiments, the targeted portion is within the region about −1 to about −500 relative to the 3′ splice site (or 5′ end) of the exon. In some embodiments, the ASOs are complementary to a targeted portion that is within the region −1 to −40,000 relative to the 3′ splice site of the included exon. In some aspects, the ASOs are complementary to a targeted portion that is within the region about −1 to about −40,000, about −1 to about −30,000, −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 3′ splice site of the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to (and bind to) a targeted portion of a pre-mRNA that that comprises an exon containing an inefficient translation region or a PTC followed by an alternative start codon, and the targeted portion is downstream (in the 3′ direction) of the 3′ splice site (5′ end) of the exon. In some embodiments, the ASOs are complementary to a targeted portion that is within the region of about +1 to about +40,000 relative to the 3′ splice site of the exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the exon.

In some embodiments, the targeted portion is within the region +100 relative to the 5′ splice site (3′ end) of the exon to −100 relative to the 3′ splice site (5′ end) of the exon. In some embodiments, the targeted portion is within the exon that contains an inefficient translation region or a PTC followed by an alternative start codon. In some embodiments, the targeted portion comprises an exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the pre-mRNA are used. In some embodiments, two or more ASOs that are complementary to different targeted portions of the pre-mRNA are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In embodiments, the antisense oligonucleotide is administered in combination with another drug or therapeutic agent.

### Combination Therapies

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

Suitable routes for administration of ASOs of the present disclosure may vary depending on cell type to which delivery of the ASOs is desired. The ASOs of the present disclosure may be administered to patients parenterally, for example, by intrathecal injection, intracerebroventricular injection, intraperitoneal injection, intramuscular injection, subcutaneous injection, or intravenous injection.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, an ASO of the disclosure is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In some embodiments, subjects treated using the methods and compositions are evaluated for improvement in condition using any methods known and described in the art.

### EXAMPLES

The present disclosure will be more specifically illustrated by the following Examples. However, it should be understood that the present disclosure is not limited by these examples in any manner.

### Example 1: Confirmation of Exon 2 Inclusion Event in MECP2 mRNA Processing

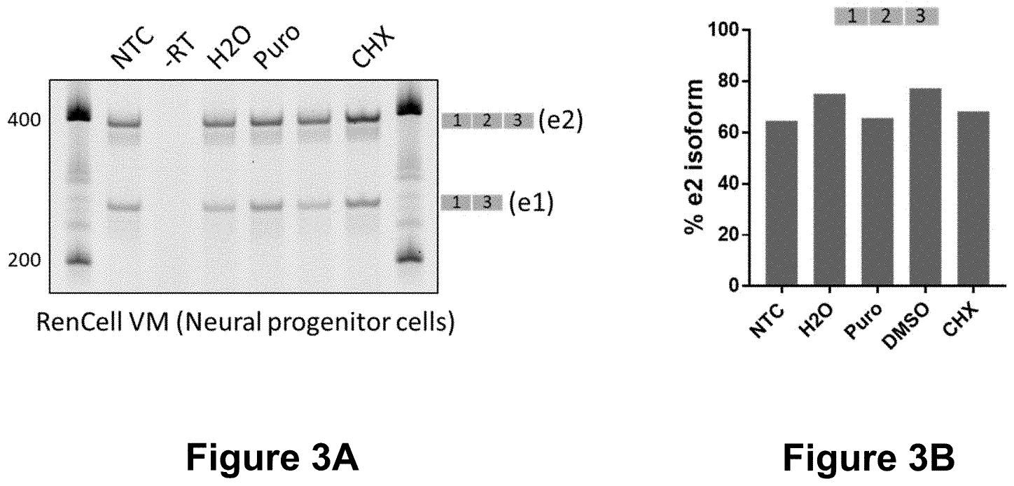

RT-PCR analysis using cytoplasmic RNA from DMSO-treated or puromycin (Puro) or cycloheximide (CHX)-treated RenCellsVM (Neural progenitor cells) in exons can confirm the presence of a band corresponding to an exon that experiences alternative splicing during mRNA processing. Primers were designed to target exon 1 and exon 3 of MECP2 gene. FIG. 3A shows the bands corresponding to e2 mRNA isoform—having exon 1, 2, and 3, and e1 mRNA isoform—having exon 1 and 3, respectively. Densitometry analysis of the bands was performed to calculate percent exon inclusion of total transcript. Treatment of cells with cycloheximide or puromycin did not lead to a significant change in e2 mRNA isoform, as shown in FIG. 3B.

### Example 2: ASO Walk for Exon 2 Region of MECP2

An ASO walk was performed for exon 2 region of MCEP2 pre-mRNA with various ASOs targeting sequences immediately upstream of the 3′ splice site, across the 3′splice site, exon 2, across the 5′ splice site, and downstream of the 5′ splice site using 18mer 2′-MOE ASOs, PS backbone. ASOs were designed to cover these regions by shifting 5 nucleotides at a time.

### Example 3: ASO Walk Evaluated by RT-PCR

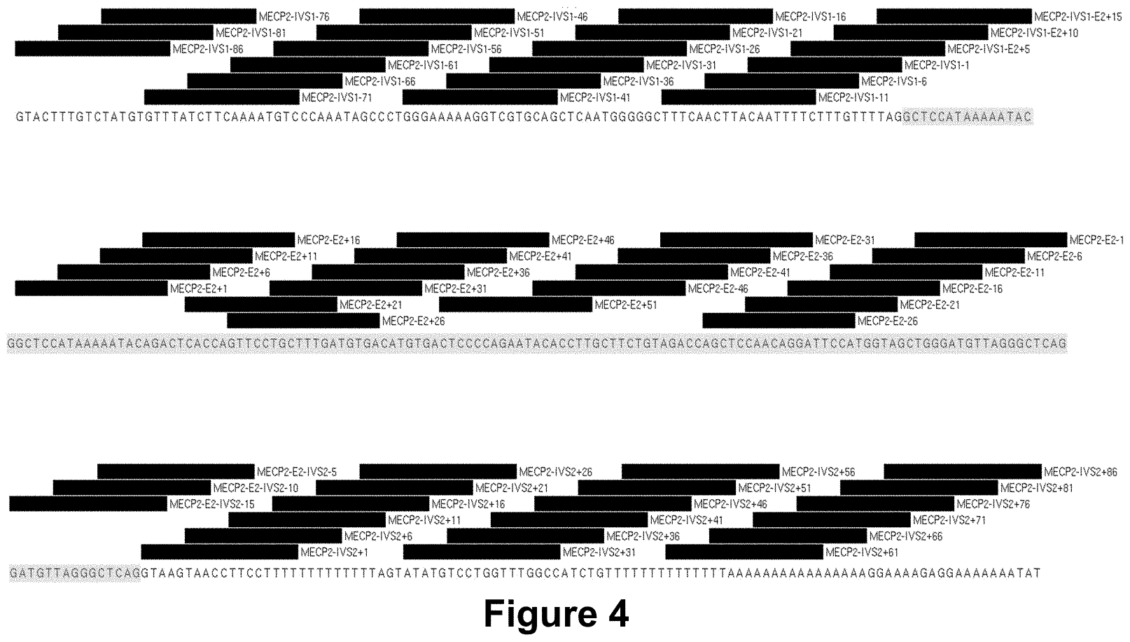

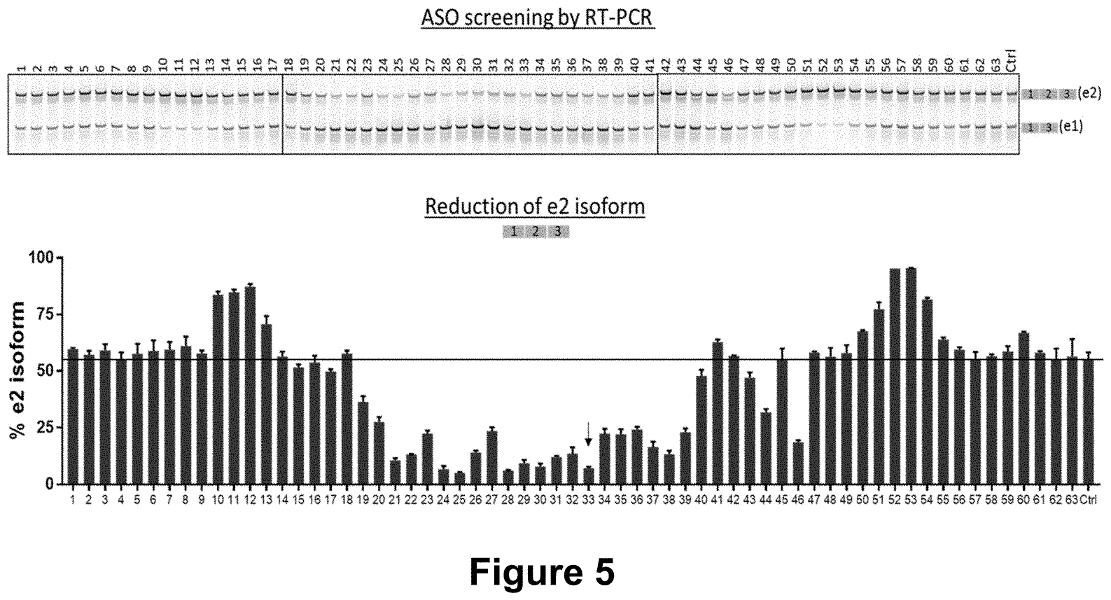

ASO walk sequences were evaluated by for example RT-PCR. RT-PCR using RNA from HEK293 cells transfected for 24 hrs with 80 nM ASOs that are depicted in FIG. 4. Primers for the RT-PCR analysis were positioned in exon 1 and 3. Quantification of the RT-PCR products was plotted as percentage of e2 isoform (e2/(e2+e1)*100), as shown in FIG. 5 (N=2).

### Example 4: Exemplary ASO Induces MECP2 Exon 2 Skipping and Increases MeCP2 Protein Expression

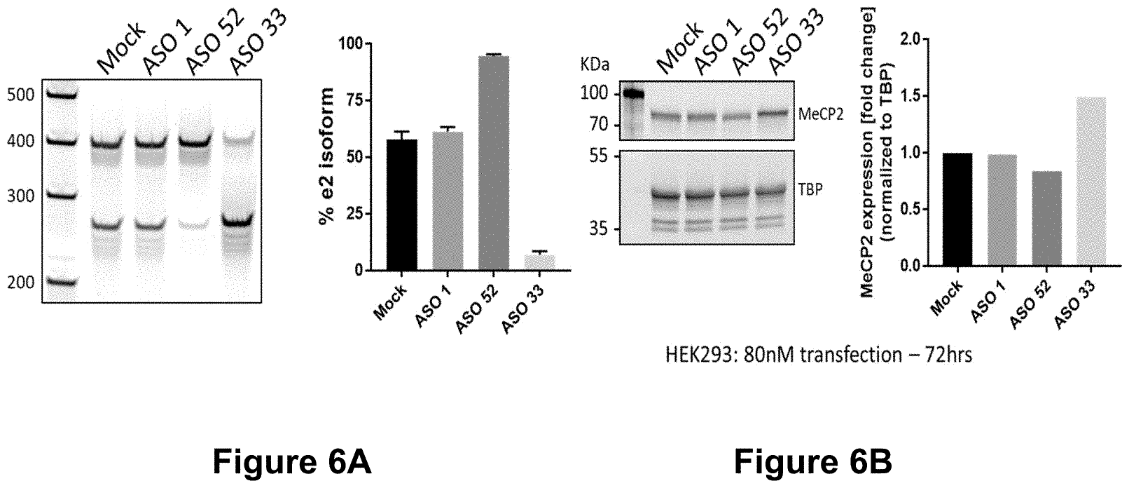

RT-PCR was performed using RNA from HEK293 cells transfected for 24 hrs with 80 nM ASOs (ASO1, 52, or 33) or not transfected (mock). FIG. 6A shows that HEK293 cells treated with ASOs against MECP2 induced either exon 2 inclusion (ASO52) or exon 2 skipping (ASO33). ASO1 targets MECP2 but had no effect on exon 2 splicing. Bar chart represents mean±SE. n=3. FIG. 6B shows that cells treated with exon skipping ASO33 exhibited an isoform switch and increased total MeCP2 protein by western blot, which examined whole cell lysates of cells treated with ASOs 1, 52 and 33, as compared to mock at 72 hr post-transfection. Anti-MeCP2 antibody detects isoforms e1 (top band) & e2 (bottom band). Quantification of bands corresponding to MeCP2 were normalized to TBP (TATA-binding protein) loading control and plotted as fold change over control (Mock).

### Example 5: Quantification of Total MECP2 mRNA (e1+e2)

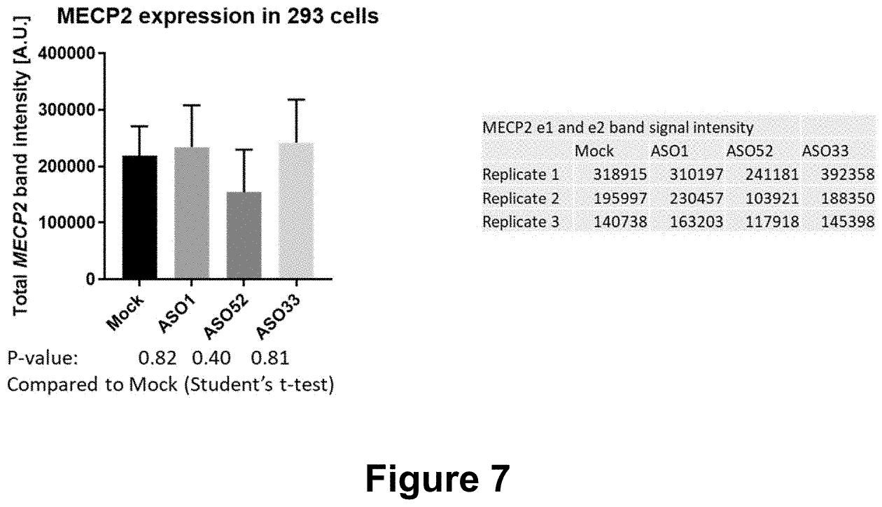

RT-PCR products from FIG. 6A were quantified and plotted as the sum of e1+e2 (FIG. 7). ASO treatment does not increase total mRNA levels.

### Example 6. Test of Exemplary ASOs in Murine Cells

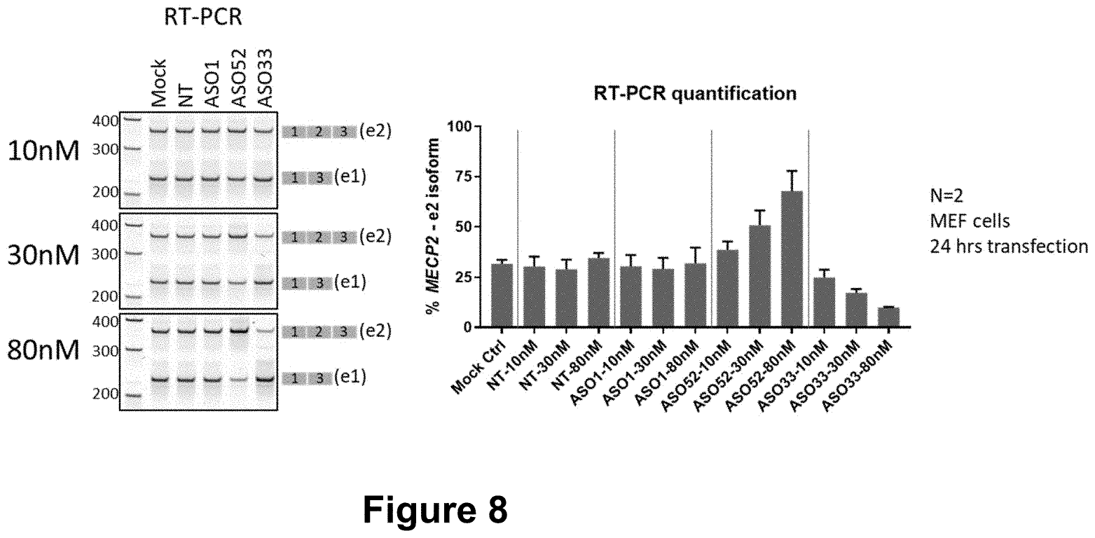

In parallel to screening ASO in human cell lines, ASOs were screened in murine cells. The exonic as well as flanking intronic sequence of MECP2 exon 2 is almost fully conserved between human and mouse. Lead candidates targeting a conserved region of the human transcript can retain their potency in murine cells (mouse embryonic fibroblasts, MEFs). RT-PCR was performed using RNA from MEF cells transfected for 24 hrs with 10, 30, or 80 nM ASOs (NT, ASO1, 52, or 33) or not transfected (mock). FIG. 8 shows that MEF cells treated with ASOs against MECP2 induced either exon 2 inclusion (ASO52) or exon 2 skipping (ASO33) (left panel). ASO1 targets MECP2 but had no effect on exon 2 splicing. FIG. 8 also shows a bar chart that plots the quantification of the RT-PCR assay and represents mean±SD, n=2 (right panel). The effect is dose-responsive.

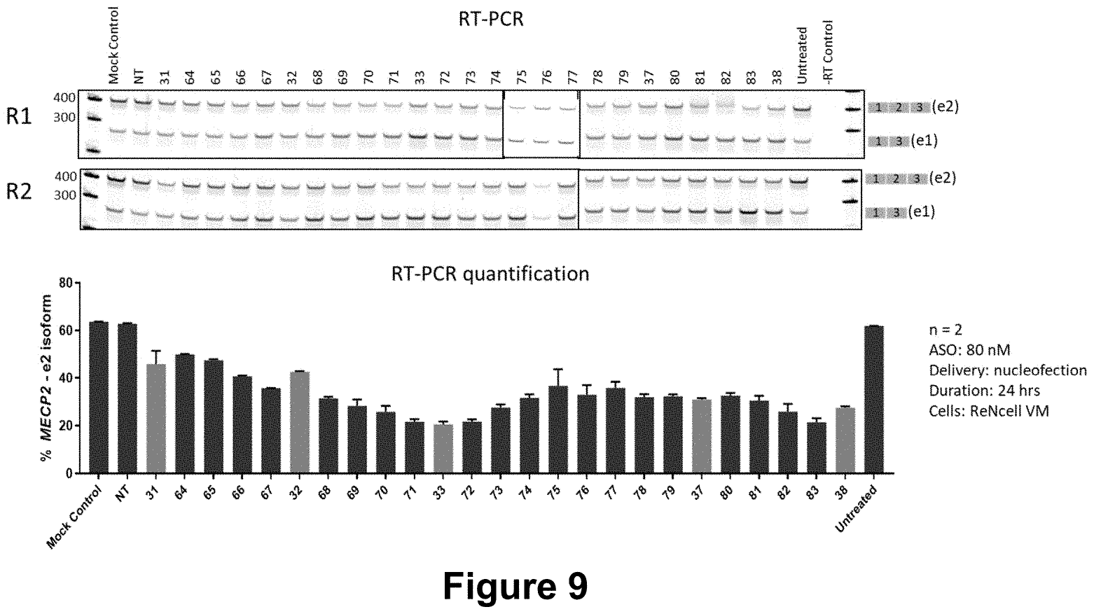

ASO microwalk sequences were evaluated by for example RT-PCR. RT-PCR using RNA from ReNcell VM nucleofected for 24 hrs with 80 nM ASOs that span a region around ASOs 31 and 33, and 37 and 38 (FIG. 9), in 1-nt step. Primers for the RT-PCR analysis were positioned in exon 1 and 3. Quantification of the RT-PCR products was plotted as percentage of e2 isoform (e2/(e2+e1)*100), as shown in FIG. 9A (N=2).

### Example 7. Test of Exemplary ASOs in Animal Model of Rett Syndrome

Select MECP2-targeting ASOs are tested in several mouse models to test the effect of (a) increased MeCP2 expression in wild-type mice; (b) increased MeCP2 expression in heterozygous null mice; and (c) increase of MeCP2 expression in mice carrying the partially functional MECP2 p.A140V variant.

ASOs are delivered to neonate wild-type mice by bolus intracerebroventricular (icv)-injection. The effect of ASOs on Mecp2 exon 2 skipping and protein expression is assessed 2-14 days later. Once the duration and magnitude of the effect is established, the lead ASOs are tested in functional studies using mouse models of Rett syndrome. There can be concern of inducing MECP2 duplication syndrome using TANGO-ASOs, thus the effect of therapeutic ASOs is compared in wild type and heterozygous null animals (JAX B6.129P2(C)-Mecp2^{tm1.1Bird}/J) using a battery of neurological and behavioral assessments. Studies using heterozygous null animals can also establish whether increase of MeCP2 in a subset of neurons is beneficial and can alleviate the phenotype. Mice lacking Mecp2 expression can recapitulate several disease phenotypes of Rett patients including muscle weakness and neurological phenotypes (PsychoGenics Inc.). Grip strength of 8-12-week-old heterozygous Mecp2-null female mice as well as their latency to fall from a rotarod can be significantly lower compared to wild-type littermates. 16-week-old heterozygous female mice can have breathing abnormalities (breath variance and apnea duration) and show decreased expression of brain-derived neurotrophic factor (BNDF) compared to mice harboring two copies of Mecp2.

The effect of ASO-mediated increase of MeCP2 expression is also tested in a mouse model expressing MECP2 p.A140V (JAX BT6N.129 Mecp2^{tm1.1Vnar}/J) Hippocampal neurons in female Mecp2^{A140/y} animals can be significantly smaller compared to wild-type control. Furthermore, mTOR signaling can be deregulated as shown by reduced expression of the mTORC2 subunit RICTOR, as well as reduced phosphorylation of mTOR and 4E-BP1. The effect of the ASOs that increase MeCP2 expression is tested on the morphology of hippocampal neurons as well as mTOR signaling in the brain of these mice.

TABLE 3
Sequences of exemplary ASOs.

| | SEQ | | | Genomic Coordinates | | |
|---|---|---|---|---|---|---|
| | ID | | | (GRCh38/hg38) | | |
| ASO# | NO. | ID | Sequence (5′-3′) | Chromosome | Start | End |
| 1 | 35 | MECP2-IVS1-86 | AACACATAGACAAAGTAC | chrX | 154092392 | 154092410 |
| 2 | 36 | MECP2-IVS1-81 | AGATAAACACATAGACAA | chrX | 154092387 | 154092405 |
| 3 | 37 | MECP2-IVS1-76 | TTTGAAGATAAACACATA | chrX | 154092382 | 154092400 |
| 4 | 38 | MECP2-IVS1-71 | GACATTTTGAAGATAAAC | chrX | 154092377 | 154092395 |
| 5 | 39 | MECP2-IVS1-66 | TTTGGGACATTTTGAAGA | chrX | 154092372 | 154092390 |
| 6 | 40 | MECP2-IVS1-61 | GGCTATTTGGGACATTTT | chrX | 154092367 | 154092385 |
| 7 | 41 | MECP2-IVS1-56 | CCCAGGGCTATTTGGGAC | chrX | 154092362 | 154092380 |
| 8 | 42 | MECP2-IVS1-51 | TTTTTCCCAGGGCTATTT | chrX | 154092357 | 154092375 |
| 9 | 43 | MECP2-IVS1-46 | CGACCTTTTTCCCAGGGC | chrX | 154092352 | 154092370 |
| 10 | 44 | MECP2-IVS1-41 | CTGCACGACCTTTTTCCC | chrX | 154092347 | 154092365 |
| 11 | 45 | MECP2-IVS1-36 | TTGAGCTGCACGACCTTT | chrX | 154092342 | 154092360 |
| 12 | 46 | MECP2-IVS1-31 | CCCCATTGAGCTGCACGA | chrX | 154092337 | 154092355 |
| 13 | 47 | MECP2-IVS1-26 | AAAGCCCCCATTGAGCTG | chrX | 154092332 | 154092350 |
| 14 | 48 | MECP2-IVS1-21 | AGTTGAAAGCCCCCATTG | chrX | 154092327 | 154092345 |
| 15 | 49 | MECP2-IVS1-16 | TTGTAAGTTGAAAGCCCC | chrX | 154092322 | 154092340 |
| 16 | 50 | MECP2-IVS1-11 | GAAAATTGTAAGTTGAAA | chrX | 154092317 | 154092335 |
| 17 | 51 | MECP2-IVS1-6 | ACAAAGAAAATTGTAAGT | chrX | 154092312 | 154092330 |
| 18 | 52 | MECP2-IVS1-1 | CTAAAACAAAGAAAATTG | chrX | 154092307 | 154092325 |
| 19 | 53 | MECP2-IVS1-E2+5 | GGAGCCTAAAACAAAGAA | chrX | 154092302 | 154092320 |
| 20 | 54 | MECP2-IVS1- | TTTATGGAGCCTAAAACA | chrX | 154092297 | 154092315 |
| | | E2+10 | | | | |
| 21 | 55 | MECP2-IVS1- | GTATTTTTATGGAGCCTA | chrX | 154092292 | 154092310 |
| | | E2+15 | | | | |
| 22 | 56 | MECP2-E2+1 | TCTGTATTTTTATGGAGC | chrX | 154092289 | 154092307 |
| 23 | 57 | MECP2-E2+6 | GTGAGTCTGTATTTTTAT | chrX | 154092284 | 154092302 |
| 24 | 58 | MECP2-E2+11 | AACTGGTGAGTCTGTATT | chrX | 154092279 | 154092297 |
| 25 | 59 | MECP2-E2+16 | GCAGGAACTGGTGAGTCT | chrX | 154092274 | 154092292 |
| 26 | 60 | MECP2-E2+21 | TCAAAGCAGGAACTGGTG | chrX | 154092269 | 154092287 |
| 27 | 61 | MECP2-E2+26 | TCACATCAAAGCAGGAAC | chrX | 154092264 | 154092282 |
| 28 | 62 | MECP2-E2+31 | ACATGTCACATCAAAGCA | chrX | 154092259 | 154092277 |
| 29 | 63 | MECP2-E2+36 | GAGTCACATGTCACATCA | chrX | 154092254 | 154092272 |
| 30 | 64 | MECP2-E2+41 | CTGGGGAGTCACATGTCA | chrX | 154092249 | 154092267 |
| 31 | 65 | MECP2-E2+46 | GTATTCTGGGGAGTCACA | chrX | 154092244 | 154092262 |
| 32 | 66 | MECP2-E2+51 | AAGGTGTATTCTGGGGAG | chrX | 154092239 | 154092257 |
| 33 | 67 | MECP2-E2-46 | TCTACAGAAGCAAGGTGT | chrX | 154092228 | 154092246 |
| 34 | 68 | MECP2-E2-41 | GCTGGTCTACAGAAGCAA | chrX | 154092223 | 154092241 |
| 35 | 69 | MECP2-E2-36 | TTGGAGCTGGTCTACAGA | chrX | 154092218 | 154092236 |
| 36 | 70 | MECP2-E2-31 | TCCTGTTGGAGCTGGTCT | chrX | 154092213 | 154092231 |
| 37 | 71 | MECP2-E2-26 | TGGAATCCTGTTGGAGCT | chrX | 154092208 | 154092226 |
| 38 | 72 | MECP2-E2-21 | TACCATGGAATCCTGTTG | chrX | 154092203 | 154092221 |
| 39 | 73 | MECP2-E2-16 | CCAGCTACCATGGAATCC | chrX | 154092198 | 154092216 |
| 40 | 74 | MECP2-E2-11 | ACATCCCAGCTACCATGG | chrX | 154092193 | 154092211 |
| 41 | 75 | MECP2-E2-6 | CCCTAACATCCCAGCTAC | chrX | 154092188 | 154092206 |
| 42 | 76 | MECP2-E2-1 | CTGAGCCCTAACATCCCA | chrX | 154092183 | 154092201 |
| 43 | 77 | MECP2-E2-IVS2-15 | TACCTGAGCCCTAACATC | chrX | 154092180 | 154092198 |
| 44 | 78 | MECP2-E2-IVS2-10 | TTACTTACCTGAGCCCTA | chrX | 154092175 | 154092193 |
| 45 | 79 | MECP2-E2-IVS2-5 | GAAGGTTACTTACCTGAG | chrX | 154092170 | 154092188 |
| 46 | 80 | MECP2-IVS2+1 | AAAAGGAAGGTTACTTAC | chrX | 154092165 | 154092183 |
| 47 | 81 | MECP2-IVS2+6 | AAAAAAAAAGGAAGGTTA | chrX | 154092160 | 154092178 |
| 48 | 82 | MECP2-IVS2+11 | TAAAAAAAAAAAAAGGAA | chrX | 154092155 | 154092173 |
| 49 | 83 | MECP2-IVS2+16 | TATACTAAAAAAAAAAAA | chrX | 154092150 | 154092168 |
| 50 | 84 | MECP2-IVS2+21 | GGACATATACTAAAAAAA | chrX | 154092145 | 154092163 |
| 51 | 85 | MECP2-IVS2+26 | AACCAGGACATATACTAA | chrX | 154092140 | 154092158 |
| 52 | 86 | MECP2-IVS2+31 | GGCCAAACCAGGACATAT | chrX | 154092135 | 154092153 |
| 53 | 87 | MECP2-IVS2+36 | CAGATGGCCAAACCAGGA | chrX | 154092130 | 154092148 |
| 54 | 88 | MECP2-IVS2+41 | AAAAACAGATGGCCAAAC | chrX | 154092125 | 154092143 |
| 55 | 89 | MECP2-IVS2+46 | AAAAAAAAAACAGATGGC | chrX | 154092120 | 154092138 |
| 56 | 90 | MECP2-IVS2+51 | TAAAAAAAAAAAAAACAG | chrX | 154092115 | 154092133 |
| 57 | 91 | MECP2-IVS2+56 | TTTTTTAAAAAAAAAAAA | chrX | 154092110 | 154092128 |
| 58 | 92 | MECP2-IVS2+61 | TTTTTTTTTTTAAAAAAA | chrX | 154092105 | 154092123 |
| 59 | 93 | MECP2-IVS2+66 | TTTTTTTTTTTTTTTTAA | chrX | 154092100 | 154092118 |
| 60 | 94 | MECP2-IVS2+71 | TTTCCTTTTTTTTTTTTT | chrX | 154092095 | 154092113 |
| 61 | 95 | MECP2-IVS2+76 | CCTCTTTTCCTTTTTTTT | chrX | 154092090 | 154092108 |
| 62 | 96 | MECP2-IVS2+81 | TTTTTCCTCTTTTCCTTT | chrX | 154092085 | 154092103 |
| 63 | 97 | MECP2-IVS2+86 | ATATTTTTTTCCTCTTTT | chrX | 154092080 | 154092098 |
| 64 | 98 | MECP2-E2+32 | CACATGTCACATCAAAGC | chrX | 154092259 | 154092276 |
| 65 | 99 | MECP2-E2+33 | TCACATGTCACATCAAAG | chrX | 154092258 | 154092275 |
| 66 | 100 | MECP2-E2+34 | GTCACATGTCACATCAAA | chrX | 154092257 | 154092274 |
| 67 | 101 | MECP2-E2+35 | AGTCACATGTCACATCAA | chrX | 154092256 | 154092273 |
| 68 | 102 | MECP2-E2+37 | GGAGTCACATGTCACATC | chrX | 154092254 | 154092271 |
| 69 | 103 | MECP2-E2+38 | GGGAGTCACATGTCACAT | chrX | 154092253 | 154092270 |
| 70 | 104 | MECP2-E2+39 | GGGGAGTCACATGTCACA | chrX | 154092252 | 154092269 |
| 71 | 105 | MECP2-E2+40 | TGGGGAGTCACATGTCAC | chrX | 154092251 | 154092268 |
| 72 | 106 | MECP2-E2+42 | TCTGGGGAGTCACATGTC | chrX | 154092249 | 154092266 |
| 73 | 107 | MECP2-E2+43 | TTCTGGGGAGTCACATGT | chrX | 154092248 | 154092265 |
| 74 | 108 | MECP2-E2+44 | ATTCTGGGGAGTCACATG | chrX | 154092247 | 154092264 |
| 75 | 109 | MECP2-E2+45 | TATTCTGGGGAGTCACAT | chrX | 154092246 | 154092263 |
| 76 | 110 | MECP2-E2+47 | TGTATTCTGGGGAGTCAC | chrX | 154092244 | 154092261 |
| 77 | 111 | MECP2-E2+48 | GTGTATTCTGGGGAGTCA | chrX | 154092243 | 154092260 |
| 78 | 112 | MECP2-E2+49 | GGTGTATTCTGGGGAGTC | chrX | 154092242 | 154092259 |
| 79 | 113 | MECP2-E2+50 | AGGTGTATTCTGGGGAGT | chrX | 154092241 | 154092258 |
| 80 | 114 | MECP2-E2+52 | CAAGGTGTATTCTGGGGA | chrX | 154092239 | 154092256 |
| 81 | 115 | MECP2-E2+53 | GCAAGGTGTATTCTGGGG | chrX | 154092238 | 154092255 |
| 82 | 116 | MECP2-E2+54 | AGCAAGGTGTATTCTGGG | chrX | 154092237 | 154092254 |
| 83 | 117 | MECP2-E2+55 | AAGCAAGGTGTATTCTGG | chrX | 154092236 | 154092253 |
| 84 | 118 | MECP2-E2-50 | CAGAAGCAAGGTGTATTC | chrX | 154092233 | 154092250 |
| 85 | 119 | MECP2-E2-49 | ACAGAAGCAAGGTGTATT | chrX | 154092232 | 154092249 |
| 86 | 120 | MECP2-E2-48 | TACAGAAGCAAGGTGTAT | chrX | 154092231 | 154092248 |
| 87 | 121 | MECP2-E2-47 | CTACAGAAGCAAGGTGTA | chrX | 154092230 | 154092247 |
| 88 | 122 | MECP2-E2-45 | GTCTACAGAAGCAAGGTG | chrX | 154092228 | 154092245 |
| 89 | 123 | MECP2-E2-44 | GGTCTACAGAAGCAAGGT | chrX | 154092227 | 154092244 |
| 90 | 124 | MECP2-E2-43 | TGGTCTACAGAAGCAAGG | chrX | 154092226 | 154092243 |
| 91 | 125 | MECP2-E2-42 | CTGGTCTACAGAAGCAAG | chrX | 154092225 | 154092242 |
| 92 | 126 | MECP2-E2-40 | AGCTGGTCTACAGAAGCA | chrX | 154092223 | 154092240 |
| 93 | 127 | MECP2-E2-39 | GAGCTGGTCTACAGAAGC | chrX | 154092222 | 154092239 |
| 94 | 128 | MECP2-E2-38 | GGAGCTGGTCTACAGAAG | chrX | 154092221 | 154092238 |
| 95 | 129 | MECP2-E2-37 | TGGAGCTGGTCTACAGAA | chrX | 154092220 | 154092237 |

TABLE 4
Sequences of exemplary ASOs in microwalk

| | ASO | | |
|---|---|---|---|
| | # | | |
| SEQ | in | | |
| ID | FIG. | | |
| NO. | 9 | Sequence (5′-3′) | Chemistry |
| 129 | 64 | TGGAGCTGGTCTACAGAA | MOE (Phosphorothioate) |
| 128 | 65 | GGAGCTGGTCTACAGAAG | MOE (Phosphorothioate) |
| 127 | 66 | GAGCTGGTCTACAGAAGC | MOE (Phosphorothioate) |
| 126 | 67 | AGCTGGTCTACAGAAGCA | MOE (Phosphorothioate) |
| 125 | 68 | CTGGTCTACAGAAGCAAG | MOE (Phosphorothioate) |
| 124 | 69 | TGGTCTACAGAAGCAAGG | MOE (Phosphorothioate) |
| 123 | 70 | GGTCTACAGAAGCAAGGT | MOE (Phosphorothioate) |
| 122 | 71 | GTCTACAGAAGCAAGGTG | MOE (Phosphorothioate) |
| 121 | 72 | CTACAGAAGCAAGGTGTA | MOE (Phosphorothioate) |
| 120 | 73 | TACAGAAGCAAGGTGTAT | MOE (Phosphorothioate) |
| 119 | 74 | ACAGAAGCAAGGTGTATT | MOE (Phosphorothioate) |
| 118 | 75 | CAGAAGCAAGGTGTATTC | MOE (Phosphorothioate) |
| 117 | 76 | AAGCAAGGTGTATTCTGG | MOE (Phosphorothioate) |
| 116 | 77 | AGCAAGGTGTATTCTGGG | MOE (Phosphorothioate) |
| 115 | | GCAAGGTGTATTCTGGGG | MOE (Phosphorothioate) |
| 114 | | CAAGGTGTATTCTGGGGA | MOE (Phosphorothioate) |
| 113 | | AGGTGTATTCTGGGGAGT | MOE (Phosphorothioate) |
| 112 | | GGTGTATTCTGGGGAGTC | MOE (Phosphorothioate) |
| 111 | | GTGTATTCTGGGGAGTCA | MOE (Phosphorothioate) |
| 110 | | TGTATTCTGGGGAGTCAC | MOE (Phosphorothioate) |
| 109 | | TATTCTGGGGAGTCACAT | MOE (Phosphorothioate) |
| 108 | | ATTCTGGGGAGTCACATG | MOE (Phosphorothioate) |
| 107 | | TTCTGGGGAGTCACATGT | MOE (Phosphorothioate) |
| 106 | | TCTGGGGAGTCACATGTC | MOE (Phosphorothioate) |
| 105 | | TGGGGAGTCACATGTCAC | MOE (Phosphorothioate) |
| 104 | | GGGGAGTCACATGTCACA | MOE (Phosphorothioate) |
| 103 | 78 | GGGAGTCACATGTCACAT | MOE (Phosphorothioate) |
| 102 | 79 | GGAGTCACATGTCACATC | MOE (Phosphorothioate) |
| 101 | 80 | AGTCACATGTCACATCAA | MOE (Phosphorothioate) |
| 100 | 81 | GTCACATGTCACATCAAA | MOE (Phosphorothioate) |
| 99 | 82 | TCACATGTCACATCAAAG | MOE (Phosphorothioate) |
| 98 | 83 | CACATGTCACATCAAAGC | MOE (Phosphorothioate) |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-83. (canceled)

84. A method of modulating expression of a target peptide sequence by a cell having a pre-mRNA that encodes the target peptide sequence and that comprises an inefficient translation region, the method comprising contacting the cell with a therapeutic agent or a vector encoding the therapeutic agent,
wherein the therapeutic agent binds to a targeted region of the pre-mRNA encoding the target peptide sequence, whereby splicing of the inefficient translation region from the pre-mRNA is modulated, thereby modulating a level of a first processed mRNA that is devoid of the inefficient translation region and that encodes a first protein comprising the target peptide sequence, and thereby modulating the expression of the target peptide sequence in the cell,
wherein the first processed mRNA has a higher translation efficiency for producing the first protein in the cell as compared to a translation efficiency of a second processed mRNA for producing a second protein in the cell, and wherein the second protein comprises the target peptide sequence and the second processed mRNA comprises the inefficient translation region.

85. The method of claim 84, wherein the pre-mRNA encoding the target peptide sequence comprises a first start codon, a second start codon, and a premature termination codon (PTC) located downstream of the first start codon and upstream of the second start codon, and wherein the inefficient translation region comprises the premature termination codon (PTC) and the second start codon.

86. The method of claim 85, wherein the first protein comprising the target peptide sequence is translated starting from the first start codon on the first processed mRNA, and the second protein is translated starting from the second start codon on the second processed mRNA.

87. The method of claim 85, wherein at least a portion of the targeted region of the pre-mRNA is upstream or downstream of the inefficient translation region or an exon comprising the PTC and the second start codon.

88. The method of claim 85, wherein at least a portion of the targeted region of the pre-mRNA is within the inefficient translation region or an exon comprising the PTC and the second start codon.

89. The method of claim 84, wherein the inefficient translation region comprises a region that encodes a proline-rich peptide sequence.

90. The method of claim 84, wherein the therapeutic agent increases splicing of the inefficient translation region from the pre-mRNA, thereby increasing a level of the first processed mRNA and increasing expression of the target peptide sequence in the cell.

91. The method of claim 84, wherein the target peptide sequence has at least 100 amino acids.

92. The method of claim 84, wherein the method comprises contacting the cell with the vector encoding the therapeutic agent, and wherein the vector is a viral vector.

93. The method of claim 84, wherein the method comprises contacting the cell with the therapeutic agent, and wherein the therapeutic agent is an antisense oligomer (ASO).

94. The method of claim 93, wherein the antisense oligomer comprises a backbone modification, a modified sugar moiety, or both.

95. The method of claim 93, wherein the antisense oligomer has a sequence that is complementary to a sequence with at least 80% sequence identity to a sequence selected from the group consisting of SEQ ID NOs: 28-31.

96. The method of claim 93, wherein the antisense oligomer has a sequence with at least about 80% sequence identity to a sequence selected from the group consisting of SEQ ID NOs: 35-129 or 4548-4611.

97. The method of claim 84, wherein the pre-mRNA is an MeCP2 pre-mRNA.

98. The method of claim 97, wherein the MeCP2 pre-mRNA comprises a sequence with at least 80% sequence identity to a sequence selected from the group consisting of SEQ ID NOs: 2-27.

99. The method of claim 84, wherein the target peptide sequence is a portion of an MeCP2 protein isoform.

100. The method of claim 84, wherein the targeted region is within a sequence with at least 80% sequence identity to a sequence selected from the group consisting of SEQ ID NOs: 28-31.

101. The method of claim 84, wherein the target peptide sequence is translated from a sequence selected from the group consisting of SEQ ID NOs: 32-34.

102. The method of claim 84, wherein the therapeutic agent decreases a level of an e2 isoform of MeCP2 mRNA and increases a level of an e1 isoform of MeCP2 mRNA in the cell.

103. The method of claim 90, wherein the first processed mRNA encodes a protein having at least 80% sequence identity to SEQ ID NO: 8204 or 8205.

104. The method of claim 84, wherein the therapeutic agent decreases a level of a second processed mRNA encoding a protein having at least 80% sequence identity to SEQ ID NO: 440 or 441.

105. The method of claim 97, wherein the targeted region of the pre-mRNA
(a) comprises at least 10 contiguous nucleobases of SEQ ID NO: 238;
(b) is within a sequence between a pair of genomic sites selected from the group consisting of GRCh38/hg38: chrX 154092307 and GRCh38/hg38: chrX 154092184;
(c) is at least 50 nucleotides downstream of genomic site GRCh38/hg38: chrX 154092184;
(d) is at most 1500 nucleotides downstream of genomic site GRCh38/hg38: chrX 154092184;
(e) is at least 50 nucleotides upstream of genomic site GRCh38/hg38: chrX 154092307; or
(f) is at most 1500 nucleotides upstream of genomic site GRCh38/hg38: chrX 154092307.

106. The method of claim 84, wherein the target peptide sequence is a portion of a full-length protein.

107. The method of claim 84, wherein the target peptide sequence produced is a fully functional protein.

108. The method of claim 84, wherein the target peptide sequence is an N-terminal portion of a full-length protein.

109. The method of claim 84, wherein the target peptide sequence is a C-terminal portion of a full-length protein.

110. The method of claim 84, wherein the target peptide sequence is coded by a portion of the pre-mRNA upstream of the inefficient translation region.

111. The method of claim 84, wherein the target peptide sequence is coded by a portion of the pre-mRNA downstream of the inefficient translation region or the PTC.

112. The method of claim 84, wherein the inefficient translation region is in a 3′ untranslated region (3′ UTR) of the second processed mRNA.

113. The method of claim 84, wherein the method treats a subject suffering from a disease or condition that comprises Rett Syndrome; Rett Syndrome, Preserved Speech Variant; Rett Syndrome, Atypical; or Rett Syndrome, Zappella Variant.

114. A method of treating a disease or a condition in a subject in need thereof by modulating expression of a target peptide sequence in a cell of the subject, the cell having a pre-mRNA that encodes the target peptide sequence and that comprises an inefficient translation region, the method comprising administering to the subject a therapeutically effective amount of the therapeutic agent or a vector encoding the therapeutic agent,
wherein the therapeutic agent binds to a targeted region of the pre-mRNA and modulates splicing of the inefficient translation region from the pre-mRNA, thereby modulating a level of a first processed mRNA that (i) is a splicing product of the pre-mRNA, (ii) is devoid of the inefficient translation region and (iii) encodes a first protein comprising the target peptide sequence, and thereby modulating expression of the target peptide sequence in the cell of the subject,
wherein the first processed mRNA has a higher translation efficiency for producing the first protein in the cell as compared to a translation efficiency of a second processed mRNA for producing a second protein in the cell, and wherein the second protein comprises the target peptide sequence and the second processed mRNA is a splicing product of the pre-mRNA and is otherwise identical to the first processed mRNA but comprises the inefficient translation region.

115. A composition comprising a modified antisense oligonucleotide sequence or a vector encoding a polynucleotide comprising an antisense oligonucleotide sequence, wherein the antisense oligomer sequence has at least about 80% sequence identity to a sequence selected from the group consisting of SEQ ID NOs: 35-129 and 534-8095.
